SEQUENCE LISTING (77 sequences)

SEQ ID NO: 1 - DNA, 1830 bp, Homo sapiens
atgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaa

SEQ ID NO: 2 - protein, 590 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 3 - DNA, 1946 bp, Artificial Sequence
  Description of Artificial Sequence Synthetic construct
  CDS (91)..(1929)
cagagctctctggctaactagagaacccactgcttactggcttatcgaaattaatacgactcactatagggagacccaagatccgccaccatgcgccctacctgggcctggtggctgttcctggtgctgctgctggcactgtgggctccagcccgcggcaccttgcataggaatgaatatggaatagctagcatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgccttaggagtttaaggcgcgccggtacc

SEQ ID NO: 4 - protein, 613 aa, Artificial Sequence
  Description of Artificial Sequence Synthetic construct
MRPTWAWWLFLVLLLALWAPARGTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 5 - DNA, 1842 bp, Artificial Sequence
  Description of Artificial Sequence Synthetic construct
atgcgccctacctgggcctggtggctgttcctggtgctgctgctggcactgtgggctccagcccgcggcaccttgcataggaatgaatatggaatagctagcatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgccttaggagtttaa

SEQ ID NO: 6 - protein, 613 aa, Artificial Sequence; identical to SEQ ID NO: 4
  Description of Artificial Sequence Synthetic construct

SEQ ID NO: 7 - protein, 787 aa, Artificial Sequence [sequence omitted]

SEQ ID NO: 8 - protein, 746 aa, Artificial Sequence [sequence omitted]

SEQ ID NO: 9 - protein, 1110 aa, Artificial Sequence [sequence omitted]

SEQ ID NO: 10 - protein, 1142 aa, Artificial Sequence [sequence omitted]

SEQ ID NO: 11 - DNA, 455 bp, Artificial Sequence
  Description of Artificial Sequence Synthetic construct
gggctccagcccgcggcgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactaccttgcataggaatgaatatggaatagctagcatattg

SEQ ID NO: 12 - DNA, 2261 bp, Artificial Sequence
  Description of Artificial Sequence Synthetic construct
gggctccagcccgcggcgaagttaaacaggaaaaccgtctgctcaacgaatctgagtcttcctctcagggcctgctgggttactatttctctgacctgaacttccaggcaccgatggttgtaacttcttccaccaccggcgacctgtctattccgtcttctgaactggagaacatcccgtctgaaaaccagtacttccagtctgctatctggtctggtttcattaaagttaagaaatctgacgaatacaccttcgctacttctgcagataaccacgttactatgtgggtagacgaccaggaagttatcaacaaagcttctaactctaacaaaatccgtctggaaaaaggccgtctgtaccagatcaagattcaataccaacgtgaaaacccgaccgagaaaggtctggacttcaaactgtactggaccgactctcagaacaagaaagaagttatctcttccgacaacctgcagctgccggaactgaaacagaaatcttccaactctcgtaaaaagcgttctacttctgctggtccgaccgttccggaccgtgataacgacggtattccggactctctggaagttgaaggctacaccgtagacgttaaaaacaaacgtaccttcctgtctccgtggatctctaacatccacgaaaagaaaggtctgaccaaatacaaatcttccccggagaaatggtctaccgcttctgatccgtactctgacttcgaaaaagttactggtcgtatcgacaaaaacgtttctccggaagctcgtcacccgctggtagcagcgtacccgatcgttcacgttgacatggaaaacattatcctgtctaaaaacgaagaccagtctacccagaacaccgactctcaaactcgtaccatctctaaaaacacctctacctctcgtactcacacctctgaagttcacggtaacgctgaggttcacgcttctttctttgacatcggtggctctgtatctgctggtttctctaactctaactcttctaccgttgcaatcgaccactctctgtctctggctggtgaacgtacctgggctgaaactatgggcctgaacaccgcagacaccgctcgtctgaacgctaacatccgttacgttaacaccggcaccgctccgatctacaacgttctgccgactacctctctggtactgggtaaaaaccagaccctggcaaccatcaaagctaaagaaaaccagctgtctcagatcctggctccgaacaactactatccgtctaaaaacctggctccgattgcactgaacgctcaggacgacttctcttccaccccgatcactatgaactacaaccagttcctggaactggagaaaaccaaacagctgcgtctggacaccgaccaggtttacggtaacatcgctacctacaacttcgaaaacggtcgtgttcgtgtagacaccggctctaactggtctgaagttctgccgcagatccaggaaaccactgctcgtattatcttcaacggtaaagacctgaacctggttgaacgtcgtatcgctgcagtaaacccgtctgacccgctggaaaccactaaaccggacatgaccctgaaagaagctctgaaaatcgctttcggtttcaacgaaccgaacggcaacctgcagtaccagggtaaagatatcaccgaattcgactttaacttcgaccagcaaacctctcagaacatcaaaaaccagctggctgaactgaacgctaccaacatctacaccgttctggacaaaatcaagctgaacgctaaaatgaacattctgatccgtgataaacgtttccactacgaccgtaacaacatcgctgttggtgctgacgaatctgtagttaaagaagctcaccgtgaggttatcaactcttccaccgaaggtctgctcctgaacatcgacaaagatattcgtaaaatcctgtctggttacatcgttgaaatcgaagacaccgagggcctgaaagaagttatcaacgaccgttacgatatgctgaacatctcttccctgcgtcaggacggtaaaaccttcatcgacttcaaaaagtacaacgataaactgccgctgtacatctctaacccgaactacaaagtaaacgtttacgctgttaccaaagaaaacaccattatcaacccgtctgaaaacggtgacacctctaccaacggtatcaaaaagatcctgatcttctctaagaaaggctacgaaatcggtaccttgcataggaatgaatatggaatagctagcatattg

SEQ ID NO: 13 - DNA, 1962 bp, Artificial Sequence
  Description of Artificial Sequence Synthetic construct
  CDS (1)..(1959)
atgaagtgggtaagctttatttcccttctttttctctttagctcggcttattccaggagcctcgacaaaagacacggtgaaggtactttcacttctgatttgtctaagcaaatggaagaagaagctgttagattgttcattgaatggttgaagaacggtggtccatcttctggtgctccaccaccatctaccctgcaccgtaacgaatacggtatcgccagcatcctggacagctaccagtgcaccgccgaaatcagcctggccgacctggccaccatcttcttcgcccagttcgttcaggaagccacctacaaagaagttagcaaaatggttaaagacgccctgaccgccatcgaaaaaccgaccggtgacgaacagagcagcggttgcctggaaaaccagctgccggccttcctggaagaactgtgccacgaaaaagaaatcctggaaaaatacggtcacagcgactgctgcagccagagcgaagaaggtcgtcacaactgcttcctggcccacaaaaaaccgaccccggccagcatcccgctgttccaggttccggaaccggttaccagctgcgaagcctacgaagaagaccgtgaaaccttcatgaacaaattcatctacgaaatcgcccgtcgtcacccgttcctgtacgccccgaccatcctgctgtgggccgcccgttacgacaaaatcatcccgagctgctgcaaagccgaaaacgccgttgaatgcttccagaccaaagccgccaccgttaccaaagaactgcgtgaaagcagcctgctgaaccagcacgcctgcgccgttatgaaaaacttcggcacccgtaccttccaggccatcaccgttaccaaactgagccagaaattcaccaaagttaacttcaccgaaatccagaaactggttctggacgttgcccacgttcacgaacactgctgccgtggtgacgttctggactgcctgcaggacggtgaaaaaatcatgagctacatctgcagccagcaggacaccctgagcaacaaaatcaccgaatgctgcaaactgaccaccctggaacgtggtcagtgcatcatccacgccgaaaacgacgaaaaaccggaaggtctgagcccgaacctgaaccgtttcctgggtgaccgtgacttcaaccagttcagcagcggtgaaaaaaacatcttcctggccagcttcgttcacgaatacagccgtcgtcacccgcagctggccgttagcgttatcctgcgtgttgccaaaggttaccaggaactgctggaaaaatgcttccagaccgaaaacccgctggaatgccaggacaaaggtgaagaagaactgcagaaatacatccaggaaagccaggccctggccaaacgtagctgcggtctgttccagaaactgggtgaatactacctgcagaacgccttcctggttgcctacaccaaaaaagccccgcagctgaccagcagcgaactgatggccatcacccgtaaaatggccgccaccgccgccacctgctgccagctgagcgaagacaaactgctggcctgcggtgaaggtgccgccgacatcatcatcggtcacctgtgcatccgtcacgaaatgaccccggttaacccgggtgttggtcagtgctgcaccagcagctacgccaaccgtcgtccgtgcttcagcagcctggttgttgacgaaacctacgttccgccggccttcagcgacgacaaattcatcttccacaaagacctgtgccaggcccagggtgttgccctgcagaccatgaaacaggaattcctgatcaacctggttaaacagaaaccgcagatcaccgaagaacagctggaagccgttatcgccgacttcagcggtctgctggaaaaatgctgccagggtcaggaacaggaagtttgcttcgccgaagaaggtcagaaactgatcagcaaaacccgtgccgccctgggtgtttaa

SEQ ID NO: 14 - protein, 653 aa, Artificial Sequence
  Description of Artificial Sequence Synthetic construct
MKWVSFISLLFLFSSAYSRSLDKRHGEGTFTSDLSKQMEEEAVRLFIEWLKNGGPSSGAPPPSTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 15 - DNA, 2079 bp, Artificial Sequence
  Description of Artificial Sequence Synthetic construct
  CDS (1)..(2076)
atgaagtgggtttcctttatttctttgttattcttgttttcctctgcttattccagatctttggataaaagacacggtgaaggtactttcacttctgatttgtctaagcaaatggaagaagaagctgttagattgttcattgaatggttgaagaacggtggtccatcttctggtgctccaccaccatctcatggagagggaacatttacatctgatttgtctaagcaaatggaagaagaagctgttagattgttcattgaatggttgaagaacggtggtccatcttcaggtgcacctccacctagtaccctgcaccgtaacgaatacggtatcgctagcatcctggacagctaccagtgcaccgccgaaatcagcctggccgacctggccaccatcttcttcgcccagttcgttcaggaagccacctacaaagaagttagcaaaatggttaaagacgccctgaccgccatcgaaaaaccgaccggtgacgaacagagcagcggttgcctggaaaaccagctgccggccttcctggaagaactgtgccacgaaaaagaaatcctggaaaaatacggtcacagcgactgctgcagccagagcgaagaaggtcgtcacaactgcttcctggcccacaaaaaaccgaccccggccagcatcccgctgttccaggttccggaaccggttaccagctgcgaagcctacgaagaagaccgtgaaaccttcatgaacaaattcatctacgaaatcgcccgtcgtcacccgttcctgtacgccccgaccatcctgctgtgggccgcccgttacgacaaaatcatcccgagctgctgcaaagccgaaaacgccgttgaatgcttccagaccaaagccgccaccgttaccaaagaactgcgtgaaagcagcctgctgaaccagcacgcctgcgccgttatgaaaaacttcggcacccgtaccttccaggccatcaccgttaccaaactgagccagaaattcaccaaagttaacttcaccgaaatccagaaactggttctggacgttgcccacgttcacgaacactgctgccgtggtgacgttctggactgcctgcaggacggtgaaaaaatcatgagctacatctgcagccagcaggacaccctgagcaacaaaatcaccgaatgctgcaaactgaccaccctggaacgtggtcagtgcatcatccacgccgaaaacgacgaaaaaccggaaggtctgagcccgaacctgaaccgtttcctgggtgaccgtgacttcaaccagttcagcagcggtgaaaaaaacatcttcctggccagcttcgttcacgaatacagccgtcgtcacccgcagctggccgttagcgttatcctgcgtgttgccaaaggttaccaggaactgctggaaaaatgcttccagaccgaaaacccgctggaatgccaggacaaaggtgaagaagaactgcagaaatacatccaggaaagccaggccctggccaaacgtagctgcggtctgttccagaaactgggtgaatactacctgcagaacgccttcctggttgcctacaccaaaaaagccccgcagctgaccagcagcgaactgatggccatcacccgtaaaatggccgccaccgccgccacctgctgccagctgagcgaagacaaactgctggcctgcggtgaaggtgccgccgacatcatcatcggtcacctgtgcatccgtcacgaaatgaccccggttaacccgggtgttggtcagtgctgcaccagcagctacgccaaccgtcgtccgtgcttcagcagcctggttgttgacgaaacctacgttccgccggccttcagcgacgacaaattcatcttccacaaagacctgtgccaggcccagggtgttgccctgcagaccatgaaacaggaattcctgatcaacctggttaaacagaaaccgcagatcaccgaagaacagctggaagccgttatcgccgacttcagcggtctgctggaaaaatgctgccagggtcaggaacaggaagtttgcttcgccgaagaaggtcagaaactgatcagcaaaacccgtgccgccttaggtgtttaa

SEQ ID NO: 16 - protein, 692 aa, Artificial Sequence
  Description of Artificial Sequence Synthetic construct
MKWVSFISLLFLFSSAYSRSLDKRHGEGTFTSDLSKQMEEEAVRLFIEWLKNGGPSSGAPPPSHGEGTFTSDLSKQMEEEAVRLFIEWLKNGGPSSGAPPPSTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 17 - DNA, 2274 bp, Artificial Sequence
  Description of Artificial Sequence Synthetic construct
  CDS (1)..(2271)
  modified_base (165): a, c, g, t, unknown or other
atgaagtgggtttcctttatttctttgttattcttgttttcctctgcttattccagatctttggataaaagacacggtgaaggtactttcacttctgatttgtctaagcaaatggaagaagaagctgttagattgttcattgaatggttgaagaacggtggtccntcttctggtgctccaccaccatctcatggagagggaacatttacatctgatttgtctaagcaaatggaagaagaagctgttagattgttcattgaatggttgaagaacggtggtccatcttctggtgctccaccaccatctcatggagagggaacatttacatctgatttgtctaagcaaatggaagaagaagctgttagattgttcattgaatggttgaagaacggtggtccatcttctggtgctccaccaccatctcatggagagggaacatttacatctgatttgtctaagcaaatggaagaagaagctgttagattgttcattgaatggttgaagaacggtggtccatcttcaggtgcacctccacctagtgacagctaccagtgcaccgccgaaatcagcctggccgacctggccaccatcttcttcgcccagttcgttcaggaagccacctacaaagaagttagcaaaatggttaaagacgccctgaccgccatcgaaaaaccgaccggtgacgaacagagcagcggttgcctggaaaaccagctgccggccttcctggaagaactgtgccacgaaaaagaaatcctggaaaaatacggtcacagcgactgctgcagccagagcgaagaaggtcgtcacaactgcttcctggcccacaaaaaaccgaccccggccagcatcccgctgttccaggttccggaaccggttaccagctgcgaagcctacgaagaagaccgtgaaaccttcatgaacaaattcatctacgaaatcgcccgtcgtcacccgttcctgtacgccccgaccatcctgctgtgggccgcccgttacgacaaaatcatcccgagctgctgcaaagccgaaaacgccgttgaatgcttccagaccaaagccgccaccgttaccaaagaactgcgtgaaagcagcctgctgaaccagcacgcctgcgccgttatgaaaaacttcggcacccgtaccttccaggccatcaccgttaccaaactgagccagaaattcaccaaagttaacttcaccgaaatccagaaactggttctggacgttgcccacgttcacgaacactgctgccgtggtgacgttctggactgcctgcaggacggtgaaaaaatcatgagctacatctgcagccagcaggacaccctgagcaacaaaatcaccgaatgctgcaaactgaccaccctggaacgtggtcagtgcatcatccacgccgaaaacgacgaaaaaccggaaggtctgagcccgaacctgaaccgtttcctgggtgaccgtgacttcaaccagttcagcagcggtgaaaaaaacatcttcctggccagcttcgttcacgaatacagccgtcgtcacccgcagctggccgttagcgttatcctgcgtgttgccaaaggttaccaggaactgctggaaaaatgcttccagaccgaaaacccgctggaatgccaggacaaaggtgaagaagaactgcagaaatacatccaggaaagccaggccctggccaaacgtagctgcggtctgttccagaaactgggtgaatactacctgcagaacgccttcctggttgcctacaccaaaaaagccccgcagctgaccagcagcgaactgatggccatcacccgtaaaatggccgccaccgccgccacctgctgccagctgagcgaagacaaactgctggcctgcggtgaaggtgccgccgacatcatcatcggtcacctgtgcatccgtcacgaaatgaccccggttaacccgggtgttggtcagtgctgcaccagcagctacgccaaccgtcgtccgtgcttcagcagcctggttgttgacgaaacctacgttccgccggccttcagcgacgacaaattcatcttccacaaagacctgtgccaggcccagggtgttgccctgcagaccatgaaacaggaattcctgatcaacctggttaaacagaaaccgcagatcaccgaagaacagctggaagccgttatcgccgacttcagcggtctgctggaaaaatgctgccagggtcaggaacaggaagtttgcttcgccgaagaaggtcagaaactgatcagcaaaacccgtgccgccttaggtgtttaa

SEQ ID NO: 18 - protein, 757 aa, Artificial Sequence
  Description of Artificial Sequence Synthetic construct
MKWVSFISLLFLFSSAYSRSLDKRHGEGTFTSDLSKQMEEEAVRLFIEWLKNGGPSSGAPPPSHGEGTFTSDLSKQMEEEAVRLFIEWLKNGGPSSGAPPPSHGEGTFTSDLSKQMEEEAVRLFIEWLKNGGPSSGAPPPSHGEGTFTSDLSKQMEEEAVRLFIEWLKNGGPSSGAPPPSDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 19 - DNA, 1962 bp, Artificial Sequence
  Description of Artificial Sequence Synthetic construct
  CDS (1)..(1959)
atgaagtgggtaagctttatttcccttctttttctctttagctcggcttattccaggagcctcgacaaaagacacggtgaaggtactttcacttctgatttgtctaagcaaatggaagaagaagctgttagattgttcattgaatggttgaagaacggtggtccatcttctggtgctccaccaccatctaccctgcaccgtaacgaatacggtatcgccagcatcctggacagctaccagtgcaccgccgaaatcagcctggccgacctggccaccatcttcttcgcccagttcgttcaggaagccacctacaaagaagttagcaaaatggttaaagacgccctgaccgccatcgaaaaaccgaccggtgacgaacagagcagcggttgcctggaaaaccagctgccggccttcctggaagaactgtgccacgaaaaagaaatcctggaaaaatacggtcacagcgactgctgcagccagagcgaagaaggtcgtcacaactgcttcctggcccacaaaaaaccgaccccggccagcatcccgctgttccaggttccggaaccggttaccagctgcgaagcctacgaagaagaccgtgaaaccttcatgaacaaattcatctacgaaatcgcccgtcgtcacccgttcctgtacgccccgaccatcctgctgtgggccgcccgttacgacaaaatcatcccgagctgctgcaaagccgaaaacgccgttgaatgcttccagaccaaagccgccaccgttaccaaagaactgcgtgaaagcagcctgctgaaccagcacgcctgcgccgttatgaaaaacttcggcacccgtaccttccaggccatcaccgttaccaaactgagccagaaattcaccaaagttcaattcaccgaaatccagaaactggttctggacgttgcccacgttcacgaacactgctgccgtggtgacgttctggactgcctgcaggacggtgaaaaaatcatgagctacatctgcagccagcaggacaccctgagcaacaaaatcaccgaatgctgcaaactgaccaccctggaacgtggtcagtgcatcatccacgccgaaaacgacgaaaaaccggaaggtctgagcccgaacctgaaccgtttcctgggtgaccgtgacttcaaccagttcagcagcggtgaaaaaaacatcttcctggccagcttcgttcacgaatacagccgtcgtcacccgcagctggccgttagcgttatcctgcgtgttgccaaaggttaccaggaactgctggaaaaatgcttccagaccgaaaacccgctggaatgccaggacaaaggtgaagaagaactgcagaaatacatccaggaaagccaggccctggccaaacgtagctgcggtctgttccagaaactgggtgaatactacctgcagaacgccttcctggttgcctacaccaaaaaagccccgcagctgaccagcagcgaactgatggccatcacccgtaaaatggccgccaccgccgccacctgctgccagctgagcgaagacaaactgctggcctgcggtgaaggtgccgccgacatcatcatcggtcacctgtgcatccgtcacgaaatgaccccggttaacccgggtgttggtcagtgctgcaccagcagctacgccaaccgtcgtccgtgcttcagcagcctggttgttgacgaaacctacgttccgccggccttcagcgacgacaaattcatcttccacaaagacctgtgccaggcccagggtgttgccctgcagaccatgaaacaggaattcctgatcaacctggttaaacagaaaccgcagatcaccgaagaacagctggaagccgttatcgccgacttcagcggtctgctggaaaaatgctgccagggtcaggaacaggaagtttgcttcgccgaagaaggtcagaaactgatcagcaaaacccgtgccgccctgggtgtttaa

SEQ ID NO: 20 - protein, 653 aa, Artificial Sequence
  Description of Artificial Sequence Synthetic construct
MKWVSFISLLFLFSSAYSRSLDKRHGEGTFTSDLSKQMEEEAVRLFIEWLKNGGPSSGAPPPSTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVQFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 21 - protein, 21 aa, Homo sapiens
MKVSVAALSCLMLVTALGSQA

SEQ ID NO: 22 - protein, 17 aa, Artificial Sequence
  Description of Artificial Sequence Synthetic construct
MLQNSAVLLLLVISASA

SEQ ID NO: 23 - protein, 19 aa, Artificial Sequence
  Description of Artificial Sequence Synthetic construct
MLLQAFLFLLAGFAAKISA

SEQ ID NO: 24 - protein, 86 aa, Saccharomyces cerevisiae
MRFPSIFTAVLAFAASSALAAPVNTTTEDETAQIPAEAVIGYSDLEGDFDVAVLPFSNSTNNGLLFINTTIASIAAKEEGVSLEKR

SEQ ID NO: 25 - protein, 85 aa, Saccharomyces cerevisiae
MRFPSHTAVLAFAASSALAAPVNTITEDETAQIPAEAVIGYSDLEGDFDVAVLPFSNSTNNGLLFINTTIASIAAKEEGVSLDKR

SEQ ID NO: 26 - protein, 24 aa, Artificial Sequence
  Description of Artificial Sequence Synthetic construct
MKWVSFISLLFLFSSAYSRSLEKR

SEQ ID NO: 27 - protein, 21 aa, Kluyveromyces lactis
MNIFYIFLFLLSFVQGSLDKR

SEQ ID NO: 28 - protein, 19 aa, Artificial Sequence
  Description of Artificial Sequence Synthetic construct
MGWSCIILFLVATATGVHS

SEQ ID NO: 29 - protein, 29 aa, Artificial Sequence
  Description of Artificial Sequence Synthetic construct
MERAAPSRRVPLPLLLLGGLALLAAGVDA

SEQ ID NO: 30 - protein, 22 aa, Artificial Sequence
  Description of Artificial Sequence Synthetic construct
MMKTLLLFVGLLLTWESGQVLG

SEQ ID NO: 31 - protein, 21 aa, Artificial Sequence
  Description of Artificial Sequence Synthetic construct
MLPLCLVAALLLAAGPGPSLG

SEQ ID NO: 32 - protein, 22 aa, Artificial Sequence
  Description of Artificial Sequence Synthetic construct
MPTWAWWLFLVLLLALWAPARG

SEQ ID NO: 33 - protein, 17 aa, Artificial Sequence
  Description of Artificial Sequence Synthetic construct
MFKSVVYSILAASLANA

SEQ ID NO: 34 - protein, 29 aa, Kluyveromyces lactis
MNIFYIFLFLLSFVQGLEHTHRRGSLDKR

SEQ ID NO: 35 - protein, 23 aa, Artificial Sequence
  Description of Artificial Sequence Synthetic construct
MKLAYSLLLPLAGVSASVINYKR

SEQ ID NO: 36 - protein, 65 aa, Artificial Sequence
  Description of Artificial Sequence Synthetic construct
MKLKTVRSAVLSSLFASQVLGQPIDDTESQTTSVNLMADDTESAFATQTNSGGLDVVGLISMAKR

SEQ ID NO: 37 - protein, 70 aa, Artificial Sequence
  Description of Artificial Sequence Synthetic construct
MKLKTVRSAVLSSLFASQVLGQPIDDTESQTTSVNLMADDTESAFATQTNSGGLDVVGLISMAEEGEPKR

SEQ ID NO: 38 - protein, 21 aa, Artificial Sequence
  Description of Artificial Sequence Synthetic construct
MWWRLWWLLLLLLLLWPMVWA

SEQ ID NO: 39 - protein, 24 aa, Artificial Sequence
  Description of Artificial Sequence Synthetic construct
MKWVSFISLLFLFSSAYSRSLDKR

SEQ ID NO: 40 - protein, 24 aa, Artificial Sequence
  Description of Artificial Sequence Synthetic construct
MKWVSFISLLFLFSSAYSGSLDKR

SEQ ID NO: 41 - protein, 86 aa, Saccharomyces cerevisiae
MRFPSIFTAVLAFAASSALAAPVNTTTEDETAQIPAEAVIGYSDLEGDFDVAVLPFSNSTNNGLLFINTFIASIAAKEEGVSLDKR

SEQ ID NO: 42 - DNA, 35 bp, Artificial Sequence
  Description of Artificial Sequence Synthetic oligonucleotide
cccgggatcccttaggcttaacctgtgaagcctgc

SEQ ID NO: 43 - DNA, 33 bp, Artificial Sequence
  Description of Artificial Sequence Synthetic oligonucleotide
cccgggatccaagcttagacttgtgccatgtcg

SEQ ID NO: 44 - DNA, 32 bp, Artificial Sequence
  Description of Artificial Sequence Synthetic oligonucleotide
cccgggatccaagcttagtcctccacatacag

SEQ ID NO: 45 - DNA, 105 bp, Artificial Sequence
  Description of Artificial Sequence Synthetic oligonucleotide
ccttaggcttaacctgtgaagcctgccaggagccgggaggcctggtggtgcctcccacagatgccccggtgagccccaccactctgtatgtggaggactaagctt

SEQ ID NO: 46 - DNA, 59 bp, Artificial Sequence
  Description of Artificial Sequence Synthetic oligonucleotide
ttaggcctctgtgaccttgcccctgaagcccctcctcctactctgcccccctaagctta

SEQ ID NO: 47 - DNA, 60 bp, Artificial Sequence
  Description of Artificial Sequence Synthetic oligonucleotide
gatctaagcttaggggggcagagtaggaggagggccttcaggggcaaggtcacagaggcc

SEQ ID NO: 48 - DNA, 35 bp, Artificial Sequence
  Description of Artificial Sequence Synthetic oligonucleotide
cccgggatcccttaggcttaaccggtgaagccggc

SEQ ID NO: 49 - DNA, 39 bp, Artificial Sequence
  Description of Artificial Sequence Synthetic oligonucleotide
ggatccttagggctgtgcagcaggctactggacctggtc

SEQ ID NO: 50 - DNA, 39 bp, Artificial Sequence
  Description of Artificial Sequence Synthetic oligonucleotide
gaattcaagcttaacagaggtagctaacgatctcgtccc

SEQ ID NO: 51 - DNA, 38 bp, Artificial Sequence
  Description of Artificial Sequence Synthetic oligonucleotide
caaggatccaagcttcagggctgcgcaaggtggcgtag

SEQ ID NO: 52 - DNA, 39 bp, Artificial Sequence
  Description of Artificial Sequence Synthetic oligonucleotide
cggggtaccttaggcttaacccccctgggccctgccagc

SEQ ID NO: 53 - DNA, 66 bp, Artificial Sequence
  Description of Artificial Sequence Synthetic oligonucleotide
gttctacgccaccttgcgcagcccggtggaggcggtgatgcacacaagagtgaggttgctcatcgg

SEQ ID NO: 54 - DNA, 60 bp, Artificial Sequence
  Description of Artificial Sequence Synthetic oligonucleotide
cagggagctggcagggcccaggggggttcgacgaaacacacccctggaataagccgagct

SEQ ID NO: 55 - protein, 23 aa, Artificial Sequence
  Description of Artificial Sequence Synthetic peptide
MRPTWAWWLFLVLLLALWAPARG

SEQ ID NO: 56 - DNA, 51 bp, Artificial Sequence
  Description of Artificial Sequence Synthetic primer
tgcgccctacctgggcctggtggctgttcctggtgctgctgctggcactgt

SEQ ID NO: 57 - DNA, 53 bp, Artificial Sequence
  Description of Artificial Sequence Synthetic primer
ttcattcctatgcaaggtgccgcgggctggagcccacagtgccagcagcagca

SEQ ID NO: 58 - DNA, 39 bp, Artificial Sequence
  Description of Artificial Sequence Synthetic primer
ctagaggatccgccaccatgcgccctacctgggcctggt

SEQ ID NO: 59 - DNA, 28 bp, Artificial Sequence
  Description of Artificial Sequence Synthetic primer
ctattccatattcattcctatgcaaggt

SEQ ID NO: 60 - DNA, 35 bp, Artificial Sequence
  Description of Artificial Sequence Synthetic primer
aggatccgccaccatgcgccctacctgggcctggt

SEQ ID NO: 61 - DNA, 32 bp, Artificial Sequence
  Description of Artificial Sequence Synthetic primer
ctagctattccatattcattcctatgcaaggt

SEQ ID NO: 62 - DNA, 32 bp, Artificial Sequence
  Description of Artificial Sequence Synthetic primer
tatggaatagctagcatattggattcttacca

SEQ ID NO: 63 - DNA, 50 bp, Artificial Sequence
  Description of Artificial Sequence Synthetic primer
gctagcagatctggtaccggcgcgccttaaactcctaaggcagcacgagt

SEQ ID NO: 64 - DNA, 18 bp, Artificial Sequence
  Description of Artificial Sequence Synthetic primer
cgtgctgccttaggagtt

SEQ ID NO: 65 - DNA, 15 bp, Artificial Sequence
  Description of Artificial Sequence Synthetic primer
taccggcgcgcctta

SEQ ID NO: 66 - DNA, 17 bp, Artificial Sequence
  Description of Artificial Sequence Synthetic primer
gggctccagcccgcggc

SEQ ID NO: 67 - DNA, 39 bp, Artificial Sequence
  Description of Artificial Sequence Synthetic primer
caatatgctagctattccatattcattcctatgcaaggt

SEQ ID NO: 68 - DNA, 41 bp, Artificial Sequence
  Description of Artificial Sequence Synthetic primer
gggctccagcccgcggcgcacctacttcaagttctacaaag

SEQ ID NO: 69 - DNA, 63 bp, Artificial Sequence
  Description of Artificial Sequence Synthetic primer
caatatgctagctattccatattcattcctatgcaaggtagtcagtgttgagatgatgctttg

SEQ ID NO: 70 - DNA, 41 bp, Artificial Sequence
  Description of Artificial Sequence Synthetic primer
gggctccagcccgcggcgaagttaaacaggaaaaccgtctg

SEQ ID NO: 71 - DNA, 62 bp, Artificial Sequence
  Description of Artificial Sequence Synthetic primer
caatatgctagctattccatattcattcctatgcaaggtaccgatttcgtagcctttcttag

SEQ ID NO: 72 - DNA, 42 bp, Artificial Sequence
  Description of Artificial Sequence Synthetic primer
cgtgctgccttaggagttgaagttaaacaggaaaaccgtctg

SEQ ID NO: 73 - DNA, 38 bp, Artificial Sequence
  Description of Artificial Sequence Synthetic primer
taccggcgcgccttaaccgatttcgtagcctttcttag

SEQ ID NO: 74 - DNA, 33 bp, Artificial Sequence
  Description of Artificial Sequence Synthetic primer
gggctccagcccgcggcgcccgcccctgcatcc

SEQ ID NO: 75 - DNA, 58 bp, Artificial Sequence
  Description of Artificial Sequence Synthetic primer
caatatgctagctattccatattcattcctatgcaaggtctggcgacgccacaggtag

SEQ ID NO: 76 - protein, 6 aa, Artificial Sequence
  Description of Artificial Sequence Synthetic 6xHis tag
HHHHHH

SEQ ID NO: 77 - protein, 15 aa, Artificial Sequence
  Description of Artificial Sequence Synthetic peptide
GGGGSGGGGSGGGGS

(19) United States
(12) Patent Application Publication
(10) Pub. No.: US 2007/0253973 A1
(43) Pub. Date: Nov. 1, 2007

(54) Fusion proteins comprising alpha fetoprotein

(75) Inventors: Craig A. Rosen, Laytonsville, MD (US); Adam C. Bell, Germantown, MD (US); Indrajit Sanyal, Bethesda, MD (US)

Correspondence Address: FOLEY AND LARDNER LLP;SUITE 500, 3000 K STREET NW, WASHINGTON, DC, 20007, US

(73) Assignee: CoGenesys, Inc.

(21) Appl. No.: 11/714,918

(22) Filed: Mar. 7, 2007

Related U.S. Application Data
(60) Provisional application No. 60/787,209, filed on Mar. 30, 2006.

(51) Int. Cl. A61K 39/29; A61P 43/00; C07H 21/04; C12N 5/00; C12N 5/06; C12P 21/08

(52) U.S. Cl. 424/189.1; 435/325; 435/69.1; 530/387.3; 536/23.4

(57) ABSTRACT

The invention relates generally to fusion proteins comprising at least one therapeutic protein or vaccine antigen and alpha fetoprotein. The therapeutic protein or vaccine antigen can be, for example, a peptide, antibody, fragment thereof, or variant thereof. The therapeutic protein or vaccine antigen is fused to alpha-fetoprotein, an alpha-fetoprotein fragment, or an alpha-fetoprotein variant. Also encompassed by the invention are nucleic acids encoding the fusion proteins of the invention, vectors comprising such nucleic acids, and host cells transformed with such nucleic acids and/or vectors. Methods of making and using the fusion proteins, nucleic acids, vectors, and host cells are also encompassed by the invention.

### CROSS-REFERENCE TO RELATED APPLICATION

This application claims priority from U.S. Provisional Application No. 60/787,209, filed Mar. 30, 2006. The contents of which are hereby incorporated by reference.

### FIELD OF THE INVENTION

The invention relates generally to fusion proteins comprising at least one therapeutic protein or a vaccine antigen and alpha-fetoprotein (AFP). The therapeutic protein or vaccine antigen can be, for example, a peptide, antibody, fragment thereof, or variant thereof. The therapeutic protein or vaccine antigen is fused to alpha-fetoprotein, an alpha-fetoprotein fragment, or an alpha-fetoprotein variant. Also encompassed by the invention are nucleic acids encoding the fusion proteins of the invention, vectors comprising such nucleic acids, and host cells transformed with such nucleic acids and/or vectors. Methods of making and using the fusion proteins, nucleic acids, vectors, and host cells are also encompassed by the invention.

### BACKGROUND

Alpha-fetoprotein (AFP), a protein of 609 amino acids, is synthesized in the embryonic liver and is found in fetal serum. AFP is not normally detectable after birth in significant levels. High levels of this protein are found in the developing fetus, but low levels exist in the amniotic fluid and maternal serum.

Therapeutic proteins and certain vaccine antigens in their native state or when recombinantly produced, such as interferons and growth hormones, are typically labile molecules exhibiting short shelf-lives, particularly when formulated in aqueous solutions. The instability in these molecules when formulated for administration dictates that many of the molecules must be lyophilized and refrigerated at all times during storage, thereby rendering the molecules difficult to transport and/or store. Storage problems are particularly acute when pharmaceutical formulations must be stored and dispensed outside of the hospital environment. Moreover, such proteins tend to have short in vivo half lives, which can limit their effectiveness in therapeutic compositions and vaccines.

Few practical solutions to the storage problems of labile protein molecules have been proposed. Prior methods of increasing the in vivo half life of proteins includes fusing the proteins to carrier molecules such as albumin. See U.S. Pat. Nos. 6,994,857; 6,946,134; 6,926,898; and 6,905,688, all for “Albumin Fusion Proteins.” However, not all proteins may be successfully stabilized using albumin. For example, fusion to albumin may unacceptably decrease the activity of the protein. Also, for certain types of formulations, such as vaccines, albumin may not be desirable as a carrier molecule. A further concern regarding the use of albumin fusion proteins is the observation that such proteins can generate anti-albumin antibodies which could influence the residence time of the conjugate in the blood circulation, or even promote allergic reactions.

Other technology for increasing the residence time of an administered therapeutic protein includes PEGylation, which refers to attaching poly(ethyleneglycol) (PEG) moieties to a protein of interest. See Francis et al., Focus on Growth Factors, 3:4-10 (1992); European Patent Publication Nos. 154316 and 401384; and U.S. Pat. No. 4,179,337. In general, pegylation can be carried out via an acylation reaction or an alkylation reaction with a reactive polyethylene glycol molecule (or an analogous reactive water-soluble polymer). However, this manufacturing process tends to be expensive. Moreover, PEGylation can render proteins largely inactive. For example, in the case of a PEGylated interferon (PEGASYS®), over 95 percent of the activity of interferon conjugated to branched PEG is lost, and a similar observation was made for PEGylated growth hormone. Furthermore, toxicity becomes even more relevant in the case of protein therapeutics which, unlike presently approved PEGylated products (such as PEG Intron, PEGASYS®) injected in microgram quantities, must be administered in far greater (milligram) quantities, and often chronically (for example antibodies and their fragments, insulin, etc.). In such cases, the issue of toxicity is principally related to the nature of PEG, which is a non-biodegradable material. When PEG is conjugated to peptides or proteins which must retain their PEG moiety to exhibit their superior pharmacodynamics, the polymer will sooner or later and, regardless of its size, end up in tissues intracellularly via endocytosis of the conjugate. Upon entry into the lysosomes and subsequent degradation of its therapeutic moiety, PEG will accumulate and possibly create in the long-term a “lysosomal storage disease”. Evidence to that effect has been obtained by, for instance, the experimental use of PEGylated tumour necrosis factor binding protein in rats. Significant vacuolation of renal cortical tubular epithelium due to accumulated PEG was detected. A further concern on the use of PEGylation is the observation that PEGylated proteins can generate anti-PEG antibodies which could influence the residence time of the conjugate in the blood circulation, or even promote allergic reactions.

Accordingly, there is a need for new methods for stabilized, long lasting formulations of proteinaceous therapeutic molecules, as well as long lasting formulations of vaccine antigens, providing longer in vivo residence times. The present invention satisfies these needs.

### SUMMARY

The invention encompasses alpha-fetoprotein fusion proteins comprising: (1) at least one therapeutic protein or vaccine antigen, such as a peptide, antibody, fragment thereof, or variant thereof, fused to (2) alpha-fetoprotein, a fragment of alpha-fetoprotein, or a variant of alpha-fetoprotein. Preferably, the fusion proteins of the invention prolong the shelf life of the therapeutic protein or vaccine antigen, and/or prolong the in vivo residence time of the therapeutic protein or vaccine antigen. In another embodiment, the fusion proteins of the invention are more stable than the non-fused therapeutic protein or vaccine antigen. In yet another embodiment, the fusion proteins of the invention exhibit improved activity in solution or in a pharmaceutical composition, in vitro or in vivo, as compared to the non-fused therapeutic protein or vaccine antigen.

The invention also encompasses polynucleotides comprising nucleic acid molecules encoding a fusion protein according to the invention, vectors comprising such polynucleotides, and host cells transformed with such polynucleotides and/or vectors.

In one aspect of the invention, alpha-fetoprotein fusion proteins include, but are not limited to human growth hormone, interleukin-2 (IL-2), granulocyte macrophage colony stimulating factor (GM-CSF), granulocyte colony stimulating factor (G-CSF), butyrylcholinesterase, glucocerebrosidase (GBA), calcitonin, interferon-beta, interferon alpha, parathyroid hormone (PTH(1-34) and PTH(1-84)), Glucagon-like peptide (GLP-1 and GLP-2), PA toxin (anthrax), exendin-4, and the polynucleotides encoding such proteins.

The invention also encompasses pharmaceutical formulations comprising an alpha-fetoprotein fusion protein of the invention and a pharmaceutically acceptable diluent or carrier. Such formulations may be in a kit or container. Such a kit or container may be packaged with instructions pertaining to the extended shelf life of the therapeutic protein.

The invention further encompasses transgenic organisms modified to comprise the nucleic acid molecules of the invention, preferably modified to express an alpha-fetoprotein fusion protein of the invention.

In other embodiments, the invention encompasses methods of making the alpha-fetoprotein fusion proteins of the invention.

Finally, the invention encompasses methods of preventing, treating, or ameliorating a disease or disorder utilizing the alpha-fetoproteins of the invention. An exemplary method comprises administering to a subject in which such treatment, prevention or amelioration is desired an alpha-fetoprotein fusion protein of the invention in an amount effective to treat, prevent or ameliorate the disease or disorder.

Both the foregoing general description and the following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 shows the nucleotide sequence of alpha-fetoprotein (GenBank BC027881).

FIG. 2 shows the amino acid sequence of alpha-fetoprotein.

FIG. 3 shows annotated amino acid and nucleotide sequences of the engineered SPCON2.AFP ORF.

FIG. 4 shows the full ORF (nucleotide sequence) with silent restriction sites for the engineered SPcon2.AFP full ORF.

FIG. 5 shows the amino acid sequence of SPcon2.AFP, with the signal peptide underlined.

FIG. 6 shows the amino acid sequence of a fusion protein between G-CSF and AFP (AFP-G-CSF); the signal peptide is underlined and G-CSF is double underlined.

FIG. 7 shows the amino acid sequence of the fusion protein comprising AFP and IL2; (AFP-IL2); the signal peptide is underlined and IL2 is double underlined.

FIG. 8 shows the amino acid sequence of the fusion protein comprising AFP and glucocerebrosidase (GBA) (AFP-GBA); the signal peptide is underlined and glucocerebrosidase is double underlined.

FIG. 9 shows the amino acid sequence of the fusion protein comprising AFP and butyrycholinesterase (BChE) (AFP-BChE); the signal peptide is underlined and butyrycholinesterase is double underlined.

FIG. 10 shows an IL2 N-terminal PCR fusion product.

FIG. 11 shows an anthrax PA antigen N-terminal fusion PCR product.

FIG. 12 shows the results of expression of AFP and the fusion proteins GBA-AFP, AFP-G-CSF, IL2-AFP, following transfection of HEK293 cells with vectors encoding AFP and the AFP fusion proteins.

FIG. 13 shows the results of a cell proliferation assay with AML-193 cells, which is a GM-CSF and G-CSF dependent cell line, demonstrating expression of the fusion protein AFP-GCSF. The GM-CSF-dependent cell line AML-193 (P4) was starved of GM-CSF for 24 hours in 50 ul of “blank media” (20,000/well). To measure proliferation in response to G-CSF and ALF.G-CSG fusion protein, triplicate wells were treated with an equal volume of “charged” media, blank media, or serial 2-fold dilutions of charged media, and grown for 48 hours. Charged media was collected from HEK293T cells transiently transfected with ALF-G-CSF or ALF expressing pcDNA3.1. Cell titers were measured by luminescence using the Cell-Titer-Glow Assay (Promega).

FIG. 14 shows the results of a cell proliferation assay utilizing the IL2 dependent cell line CTLL-2, demonstrating the expression of the fusion protein IL2-AFP. The IL-2-dependent cell line CTLL-2 (P6) was starved of IL-2 for 24 hours in 50 ul of growth medium recommended by ATCC (20,000/well). To measure IL-2-dependent proliferation, cells were treated with an equal volume of “charged” media from a 293T culture containing cells transiently transfected with pcDNA vectors expressing ALF or IL2-ALF 48 hours prior to media collection or the same “blank” media spiked with 5 ng/ml of recombinant human IL2. Charged media was serially diluted in 2-fold steps across a 96-well plate and then the cells were grown for 48 hours. Treatments were made in triplicate. Cell titers were measured by luminescence using the Cell-Titer-Glow Assay (Promega).

FIG. 15 shows the results of a 4-MUG (4-methylumbelliferyl-beta-D-glucoside) assay demonstrating the activity of the fusion protein GBA-AFP.

FIG. 16 shows the results of an assay measuring the expression of secreted alkaline phosphatase (SEAP), which demonstrates the activity of the fusion protein AFP-IFNalpha.

FIG. 17 shows the results of an Ellman-based DTNB assay for esterase activity, which is a bioassay used to detect butyrylcholinesterase (BChE) activity.

FIG. 18 shows the DNA sequence and the encoded ORF for the ExendinAFP Construct ID No. 4680.

FIG. 19 shows the ORF encoded by ExendinAFP Construct ID No. 4680

FIG. 20 shows the DNA sequence and the encoded ORF for the Exendin(2×)AFP Construct ID No. 4767.

FIG. 21 shows the ORF encoded by Exendin(2×)AFP Construct ID No. 4767.

FIG. 22 shows the DNA sequence and the encoded ORF for the EXN(4×)AFP(D33-V609) Construct ID No. 4792.

FIG. 23 shows the ORF encoded by EXN(4×)AFP(D33-V609) Construct ID No. 4792.

FIG. 24 shows the DNA sequence and the encoded ORF for the ExendinAFP(T20-V609)(N251Q) Construct ID No. 4798.

FIG. 25 shows the ORF for the ExendinAFP(T20-V609)(N251Q) Construct ID No. 4798.

FIG. 26 shows that ExendinAFP fusion proteins activate the GLP-1 receptor and induce cAMP production. 293/GLP-1 receptor expressing cells were treated for 30 min with increasing concentrations of Exendin-AFP, Exendin(2×)-AFP, Exendin (4×)-AFP, Exendin-AFP(glycosylation mutant), Exendin peptide or AFP protein controls. Levels of intracellular cAMP were measured by enzymatic bioluminescent cAMP-Glo assay. Values are expressed as means±SD of triplicate wells. The measured signal was plotted using a four-parameter logistic model and the signal was inversely proportional to intracellular cAMP production.

FIG. 27 shows that Exendin-AFP dose dependently inhibits cumulative food intake and induces temporary weight loss in C57BL/6 mice: (A) C57BL/6 mice were given a subcutaneous injection of ExendinAFP at either 2 (diamonds) or 6 mg/kg (circles) or equal volume of saline control (squares). After injection mice were given free access to water and High Fat Diet (HFD) chow. Mouse food intake (grams) was measured at 4, 8, 12, 24, 30, 36, and 48 hours following injection, and cumulative food intake up to 48 h was calculated; (B) The cumulative changes in mouse weight (grams, n=8) were determined at 24 and 48 hours after subcutaneous injection of ExendinAFP or saline control.

FIG. 28 shows that ExendinAFP and Exendin(2×)AFP inhibit food intake, induce weight loss, and decrease serum glucose levels with activity comparable to Exendin peptide. ExendinAFP (3 mg/kg, triangles); Exendin(2×)AFP (3 mg/kg, circles), Exendin peptide (0.175 mg/kg which is equalmolar to ExendinAFP, diamonds); and saline control (squares) were injected subcutaneously into C57BL6 mice. Cumulative food intake, cumulative weight change and serum glucose levels were measured at indicated times following injection.

### DETAILED DESCRIPTION

I. Overview

The invention relates generally to alpha-fetoprotein fusion proteins, polynucleotides encoding alpha-fetoprotein fusion proteins, and methods of treating, preventing, or ameliorating diseases or disorders using alpha-fetoprotein fusion proteins or polynucleotides encoding alpha-fetoprotein fusion proteins. As used herein, “alpha-fetoprotein fusion protein” refers to a protein formed by the fusion of: (1) at least one molecule of alpha-fetoprotein, a biologically active alpha-fetoprotein fragment, or biologically active alpha-fetoprotein variant to (2) at least one molecule of at least one therapeutic protein, biologically active and/or therapeutically active therapeutic protein fragment, biologically active and/or therapeutically active therapeutic protein variant, or vaccine antigen. The alpha-fetoprotein, fragment, or variant thereof is fused to the therapeutic protein, fragment, or variant thereof, or vaccine antigen, by genetic fusion; i.e., the alpha-fetoprotein fusion protein is generated by translation of a nucleic acid in which a polynucleotide encoding all or a portion of the therapeutic protein or vaccine antigen is joined in-frame with a polynucleotide encoding all or a portion of the alpha-fetoprotein. The therapeutic protein or vaccine antigen and alpha-fetoprotein, once part of the alpha-fetoprotein fusion protein, may each be referred to as a “portion”, “region” or “moiety” of the alpha-fetoprotein fusion protein (e.g., a “therapeutic protein portion” or “vaccine antigen portion” or an “alpha-fetoprotein protein portion”). The alpha-fetoprotein fusion protein may include multiple copies of the therapeutic protein, therapeutic protein fragment, or therapeutic protein variant in tandem (e.g., 2, 3, 4, or more copies).

A fragment of the alpha-fetoprotein may include a portion that does not include the signal peptide. In some embodiments, a fragment of the alpha-fetoprotein includes amino acids T20-V609 or D33-V609. A fragment typically has alpha-fetoprotein activity.

A variant of the alpha-fetoprotein may have one or more amino acid substitutions. For example, a variant may have at least about 95% amino acid sequence identity to alpha-fetoprotein (or at least about 96%, 97%, 98%, or 99% amino acid sequence identity), and alpha-fetoprotein functional activity. In some embodiments, the variant includes one or more amino acid substitutions at a glycosylation site (e.g., an asparagine (N) substitution such as N251Q). In some embodiments, a fragment of alpha-fetoprotein may include one or more amino acid substitutions (e.g., an asparagine (N) substitution such as N251Q).

In a further preferred embodiment, an alpha-fetoprotein fusion protein of the invention is processed by a host cell and secreted into the surrounding culture medium. Processing of the nascent alpha-fetoprotein fusion protein that occurs in the secretory pathways of the host used for expression may include, but is not limited to, signal peptide cleavage (e.g., of the native alpha-fetoprotein signal peptide or of a heterologous signal peptide as described herein); formation of disulfide bonds; proper folding; addition and processing of carbohydrates (such as for example, N- and O-linked glycosylation); specific proteolytic cleavages; and assembly into multimeric proteins. An alpha-fetoprotein fusion protein of the invention is preferably in the processed form. In one embodiment, the “processed form of an alpha-fetoprotein fusion protein” refers to an alpha-fetoprotein fusion protein product which has undergone N-terminal signal peptide cleavage (e.g., of the native alpha-fetoprotein signal peptide or of a heterologous signal peptide as described herein), also referred to as a “mature alpha-fetoprotein fusion protein”.

The therapeutic protein portion of the alpha-fetoprotein fusion protein can be the extracellular soluble domain of the therapeutic protein. In an alternative embodiment, the therapeutic protein portion of the alpha-fetoprotein fusion protein is the active form of the therapeutic protein.

II. Definitions

The following definitions are provided to facilitate understanding of certain terms used throughout this specification.

As used herein, “alpha-fetoprotein fusion construct” refers to a nucleic acid molecule comprising a polynucleotide encoding at least one molecule of alpha-fetoprotein, a fragment of alpha-fetoprotein, or an alpha-fetoprotein variant, joined in frame to at least one polynucleotide encoding at least one molecule of a therapeutic protein, a therapeutic protein fragment, a therapeutic protein variant, or a vaccine antigen. As used herein, the term “therapeutic protein” encompasses vaccine antigens. The alpha-fetoprotein fusion construct can further comprise, for example, one or more of the following elements: (1) a functional self-replicating vector (including but not limited to, a shuttle vector, an expression vector, an integration vector, and/or a replication system), (2) a region for initiation of transcription (e.g., a promoter region, such as for example, a regulatable or inducible promoter, a constitutive promoter), (3) a region for termination of transcription, (4) a leader sequence, and/or (5) a selectable marker. The polynucleotide encoding the therapeutic protein and alpha-fetoprotein protein, once part of the alpha-fetoprotein fusion construct, may each be referred to as a “portion,” “region” or “moiety” of the alpha-fetoprotein fusion construct.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

III. Compositions

A. Alpha-Fetoprotein

As described above, an alpha-fetoprotein fusion protein of the invention comprises at least a fragment or variant of a therapeutic protein and at least a fragment or variant of alpha-fetoprotein, which are associated with one another, preferably by genetic fusion.

An additional embodiment comprises at least a fragment or variant of a therapeutic protein and at least a fragment or variant of alpha-fetoprotein, which are linked to one another by chemical conjugation.

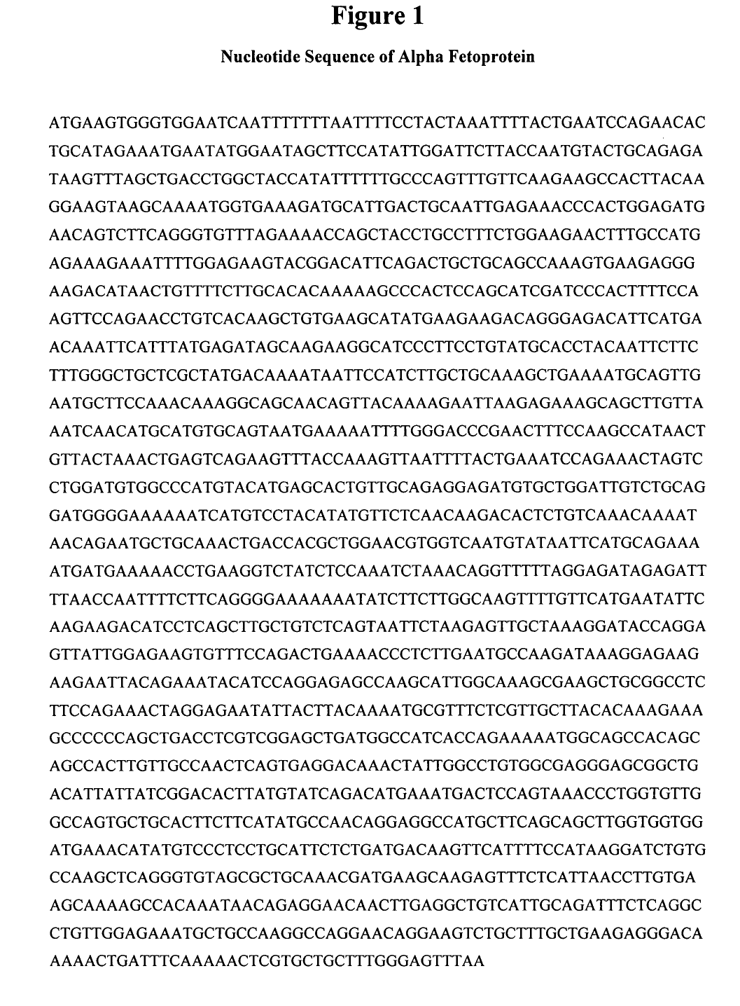

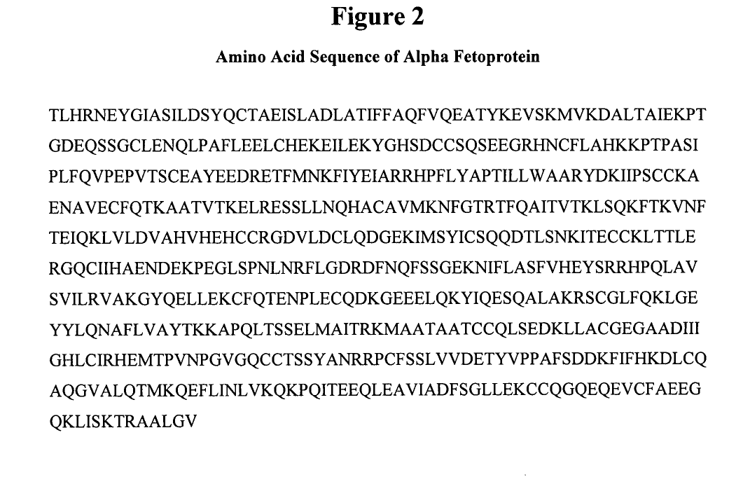

As used herein, “alpha-fetoprotein” refers collectively to alpha-fetoprotein or amino acid sequence, or an alpha-fetoprotein fragment or variant, having one or more functional activities (e.g., biological activities) of alpha-fetoprotein. In particular, “alpha-fetoprotein” refers to alpha-fetoprotein or fragments thereof as shown in FIG. 1 (nucleic acid sequence encoding alpha-fetoprotein) and FIG. 2 (amino acid sequence of alpha-fetoprotein), or alpha-fetoprotein from other vertebrates or fragments thereof, or analogs or variants of these molecules or fragments thereof.

As described herein, the use of alpha-fetoprotein will provide considerable benefit in the invention. Alpha-fetoprotein is a naturally occurring molecule in human development and, consequently, the immunological tolerance for this protein enables the present invention to have considerable advantage over other carrier proteins that may cause an immunological response when introduced to the body. The high tolerance to alpha-fetoprotein makes it unlikely that adults will raise antibodies to alpha-fetoprotein. This is particularly beneficial for fusion proteins of the invention to be used in vaccine applications.

In a first example, an antigen for a vaccine is coupled to at least one terminus of AFP. Any antigen that elicits an immune response can be utilized in the compositions of the invention. Exemplary vaccine antigens include, but are not limited to, bacterial antigens, mycotic antigens, prion antigens, parasites, PA-toxin (e.g., anthrax), Human Immunodeficiency Virus (HIV-1 and HIV-2), Avian Flu antigen (e.g., H5N1), hepatitis, cancer, Severe Acute Respiratory Syndrome (SARS), and tuberculosis. Other exemplary vaccine antigens are described herein.

In a second example, a vaccine antigen is coupled to at least one end of AFP and an immunoadjuvant is coupled to at least one end of AFP (optionally the other end). Examples of adjuvants useful in vaccines include, but are not limited to, cytokines such as IL-2, Lipid A, including monophosphoryl lipid A, bacterial products, endotoxins, cholesterol, fatty acids, aliphatic amines, paraffinic and vegetable oils, threonyl derivative, and muramyl dipeptide.

As described below, the alpha-fetoprotein fusion proteins may be used in vaccines. By utilizing the alpha-fetoprotein fusion protein approach this invention enhances the residence time of antigens in the body and allows for more effective vaccines. By allowing additional time before the degradation of the antigen, the body is better able to mount an immunological response to the antigen and become immunized against future infection.

As noted below, the invention relates both to the alpha-fetoprotein fusion proteins, and vectors, constructs, and organisms comprising the polynucleotides encoding these proteins. As noted above, the vectors of the invention may comprise expression cassettes so that portions of the cassette may be easily changed. This benefit will be useful in, for example, the design of vectors to treat disease or act in a vaccine. Although mutations may occur in, for example, viruses, changes may be easily made by one of skill in the art to the vectors to produce molecules identical to the mutated virus proteins. After these changes, the expression and purification protocols will generally be identical or very similar to the unmutated construct. Such alterations are well known to one of ordinary skill in the art.

Additionally, the invention also includes the addition of either T or B cell stimulating epitopes to the N or C terminus of the alpha-fetoprotein fusion protein in, for example, the case of vaccines.

As used herein, a portion of alpha-fetoprotein sufficient to prolong the therapeutic activity or shelf-life of the therapeutic protein refers to a portion of alpha-fetoprotein sufficient in length or structure to stabilize or prolong the therapeutic activity of the protein so that the shelf life of the therapeutic protein portion of the alpha-fetoprotein fusion protein is prolonged or extended compared to the shelf-life in the non-fusion state. The alpha-fetoprotein portion of the alpha-fetoprotein fusion proteins may comprise the full length of the alpha-fetoprotein sequence as described above, or may include one or more fragments thereof that are capable of stabilizing or prolonging the therapeutic activity.

In one embodiment of the invention, the AFP portion comprises at least half of the full length AFP protein. In other embodiments, such fragments may be of about 10 or more amino acids in length or may include about 15, about 20, about 25, about 30, about 50, about 70, about 90, about 110, about 130, about 150, about 170, about 190, about 210, about 230, about 250, about 270, about 290, about 310, about 330, about 350, about 370, about 390, about 410, about 430, about 450, about 470, about 490, about 510, about 530, about 550, about 570, about 590, about 600, about 605 or more contiguous amino acids from the alpha-fetoprotein sequence or may include part or all of specific domains of alpha-fetoprotein. The AFP portion may include amino acids T20-V609 or D33-V609 and optionally may include one or more amino acid substitutions (e.g., at one or more glycosylation sites such as N251Q).

The alpha-fetoprotein portion of the alpha-fetoprotein fusion proteins of the invention may be a variant of normal alpha-fetoprotein. The therapeutic protein portion of the alpha-fetoprotein fusion proteins of the invention may also be variants of the therapeutic proteins as described herein. The term “variants” includes insertions, deletions and substitutions, either conservative or non conservative, where such changes do not substantially alter one or more of the oncotic, useful ligand-binding and non-immunogenic properties of alpha-fetoprotein, or the active site, or active domain which confers the therapeutic activities of the therapeutic proteins. For example, variants may include proteins having at least about 95% sequence identity (or 96%, 97%, 98%, or 99% sequence identity) and retaining at least one functional activity of alpha-fetoprotein or the therapeutic protein.

In particular, the alpha-fetoprotein fusion proteins of the invention may include naturally occurring polymorphic variants of human alpha-fetoprotein and fragments of human alpha-fetoprotein. The alpha-fetoprotein may be derived from any vertebrate, especially any mammal, for example human, cow, sheep, or pig. The alpha-fetoprotein portion of the alpha-fetoprotein fusion protein may be from a different animal than the therapeutic protein portion.

Generally speaking, an alpha-fetoprotein fragment or variant will be at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 580, at least about 590, at least about 600, at least about 601, at least about 602, at least about 603, at least about 604, at least about 605, at least about 606, at least about 607, at least about 609, at least about 609 amino acids long. A fragment or variant may include amino acids T20-V609 or D33-V609 and optionally may include one or more amino acid substitutions (e.g., at one or more glycosylation sites such as N251Q).

Preferably, the alpha-fetoprotein portion of an alpha-fetoprotein fusion protein of the invention comprises at least one subdomain or domain of alpha-fetoprotein or conservative modifications thereof. If the fusion is based on subdomains, some or all of the adjacent linker is preferably used to link to the therapeutic protein moiety.

B. Therapeutic Protein(s) and/or Vaccine Antigen(s)

As used herein, “therapeutic protein” and “vaccine antigen”, which in this application can collectively be referred to as a “therapeutic protein,” refers to proteins, polypeptides, antibodies, peptides or fragments or variants thereof, having one or more therapeutic and/or biological activities. Therapeutic proteins and vaccine antigens encompassed by the invention include but are not limited to proteins, polypeptides, peptides, antibodies, and biologics. (The terms peptides, proteins, and polypeptides are used interchangeably herein.) It is specifically contemplated that the term “therapeutic protein” and “vaccine antigen” encompasses antibodies and fragments and variants thereof. Thus, a protein of the invention may comprise at least a fragment or variant of a therapeutic protein or vaccine antigen, and/or at least a fragment or variant of an antibody. Additionally, the terms “therapeutic protein” and “vaccine antigen” may refer to the endogenous or naturally occurring correlate of a therapeutic protein.

By a polypeptide displaying a “therapeutic activity” or a protein that is “therapeutically active” is meant a polypeptide that possesses one or more known biological and/or therapeutic activities associated with a therapeutic protein, such as one or more of the therapeutic proteins described herein or otherwise known in the art. As a non-limiting example, a “therapeutic protein” is a protein that is useful to treat, prevent or ameliorate a disease, condition or disorder. As a non-limiting example, a “therapeutic protein” may be one that binds specifically to a particular cell type (normal (e.g., lymphocytes) or abnormal e.g., (cancer cells)) and therefore may be used to target a compound (drug, or cytotoxic agent) to that cell type specifically.

The proteins and antigens useful in the fusion proteins of the invention are not limited to human sequences. For examples, conatoxins, derived from mollusks, can be useful in fusion proteins adapted for pain relief, and fusion proteins comprising exendin-4 (an incretin mimetic exenatide), which can be useful in the treatment of diabetes.

For example, a non-exhaustive list of “therapeutic proteins” which may be utilized in the invention includes, but is not limited to, human growth hormone, interleukin-2 (IL-2), granulocyte macrophage colony stimulating factor (GM-CSF), calcitonin, interferon-beta, interferon-alpha, granulocyte colony stimulating factor (G-CSF), glucagon like peptide 1 (GLP-1), glucagon like peptide 2 (GLP-2), PA toxin (anthrax), parathyroid hormone (PTH, including PTH(1-34) and PTH(1-84)), butyrylcholinesterase, glucocerebrosidase, and exendin-4. G-CSF is useful, for example, in treating neutropenia; IL-2 is useful in enhancing the antigenicity of AFP fusion proteins for vaccine development, and the fusion protein alone as a treatment for cancer; butyrylcholinesterase is useful in the treatment of cocaine toxicity and organophosphate poisoning; glucocerebrosidase is useful in the treatment of Gaucher's disease; and IFN-alpha is useful in the treatment of hepatitis C and other viral diseases (and possibly multiple sclerosis).

Exendin-4 is a 39 amino acid peptide isolated from the saliva of the Gila monster (Heloderma suspectum). Exendin-4 is approximately 50% homologous at the amino acid level with human glucagon-like peptide 1 (GLP-1) and is a GLP-1 receptor agonist useful for treating diabetes (e.g., type II diabetes). Exendin-4 binds to GLP-1 receptors and induces a cAMP signaling response in cells expressing GLP-1 receptor. In vivo, exendin-4 improves glucose homeostasis by mimicking the actions of naturally occurring GLP-1. Exendin-4 regulates glycemic control by a combination of mechanisms including, sensitizing glucose dependent insulin secretion from the pancreas, suppressing glucagon secretion, decreasing food intake, and delaying gastric emptying. The therapeutic protein of the present alpha-fetoprotein fusion proteins may include exendin-4, an exendin-4 fragment, an exendin-4 variant, or an exendin-4 analogue, as disclosed in U.S. Pat. Nos. 6,989,366; 6,924,264; 6,902,744; 6,593,295; 6,528,486, which are incorporated herein by reference.

Therapeutic proteins of the present alpha-fetoprotein fusion proteins may include therapeutic enzymes. Some therapeutic enzymes such as ceredase (glucocerebrosidase) only bind to and enter cells through a mannose receptor. A synthetic and inefficient approach is currently used to convert the ceredase sugars to have a terminal mannose. Unlike albumin, which has previously been used as a carrier for therapeutic proteins, AFP is glycosylated and has mannose as part of the oligosaccharide mix. That and the fact that AFP receptors are abundant on monocytes/macrophages makes AFP an ideal carrier for this therapeutic enzyme and other enzymes that can bind to cells and enter through a mannose binding receptor.

In another non-limiting example, a “therapeutic protein” is a protein that has a biological activity, and in particular, a biological activity that is useful for treating, preventing or ameliorating a disease. A non-inclusive list of biological activities that may be possessed by a therapeutic protein includes, inhibition of HIV-1 infection of cells, stimulation of intestinal epithelial cell proliferation, reducing intestinal epithelial cell permeability, stimulating insulin secretion, induction of bronchodilation and vasodilation, inhibition of aldosterone and renin secretion, blood pressure regulation, promoting neuronal growth, enhancing an immune response, enhancing inflammation, or suppression of appetite.

Therapeutic proteins corresponding to a therapeutic protein portion of an alpha-fetoprotein fusion protein of the invention, such as cell surface and secretory proteins, are often modified by the attachment of one or more oligosaccharide groups. The modification, referred to as glycosylation, can dramatically affect the physical properties of proteins and can be important in protein stability, secretion, and localization. Glycosylation occurs at specific locations along the polypeptide backbone. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser or Asn-X-Thr sequence, where X can be any amino acid except proline. N-acetylneuramic acid (also known as sialic acid) is usually the terminal residue of both N-linked and O-linked oligosaccharides. Variables such as protein structure and cell type influence the number and nature of the carbohydrate units within the chains at different glycosylation sites. Glycosylation isomers are also common at the same site within a given cell type.

Therapeutic proteins corresponding to a therapeutic protein portion of an alpha-fetoprotein fusion protein of the invention, as well as analogs and variants thereof, may be modified so that glycosylation at one or more sites is altered as a result of manipulation(s) of their nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that will not glycosylate them, e.g., in E. coli or glycosylation-deficient yeast. These approaches are described in more detail below and are known in the art.

In a further embodiment of the invention, an “expression cassette” comprising one or more of: (1) a polynucleotide encoding a given alpha-fetoprotein fusion protein, (2) a leader sequence, (3) a promoter region, and (4) a transcriptional terminator can be moved or “subcloned” from one vector into another. Fragments to be subcloned may be generated by methods well known in the art, such as, for example, PCR amplification and/or restriction enzyme digestion.

In one embodiment, a polynucleotide of the invention which encodes the alpha-fetoprotein portion of an alpha-fetoprotein fusion protein is optimized for expression in human, bacterial, fungal, yeast, or mammalian cells. Additionally, the therapeutic peptide may be synthetically made. In a further preferred embodiment, a polynucleotide of the invention which encodes the therapeutic protein portion of an alpha-fetoprotein fusion protein is optimized for expression in the above mentioned cells. In a still further preferred embodiment, a polynucleotide encoding an alpha-fetoprotein fusion protein of the invention is optimized for expression in the above mentioned cells.

In an alternative embodiment, a codon optimized polynucleotide which encodes a therapeutic protein portion of an alpha-fetoprotein fusion protein does not hybridize to the wild type polynucleotide encoding the therapeutic protein under stringent hybridization conditions, as described herein. In a further embodiment, a codon optimized polynucleotide which encodes an alpha-fetoprotein portion of an alpha-fetoprotein fusion protein does not hybridize to the wild type polynucleotide encoding the alpha-fetoprotein protein under stringent hybridization conditions as described herein. In another embodiment, a codon optimized polynucleotide which encodes an alpha-fetoprotein fusion protein does not hybridize to the wild type polynucleotide encoding the therapeutic protein portion or the alpha-fetoprotein protein portion under stringent hybridization conditions as described herein.

The polypeptide of the invention can be composed of amino acids joined to each other by peptide bonds or modified peptide bonds, i.e., peptide isosteres, and may comprise amino acids other than the 20 gene-encoded amino acids naturally observed in humans. The polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may comprise many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include but are not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifler et al., Meth. Enzymol. 182:626-646 (1990); Rattan et al., Ann. N.Y. Acad. Sci. 663:48-62 (1992)).

An additional embodiment includes a polynucleotide encoding a protein comprising at least a fragment or variant of a therapeutic protein and at least a fragment or variant of alpha-fetoprotein, which are linked with one another by chemical conjugation.

1. Therapeutic Protein Fragments and Variants

a. Fragments

The invention is further directed to fragments of the therapeutic proteins, alpha-fetoprotein proteins, and/or alpha-fetoprotein fusion proteins of the invention. The invention is also directed to polynucleotides encoding fragments of the therapeutic proteins, alpha-fetoprotein proteins, and/or alpha-fetoprotein fusion proteins of the invention.

In one embodiment, the fusion proteins of the invention comprise at least half of the amino acid sequence of AFP, i.e., at least about 304 consecutive amino acids of the complete 609 amino acid sequence of AFP. In other embodiments, the fusion proteins of the invention comprise at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 580, at least about 590, at least about 599, at least about 600, at least about 601, at least about 602, at least about 603, at least about 604, at least about 605, at least about 606, at least about 607, or at least about 608 consecutive amino acids of the complete 609 amino acid sequence of AFP. The fusion protein may include amino acids T20-V609 or D33-V609 of AFP and optionally may include one or more amino acid substitutions (e.g., at one or more glycosylation sites such as N251Q).

In another embodiment, the polynucleotides encoding the fusion proteins of the invention comprise at least half of the nucleotide sequence of AFP, i.e., at least about 913 consecutive nucleotides of the complete 1827 nucleotide sequence of AFP. In other embodiments, the fusion proteins of the invention comprise at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1810, at least about 1814, at least about 1818, at least about 1821, or at least about 1824 consecutive nucleotides of the complete 1827 nucleotide sequence of AFP. The polynucleotides may comprise polynucleotide that encode amino acids T20-V609 or D33-V609 of AFP, which optionally may include one or more amino acid substitutions (e.g., at one or more glycosylation sites such as N251Q).

Even if deletion of one or more amino acids from the N-terminus, C-terminus, or both termini of a protein results in modification or loss of one or more biological functions of the therapeutic protein, alpha-fetoprotein protein, and/or alpha-fetoprotein fusion protein of the invention, other therapeutic activities and/or functional activities (e.g., biological activities, ability to multimerize, ability to bind a ligand) may still be retained. For example, the ability of polypeptides with N-terminal, C-terminal or both types of deletions to induce and/or bind to antibodies which recognize the complete or mature forms of the polypeptides generally will be retained when less than the majority of the residues of the complete polypeptide are removed from the N-terminus and/or C-terminus. Whether a particular polypeptide lacking N-terminal and/or C-terminal residues of a complete polypeptide retains such immunologic activities can readily be determined by routine methods described herein and otherwise known in the art. It is not unlikely that a mutein with a large number of deleted N-terminal and/or C-terminal amino acid residues may retain some biological or immunogenic activities. In fact, peptides composed of as few as six amino acid residues may often evoke an immune response.

Accordingly, fragments of a therapeutic protein corresponding to a therapeutic protein portion of an alpha-fetoprotein fusion protein of the invention include the full length protein as well as polypeptides having one or more residues deleted from the amino and/or carboxy terminus of the amino acid sequence of the polypeptide. Polynucleotides encoding these polypeptides are also encompassed by the invention. In some embodiments, the fragments may comprise at least about 95% of the full-length therapeutic protein and may have at least one functional activity of the full-length therapeutic protein.

In addition, fragments of alpha-fetoprotein polypeptides corresponding to an alpha-fetoprotein protein portion of an alpha-fetoprotein fusion protein of the invention include the full length protein as well as polypeptides having one or more residues deleted from the amino and/or carboxy terminus of the amino acid sequence of the polypeptide. Polynucleotides encoding these polypeptides are also encompassed by the invention. In some embodiments, the fragments may comprise at least about 95% of the full-length AFP protein and may have at least one functional activity of the full-length AFP.

Moreover, fragments of alpha-fetoprotein fusion proteins of the invention include the full length alpha-fetoprotein fusion protein as well as polypeptides having one or more residues deleted from the amino and/or carboxy terminus of the alpha-fetoprotein fusion protein. Polynucleotides encoding these polypeptides are also encompassed by the invention.

Also as mentioned above, even if deletion of one or more amino acids from the N-terminus and/or C-terminus of a reference polypeptide (e.g., a therapeutic protein, alpha-fetoprotein protein, or alpha-fetoprotein fusion protein of the invention) results in modification or loss of one or more biological functions of the protein, other functional activities (e.g., biological activities, ability to multimerize, ability to bind a ligand) and/or therapeutic activities may still be retained. Therapeutic activity can readily be determined by routine methods described herein and/or otherwise known in the art.

The present application is also directed to proteins comprising polypeptides at least about 60%, at least about 65%, at least about 70%, at least about 75%, at least about 80%, at least about 85%, at least about 90%, at least about 95%, at least about 96%, at least about 97%, at least about 98% or at least about 99% identical to a reference polypeptide sequence. Polynucleotides encoding these polypeptides are also encompassed by the invention. The polypeptides may have at least one functional activity of the full-length polypeptide.

Preferred polypeptide fragments of the invention are fragments comprising, or alternatively, consisting of, an amino acid sequence that displays a therapeutic activity and/or functional activity (e.g. biological activity) of the polypeptide sequence of the therapeutic protein or alpha-fetoprotein protein of which the amino acid sequence is a fragment. Other preferred polypeptide fragments are biologically active fragments. Biologically active fragments are those exhibiting activity similar, but not necessarily identical, to an activity of the polypeptide of the invention. The biological activity of the fragments may include an improved desired activity, or a decreased undesirable activity.

b. Variants

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant”, refers to a therapeutic protein portion of an alpha-fetoprotein fusion protein of the invention, alpha-fetoprotein portion of an alpha-fetoprotein fusion protein of the invention, or alpha-fetoprotein fusion protein of the invention differing in sequence from a therapeutic protein, alpha-fetoprotein protein, and/or alpha-fetoprotein fusion protein, respectively, but retaining at least one functional and/or therapeutic property thereof as described elsewhere herein or otherwise known in the art. Generally, variants are overall very similar, and, in many regions, identical to the amino acid sequence of the therapeutic protein corresponding to a therapeutic protein portion of an alpha-fetoprotein fusion protein, alpha-fetoprotein protein corresponding to an alpha-fetoprotein protein portion of an alpha-fetoprotein fusion protein, and/or alpha-fetoprotein fusion protein. Nucleic acids encoding these variants are also encompassed by the invention.

The invention is also directed to proteins which comprise an amino acid sequence which is at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% identical to, for example, (i) the amino acid sequence of a therapeutic protein corresponding to a therapeutic protein portion of an alpha-fetoprotein fusion protein of the invention (e.g., the amino acid sequence of a therapeutic protein, or the amino acid sequence of a therapeutic protein portion of an alpha-fetoprotein fusion protein encoded by a polynucleotide or alpha-fetoprotein fusion construct, or fragments or variants thereof), (ii) alpha-fetoprotein proteins corresponding to an alpha-fetoprotein protein portion of an alpha-fetoprotein fusion protein of the invention, and/or (iii) alpha-fetoprotein fusion proteins. Fragments of these polypeptides are also provided (e.g., those fragments described herein). A variant may have at least one functional activity of the reference polypeptide.

Further polypeptides encompassed by the invention are polypeptides encoded by polynucleotides that hybridize to the complement of a nucleic acid molecule encoding an alpha-fetoprotein fusion protein of the invention under stringent hybridization conditions (e.g., hybridization to filter bound DNA in 6× Sodium chloride/Sodium citrate (SSC) at about 45° Celsius, followed by one or more washes in 0.2×SSC, 0.1% SDS at about 50-65° Celsius), under highly stringent conditions (e.g., hybridization to filter bound DNA in 6× sodium chloride/Sodium citrate (SSC) at about 45° Celsius, followed by one or more washes in 0.1×SSC, 0.2% SDS at about 68° Celsius), or under other stringent hybridization conditions which are known to those of skill in the art (see Ausubel et al., eds., Current Protocol in Molecular Biology, pages 6.3.1-6.3.6 and 2.10.3 (Green publishing associates, Inc., and John Wiley & Sons Inc., New York, 1989). Polynucleotides encoding these polypeptides are also encompassed by the invention.

By a polypeptide having an amino acid sequence at least about, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 60%, is at least about 65%, is at least about 70%, is at least about 75%, is at least about 80%, at least about 85%, at least about 90%, at least about 95%, at least about 96%, at least about 97%, at least about 98%, or at least about 99% identical to, for instance, the amino acid sequence of an alpha-fetoprotein fusion protein of the invention or a fragment thereof (such as a therapeutic protein portion of the alpha-fetoprotein fusion protein or an alpha-fetoprotein portion of the alpha-fetoprotein fusion protein), can be determined conventionally using known computer programs.

A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci., 6:237-245 (1990)). In a sequence alignment the query and subject sequences are either both nucleotide sequences or both amino acid sequences. The result of the global sequence alignment is expressed as percent identity. Preferred parameters used in a FASTDB amino acid alignment are: Matrix=PAM 0, k-tuple=2, Mismatch Penalty=11, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The variant will usually have at least about 60%, at least about 65%, at least about 7.0%, at least about 75%, at least about 80%, at least about 90%, at least about 95% or at least about 99% sequence identity with a length of normal alpha-fetoprotein or therapeutic protein which is the same length as the variant. Homology or identity at the nucleotide or amino acid sequence level is determined by BLAST (Basic Local Alignment Search Tool) analysis using the algorithm employed by the programs blastp, blastn, blastx, tblastn and tblastx (Karlin et al., Proc. Natl. Acad. Sci. USA, 87: 2264-2268 (1990) and Altschul, J. Mol. Evol, 36: 290-300 (1993), incorporated by reference) which are tailored for sequence similarity searching.

The approach used by the BLAST program is to first consider similar segments between a query sequence and a database sequence, then to evaluate the statistical significance of all matches that are identified, and finally to summarize only those matches which satisfy a preselected threshold of significance. For a discussion of basic issues in similarity searching of sequence databases, see Altschul et al., Nature Genetics, 6: 119-129 (1994), incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA, 89: 10915-10919 (1992), incorporated by reference). For blastn, the scoring matrix is set by the ratios of M (i.e., the reward score for a pair of matching residues) to N (i.e., the penalty score for mismatching residues), wherein the default values for M and N are 5 and 4, respectively. Four blastn parameters may be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The polynucleotide variants of the invention may comprise alterations in the coding regions, non-coding regions, or both. Especially preferred are polynucleotide variants comprising alterations which produce silent substitutions, additions, or deletions, but do not alter the properties or activities of the encoded polypeptide. Nucleotide variants produced by silent substitutions due to the degeneracy of the genetic code are preferred. Moreover, polypeptide variants in which less than about 50, less than about 45, less than about 40, less than about 35, less than about 30, less than about 25, less than about 20, less than about 15, less than about 10, less than about 9, less than about 8, less than about 7, less than about 6, less than about 5, less than about 4, less than about 3, less than about 2, or less than about 1 amino acids are substituted, deleted, or added in any combination are also preferred. Polynucleotide variants can be produced for a variety of reasons, e.g., to optimize codon expression for a particular host (change codons in the human mRNA to those preferred by a bacterial host, such as, yeast or E. coli).

In an additional embodiment, a polynucleotide which encodes a therapeutic protein portion of an alpha-fetoprotein fusion protein does not comprise the naturally occurring sequence of that therapeutic protein. In a further embodiment, a polynucleotide which encodes an alpha-fetoprotein protein portion of an alpha-fetoprotein fusion protein does not comprise the naturally occurring sequence of alpha-fetoprotein protein. In an alternative embodiment, a polynucleotide which encodes an alpha-fetoprotein fusion protein does not comprise the naturally occurring sequence of a therapeutic protein portion or the alpha-fetoprotein protein portion.

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the invention. For instance, one or more amino acids can be deleted from the N-terminus and/or C-terminus of the polypeptide of the invention without substantial loss of biological function. As an example, Ron et al. (J. Biol. Chem., 268: 2984-2988 (1993)) reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology, 7:199-216 (1988)).

Moreover, ample evidence demonstrates that variants often retain a biological activity similar to that of the naturally occurring protein. For example, Gayle et al. (J. Biol. Chem., 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” In fact, only 23 unique amino acid sequences, out of more than 3,500 nucleotide sequences examined, produced a protein that significantly differed in activity from wild-type.

Furthermore, even if deleting one or more amino acids from the N-terminus and/or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which have a functional activity (e.g., biological activity and/or therapeutic activity). In one embodiment, the invention provides variants of alpha-fetoprotein fusion proteins that have a functional activity (e.g., biological activity and/or therapeutic activity) that corresponds to one or more biological and/or therapeutic activities of the therapeutic protein corresponding to the therapeutic protein portion of the alpha-fetoprotein fusion protein. In another embodiment, the invention provides variants of alpha-fetoprotein fusion proteins that have a functional activity (e.g., biological activity and/or therapeutic activity) that corresponds to one or more biological and/or therapeutic activities of the therapeutic protein corresponding to the therapeutic protein portion of the alpha-fetoprotein fusion protein. Such variants include deletions, insertions, inversions, repeats, and substitutions selected according to general rules known in the art so as have little effect on activity. Polynucleotides encoding such variants are also encompassed by the invention.

In preferred embodiments, the variants of the invention have conservative substitutions. By “conservative substitutions” is intended swaps within groups such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and H is; replacement of the aromatic residues Phe, Tyr, and Trp, and replacement of the small-sized amino acids Ala, Ser, Thr, Met, and Gly.

Guidance concerning how to make phenotypically silent amino acid substitutions is provided, for example, in Bowie et al., “Deciphering the Message in Protein Sequences: Tolerance to Amino Acid Substitutions,” Science, 247:1306-1310 (1990), wherein the authors indicate that there are two main strategies for studying the tolerance of an amino acid sequence to change. The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein. The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. See Cunningham and Wells, Science, 244:1081-1085 (1989). The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu, and Ile; replacement of the hydroxyl residues Ser and Thr; replacement of the acidic residues Asp and Glu; replacement of the amide residues Asn and Gln, replacement of the basic residues Lys, Arg, and H is; replacement of the aromatic residues Phe, Tyr, and Trp, and replacement of the small-sized amino acids Ala, Ser, Thr, Met, and Gly. Besides conservative amino acid substitution, variants of the present invention include: (i) polypeptides comprising substitutions of one or more of the non-conserved amino acid residues, where the substituted amino acid residues may or may not be one encoded by the genetic code, or (ii) polypeptides comprising substitutions of one or more of the amino acid residues having a substituent group, or (iii) polypeptides which have been fused with or chemically conjugated to another compound, such as a compound to increase the stability and/or solubility of the polypeptide (for example, polyethylene glycol), or (iv) polypeptides comprising additional amino acids, such as, for example, an IgG Fc fusion region peptide. Such variant polypeptides are deemed to be within the scope of those skilled in the art from the teachings herein.

For example, polypeptide variants comprising amino acid substitutions of charged amino acids with other charged or neutral amino acids may produce proteins with improved characteristics, such as less aggregation. Aggregation of pharmaceutical formulations reduces activity and increases clearance due to the aggregate's immunogenic activity. See Pinckard et al., Clin. Exp. Immunol., 2:331-340 (1967); Robbins et al., Diabetes, 36:838-845 (1987); and Cleland et al., Crit. Rev. therapeutic Drug Carrier Systems, 10:307-377 (1993).

In specific embodiments, the polypeptides of the invention comprise fragments or variants of the amino acid sequence of an alpha-fetoprotein fusion protein, the amino acid sequence of a therapeutic protein and/or alpha-fetoprotein, wherein the fragments or variants have about 1, about 2 or less, about 3 or less, about 4 or less, about 5 or less, about 10 or less, about 15 or less, about 20 or less, about 25 or less, about 30 or less, about 35 or less, about 40 or less, about 45 or less, about 50 or less, about 55 or less, about 60 or less, about 65 or less, about 70 or less, about 75 or less, about 80 or less, about 85 or less, about 90 or less, about 95 or less, about 100 or less, about 105 or less, about 110 or less, about 115 or less, about 120 or less, about 125 or less, about 130 or less, about 135 or less, about 140 or less, about 145 or less, or about 150 or less amino acid residue additions, substitutions, and/or deletions when compared to the reference amino acid sequence. In preferred embodiments, the amino acid substitutions are conservative. Nucleic acids encoding these polypeptides are also encompassed by the invention.

2. Functional Activity:

“A polypeptide having functional activity” refers to a polypeptide capable of displaying one or more known functional activities associated with the full-length, pro-protein, and/or mature form of a therapeutic protein. Such functional activities include, but are not limited to, biological activity, antigenicity [ability to bind (or compete with a polypeptide for binding) to an anti-polypeptide antibody], immunogenicity (ability to generate antibody which binds to a specific polypeptide of the invention), ability to form multimers with polypeptides of the invention, and ability to bind to a receptor or ligand for a polypeptide (and optionally activate or inhibitor receptor activity, e.g., G-protein couple receptor activity).

“A polypeptide having biological activity” refers to a polypeptide exhibiting activity similar to, but not necessarily identical to, an activity of a therapeutic protein of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the invention (i.e., the candidate polypeptide will exhibit greater activity or not more than about 25-fold less, not more than about tenfold less activity, or not more than about three-fold less activity relative to the polypeptide of the invention).

“A vaccine antigen” refers to a compound capable of eliciting an immune response.

In preferred embodiments, an alpha-fetoprotein fusion protein of the invention has at least one biological and/or therapeutic activity associated with the therapeutic protein portion (or fragment or variant thereof) when it is not fused to alpha-fetoprotein.

The alpha-fetoprotein fusion proteins of the invention can be assayed for functional activity (e.g., biological activity) using or routinely modifying assays known in the art, as well as assays described herein. Additionally, one of skill in the art may routinely assay fragments of a therapeutic protein corresponding to a therapeutic protein portion of an alpha-fetoprotein fusion protein, for activity using assays. Further, one of skill in the art may routinely assay fragments of an alpha-fetoprotein protein corresponding to an alpha-fetoprotein protein portion of an alpha-fetoprotein fusion protein, for activity using assays known in the art and/or as described below.

For example, in one embodiment where one is assaying for the ability of an alpha-fetoprotein fusion protein to bind or compete with a therapeutic protein for binding to an anti-therapeutic polypeptide antibody and/or anti-alpha-fetoprotein antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the invention.

In a preferred embodiment, where a binding partner (e.g., a receptor or a ligand) of a therapeutic protein is identified, binding to that binding partner by an alpha-fetoprotein fusion protein which comprises that therapeutic protein as the therapeutic protein portion of the fusion can be assayed, e.g., by means well-known in the art, such as, for example, reducing and non-reducing gel chromatography, protein affinity chromatography, and affinity blotting. See generally, Phizicky et al., Microbiol. Rev., 59:94-123 (1995). In another embodiment, the ability of physiological correlates of an alpha-fetoprotein fusion protein to bind to a substrate(s) of the therapeutic polypeptide corresponding to the therapeutic protein portion of the fusion can be routinely assayed using techniques known in the art.

In an alternative embodiment, where the ability of an alpha-fetoprotein fusion protein to multimerize is being evaluated, association with other components of the multimer can be assayed, e.g., by means well-known in the art, such as, for example, reducing and non-reducing gel chromatography, protein affinity chromatography, and affinity blotting. See generally, Phizicky et al., supra.

In exemplary embodiments, an alpha-fetoprotein fusion protein comprising all or a portion of an antibody that binds a therapeutic protein, has at least one biological and/or therapeutic activity (e.g., to specifically bind a polypeptide or epitope) associated with the antibody that binds a therapeutic protein (or fragment or variant thereof) when it is not fused to alpha-fetoprotein. In other embodiments, the biological activity and/or therapeutic activity of an alpha-fetoprotein fusion protein comprising all or a portion of an antibody that binds a therapeutic protein is the inhibition (i.e., antagonism) or activation (i.e., agonism) of one or more of the biological activities and/or therapeutic activities associated with the polypeptide that is specifically bound by antibody that binds a therapeutic protein.

Alpha-fetoprotein fusion proteins comprising at least a fragment or variant of an antibody that binds a therapeutic protein may be characterized in a variety of ways. In particular, alpha-fetoprotein fusion proteins comprising at least a fragment or variant of an antibody that binds a therapeutic protein may be assayed for the ability to specifically bind to the same antigens specifically bound by the antibody that binds a therapeutic protein corresponding to the therapeutic protein portion of the alpha-fetoprotein fusion protein using techniques described herein or routinely modifying techniques known in the art.

Assays for the ability of the alpha-fetoprotein fusion proteins (e.g., comprising at least a fragment or variant of an antibody that binds a therapeutic protein) to (specifically) bind a specific protein or epitope may be performed in solution (e.g. Houghten, Bio/Techniques, 13:412-421(1992)), on beads (e.g., Lam, Nature, 354:82-84 (1991)), on chips (e.g., Fodor, Nature, 364:555-556 (1993)), on bacteria (e.g., U.S. Pat. No. 5,223,409), on spores (e.g., U.S. Pat. Nos. 5,571,698; 5,403,484; and 5,223,409), on plasmids (e.g., Cull et al., Proc. Natl. Acad. Sci. USA, 89:1865-1869 (1992)) or on phage (e.g., Scott and Smith, Science, 249:386-390 (1990); Devlin, Science, 249:404-406 (1990); Cwirla et al., Proc. Natl. Acad. Sci. USA, 87:6378-6382 (1990); and Felici, J. Mol. Biol., 222:301-310 (1991)) (each of these references is incorporated herein). Alpha-fetoprotein fusion proteins comprising at least a fragment or variant of a therapeutic antibody may also be assayed for their specificity and affinity for a specific protein or epitope using or routinely modifying techniques described herein or otherwise known in the art.

The alpha-fetoprotein fusion proteins comprising at least a fragment or variant of an antibody that binds a therapeutic protein may be assayed for cross-reactivity with other antigens (e.g., molecules that have sequence and/or structure conservation with the molecule(s) specifically bound by the antibody that binds a therapeutic protein (or fragment or variant thereof) corresponding to the therapeutic protein portion of the alpha-fetoprotein fusion protein of the invention) by any method known in the art. Immunoassays that can be used to analyze (immunospecific) binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g. Ausubel et al, eds, Current Protocols in Molecular Biology, Vol. 1 (John Wiley & Sons, Inc., New York, 1994), which is incorporated by reference). Exemplary immunoassays are described briefly below, but are not intended by way of limitation.

Immunoprecipitation protocols generally comprise lysing a population of cells in a lysis buffer such as RIPA buffer (1% NP40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the alpha-fetoprotein fusion protein of the invention to the cell lysate, incubating for a period of time (e.g., about 1 to about 4 hours) at 40° C., adding sepharose beads coupled to an anti-alpha-fetoprotein antibody, for example, to the cell lysate, incubating for about an hour or more at 40° C., washing the beads in lysis buffer and resuspending the beads in SDS/sample buffer. The ability of the alpha-fetoprotein fusion protein to immunoprecipitate a particular antigen can be assessed by, e.g., Western blot analysis. One of skill in the art would be knowledgeable as to the parameters that can be modified to increase the binding of the alpha-fetoprotein fusion protein to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, at 10.16.1 (John Wiley & Sons, Inc., New York, 1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween® 20), applying the alpha-fetoprotein fusion protein of the invention (diluted in blocking buffer) to the membrane, washing the membrane in washing buffer, applying a secondary antibody (which recognizes the alpha-fetoprotein fusion protein, e.g., an anti-alpha-fetoprotein antibody) conjugated to an enzymatic substrate (e.g., horseradish peroxidase or alkaline phosphatase) or radioactive molecule (e.g., ³²P or ¹²⁵I diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g. Ausubel et al, eds, Current Protocols in Molecular Biology, Vol. 1, at 10.8.1 (John Wiley & Sons, Inc., New York, 1994).

ELISAs comprise preparing antigen, coating the well of a 96-well microtiter plate with the antigen, washing away antigen that did not bind the wells, adding the alpha-fetoprotein fusion protein (e.g., comprising at least a fragment or variant of an antibody that binds a therapeutic protein) of the invention conjugated to a detectable compound such as an enzymatic substrate (e.g., horseradish peroxidase or alkaline phosphatase) to the wells and incubating for a period of time, washing away unbound or non-specifically bound alpha-fetoprotein fusion proteins, and detecting the presence of the alpha-fetoprotein fusion proteins specifically bound to the antigen coating the well. In ELISAs the alpha-fetoprotein fusion protein does not have to be conjugated to a detectable compound; instead, a second antibody (which recognizes alpha-fetoprotein fusion protein) conjugated to a detectable compound may be added to the well. Further, instead of coating the well with the antigen, the alpha-fetoprotein fusion protein may be coated to the well. In this case, the detectable molecule could be the antigen conjugated to a detectable compound such as an enzymatic substrate (e.g., horseradish peroxidase or alkaline phosphatase). One of skill in the art would be knowledgeable as to the parameters that can be modified to increase the signal detected as well as other variations of ELISAs known in the art. For further discussion regarding ELISAs see, e.g., Ausubel et al, eds, Current Protocols in Molecular Biology, Vol. 1, at 11.2.1 (John Wiley & Sons, Inc., New York, 1994).

The binding affinity of an alpha-fetoprotein fusion protein to a protein, antigen, or epitope and the off-rate of an alpha-fetoprotein fusion protein-protein antigen/epitope interaction can be determined by competitive binding assays. One example of a competitive binding assay is a radioimmunoassay comprising the incubation of labeled antigen with the alpha-fetoprotein fusion protein of the invention in the presence of increasing amounts of unlabeled antigen, and the detection of the antibody bound to the labeled antigen. The affinity of the alpha-fetoprotein fusion protein for a specific protein, antigen, or epitope and the binding off-rates can be determined from the data by Scatchard plot analysis. Competition with a second protein that binds the same protein, antigen or epitope as the alpha-fetoprotein fusion protein, can also be determined using radioimmunoassays. In this case, the protein, antigen or epitope is incubated with an alpha-fetoprotein fusion protein conjugated to a labeled compound in the presence of increasing amounts of an unlabeled second protein that binds the same protein, antigen, or epitope as the alpha-fetoprotein fusion protein of the invention.

In an exemplary embodiment, BIAcore kinetic analysis is used to determine the binding on and off rates of alpha-fetoprotein fusion proteins of the invention to a protein, antigen or epitope. BIAcore kinetic analysis comprises analyzing the binding and dissociation of alpha-fetoprotein fusion proteins, or specific polypeptides, antigens or epitopes from chips with immobilized specific polypeptides, antigens or epitopes or alpha-fetoprotein fusion proteins, respectively, on their surface.

Antibodies that bind a therapeutic protein corresponding to the therapeutic protein portion of an alpha-fetoprotein fusion protein may also be described or specified in terms of their binding affinity for a given protein or antigen, preferably the antigen which they specifically bind. Exemplary binding affinities include those with a dissociation constant or Kd less than about 5×10⁻² M, less than about 10⁻² M, less than about 5×10⁻³ M, less than about 10⁻³ M, less than about 5×10⁻⁴ M, or less than about 10⁻⁴ M. Additional exemplary binding affinities include those with a dissociation constant or Kd less than about 5×10⁻⁵ M, less than about 10⁻⁵ M, less than about 5×10⁻⁶ M, less than about 10⁻⁶ M, less than about 5×10⁻⁷ M, less than about 10⁻⁷ M, less than about 5×10⁻⁸ M, less than about 10⁻⁸ M, less than about 5×10⁻⁹ M, less than about 10⁻⁹ M, less than about 5×10⁻¹⁰ M, less than about 10⁻¹⁰ M, less than about 5×10⁻¹¹ M, less than about 10⁻¹¹ M, less than about 5×10⁻¹² M, less than about 10⁻¹² M, less than about 5×10⁻¹³ M, less than about 10⁻¹³ M, less than about 5×10⁻¹⁴ M, less than about 10⁻¹⁴ M, less than about 5×10⁻¹⁵ M, or less than about 10⁻¹⁵ M. In exemplary embodiments, alpha-fetoprotein fusion proteins comprising at least a fragment or variant of an antibody that binds a therapeutic protein, has an affinity for a given protein or epitope similar to that of the corresponding antibody (not fused to alpha-fetoprotein) that binds a therapeutic protein, taking into account the valency of the alpha-fetoprotein fusion protein (comprising at least a fragment or variant of an antibody that binds a therapeutic protein) and the valency of the corresponding antibody. In addition, assays described herein and otherwise known in the art may routinely be applied to measure the ability of alpha-fetoprotein fusion proteins and fragments, variants and derivatives thereof to elicit biological activity and/or therapeutic activity (either in vitro or in vivo) related to either the therapeutic protein portion and/or alpha-fetoprotein portion of the alpha-fetoprotein fusion protein. Other methods will be known to the skilled artisan and are within the scope of the invention.

C. Antibodies

Antibodies that specifically bind therapeutic proteins are also considered therapeutic proteins and vaccine antigens.

The invention also encompasses alpha-fetoprotein fusion proteins that comprise at least a fragment or variant of an antibody that specifically binds a therapeutic protein and/or vaccine antigen. It is specifically contemplated that the term “therapeutic protein” encompasses antibodies that bind a therapeutic protein and fragments and variants thereof. Thus an alpha-fetoprotein fusion protein of the invention may comprise at least a fragment or variant of a therapeutic protein, and/or at least a fragment or variant of an antibody that binds a therapeutic protein.

Antibodies that bind to a therapeutic protein and that may correspond to a therapeutic protein portion of an alpha-fetoprotein fusion protein of the invention may be used, for example, to purify, detect, and target therapeutic proteins, including both in vitro and in vivo diagnostic and therapeutic methods. For example, the antibodies have utility in immunoassays for qualitatively and quantitatively measuring levels of the therapeutic protein in biological samples. See, e.g., Harlow et al., Antibodies: A Laboratory Manual (Cold Spring Harbor Laboratory Press, 2nd ed. 1988); incorporated by reference. Likewise, alpha-fetoprotein fusion proteins comprising at least a fragment or variant of an antibody that binds a therapeutic protein may be used, for example, to purify, detect, and target therapeutic proteins, including both in vitro and in vivo diagnostic and therapeutic methods.

Antibodies that bind to a therapeutic protein and that may correspond to a therapeutic protein portion of an alpha-fetoprotein fusion protein include derivatives that are modified, i.e., by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids. Alpha-fetoprotein fusion proteins of the invention may also be modified as described above.

1. Antibody Structure and Background

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. See generally, Fundamental Immunology, Chapters 3-5 (Paul, W., ed., 4th ed. Raven Press, N.Y. (1998)) (incorporated by reference). The variable regions of each light/heavy chain pair form the antibody binding site. Thus, an intact IgG antibody has two binding sites. Except in bifunctional or bispecific antibodies, the two binding sites are the same.

The chains all exhibit the same general structure of relatively conserved framework regions (FR) joined by three hypervariable regions, also called complementarity determining regions or CDRs. The CDR regions, in general, are the portions of the antibody which make contact with the antigen and determine its specificity. The CDRs from the heavy and the light chains of each pair are aligned by the framework regions, enabling binding to a specific epitope. From N-terminal to C-terminal, both light and heavy chains variable regions comprise the domains FR1, CDR1, FR2, CDR2, FR3, CDR3 and FR4. The variable regions are connected to the heavy or light chain constant region. The assignment of amino acids to each domain is in accordance with the definitions of Kabat Sequences of proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989)).

As used herein, “antibody” refers to immunoglobulin molecules and immunologically active portions of immunoglobulin molecules, i.e., molecules that comprise an antigen binding site that specifically binds an antigen (e.g., a molecule containing one or more CDR regions of an antibody). Antibodies that may correspond to a therapeutic protein portion of an alpha-fetoprotein fusion protein include, but are not limited to, monoclonal, multispecific, human, humanized or chimeric antibodies, single chain antibodies (e.g., single chain Fvs), Fab fragments, F(ab′) fragments, fragments produced by a Fab expression library, anti-idiotypic (anti-Id) antibodies (including, e.g., anti-Id antibodies specific to antibodies of the invention), and epitope-binding fragments of any of the above (e.g., VH domains, VL domains, or one or more CDR regions).

The present invention encompasses alpha-fetoprotein fusion proteins that comprise at least a fragment or variant of an antibody that binds a therapeutic protein or fragment or variant thereof.

Antibodies that bind a therapeutic protein (or fragment or variant thereof) may be from any animal origin, including birds and mammals. Preferably, the antibodies are human, murine (e.g., mouse and rat), donkey, sheep, rabbit, goat, guinea pig, camel, horse, or chicken antibodies. Most preferably, the antibodies are human antibodies. As used herein, “human” antibodies include antibodies having the amino acid sequence of a human immunoglobulin and include antibodies isolated from human immunoglobulin libraries and xenomice or other organisms that have been genetically engineered to produce human antibodies.

The antibody molecules that bind to a therapeutic protein and that may correspond to a therapeutic protein portion of an alpha-fetoprotein fusion protein of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In preferred embodiments, the antibody molecules that bind to a therapeutic protein and that may correspond to a therapeutic protein portion of an alpha-fetoprotein fusion protein are IgG1. In other preferred embodiments, the immunoglobulin molecules that bind to a therapeutic protein and that may correspond to a therapeutic protein portion of an alpha-fetoprotein fusion protein are IgG2. In other preferred embodiments, the immunoglobulin molecules that bind to a therapeutic protein and that may correspond to a therapeutic protein portion of an alpha-fetoprotein fusion protein are IgG4.

Most preferably the antibodies that bind to a therapeutic protein and that may correspond to a therapeutic protein portion of an alpha-fetoprotein fusion protein are human antigen-binding antibody fragments of the present invention and include, but are not limited to, Fab, Fab′ and F(ab)₂, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains.

The antibodies that bind to a therapeutic protein and that may correspond to a therapeutic protein portion of an alpha-fetoprotein fusion protein may be monospecific, bispecific, trispecific or of greater multispecificity. Multispecific antibodies may be specific for different epitopes of a therapeutic protein or may be specific for both a therapeutic protein as well as for a heterologous epitope, such as a heterologous polypeptide or solid support material. See, WO 93/17715; WO 92/08802; WO 91/00360; WO 92/05793; Tutt, et al., J. Immunol., 147:60-69 (1991); U.S. Pat. Nos. 4,474,893; 4,714,681; 4,925,648; 5,573,920; 5,601,819; Kostelny et al., J. Immunol., 148:1547-1553 (1992).

Antibodies that bind a therapeutic protein (or fragment or variant thereof) may be bispecific or bifunctional which means that the antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547 1553 (1992). In addition, bispecific antibodies may be formed as “diabodies” (Holliger et al. “‘Diabodies’: small bivalent and bispecific antibody fragments” PNAS USA 90:6444-6448 (1993)) or “Janusins” (Traunecker et, al. “Bispecific single chain molecules (Janusins) target cytotoxic lymphocytes on HIV infected cells” EMBO J. 10:3655-3659 (1991) and Traunecker et al. “Janusin: new molecular design for bispecific reagents” Int J Cancer Suppl 7:51-52 (1992)).

a. Antibody Fragments and Variants

The present invention also provides alpha-fetoprotein fusion proteins that comprise, fragments or variants (including derivatives) of an antibody described herein or known elsewhere in the art. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than about 50 amino acid substitutions, less than about 40 amino acid substitutions, less than about 30 amino acid substitutions, less than about 25 amino acid substitutions, less than about 20 amino acid substitutions, less than about 15 amino acid substitutions, less than about 10 amino acid substitutions, less than about 5 amino acid substitutions, less than about 4 amino acid substitutions, less than about 3 amino acid substitutions, or less than about 2 amino acid substitutions relative to the reference VH domain, VHCDR1, VHCDR2, VHCDR3, VL domain, VLCDR1, VLCDR2, or VLCDR3. In specific embodiments, the variants encode substitutions of VHCDR3. In a preferred embodiment, the variants have conservative amino acid substitutions at one or more predicted non-essential amino acid residues.

b. Antibody Binding, Specificity, and Inhibition

Antibodies that bind to a therapeutic protein and that may correspond to a therapeutic protein portion of an alpha-fetoprotein fusion protein may be described or specified in terms of the epitope(s) or portion(s) of a therapeutic protein which they recognize or specifically bind. Antibodies which specifically bind a therapeutic protein or a specific epitope of a therapeutic protein may also be excluded. Therefore, the present invention encompasses antibodies that specifically bind therapeutic proteins, and allows for the exclusion of the same. In preferred embodiments, alpha-fetoprotein fusion proteins comprising at least a fragment or variant of an antibody that binds a therapeutic protein, binds the same epitopes as the unfused fragment or variant of that antibody itself.

Antibodies that bind to a therapeutic protein and that may correspond to a therapeutic protein portion of an alpha-fetoprotein fusion protein may also be described or specified in terms of their cross-reactivity. Antibodies that do not bind any other analog, ortholog, or homolog of a therapeutic protein are included. Antibodies that bind polypeptides with at least about 99%, at least about 95%, at least about 90%, at least about 85%, at least about 80%, at least about 75%, at least about 70%, at least about 65%, at least about 60%, at least about 55%, or at least about 50% sequence identity (as calculated using methods known in the art and described herein) to a therapeutic protein are also included in the present invention. In specific embodiments, antibodies that bind to a therapeutic protein and that may correspond to a therapeutic protein portion of an alpha-fetoprotein fusion protein cross-react with murine, rat and/or rabbit homologs of human proteins and the corresponding epitopes thereof. Antibodies that do not bind polypeptides with at least about 99%, less than about 95%, less than about 90%, less than about 85%, less than about 80%, less than about 75%, less than about 70%, less than about 65%, less than about 60%, less than about 55%, or less than about 50% sequence identity (as calculated using methods known in the art and described herein) to a therapeutic protein are also included in the present invention. In a specific embodiment, the above-described cross-reactivity is with respect to any single specific antigenic or immunogenic polypeptide, or combination(s) of about 2, about 3, about 4, about 5, or more of the specific antigenic and/or immunogenic polypeptides disclosed herein. In preferred embodiments, alpha-fetoprotein fusion proteins comprising at least a fragment or variant of an antibody that binds a therapeutic protein, has similar or substantially identical cross reactivity characteristics compared to the fragment or variant of that particular antibody itself.

Further included in the present invention are antibodies which bind polypeptides encoded by polynucleotides which hybridize to a polynucleotide encoding a therapeutic protein under stringent hybridization conditions (as described herein). Antibodies that bind to a therapeutic protein and that may correspond to a therapeutic protein portion of an alpha-fetoprotein fusion protein of the invention may also be described or specified in terms of their binding affinity to a polypeptide of the invention. Exemplary binding affinities include those with a dissociation constant or Kd less than about 5×10⁻² M, less than about 10⁻² M, less than about 5×10⁻³ M, less than about 10⁻³ M, less than about 5×10⁻⁴ M, or less than about 10⁻⁴ M. More exemplary binding affinities include those with a dissociation constant or Kd less than about 5×10⁻⁵ M, less than about 10⁻⁵ M, less than about 5×10⁻⁶ M, less than about 10⁻⁶ M, less than about 5×10⁻⁷ M, less than about 10⁻⁷ M, less than about 5×10⁻⁸ M, or less than about 10⁻⁸ M. Even more exemplary binding affinities include those with a dissociation constant or Kd less than about 5×10⁻⁹ M, less than about 10⁻⁹ M, less than about 5×10⁻¹⁰ M, less than about 10⁻¹⁰ M, less than about 5×10⁻¹¹ M, less than about 10⁻¹¹ M, less than about 5×10⁻¹² M, less than about 10⁻¹² M, less than about 5×10⁻¹³ M, less than about 10⁻¹³ M, less than about 5×10⁻¹⁴ M, less than about 10⁻¹⁴ M, less than about 5×10⁻¹⁵ M, or less than about 10⁻¹⁵ M. In preferred embodiments, alpha-fetoprotein fusion proteins comprising at least a fragment or variant of an antibody that binds a therapeutic protein, has an affinity for a given protein or epitope similar to that of the corresponding antibody (not fused to alpha-fetoprotein) that binds a therapeutic protein, taking into account the valency of the alpha-fetoprotein fusion protein (comprising at least a fragment or variant of an antibody that binds a therapeutic protein) and the valency of the corresponding antibody.

The invention also provides antibodies that competitively inhibit binding of an antibody to an epitope of a therapeutic protein as determined by any method known in the art for determining competitive binding, for example, the immunoassays described herein. In preferred embodiments, the antibody competitively inhibits binding to the epitope by at least about 99%, at least about 95%, at least about 90%, at least about 85%, at least about 80%, at least about 75%, at least about 70%, at least about 60%, or at least about 50%. In preferred embodiments, alpha-fetoprotein fusion proteins comprising at least a fragment or variant of an antibody that binds a therapeutic protein, competitively inhibits binding of a second antibody to an epitope of a therapeutic protein. In other preferred embodiments, alpha-fetoprotein fusion proteins comprising at least a fragment or variant of an antibody that binds a therapeutic protein, competitively inhibits binding of a second antibody to an epitope of a therapeutic protein by at least about 99%, at least about 95%, at least about 90%, at least about 85%, at least about 80%, at least about 75%, at least about 70%, at least about 60%, or at least about 50%.

c. Antibodies Having Agonist or Antagonist properties

Antibodies that bind to a therapeutic protein and that may correspond to a therapeutic protein portion of an alpha-fetoprotein fusion protein of the invention may act as agonists or antagonists of the therapeutic protein. For example, the present invention includes antibodies which disrupt the receptor/ligand interactions with the polypeptides of the invention either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by Western blot analysis (for example, as described supra). In specific embodiments, antibodies are provided that inhibit ligand activity or receptor activity by at least about 99%, at least about 95%, at least about 90%, at least about 85%, at least about 80%, at least about 75%, at least about 70%, at least about 60%, or at least about 50% of the activity in absence of the antibody. In preferred embodiments, alpha-fetoprotein fusion proteins comprising at least a fragment or variant of an antibody that binds a therapeutic protein, has similar or substantially similar characteristics with regard to preventing ligand binding and/or preventing receptor activation compared to an un-fused fragment or variant of the antibody that binds the therapeutic protein.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the therapeutic proteins. The above antibody agonists can be made using methods known in the art. See, e.g., WO 96/40281; U.S. Pat. No. 5,811,097; Deng et al., Blood, 92(6):1981-1988 (1998); Chen et al., Cancer Res., 58(16):3668-3678 (1998); Harrop et al., J. Immunol., 161(4):1786-1794 (1998); Zhu et al., Cancer Res., 58(15):3209-3214 (1998); Yoon et al., J. Immunol., 160(7):3170-3179 (1998); Prat et al., J. Cell. Sci., 111(Pt2):237-247 (1998); Pitard et al., J. Immunol. Methods, 205(2):177-190 (1997); Liautard et al., Cytokine, 9(4):233-241 (1997); Carlson et al., J. Biol. Chem., 272(17): 11295-11301 (1997); Taryman et al., Neuron, 14(4):755-762 (1995); Muller et al., Structure, 6(9):1153-1167 (1998); Bartunek et al., Cytokine, 8(1):14-20 (1996). In preferred embodiments, alpha-fetoprotein fusion proteins comprising at least a fragment or variant of an antibody that binds a therapeutic protein, have similar or substantially identical agonist or antagonist properties as an un-fused fragment or variant of the antibody that binds the therapeutic protein.

2. Methods of Producing Antibodies that Bind Therapeutic Proteins

The antibodies that bind to a therapeutic protein and that may correspond to a therapeutic protein portion of an alpha-fetoprotein fusion protein of the invention may be generated by any suitable method known in the art. Polyclonal antibodies to an antigen-of-interest can be produced by various procedures well known in the art. For example, a therapeutic protein may be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling, et al., in: Monoclonal Antibodies and T-Cell Hybridomas, 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with a therapeutic protein or fragment or variant thereof, an alpha-fetoprotein fusion protein, or a cell expressing such a therapeutic protein or fragment or variant thereof or alpha-fetoprotein fusion protein. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Another well known method for producing both polyclonal and monoclonal human B cell lines is transformation using Epstein Barr Virus (EBV). Protocols for generating EBV-transformed B cell lines are commonly known in the art, such as, for example, the protocol outlined in Chapter 7.22 of Current Protocols in Immunology, Coligan et al., Eds. (John Wiley & Sons, NY, 1994). The source of B cells for transformation is commonly human peripheral blood, but B cells for transformation may also be derived from other sources including, but not limited to, lymph nodes, tonsil, spleen, tumor tissue, and infected tissues. Tissues are generally made into single cell suspensions prior to EBV transformation. Additionally, steps may be taken to either physically remove or inactivate T cells (e.g., by treatment with cyclosporin A) in B cell-containing samples, because T cells from individuals seropositive for anti-EBV antibodies can suppress B cell immortalization by EBV.

In general, the sample containing human B cells is innoculated with EBV, and cultured for 34 weeks. A typical source of EBV is the culture supernatant of the B95-8 cell line (ATCC #VR-1492). Physical signs of EBV transformation can generally be seen towards the end of the 3-4 week culture period. By phase-contrast microscopy, transformed cells may appear large, clear, hairy and tend to aggregate in tight clusters of cells. Initially, EBV lines are generally polyclonal. However, over prolonged periods of cell cultures, EBV lines may become monoclonal or polyclonal as a result of the selective outgrowth of particular B cell clones. Alternatively, polyclonal EBV transformed lines may be subcloned (e.g., by limiting dilution culture) or fused with a suitable fusion partner and plated at limiting dilution to obtain monoclonal B cell lines. Suitable fusion partners for EBV transformed cell lines include mouse myeloma cell lines (e.g., SP2/0, X63-Ag8.653), heteromyeloma cell lines (human×mouse; e.g., SPAM-8, SBC-H20, and CB-F7), and human cell lines (e.g., GM 1500, SKO-007, RPMI 8226, and KR4). Thus, the invention also provides a method of generating polyclonal or monoclonal human antibodies against polypeptides of the invention or fragments thereof, comprising EBV-transformation of human B cells.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

Antibodies and single chain antibody binding sequences derived from phage display and synthetic peptide sequences derived from phage display or other sources can also be used in the fusion proteins of the invention. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make antibodies that bind to a therapeutic protein include those disclosed in Brinkman et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT application No. PCT/GB91/01134; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in WO 92/22324; Mullinax et al., BioTechniques, 12(6):864-869 (1992); and Sawai et al., AJRI, 34:26-34 (1995); and Better et al., Science, 240:1041-1043 (1988).

Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., PNAS, 90:7995-7999 (1993); and Skerra et al., Science, 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See Morrison, Science, 229:1202 (1985); Oi et al., BioTechniques, 4:214 (1986); Gillies et al., J. Immunol. Methods, 125:191-202 (1989); U.S. Pat. Nos. 5,807,715; 4,816,567; and 4,816,397. Humanized antibodies are antibody molecules from non-human species antibody that binds the desired antigen having one or more complementarity determining regions (CDRS) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See U.S. Pat. No. 5,585,089; Riechmann et al., Nature, 332:323 (1988).) Antibodies can be humanized using a variety of techniques known in the art including, for example, CDR-grafting (EP 239,400; WO 91/09967; U.S. Pat. Nos. 5,225,539; 5,530,101; and 5,585,089), veneering or resurfacing (EP 592,106; EP 519,596; Padlan, Molecular Immunology, 28(4/5):489-498 (1991); Studnicka et al., Protein Engineering, 7(6):805-814 (1994); Roguska. et al., PNAS, 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Llonberg and Huszar, Int. Rev. Immunol., 13:65-93 (1995). For a detailed discussion of this technology for producing human antibodies and human monoclonal antibodies and protocols for producing such antibodies, see WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology, 12:899-903 (1988)).

3. Polynucleotides Encoding Antibodies

The invention further provides polynucleotides comprising a nucleotide sequence encoding an antibody and fragments thereof. The invention also encompasses polynucleotides that hybridize under stringent or alternatively, under lower stringency hybridization conditions, e.g., as defined supra, to polynucleotides that encode an antibody, preferably, that specifically binds to a therapeutic protein, and more preferably, an antibody that binds to a polypeptide having the amino acid sequence of a therapeutic protein.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques, 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed. (Cold Spring Harbor Laboratory, Cold Spring Harbor, N.Y., 1990), and Ausubel et al., eds., Current Protocols in Molecular Biology (John Wiley & Sons, NY, 1998)), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see Chothia et al., J. Mol. Biol., 278: 457-479 (1998), for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the invention and within the skill of the art.

In addition, techniques developed for the production of “chimeric antibodies” (Morrison et al., Proc. Natl. Acad. Sci., 81:851-855 (1984); Neuberger et al., Nature, 312:604-608 (1984); Takeda et al., Nature, 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science, 242:423-42 (1988); Huston et al., Proc. Natl. Acad. Sci. USA, 85:5879-5883 (1988); and Ward et al., Nature, 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science, 242:1038-1041 (1988)).

4. Recombinant Expression of Antibodies

Recombinant expression of an antibody, or fragment, derivative or analog thereof, (e.g., a heavy or light chain of an antibody or a single chain antibody), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see WO 86/05807; WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody. Thus, the invention includes host cells containing a polynucleotide encoding an antibody of the invention, or a heavy or light chain thereof, or a single chain antibody, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101 (1986); Cocken et al., Bio/Technology, 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J, 2:1791 (1983)), in which the antibody coding sequence may be ligated individually into the vector in frame with the lac Z coding region so that a fusion protein is produced; pIN vectors (Inouye & Inouye, Nucleic Acids Res., 13:3101-3109 (1985); Van Heeke & Schuster, J. Biol. Chem., 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (see Logan & Shenk, Proc. Natl. Acad. Sci. USA, 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol., 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI 38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell, 11:223 (1977)), hypoxanthine-guanine phosphoribosyltransferase (Szybalska & Szybalski, Proc. Natl. Acad. Sci. USA, 48:202 (1992)), and adenine phosphoribosyltransferase (Lowy et al., Cell, 22:817 (1980)) genes can be employed in tk−, hgprt− or aprt-cells, respectively. Also, antimetabolite resistance can be used as the basis of selection for the following genes: dhfr, which confers resistance to methotrexate (Wigler et al., Proc. Natl. Acad. Sci. USA, 77:357 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Goldspiel et al., Clinical Pharmacy, 12:488-505 (1993); Wu and Wu, Biotherapy, 3:87-95 (1991); Tolstoshev, Ann. Rev. Pharmacol. Toxicol., 32:573-596 (1993); Mulligan, Science, 260:926-932 (1993); Morgan and Anderson, Ann. Rev. Biochem., 62:191-217 (1993); and May, 1993, TIB TECH, 11(5):155-215 (1993)); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Ausubel et al. (eds.), Current Protocols in Molecular Biology (John Wiley & Sons, NY (1993)); Kriegler, Gene Transfer and Expression, A Laboratory Manual (Stockton Press, NY (1990)); and in Chapters 12 and 13, Dracopoli et al. (eds), Current Protocols in Human Genetics (John Wiley & Sons, NY (1994)); Colberre-Garapin et al., J. Mol. Biol., 150:1 (1981).

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol., 3:257 (1983)).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NS0) which are glutamine synthase negative. Glutamine synthase expression systems can also function in glutamine synthase expressing cells (e.g. Chinese Hamster Ovary (CHO) cells) by providing additional inhibitor to prevent the functioning of the endogenous gene. A glutamine synthase expression system and components thereof are detailed in WO87/04462; WO86/05807; WO89/01036; WO89/10404; and WO91/06657. Additionally, glutamine synthase expression vectors that may be used according to the invention are commercially available from suppliers, including, for example Lonza Biologics, Inc. (Portsmouth, N.H.). Expression and production of monoclonal antibodies using a GS expression system in murine myeloma cells is described in Bebbington et al., Bio/technology, 10:169 (1992), and in Biblia and Robinson, Biotechnol. Prog., 11:1 (1995).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 322:52 (1986); Kohler, Proc. Natl. Acad. Sci. USA, 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies that bind to a therapeutic protein and that may correspond to a therapeutic protein portion of an alpha-fetoprotein fusion protein of the invention or fragments thereof can be fused to heterologous polypeptide sequences described herein or otherwise known in the art, to facilitate purification.

5. Modifications of Antibodies

Antibodies that bind a therapeutic protein or fragments or variants can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA, 86:821-824 (1989), for instance, hexa-histidine provides for convenient purification of the fusion protein. Other peptide tags useful for purification include, but are not limited to, the hemagglutinin tag (also called the “HA tag”), which corresponds to an epitope derived from the influenza hemagglutinin protein (Wilson et al., Cell, 37:767 (1984)) and the “flag” tag.

The invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See U.S. Pat. No. 4,741,900 for metal ions which can be conjugated to antibodies for use as diagnostics according to the invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc. Other examples of detectable substances have been described elsewhere herein.

Further, an antibody of the invention may be conjugated to a therapeutic moiety such as a cytotoxin, e.g., a cytostatic or cytocidal agent, a therapeutic agent or a radioactive metal ion, e.g., alpha-emitters such as, for example, ²¹³Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, camustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent, drug moiety, or vaccine antigen is not to be construed as limited to classical chemical therapeutic agents. For example, the drug moiety may be a protein or polypeptide possessing a desired biological activity. Such proteins may include, for example, a toxin such as PA toxin (anthrax), abrin, ricin A, tuberculosis, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM 1 (WO 97/33899), AIM II (97/34911), Fas Ligand (Takahashi et al., Int Immunol., 6:1567-1574 (1994)), VEGI (WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known. See Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies' 84: Biological And Clinical Applications, Pinchera et al. (eds.), pp. 475-506 (1985); “Analysis, Results, And Future Prospective Of The therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody, with or without a therapeutic moiety conjugated to it, administered alone or in combination with cytotoxic factor(s) and/or cytokine(s) can be used as a therapeutic.

6. Antibody-Alpha-Fetoprotein Fusion

Antibodies that bind to a therapeutic protein and that may correspond to a therapeutic protein portion of an alpha-fetoprotein fusion protein of the invention include, but are not limited to, antibodies that bind a therapeutic protein disclosed, or a fragment or variant thereof.

In specific embodiments, the fragment or variant of an antibody that immunospecifcally binds a therapeutic protein and that corresponds to a therapeutic protein portion of an alpha-fetoprotein fusion protein comprises, or alternatively consists of, the VH domain. In other embodiments, the fragment or variant of an antibody that immunospecifcally binds a therapeutic protein and that corresponds to a therapeutic protein portion of an alpha-fetoprotein fusion protein comprises, or alternatively consists of, one, two or three VH CDRs. In other embodiments, the fragment or variant of an antibody that immunospecifcally binds a therapeutic protein and that corresponds to a therapeutic protein portion of an alpha-fetoprotein fusion protein comprises, or alternatively consists of, the VH CDR1. In other embodiments, the fragment or variant of an antibody that immunospecifcally binds a therapeutic protein and that corresponds to a therapeutic protein portion of an alpha-fetoprotein fusion protein comprises, or alternatively consists of, the VH CDR2. In other embodiments, the fragment or variant of an antibody that immunospecifcally binds a therapeutic protein and that corresponds to a therapeutic protein portion of an alpha-fetoprotein fusion protein comprises, or alternatively consists of, the VH CDR3.

In specific embodiments, the fragment or variant of an antibody that immunospecifcally binds a therapeutic protein and that corresponds to a therapeutic protein portion of an alpha-fetoprotein fusion protein comprises, or alternatively consists of, the VL domain. In other embodiments, the fragment or variant of an antibody that immunospecifically binds a therapeutic protein and that corresponds to a therapeutic protein portion of an alpha-fetoprotein fusion protein comprises, or alternatively consists of, one, two or three VL CDRs. In other embodiments, the fragment or variant of an antibody that immunospecifically binds a therapeutic protein and that corresponds to a therapeutic protein portion of an alpha-fetoprotein fusion protein comprises, or alternatively consists of, the VL CDR1. In other embodiments, the fragment or variant of an antibody that immunospecifcally binds a therapeutic protein and that corresponds to a therapeutic protein portion of an alpha-fetoprotein fusion protein comprises, or alternatively consists of, the VL CDR2. In other embodiments, the fragment or variant of an antibody that immunospecifcally binds a therapeutic protein and that corresponds to a therapeutic protein portion of an alpha-fetoprotein fusion protein comprises, or alternatively consists of, the VL CDR3.

In other embodiments, the fragment or variant of an antibody that immunospecifcally binds a therapeutic protein and that corresponds to a therapeutic protein portion of an alpha-fetoprotein fusion protein comprises, or alternatively comprises one, two, three, four, five, or six VH and/or VL CDRs.

In preferred embodiments, the fragment or variant of an antibody that immunospecifically binds a therapeutic protein and that corresponds to a therapeutic protein portion of an alpha-fetoprotein fusion protein comprises, or alternatively consists of, an scFv comprising the VH domain of the therapeutic antibody, linked to the VL domain of the therapeutic antibody by a peptide linker such as (Gly₄Ser)₃.

7. Immunophenotyping

The antibodies of the invention or alpha-fetoprotein fusion proteins of the invention comprising at least a fragment or variant of an antibody that binds a therapeutic protein (or fragment or variant thereof) may be utilized for immunophenotyping of cell lines and biological samples. Therapeutic proteins of the invention may be useful as cell-specific markers, or more specifically as cellular markers that are differentially expressed at various stages of differentiation and/or maturation of particular cell types. Monoclonal antibodies (or alpha-fetoprotein fusion proteins comprising at least a fragment or variant of an antibody that binds a therapeutic protein) directed against a specific epitope, or combination of epitopes, will allow for the screening of cellular populations expressing the marker. Various techniques can be utilized using monoclonal antibodies (or alpha-fetoprotein fusion proteins comprising at least a fragment or variant of an antibody that binds a therapeutic protein) to screen for cellular populations expressing the marker(s), and include magnetic separation using antibody-coated magnetic beads, “panning” with antibody attached to a solid matrix (i.e., plate), and flow cytometry (See U.S. Pat. No. 5,985,660; and Morrison et al., Cell, 96:73749 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

8. Characterizing Antibodies that Bind a Therapeutic Protein and Alpha-Fetoprotein Fusion Proteins Comprising a Fragment or Variant of an Antibody that Binds a Therapeutic Protein

The antibodies of the invention or alpha-fetoprotein fusion proteins of the invention comprising at least a fragment or variant of an antibody that binds a therapeutic protein (or fragment or variant thereof) may be characterized in a variety of ways. In particular, Alpha-fetoprotein fusion proteins of the invention comprising at least a fragment or variant of an antibody that binds a therapeutic protein may be assayed for the ability to specifically bind to the same antigens specifically bound by the antibody that binds a therapeutic protein corresponding to the antibody that binds a therapeutic protein portion of the alpha-fetoprotein fusion protein using techniques described herein or routinely modifying techniques known in the art.

Assays for the ability of the antibodies of the invention or alpha-fetoprotein fusion proteins of the invention comprising at least a fragment or variant of an antibody that binds a therapeutic protein (or fragment or variant thereof) to (specifically) bind a specific protein or epitope may be performed in solution (e.g., Houghten, Bio/Techniques, 13:412-421(1992)), on beads (e.g., Lam, Nature, 354:82-84 (1991)), on chips (e.g., Fodor, Nature, 364:555-556 (1993)), on bacteria (e.g., U.S. Pat. No. 5,223,409), on spores (e.g., U.S. Pat. Nos. 5,571,698; 5,403,484; and 5,223,409), on plasmids (e.g., Cull et al., Proc. Natl. Acad. Sci. USA, 89:1865-1869 (1992)) or on phage (e.g., Scott and Smith, Science, 249:386-390 (1990); Devlin, Science, 249:404-406 (1990); Cwirla et al., Proc. Natl. Acad. Sci. USA, 87:6378-6382 (1990); and Felici, J. Mol. Biol., 222:301-310 (1991)). The antibodies of the invention or alpha-fetoprotein fusion proteins of the invention comprising at least a fragment or variant of an antibody that binds a therapeutic protein (or fragment or variant thereof) may also be assayed for their specificity and affinity for a specific protein or epitope using or routinely modifying techniques described herein or otherwise known in the art.

The alpha-fetoprotein fusion proteins of the invention comprising at least a fragment or variant of an antibody that binds a therapeutic protein may be assayed for cross-reactivity with other antigens (e.g., molecules that have sequence/structure conservation with the molecule(s) specifically bound by the antibody that binds a therapeutic protein (or fragment or variant thereof) corresponding to the therapeutic protein portion of the alpha-fetoprotein fusion protein of the invention) by any method known in the art.

Immunoassays which can be used to analyze (immunospecific) binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, Current Protocols in Molecular Biology, Vol. 1, John Wiley & Sons, Inc., New York, 1994). Exemplary immunoassays are described briefly below (but are not intended by way of limitation).

Immunoprecipitation protocols generally comprise lysing a population of cells in a lysis buffer such as RIPA buffer (1% NP40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding an antibody of the invention or alpha-fetoprotein fusion protein of the invention comprising at least a fragment or variant of an antibody that binds a therapeutic protein (or fragment or variant thereof) to the cell lysate, incubating for a period of time (e.g., 1 to 4 hours) at 40° C., adding protein A and/or protein G sepharose beads (or beads coated with an appropriate anti-idiotypic antibody or anti-alpha-fetoprotein antibody in the case when an alpha-fetoprotein fusion protein comprising at least a fragment or variant of a therapeutic antibody) to the cell lysate, incubating for about an hour or more at 40° C., washing the beads in lysis buffer and resuspending the beads in SDS/sample buffer. The ability of the antibody or alpha-fetoprotein fusion protein of the invention to immunoprecipitate a particular antigen can be assessed by, e.g., western blot analysis. One of skill in the art would be knowledgeable as to the parameters that can be modified to increase the binding of the antibody or alpha-fetoprotein fusion protein to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, at 10.16.1 (John Wiley & Sons, Inc., New York, 1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g. PBS with 3% BSA or non-fat milk), washing the membrane in washing buffer (e.g., PBS-Tween® 20), applying the antibody or alpha-fetoprotein fusion protein of the invention (diluted in blocking buffer) to the membrane, washing the membrane in washing buffer, applying a secondary antibody (which recognizes the alpha-fetoprotein fusion protein, e.g., an anti-human alpha-fetoprotein antibody) conjugated to an enzymatic substrate (e.g., horseradish peroxidase or alkaline phosphatase) or radioactive molecule (e.g., ³²P or ¹²⁵I diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, Current Protocols in Molecular Biology, Vol. 1, at 10.8.1 (John Wiley & Sons, Inc., New York (1994)).

ELISAs comprise preparing antigen, coating the well of a 96-well microtiter plate with the antigen, washing away antigen that did not bind the wells, adding the antibody or alpha-fetoprotein fusion protein (comprising at least a fragment or variant of an antibody that binds a therapeutic protein) of the invention conjugated to a detectable compound such as an enzymatic substrate (e.g., horseradish peroxidase or alkaline phosphatase) to the wells and incubating for a period of time, washing away unbound or non-specifically bound alpha-fetoprotein fusion proteins, and detecting the presence of the antibody or alpha-fetoprotein fusion proteins specifically bound to the antigen coating the well. In ELISAs the antibody or alpha-fetoprotein fusion protein does not have to be conjugated to a detectable compound; instead, a second antibody (which recognizes the antibody or alpha-fetoprotein fusion protein, respectively) conjugated to a detectable compound may be added to the well. Further, instead of coating the well with the antigen, antibody or the alpha-fetoprotein fusion protein may be coated to the well. In this case, the detectable molecule could be the antigen conjugated to a detectable compound such as an enzymatic substrate (e.g., horseradish peroxidase or alkaline phosphatase). One of skill in the art would be knowledgeable as to the parameters that can be modified to increase the signal detected as well as other variations of ELISAs known in the art. For further discussion regarding ELISAs see, e.g., Ausubel et al, eds, Current Protocols in Molecular Biology, Vol. 1, at 11.2.1 (John Wiley & Sons, Inc., New York, 1994).

The binding affinity of an alpha-fetoprotein fusion protein to a protein, antigen, or epitope and the off-rate of an antibody- or alpha-fetoprotein fusion protein-protein/antigen/epitope interaction can be determined by competitive binding assays. One example of a competitive binding assay is a radioimmunoassay comprising the incubation of labeled antigen (e.g. ³H or ¹²⁵I) with the antibody or alpha-fetoprotein fusion protein of the invention in the presence of increasing amounts of unlabeled antigen, and the detection of the antibody bound to the labeled antigen. The affinity of the antibody or alpha-fetoprotein fusion protein of the invention for a specific protein, antigen, or epitope and the binding off-rates can be determined from the data by Scatchard plot analysis. Competition with a second protein that binds the same protein, antigen or epitope as the antibody or alpha-fetoprotein fusion protein, can also be determined using radioimmunoassays. In this case, the protein, antigen or epitope is incubated with an antibody or alpha-fetoprotein fusion protein of the invention conjugated to a labeled compound in the presence of increasing amounts of an unlabeled second protein that binds the same protein, antigen, or epitope as the alpha-fetoprotein fusion protein of the invention.

In a preferred embodiment, BIAcore kinetic analysis is used to determine the binding on and off rates of antibody or alpha-fetoprotein fusion proteins of the invention to a protein, antigen or epitope. BIAcore kinetic analysis comprises analyzing the binding and dissociation of antibodies, alpha-fetoprotein fusion proteins, or specific polypeptides, antigens or epitopes from chips with immobilized specific polypeptides, antigens or epitopes, antibodies or alpha-fetoprotein fusion proteins, respectively, on their surface.

IV. Therapeutic Uses

A. Antibodies

The invention is further directed to antibody-based therapies which involve administering antibodies of the invention or alpha-fetoprotein fusion proteins of the invention comprising at least a fragment or variant of an antibody that binds a therapeutic protein to an animal, preferably a mammal, and most preferably a human, patient for treating one or more of the disclosed diseases, disorders, or conditions. therapeutic compounds of the invention include, but are not limited to, antibodies of the invention (including fragments, analogs and derivatives thereof as described herein), nucleic acids encoding antibodies of the invention (including fragments, analogs and derivatives thereof and anti-idiotypic antibodies as described herein), alpha-fetoprotein fusion proteins of the invention comprising at least a fragment or variant of an antibody that binds a therapeutic protein, and nucleic acids encoding such alpha-fetoprotein fusion proteins. The antibodies of the invention or alpha-fetoprotein fusion proteins of the invention comprising at least a fragment or variant of an antibody that binds a therapeutic protein can be used to treat, inhibit or prevent diseases, disorders or conditions associated with aberrant expression and/or activity of a therapeutic protein, including, but not limited to, any one or more of the diseases, disorders, or conditions described herein. The treatment and/or prevention of diseases, disorders, or conditions associated with aberrant expression and/or activity of a therapeutic protein includes, but is not limited to, alleviating symptoms associated with those diseases, disorders or conditions. antibodies of the invention or alpha-fetoprotein fusion proteins of the invention comprising at least a fragment or variant of an antibody that binds a therapeutic protein may be provided in pharmaceutically acceptable compositions as known in the art or as described herein.

In a specific and preferred embodiment, the invention is directed to antibody-based therapies which involve administering antibodies of the invention or alpha-fetoprotein fusion proteins of the invention comprising at least a fragment or variant of an antibody that binds a therapeutic protein to an animal, preferably a mammal, and most preferably a human, patient for treating one or more diseases, disorders, or conditions, including but not limited to: neural disorders, immune system disorders, muscular disorders, reproductive disorders, gastrointestinal disorders, pulmonary disorders, cardiovascular disorders, renal disorders, proliferative disorders, and/or cancerous diseases and conditions, and/or as described elsewhere herein. Therapeutic compounds of the invention include, but are not limited to, antibodies of the invention (e.g., antibodies directed to the full length protein expressed on the cell surface of a mammalian cell; antibodies directed to an epitope of a therapeutic protein and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a therapeutic protein, including, but not limited to, any one or more of the diseases, disorders, or conditions described herein. The treatment and/or prevention of diseases, disorders, or conditions associated with aberrant expression and/or activity of a therapeutic protein includes, but is not limited to, alleviating symptoms associated with those diseases, disorders or conditions. Antibodies of the invention or alpha-fetoprotein fusion proteins of the invention comprising at least a fragment or variant of an antibody that binds a therapeutic protein may be provided in pharmaceutically acceptable compositions as known in the art or as described herein.

A summary of the ways in which the antibodies of the invention or alpha-fetoprotein fusion proteins of the invention comprising at least a fragment or variant of an antibody that binds a therapeutic protein may be used therapeutically includes binding therapeutic proteins locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the invention or alpha-fetoprotein fusion proteins of the invention comprising at least a fragment or variant of an antibody that binds a therapeutic protein for diagnostic, monitoring or therapeutic purposes without undue experimentation.

The antibodies of the invention or alpha-fetoprotein fusion proteins of the invention comprising at least a fragment or variant of an antibody that binds a therapeutic protein may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention or alpha-fetoprotein fusion proteins of the invention comprising at least a fragment or variant of an antibody that binds a therapeutic protein may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against therapeutic proteins, fragments or regions thereof, (or the alpha-fetoprotein fusion protein correlate of such an antibody) for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Exemplary binding affinities include those with a dissociation constant or Kd less than about 5×10⁻² M, less than about 10⁻² M, less than about 5×10⁻³ M, less than about 10⁻³ M, less than about 5×10⁻⁴ M, or less than about 10⁻⁴ M. Additional exemplary binding affinities include those with a dissociation constant or Kd less than about 5×10⁻⁵ M, less than about 10⁻⁵ M, less than about 5×10⁻⁶ M, less than about 10⁻⁶ M, less than about 5×10⁻⁷ M, less than about 10⁻⁷ M, less than about 5×10⁻⁸ M, less than about 10⁻⁸ M, less than about 5×10⁻⁹ M, less than about 10⁻⁹ M, less than about 5×10⁻¹⁰ M, less than about 10⁻¹⁰ M, less than about 5×10⁻¹¹ M, less than about 10⁻¹¹ M, less than about 5×10⁻¹² M, less than about 10⁻¹² M, less than about 5×10⁻¹³ M, less than about 10⁻¹³ M, less than about 5×10⁻¹⁴ M, less than about 10⁻¹⁴ M, less than about 5×10⁻¹⁵ M, or less than about 10⁻¹⁵ M.

B. Gene Therapy

In a specific embodiment, nucleic acids comprising sequences encoding antibodies that bind therapeutic proteins or alpha-fetoprotein fusion proteins comprising at least a fragment or variant of an antibody that binds a therapeutic protein are administered to treat, inhibit or prevent a disease or disorder associated with aberrant expression and/or activity of a therapeutic protein, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described in more detail elsewhere in this application.

C. Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

D. Therapeutic/Prophylatic Administration and Composition

The invention provides methods of treatment, inhibition and prophylaxis by administration to a subject of an effective amount of a compound or pharmaceutical composition of the invention. In a preferred embodiment, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem., 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science, 249:1527-1533 (1990); Treat et al., in Liposomes in the Therapy of Infectious Disease and Cancer, Lopez-Berestein and Fidler (eds.), pp. 353-365 (Liss, N.Y., 1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref Biomed. Eng., 14:201 (1987); Buchwald et al., Surgery, 88:507 (1980); Saudek et al., N. Engl. J. Med., 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 23:61 (1983); see also Levy et al., Science, 228:190 (1985); During et al., Ann. Neurol., 25:351 (1989); Howard et al., J. Neurosurg., 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA, 88:1864-1868 (1991)), etc. Alternatively, a nucleic acid can be introduced intracellularly and incorporated within host cell DNA for expression, by homologous recombination.

The invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer hAFP-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

E. Diagnosis and Imaging

Labeled antibodies and derivatives and analogs thereof that bind a therapeutic protein (or fragment or variant thereof) (including alpha-fetoprotein fusion proteins comprising at least a fragment or variant of an antibody that binds a therapeutic protein), can be used for diagnostic purposes to detect, diagnose, or monitor diseases, disorders, and/or conditions associated with the aberrant expression and/or activity of therapeutic protein. The invention provides for the detection of aberrant expression of a therapeutic protein, comprising (a) assaying the expression of the therapeutic protein in cells or body fluid of an individual using one or more antibodies specific to the polypeptide interest and (b) comparing the level of gene expression with a standard gene expression level, whereby an increase or decrease in the assayed therapeutic protein expression level compared to the standard expression level is indicative of aberrant expression.

The invention provides a diagnostic assay for diagnosing a disorder, comprising (a) assaying the expression of the therapeutic protein in cells or body fluid of an individual using one or more antibodies specific to the therapeutic protein or alpha-fetoprotein fusion proteins comprising at least a fragment of variant of an antibody specific to a therapeutic protein, and (b) comparing the level of gene expression with a standard gene expression level, whereby an increase or decrease in the assayed therapeutic protein gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention or alpha-fetoprotein fusion proteins comprising at least a fragment of variant of an antibody specific to a therapeutic protein can be used to assay protein levels in a biological sample using classical immunohistological methods known to those of skill in the art (e.g., see Jalkanen et al., J. Cell. Biol., 101:976-985 (1985); Jalkanen et al., J. Cell. Biol., 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin.

One facet of the invention is the detection and diagnosis of a disease or disorder associated with aberrant expression of a therapeutic protein in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the therapeutic protein is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the therapeutic protein. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody, antibody fragment, or alpha-fetoprotein fusion protein comprising at least a fragment or variant of an antibody that binds a therapeutic protein will then preferentially accumulate at the location of cells which contain the specific therapeutic protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI). Antibodies that specifically detect the alpha-fetoprotein fusion protein but not alpha-fetoprotein or the therapeutic protein alone are a preferred embodiment. These can be used to detect the alpha-fetoprotein fusion protein as described throughout the specification.

F. Kits

The invention provides kits that can be used in the above methods. In one embodiment, a kit comprises an antibody, preferably a purified antibody, in one or more containers. In a specific embodiment, the kits of the invention comprise a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the invention comprise a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which the polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

V. Alpha-Fetoprotein Fusion Proteins

The invention relates generally to alpha-fetoprotein fusion proteins and methods of treating, preventing, or ameliorating diseases or disorders. As used herein, “alpha-fetoprotein fusion protein” refers to a protein formed by the fusion of at least one molecule of alpha-fetoprotein (or a fragment or variant thereof) to at least one molecule of a therapeutic protein or vaccine antigen (or fragment or variant thereof). An alpha-fetoprotein fusion protein of the invention comprises at least a fragment or variant of a therapeutic protein or vaccine antigen and at least a fragment or variant of alpha-fetoprotein, which are associated with one another, preferably by genetic fusion (i.e., the alpha-fetoprotein fusion protein is generated by translation of a nucleic acid in which a polynucleotide encoding all or a portion of a therapeutic protein or vaccine antigen is joined in-frame with a polynucleotide encoding all or a portion of alpha-fetoprotein) or to one another. The therapeutic protein or vaccine antigen and alpha-fetoprotein protein, once part of the alpha-fetoprotein fusion protein, may each be referred to as a “portion”, “region” or “moiety” of the alpha-fetoprotein fusion protein.

Preferred alpha-fetoprotein fusion proteins of the invention, include, but are not limited to, alpha-fetoprotein fusion proteins encoded by a nucleic acid molecule comprising, or alternatively consisting of, a polynucleotide encoding at least one molecule of alpha-fetoprotein (or a fragment or variant thereof) joined in frame to at least one polynucleotide encoding at least one molecule of a therapeutic protein or vaccine antigen (or fragment or variant thereof); a nucleic acid molecule comprising, or alternatively consisting of, a polynucleotide encoding at least one molecule of alpha-fetoprotein (or a fragment or variant thereof) joined in frame to at least one polynucleotide encoding at least one molecule of a therapeutic protein (or fragment or variant thereof); or a nucleic acid molecule comprising a polynucleotide encoding at least one molecule of alpha-fetoprotein (or a fragment or variant thereof) joined in frame to at least one polynucleotide encoding at least one molecule of a therapeutic protein (or fragment or variant thereof), further comprising, for example, one or more of the following elements: (1) a functional self-replicating vector (including but not limited to, a shuttle vector, an expression vector, an integration vector, and/or a replication system), (2) a region for initiation of transcription (e.g., a promoter region, such as for example, a regulatable or inducible promoter, a constitutive promoter), (3) a region for termination of transcription, (4) a leader sequence, and/or (5) a selectable marker.

In one embodiment, the invention provides an alpha-fetoprotein fusion protein comprising a therapeutic protein or vaccine antigen and an alpha-fetoprotein protein. In other embodiments, the invention provides an alpha-fetoprotein fusion protein comprising a biologically active and/or therapeutically active fragment of a therapeutic protein or vaccine antigen and an alpha-fetoprotein protein. In other embodiments, the invention provides an alpha-fetoprotein fusion protein comprising a biologically active and/or therapeutically active variant of a therapeutic protein or vaccine antigen and an alpha-fetoprotein protein. In preferred embodiments, the alpha-fetoprotein protein component of the alpha-fetoprotein fusion protein is the mature portion of alpha-fetoprotein.

In further embodiments, the invention provides an alpha-fetoprotein fusion protein comprising a therapeutic protein or vaccine antigen, and a biologically active and/or therapeutically active fragment of alpha-fetoprotein. In further embodiments, the invention provides an alpha-fetoprotein fusion protein comprising a therapeutic protein or vaccine antigen and a biologically active and/or therapeutically active variant of alpha-fetoprotein.

In further embodiments, the invention provides an alpha-fetoprotein fusion protein comprising a biologically active and/or therapeutically active fragment or variant of a therapeutic protein or vaccine antigen and a biologically active and/or therapeutically active fragment or variant of alpha-fetoprotein. In preferred embodiments, the invention provides an alpha-fetoprotein fusion protein comprising the mature portion of a therapeutic protein or vaccine antigen and the mature portion of alpha-fetoprotein.

In one embodiment, the alpha-fetoprotein fusion protein comprises alpha-fetoprotein as the N-terminal portion, and a therapeutic protein or vaccine antigen as the C-terminal portion. Alternatively, an alpha-fetoprotein fusion protein comprising alpha-fetoprotein as the C-terminal portion, and a therapeutic protein or vaccine antigen as the N-terminal portion may also be used.

In other embodiments, the alpha-fetoprotein fusion protein has a therapeutic protein or vaccine antigen fused to both the N-terminus and the C-terminus of alpha-fetoprotein. In a first embodiment, the therapeutic proteins fused at the N- and C-termini are the same therapeutic proteins. In an alternative embodiment, the therapeutic proteins fused at the N- and C-termini are different therapeutic proteins. In another preferred embodiment, the therapeutic proteins fused at the N- and C-termini are different therapeutic proteins which may be used to treat or prevent the same or a related disease, disorder, or condition. In another preferred embodiment, the therapeutic proteins fused at the N- and C-termini are different therapeutic proteins which may be used to treat, ameliorate, or prevent diseases or disorders which are known in the art to commonly occur in patients simultaneously, concurrently, or consecutively, or which commonly occur in patients in association with one another. For example, a vaccine antigen may be attached to the N or C terminal region of alpha-fetoprotein, and a vaccine adjuvant may be attached to the other end of alpha-fetoprotein.

Alpha-fetoprotein fusion proteins of the invention encompass proteins containing one, two, three, four, or more molecules of a given therapeutic protein or variant thereof fused to the N- or C-terminus of an alpha-fetoprotein fusion protein of the invention, and/or to the N- and/or C-terminus of alpha-fetoprotein or variant thereof. Molecules of a given therapeutic protein or variants thereof may be in any number of orientations, including, but not limited to, a ‘head to head’ orientation (e.g., wherein the N-terminus of one molecule of a therapeutic protein is fused to the N-terminus of another molecule of the therapeutic protein), or a ‘head to tail’ orientation (e.g., wherein the C-terminus of one molecule of a therapeutic protein is fused to the N-terminus of another molecule of therapeutic protein).

In one embodiment, one, two, three, or more tandemly oriented therapeutic protein polypeptides (or fragments or variants thereof) are fused to the N- or C-terminus of an alpha-fetoprotein fusion protein of the invention, and/or to the N- and/or C-terminus of alpha-fetoprotein or variant thereof.

Alpha-fetoprotein fusion proteins of the invention further encompass proteins containing one, two, three, four, or more molecules of a given therapeutic protein or variant thereof fused to the N- or C-terminus of an alpha-fetoprotein fusion protein of the invention, and/or to the N- and/or C-terminus of alpha-fetoprotein or variant thereof, wherein the molecules are joined through peptide linkers. Examples include those peptide linkers described in U.S. Pat. No. 5,073,627. Alpha-fetoprotein fusion proteins comprising multiple therapeutic protein polypeptides separated by peptide linkers may be produced using conventional recombinant DNA technology. Linkers are particularly important when fusing a small peptide to the alpha-fetoprotein molecule. The peptide itself can be a linker by fusing tandem copies of the peptide or other known linkers can be used.

Further, alpha-fetoprotein fusion proteins of the invention may also be produced by fusing a therapeutic protein or variants thereof to the N-terminal and/or C-terminal of alpha-fetoprotein or variants thereof in such a way as to allow the formation of intramolecular and/or intermolecular multimeric forms. In one embodiment of the invention, alpha-fetoprotein fusion proteins may be in monomeric or multimeric forms (i.e., dimers, trimers, tetramers and higher multimers). In a further embodiment of the invention, the therapeutic protein portion of an alpha-fetoprotein fusion protein may be in monomeric form or multimeric form (i.e., dimers, trimers, tetramers and higher multimers). In a specific embodiment, the therapeutic protein portion of an alpha-fetoprotein fusion protein is in multimeric form (i.e., dimers, trimers, tetramers and higher multimers), and the alpha-fetoprotein portion is in monomeric form.

In addition to alpha-fetoprotein fusion protein in which the alpha-fetoprotein portion is fused N-terminal and/or C-terminal of the therapeutic protein portion, alpha-fetoprotein fusion proteins of the invention may also be produced by inserting the therapeutic protein or peptide of interest into an internal region of alpha-fetoprotein. For instance, within the protein sequence of the alpha-fetoprotein molecule a number of loops or turns exist between the end and beginning of alpha.-helices, which are stabilized by disulphide bonds.

Peptides to be inserted may be derived from either phage display or synthetic peptide libraries screened for specific biological activity or from the active portions of a molecule with the desired function. Additionally, random peptide libraries may be generated within particular loops or by insertions of randomized peptides into particular loops of the alpha-fetoprotein molecule and in which all possible combinations of amino acids are represented.

Such library(s) could be generated on alpha-fetoprotein or domain fragments of alpha-fetoprotein by one of the following methods: randomized mutation of amino acids within one or more peptide loops of alpha-fetoprotein or alpha-fetoprotein domain fragments. Either one, more or all the residues within a loop could be mutated in this manner; replacement of, or insertion into one or more loops of alpha-fetoprotein or alpha-fetoprotein domain fragments (i.e., internal fusion) of a randomized peptide(s).

The alpha-fetoprotein or alpha-fetoprotein domain fragment may also be made multifunctional by grafting the peptides derived from different screens of different loops against different targets into the same alpha-fetoprotein or alpha-fetoprotein domain fragment.

Generally, the alpha-fetoprotein fusion proteins of the invention may have one alpha-fetoprotein-derived region and one therapeutic protein-derived region. Multiple regions of each protein, however, may be used to make an alpha-fetoprotein fusion protein of the invention. Similarly, more than one therapeutic protein may be used to make an alpha-fetoprotein fusion protein of the invention. For instance, a therapeutic protein may be fused to both the N- and C-terminal ends of the alpha-fetoprotein. In such a configuration, the therapeutic protein portions may be the same or different therapeutic protein molecules. The structure of bifunctional alpha-fetoprotein fusion proteins may be represented as: X-alpha-fetoprotein-Y or Y-alpha-fetoprotein-X.

Bi- or multi-functional alpha-fetoprotein fusion proteins may also be prepared to target the therapeutic protein portion of a fusion to a target organ or cell type via protein or peptide at the opposite terminus of alpha-fetoprotein.

As an alternative to the fusion of known therapeutic molecules, the peptides could be obtained by screening libraries constructed as fusions to the N-, C- or N- and C-termini of alpha-fetoprotein, or domain fragment of alpha-fetoprotein, of typically 6, 8, 12, 20 or 25 or X (where X is an amino acid (aa) and n equals the number of residues) randomized amino acids, and in which all possible combinations of amino acids were represented). A particular advantage of this approach is that the peptides may be selected in situ on the alpha-fetoprotein molecule and the properties of the peptide would therefore be as selected for rather than, potentially, modified as might be the case for a peptide derived by any other method then being attached to alpha-fetoprotein.

Additionally, the alpha-fetoprotein fusion proteins of the invention may include a linker peptide between the fused portions to provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically to yield the growth hormone related moiety. Preferably, the protease is one which is produced naturally by the host, for example the S. cerevisiae protease kex2 or equivalent proteases.

Therefore, as described above, the alpha-fetoprotein fusion proteins of the invention may have the following formula R1-L-R2; R2-L-R1; or R1-L-R2-L-R1, wherein R1 is at least one therapeutic protein, peptide or polypeptide sequence, and not necessarily the same therapeutic protein, L is a linker and R2 is a alpha-fetoprotein sequence.

In preferred embodiments, Alpha-fetoprotein fusion proteins of the invention comprising a therapeutic protein have extended shelf life compared to the shelf life the same therapeutic protein when not fused to alpha-fetoprotein. Shelf-life typically refers to the time period over which the therapeutic activity of a therapeutic protein in solution or in some other storage formulation, is stable without undue loss of therapeutic activity. Many of the therapeutic proteins are highly labile in their unfused state. As described below, the typical shelf-life of these therapeutic proteins is markedly prolonged upon incorporation into the alpha-fetoprotein fusion protein of the invention.

Alpha-fetoprotein fusion proteins of the invention with “prolonged” or “extended” shelf-life exhibit greater therapeutic activity relative to a standard that has been subjected to the same storage and handling conditions. The standard may be the unfused full-length therapeutic protein. When the therapeutic protein portion of the alpha-fetoprotein fusion protein is an analog, a variant, or is otherwise altered or does not include the complete sequence for that protein, the prolongation of therapeutic activity may alternatively be compared to the unfused equivalent of that analog, variant, altered peptide or incomplete sequence. As an example, an alpha-fetoprotein fusion protein of the invention may retain greater than about 100% of the therapeutic activity, or greater than about 105%, about 110%, about 120%, about 130%, about 150% or about 200% of the therapeutic activity of a standard when subjected to the same storage and handling conditions as the standard when compared at a given time point.

Shelf-life may also be assessed in terms of therapeutic activity remaining after storage, normalized to therapeutic activity when storage began. Alpha-fetoprotein fusion proteins of the invention with prolonged or extended shelf-life as exhibited by prolonged or extended therapeutic activity may retain greater than about 50% of the therapeutic activity, about 60%, about 70%, about 80%, or about 90% or more of the therapeutic activity of the equivalent unfused therapeutic protein when subjected to the same conditions.

A. Expression of Fusion Proteins

The alpha-fetoprotein fusion proteins of the invention may be produced as recombinant molecules by secretion from yeast, a microorganism such as a bacterium, or a human or animal cell line. Preferably, the polypeptide is secreted from the host cells.

A particular embodiment of the invention comprises a DNA construct encoding a signal sequence effective for directing secretion in yeast, particularly a yeast-derived signal sequence (especially one which is homologous to the yeast host), and the fused molecule of the first aspect of the invention, there being no yeast-derived pro sequence between the signal and the mature polypeptide.

The Saccharomyces cerevisiae invertase signal is a preferred example of a yeast-derived signal sequence.

The present invention also includes a cell, preferably a yeast cell transformed to express an alpha-fetoprotein fusion protein of the invention. In addition to the transformed host cells themselves, the present invention also contemplates a culture of those cells, preferably a monoclonal (clonally homogeneous) culture, or a culture derived from a monoclonal culture, in a nutrient medium. If the polypeptide is secreted, the medium will comprise the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis, and Pichia pastoris, filamentous fungi (for example Aspergillus), plant cells, animal cells and insect cells.

Preferred yeast strains to be used in the production of alpha-fetoprotein fusion proteins are D88, DXY1 and BXPIO. D88 [leu2-3, leu2-122, can1, pra1, ubc4] is a derivative of parent strain AH22his⁺ (also known as DB1; Sleep et al., Biotechnology, 8:4246 (1990)). BX10 has the following genotype: leu2-3, leu2-122, can1, pra1, ubc4, ura3, yap3:URA3, lys2, hsp150:LYS2, pmt1:URA3. In addition to the mutations isolated in DXY1, this strain also has a knockout of the PMT1 gene and the HSP150 gene. The PMT1 gene is a member of the evolutionarily conserved family of dolichyl-phosphate-D-mannose protein O-mannosyltransferases (Pmts). The transmembrane topology of Pmtlp suggests that it is an integral membrane protein of the endoplasmic reticulum with a role in 0-linked glycosylation.

The desired protein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente, Methods Enzymol., 194, 182 (1990).

Successfully transformed cells, i.e., cells that comprise a DNA construct of the invention, can be identified by well known techniques. For example, cells resulting from the introduction of an expression construct can be grown to produce the desired polypeptide. Cells can be harvested and lysed and their DNA content examined for the presence of the DNA using a method such as that described by Southern, J. Mol. Biol., 98:503 (1975), or Berent et al., Biotech., 3:208 (1985). Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (Ylps) and incorporate the yeast selectable markers HIS3, 7RP1, LEU2 and URA3. Plasmids pRS413-416 are Yeast Centromere plasmids (Ycps).

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase 1, enzymes that remove protruding, gamma-single-stranded termini with their 3′,5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers comprising a variety of restriction endonuclease sites are commercially available from a number of sources including International Biotechnologies Inc, New Haven, Conn., USA.

A desirable way to modify the DNA in accordance with the invention, if, for example, alpha-fetoprotein variants are to be prepared, is to use the polymerase chain reaction as disclosed by Saiki et al., Science, 239:487-491 (1988). In this method the DNA to be enzymatically amplified is flanked by two specific oligonucleotide primers which themselves become incorporated into the amplified DNA. The specific primers may contain restriction endonuclease recognition sites which can be used for cloning into expression vectors using methods known in the art.

Exemplary genera of yeast contemplated to be useful in the practice of the present invention as hosts for expressing the alpha-fetoprotein fusion proteins are Pichia (Hansenula), Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis, and the like. Preferred genera are those selected from the group consisting of Saccharomyces, Schizosaccharomyces, Kluyveromyces, Pichia and Torulaspora. Examples of Saccharomyces spp. are S. cerevisiae, S. italicus and S. rouxii. Examples of Kluyveromyces spp. are K. fragilis, K. lactis and K. marxianus. A suitable Torulaspora species is T. delbrueckii. Examples of Pichia (Hansenula) spp. are P. angusta (formerly H. polymorpha), P. anomala (formerly H. anomala) and P. pastoris. Methods for the transformation of S. cerevisiae are taught generally in EP 251 744, EP 258 067 and WO 90/01063.

Preferred exemplary species of Saccharomyces include S. cerevisia, S. italicus, S. diastaticus, and Zygosaccharomyces rouxii. Preferred exemplary species of Kluyveromyces include K. fragilis and K. lactis. Preferred exemplary species of Hansenula include H. polymorpha (now Pichia angusta), H. anomala (now Pichia anomala), and Pichia capsulata. Additional preferred exemplary species of Pichia include P. pastoris. Preferred exemplary species of Aspergillus include A. niger and A. nidulans. Preferred exemplary species of Yarrowia include Y. lipolytica. Many preferred yeast species are available from the ATCC. For example, the following preferred yeast species are available from the ATCC and are useful in the expression of alpha-fetoprotein fusion proteins: Saccharomyces cerevisiae Hansen, teleomorph strain BY4743 yap3 mutant (ATCC Accession No. 4022731); Saccharomyces cerevisiae Hansen, teleomorph strain BY4743 hsp15O mutant (ATCC Accession No. 4021266); Saccharomyces cerevisiae Hansen, teleomorph strain BY4743 pmtl mutant (ATCC Accession No. 4023792); Saccharomyces cerevisiae Hansen, teleomorph (ATCC Accession Nos. 20626; 44773; 44774; and 62995); Saccharomyces diastaticus Andrews et Gilliland ex van der Walt, teleomorph (ATCC Accession No. 62987); Kluyveromyces lactis (Dombrowski) van der Walt, teleomorph (ATCC Accession No. 76492); Pichia angusta (Teunisson et al.) Kurtzman, teleomorph deposited as Hansenula polymorpha de Morais et Maia, teleomorph (ATCC Accession No. 26012); Aspergillus niger van Tieghem, anamorph (ATCC Accession No. 9029); Aspergillus niger van Tieghem, anamorph (ATCC Accession No. 16404); Aspergillus nidulans (Eidam) Winter, anamorph (ATCC Accession No. 48756); and Yarrowia lipolytica (Wickerham et al.) van der Walt et von Arx, teleomorph (ATCC Accession No. 201847).

Suitable promoters for S. cerevisiae include those associated with the PGK1 gene, GAL1 or GAL10 genes, CYC1, PHO5, TRP 1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRB1 promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell, J. Biol. Chem., 265:10857-10864 (1990), and the glucose repressible jbpl gene promoter as described by Hoffman & Winston, Genetics, 124:807-816 (1990).

Methods of transforming Pichia for expression of foreign genes are taught in, for example, Cregg et al. (1993), and various Phillips patents (e.g. U.S. Pat. No. 4,857,467), and Pichia expression kits are commercially available from Invitrogen BV, Leek, Netherlands, and Invitrogen Corp., San Diego, Calif. Suitable promoters include AOX₁ and AOX₂. Gleeson et al., J. Gen. Microbiol., 132:3459-3465 (1986), include information on Hansenula vectors and transformation, suitable promoters being MOX₁ and FMD1; while EP 361 991, Fleer et al. (1991) and other-publications from Rhone-Poulenc Rorer teach how to express foreign proteins in Kluyveromyces spp., a suitable promoter being PGKI.

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADH1 gene is preferred.

The desired alpha-fetoprotein fusion protein may be initially expressed with a secretion leader sequence, which may be any leader effective in the yeast chosen. Leaders useful in yeast include any of the following:

a) the MPIF-1 signal sequence (e.g., amino acids 1-21 of GenBank Accession number AAB51134) MKVSVAALSCLMLVTALGSQA

b) the stanniocalcin signal sequence (MLQNSAVLLLLVISASA,

c) the pre-pro region of the AFP signal sequence,

d) the pre region of the AFP signal sequence or variants thereof,

e) the invertase signal sequence (e.g., MLLQAFLFLLAGFAAKISA,

f) the yeast mating factor alpha signal sequence (e.g., MRFPSIFTAVLAFAASSALAAPVNTTTEDETAQIPAEAVIGYSDLEGDFDVAVLPFSNS TNNGLLFINTTIAS-IAAKEEGVSLEKR, or MRFPSHTAVLAFAASSALAAPVNTITEDETAQIPAEAVIGYSD-LEGDFDVAVLPFSNSTNNG LLFINTTIASIAAKEEGVSLDKR,

g) K. lactis killer toxin leader sequence

h) a hybrid signal sequence (e.g., MKWVSFISLLFLFSSAYSRSLEKR,)

i) an AFP/MF alpha-1 hybrid signal sequence (also known as AFP/kex2),

j) a K. lactis killer/MFA-1 fusion leader sequence (e.g., MNIFYIFLFLLSFVQGSLDKR)

k) the Immunoglobulin Ig signal sequence (e.g., MGWSCIILFLVATATGVHS)

l) the Fibulin B precursor signal sequence (e.g., MERAAPSRRVPLPLLLLGGLALLAAGVDA)

m) the clusterin precursor signal sequence (e.g., MMKTLLLFVGLLLTWESGQVLG)

n) the insulin-like growth factor-binding protein 4 signal sequence (e.g., MLPLCLVAALLLAAGPGPSLG)

o) variants of the pre-pro-region of the AFP signal sequence

p) a consensus signal sequence (MPTWAWWLFLVLLLALWAPARG)

q) acid phosphatase (PH05) leader (e.g., MFKSVVYSILAASLANA)

r) the pre-sequence of MFoz-1

s) the pre-sequence of 0 glucanase (BGL2)

t) killer toxin leader

u) the presequence of killer toxin

v) K. lactis killer toxin prepro (29 amino acids; 16 amino acids of pre and 13 amino acids of pro) MNIFYIFLFLLSFVQGLEHTHRRGSLD-KR

w) S. diastaticus glucoamylase 11 secretion leader sequence

x) S. carisbergensis alpha-galactosidase (MEL1) secretion leader sequence

y) Candida glucoamylase leader sequence

z) The hybrid leaders disclosed in EP-A-387 319

aa) the gp67 signal sequence (in conjunction with baculoviral expression systems) (e.g., amino acids 1-19 of GenBank Accession Number AAA72759) or

bb) the natural leader of the therapeutic protein X;

cc) S. cerevisiae invertase (SUC2) leader, as disclosed in JP 62-096086 (granted as 911036516); or

dd) Inulinase—MKLAYSLLLPLAGVSASVINYKR.

ee) A modified TA57 propeptide leader variant #1—MKLKTVRSAVLSSLFASQVLGQPIDDTESQTTSVNLMADDTESA-FATQTNSGGLDVVGLISMAKR

ff) A modified TA57 propeptide leader variant #2—MKLKTVRSAVLSSLFASQVLGQPIDDTESQTTSVNLMADDTESA-FATQTNSGGLDVVGLISMAEEGEPKR

gg) A consensus signal peptide—MWWRLWWLLLLLLLLWPMVWA

hh) MKWVSFISLLFLFSSAYSRSLDKR or

ii) MKWVSFISLLFLFSSAYSGSLDKR.

1. Additional Methods of Recombinant and Synthetic Production of Alpha-Fetoprotein Fusion Proteins

The invention also relates to vectors comprising a polynucleotide encoding an alpha-fetoprotein fusion protein of the invention, host cells, and the production of alpha-fetoprotein fusion proteins by synthetic and recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides encoding alpha-fetoprotein fusion proteins of the invention may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIF5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-SI1pPIC3.5K, pPIC9K, and PAO815 (all available from Invitrogen, Carlsbad, Calif.). Other suitable vectors will be readily apparent to the skilled artisan.

In one embodiment, polynucleotides encoding an alpha-fetoprotein fusion protein of the invention may be fused to signal sequences which will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the alpha-fetoprotein fusion proteins of the invention may be fused to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, polynucleotides alpha-fetoprotein fusion proteins of the invention may be fused to the pelB pectate lyase signal sequence to increase the efficiency of expression and purification of such polypeptides in Gram-negative bacteria. See U.S. Pat. Nos. 5,576,195 and 5,846,818.

Examples of signal peptides that may be fused to an alpha-fetoprotein fusion protein of the invention to direct its secretion in mammalian cells include, but are not limited to:

a) the MPIF-1 signal sequence (e.g., amino acids 1-21 of GenBank Accession number AAB51134) MKVSVAALSCLMLVTALGSQA

b) the stanniocalcin signal sequence (MLQNSAVLLLLVISASA)

c) the pre-pro region of the AFP signal sequence,

d) the pre region of the AFP signal sequence

e) the invertase signal sequence (e.g., MLLQAFLFLLAGFAAKISA)

f) the yeast mating factor alpha signal sequence (e.g., MRFPSIFTAVLAFAASSALAAPVN-TTTEDETAQIPAEAVIGYSDLEGDFDVAVLPFSNSTNNGLLFINTTIASIAAKEEGVSLE KR, or MRFPSIFTAVLAFAASSALAAPVNTTTEDETAQIPAEAVIGYSDLEGDFDVAVLPFSNS TNNGL-LFINTFIASIAAKEEGVSLDKR,)

g) K. lactis killer toxin leader sequence

h) a hybrid signal sequence (e.g., MKWVSFISLLFLFSSAYSRSLEKR)

i) an AFP/MFac-1 hybrid signal sequence (also known as AFPAlkex2)

j) a K. lactis killer/MFa-1 fusion leader sequence (e.g., MNIFYIFLFLLSFVQGSLDKR)

k) the Immunoglobulin Ig signal sequence (e.g., MGWSCIILFLVATATGVHS)

l) the Fibulin B precursor signal sequence (e.g., MERAAPSRRVPLPLLLLGGLALLAAG-VDA)

m) the clusterin precursor signal sequence (e.g., MMKTLLLFVGLLLTWESGQVLG)

n) the insulin-like growth factor-binding protein 4 signal sequence (e.g., MLPLCLVAALLLAAGPGPSLG)

o) variants of the pre-pro-region of the AFP signal sequence

p) a consensus signal sequence (MPTWAWWLFLVLLLALWAPARG)

q) acid phosphatase (PH05) leader (e.g., MFKSVVYSILAASLANA)

r) the pre-sequence of MFoz-1

s) the pre-sequence of 0 glucanase (BGL2)

t) killer toxin leader

u) the presequence of killer toxin

v) K. lactis killer toxin prepro (29 amino acids; 16 amino acids of pre and 13 amino acids of pro) MNIFYIFLFLLSFVQGLEHTHRRGSLD-KR

w) S. diastaticus glucoamylase 11 secretion leader sequence

x) S. carisbergensis alpha-galactosidase (MEL1) secretion leader sequence

y) Candida glucoamylase leader sequence

z) The hybrid leaders disclosed in EP-A-387 319 (herein incorporated by reference)

aa) the gp67 signal sequence (in conjunction with baculoviral expression systems) (e.g., amino acids 1-19 of GenBank Accession Number AAA72759) or

bb) the natural leader of the therapeutic protein X;

cc) S. cerevisiae invertase (SUC2) leader, as disclosed in JP 62-096086 (granted as 911036516, herein incorporate by reference); or

dd) Inulinase—MKLAYSLLLPLAGVSASVIN-YKR

ee) A modified TA57 propeptide leader variant #1—MKLKTVRSAVLSSLFASQVLGQPIDDTESQTTSVNLMADDTESAFATQTNSGGLDVV GLISMAKR

ff) A modified TA57 propeptide leader variant #2—MKLKTVRSAVLS SLFASQVLGQPIDDTESQTTSVNLMADDTESAFATQTNSGGLDVV GLISMAEEGEPKR

gg) A consensus signal peptide—MWWRLWWLLLLLLLLWPM-VWA

hh) MKWVSFISLLFLFSSAYSRSLDKR or

ii) MKWVSFISLLFLFSSAYSGSLDKR.

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in WO 87/04462; WO 86/05807; WO 89/01036; WO 89/10404; and WO 91/06657. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology, 10:169 (1992), and in Biblia and Robinson, Biotechnol. Prog., 11:1 (1995).

In addition, techniques known in the art may be used to operably associate heterologous polynucleotides (e.g., polynucleotides encoding an alpha-fetoprotein protein, or a fragment or variant thereof) and/or heterologous control regions (e.g., promoter and/or enhancer) with endogenous polynucleotide sequences encoding a therapeutic protein via homologous recombination (see U.S. Pat. No. 5,641,670; WO 96/29411; WO 94/12650; Koller et al., Proc. Natl. Acad. Sci. USA, 86:8932-8935 (1989); and Zijistra et al., Nature, 342:435-438 (1989)).

2. Host Cells

The invention also relates to host cells comprising the above-described vector constructs, and additionally encompasses host cells comprising nucleotide sequences of the invention that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a therapeutic protein may be replaced with an alpha-fetoprotein fusion protein corresponding to the therapeutic protein), and/or to include genetic material (e.g., heterologous polynucleotide sequences such as for example, an alpha-fetoprotein fusion protein of the invention corresponding to the therapeutic protein may be included). The genetic material operably associated with the endogenous polynucleotide may activate, alter, and/or amplify endogenous polynucleotides.

3. Purification and Recovery of Alpha-Fetoprotein Fusion Proteins

Alpha-fetoprotein fusion proteins of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. Most preferably, high performance liquid chromatography (“HPLC”) is employed for purification.

In preferred embodiments the alpha-fetoprotein fusion proteins of the invention are purified using Anion Exchange Chromatography including, but not limited to, chromatography on Q-sepharose, DEAE sepharose, poros HQ, poros DEAE, Toyopearl Q, Toyopearl QAE, Toyopearl DEAE, Resource/Source Q and DEAE, Fractogel Q and DEAE columns.

In specific embodiments the alpha-fetoprotein fusion proteins of the invention are purified using Cation Exchange Chromatography including, but not limited to, SP sepharose, CM sepharose, poros HS, poros CM, Toyopearl SP, Toyopearl CM, Resource/Source S and CM, Fractogel S and CM columns and their equivalents and comparables.

In specific embodiments the alpha-fetoprotein fusion proteins of the invention are purified using Hydrophobic Interaction Chromatography including, but not limited to, Phenyl, Butyl, Methyl, Octyl, Hexyl-sepharose, poros Phenyl, Butyl, Methyl, Octyl, Hexyl, Toyopearl Phenyl, Butyl, Methyl, Octyl, Hexyl Resource/Source Phenyl, Butyl, Methyl, Octyl, Hexyl, Fractogel Phenyl, Butyl, Methyl, Octyl, Hexyl columns and their equivalents and comparables.

In specific embodiments the alpha-fetoprotein fusion proteins of the invention are purified using Size Exclusion Chromatography including, but not limited to, sepharose S100, S200, S300, superdex resin columns and their equivalents and comparables.

In specific embodiments the alpha-fetoprotein fusion proteins of the invention are purified using Affinity Chromatography including, but not limited to, Mimetic Dye affinity, peptide affinity and antibody affinity columns that are selective for either the alpha-fetoprotein or the “fusion target” molecules.

In preferred embodiments alpha-fetoprotein fusion proteins of the invention are purified using one or more Chromatography methods listed above. In other preferred embodiments, alpha-fetoprotein fusion proteins of the invention are purified using one or more of the following Chromatography columns, Q sepharose FF column, SP Sepharose FF column, Q Sepharose High Performance Column, Blue Sepharose FF column, Blue Column, Phenyl Sepharose FF column, DEAE Sepharose FF, or Methyl Column.

Alpha-fetoprotein fusion proteins of the invention may be recovered from products of chemical synthetic procedures; or products produced by recombinant techniques from a prokaryotic or eukaryotic host, including, for example, bacterial, yeast, higher plant, insect, and mammalian cells. Depending upon the host employed in a recombinant production procedure, the polypeptides of the invention may be glycosylated or may be non-glycosylated. In addition, alpha-fetoprotein fusion proteins of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express alpha-fetoprotein fusion proteins of the invention in a eukaryotic system. Pichia pastoris is a methylotrophic yeast which can metabolize methanol as its sole carbon source. A main step in the methanol metabolization pathway is the oxidation of methanol to formaldehyde using O₂. This reaction is catalyzed by the enzyme alcohol oxidase. To metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX₁) is highly active. In the presence of methanol, alcohol oxidase produced from the AOX₁ gene comprises up to approximately 30% of the total soluble protein in Pichia pastoris. See Ellis et al., Mol. Cell. Biol., 5:1111-21 (1985); Koutz et al., Yeast, 5:167-77 (1989); Tschopp et al., Nucl. Acids Res., 15:3859-76 (1987). Thus, a heterologous coding sequence, such as, for example, a polynucleotide of the invention, under the transcriptional regulation of all or part of the AOX₁ regulatory sequence is expressed at exceptionally high levels in Pichia yeast grown in the presence of methanol.

In one example, the plasmid vector pPIC9K is used to express DNA encoding an alpha-fetoprotein fusion protein of the invention, as set forth herein, in a Pichea yeast system essentially as described in “Pichia Protocols: Methods in Molecular Biology,” D. R. Higgins and J. Cregg, eds. (The Humana Press, Totowa, N.J., 1998). This expression vector allows expression and secretion of a polypeptide of the invention by virtue of the strong AOX₁ promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PA0815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, a polynucleotide encoding an alpha-fetoprotein fusion protein of the invention, may be achieved by cloning the heterologous polynucleotide of the invention into an expression vector such as, for example, pGAPZ or pGAPZalpha, and growing the yeast culture in the absence of methanol.

4. Chemical Synthesis of Alpha-Fetoprotein Fusion Proteins

In addition, alpha-fetoprotein fusion proteins of the invention can be chemically synthesized using techniques known in the art (Creighton, Proteins: Structures and Molecular Principles (W. H. Freeman & Co., N.Y. 1983), and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, alpha.-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

5. Chemical Synthesis/Modification of Alpha-Fetoprotein Fusion Proteins

The invention encompasses alpha-fetoprotein fusion proteins of the invention which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The alpha-fetoprotein fusion proteins may also be modified with a detectable label, such as an enzymatic, fluorescent, isotopic or affinity label to allow for detection and isolation of the protein.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (₆₅Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

In specific embodiments, alpha-fetoprotein fusion proteins of the invention or fragments or variants thereof are attached to macrocyclic chelators that associate with radiometal ions, including but not limited to, ¹⁷⁷Lu, ⁹⁰Y, ¹⁶⁶Ho, and ¹⁵³Sm. In a preferred embodiment, the radiometal ion associated with the macrocyclic chelators is ¹¹¹In. In another preferred embodiment, the radiometal ion associated with the macrocyclic chelator is ⁹⁰Y. In specific embodiments, the macrocyclic chelator is 1,4,7,10-tetraazacyclo-dodecane-N,N′,N″,N′″-tetraacetic acid (DOTA). In other specific embodiments, DOTA is attached to an antibody of the invention or fragment thereof via linker molecule. Examples of linker molecules useful for conjugating DOTA to a polypeptide are commonly known in the art (DeNardo et al., Clin. Cancer Res., 4(10):2483-90 (1998); Peterson et al., Bioconjug. Chem., 10(4):553-7 (1999); and Zimmerman et al, Nucl. Med. Biol., 26(8):943-50 (1999).

As mentioned, the alpha-fetoprotein fusion proteins of the invention may be modified by either natural processes, such as post-translational processing, or by chemical modification techniques which are well known in the art. It will be appreciated that the same type of modification may be present in the same or varying degrees at several sites in a given polypeptide. Polypeptides of the invention may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyro glutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See Proteins—Structure and Molecular Properties, 2nd Ed., T. E. Creighton (W. H. Freeman and Company, New York (1993)); Post-Translational Covalent Modification of Proteins, B. C. Johnson, Ed., pgs. 1-12 (Academic Press, New York, 1983); Seifter et al., Meth. Enzymol., 182:626-646 (1990); Rattan et al., Ann. N.Y. Acad. Sci., 663:48-62 (1992).)

Alpha-fetoprotein fusion proteins of the invention and antibodies that bind a therapeutic protein or fragments or variants thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA, 86:821-824 (1989), for instance, hexa-histidine provides for convenient purification of the fusion protein. Other peptide tags useful for purification include, but are not limited to, the “HA tag”, which corresponds to an epitope derived from the influenza hemagglutinin protein (Wilson et al., Cell, 37:767 (1984)) and the “flag” tag.

a. Conjugation of Alpha-Fetoprotein Fusion Proteins to a Therapeutic Moiety or Vaccine Antigen

Further, an alpha-fetoprotein fusion protein of the invention may be conjugated to a therapeutic moiety or vaccine antigen such as PA toxin (anthrax), Avian Flu antigen (e.g., H5N1), a cytotoxin, e.g., a cytostatic or cytocidal agent, a therapeutic agent or a radioactive metal ion, e.g., alpha-emitters such as, for example, ²¹³Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (H) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as PA toxin (anthrax), abrin, ricin A, tuberculosis antigen, SARS antigen, HIV antigen, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (WO 97/33899), AIM II (WO 97/34911), Fas Ligand (Takahashi et al., Int. Immunol., 6:1567-1574 (1994)), VEGI (WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to proteins (e.g., alpha-fetoprotein fusion proteins) are well known in the art.

Alpha-fetoprotein fusion proteins may also be attached to solid supports, which are particularly useful for immunoassays or purification of polypeptides that are bound by, that bind to, or associate with alpha-fetoprotein fusion proteins of the invention. Such solid supports include, but are not limited to, glass, cellulose, polyacrylamide, nylon, polystyrene, polyvinyl chloride or polypropylene.

Alpha-fetoprotein fusion proteins, with or without a therapeutic moiety conjugated to it, administered alone or in combination with cytotoxic factor(s) and/or cytokine(s) can be used as a therapeutic.

In embodiments where the alpha-fetoprotein fusion protein of the invention comprises only the VH domain of an antibody that binds a therapeutic protein, it may be necessary and/or desirable to coexpress the fusion protein with the VL domain of the same antibody that binds a therapeutic protein, such that the VH-alpha-fetoprotein fusion protein and VL protein will associate (either covalently or non-covalently) post-translationally. In embodiments where the alpha-fetoprotein fusion protein of the invention comprises only the VL domain of an antibody that binds a therapeutic protein, it may be necessary and/or desirable to coexpress the fusion protein with the VH domain of the same antibody that binds a therapeutic protein, such that the VL-alpha-fetoprotein fusion protein and VH protein will associate (either covalently or non-covalently) post-translationally.

Some therapeutic antibodies are bispecific antibodies, meaning the antibody that binds a therapeutic protein is an artificial hybrid antibody having two different heavy/light chain pairs and two different binding sites. To create an alpha-fetoprotein fusion protein corresponding to that therapeutic protein, it is possible to create an alpha-fetoprotein fusion protein which has an scFv fragment fused to both the N- and C-terminus of the alpha-fetoprotein protein moiety. More particularly, the scFv fused to the N-terminus of alpha-fetoprotein would correspond to one of the heavy/light (VH/VL) pairs of the original antibody that binds a therapeutic protein and the scFv fused to the C-terminus of alpha-fetoprotein would correspond to the other heavy/light (VH/VL) pair of the original antibody that binds a therapeutic protein.

b. Chemical Moieties Utilized for Chemical Modification of the Alpha-Fetoprotein Fusion Proteins

Also provided by the invention are chemically modified derivatives of the alpha-fetoprotein fusion proteins of the invention which may provide additional advantages such as increased solubility, stability and circulating time of the polypeptide, or decreased immunogenicity (U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The alpha-fetoprotein fusion proteins may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, about 500, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 95,000, or about 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol., 56:59-72 (1996); Vorobjev et al., Nucleosides Nucleotides, 18:2745-2750 (1999); and Caliceti et al., Bioconjug. Chem., 10:638646 (1999).

The polyethylene glycol molecules (or other chemical moieties) should be attached to the protein with consideration of effects on functional or antigenic domains of the protein. There are a number of attachment methods available to those skilled in the art, such as, for example, the method disclosed in EP 0 401 384 (coupling PEG to G-CSF); see also Malik et al., Exp. Hematol., 20:1028-1035 (1992), reporting pegylation of GM-CSF using tresyl chloride. For example, polyethylene glycol may be covalently bound through amino acid residues via reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, PEGylation of the alpha-fetoprotein fusion proteins of the invention may be accomplished by any number of means. For example, polyethylene glycol may be attached to the alpha-fetoprotein fusion protein either directly or by an intervening linker. Linkerless systems for attaching polyethylene glycol to proteins are described in Delgado et al., Crit. Rev. Thera. Drug Carrier Sys., 9:249-304 (1992); Francis et al., Intem. J. of Hematol., 68:1-18 (1998); U.S. Pat. No. 4,002,531; U.S. Pat. No. 5,349,052; WO 95/06058; and WO 98/32466.

One system for attaching polyethylene glycol directly to amino acid residues of proteins without an intervening linker employs tresylated MPEG, which is produced by the modification of monmethoxy polyethylene glycol (MPEG) using tresylchloride (ClSO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460 discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in WO 98/32466. Pegylated protein products produced using the reaction chemistries set out herein are included within the scope of the invention.

The number of polyethylene glycol moieties attached to each alpha-fetoprotein fusion protein of the invention (i.e., the degree of substitution) may also vary. For example, the PEGylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as about 1 to about 3, about 3 to about 5, about 4 to about 6, about 5 to about 7, about 6 to about 8, about 7 to about 9, about 8 to about 10, about 9 to about 11, about 10 to about 12, about 11 to about 13, about 12 to about 14, about 13 to about 15, about 14 to about 16, about 15 to about 17, about 16 to about 18, about 17 to about 19, or about 18 to about 20 polyethylene glycol moieties per protein molecule. Methods for determining the degree of substitution are discussed, for example, in Delgado et al., Crit. Rev. Thera. Drug Carrier Sys., 9:249-304 (1992).

The polypeptides of the invention can be recovered and purified from chemical synthesis and recombinant cell cultures by standard methods which include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Well known techniques for refolding protein may be employed to regenerate active conformation when the polypeptide is denatured during isolation and/or purification.

The presence and quantity of alpha-fetoprotein fusion proteins of the invention may be determined using ELISA, a well known immunoassay known in the art. In one ELISA protocol that would be useful for detecting/quantifying alpha-fetoprotein fusion proteins of the invention, comprises the steps of coating an ELISA plate with an anti-alpha-fetoprotein antibody, blocking the plate to prevent non-specific binding, washing the ELISA plate, adding a solution comprising the alpha-fetoprotein fusion protein of the invention (at one or more different concentrations), adding a secondary anti-therapeutic protein specific antibody coupled to a detectable label (as described herein or otherwise known in the art), and detecting the presence of the secondary antibody. In an alternate version of this protocol, the ELISA plate might be coated with the anti-therapeutic protein specific antibody and the labeled secondary reagent might be the anti-alpha-fetoprotein specific antibody.

VI. Uses of the Polynucleotides

Each of the polynucleotides identified herein can be used in numerous ways as reagents. The following description should be considered exemplary and utilizes known techniques.

The polynucleotides of the invention are useful to produce the alpha-fetoprotein fusion proteins of the invention. As described in more detail below, polynucleotides of the invention (encoding alpha-fetoprotein fusion proteins) may be used in recombinant DNA methods useful in genetic engineering to make cells, cell lines, or tissues that express the alpha-fetoprotein fusion protein encoded by the polynucleotides encoding alpha-fetoprotein fusion proteins of the invention.

Polynucleotides of the invention are also useful in gene therapy. One goal of gene therapy is to insert a normal gene into an organism having a defective gene, in an effort to correct the genetic defect. The polynucleotides disclosed of the invention offer a means of targeting such genetic defects in a highly accurate manner. Another goal is to insert a new gene that was not present in the host genome, thereby producing a new trait in the host cell. Additional non-limiting examples of gene therapy methods encompassed by the invention are more thoroughly described elsewhere herein.

VII. Uses of the Polypeptides

Each of the polypeptides identified herein can be used in numerous ways. The following description should be considered exemplary and utilizes known techniques.

Alpha-fetoprotein fusion proteins of the invention are useful to provide immunological probes for differential identification of the tissue(s) (e.g., immunohistochemistry assays such as, for example, ABC immunoperoxidase (Hsu et al., J. Histochem. Cytochem., 29:577-580 (1981)) or cell type(s) (e.g., immunocytochemistry assays).

Alpha-fetoprotein fusion proteins can be used to assay levels of polypeptides in a biological sample using classical immunohistological methods known to those of skill in the art (e.g., see Jalkanen et al., J. Cell. Biol., 101:976-985 (1985); Jalkanen, et al., J. Cell. Biol., 105:3087-3096 (1987)). Other methods useful for detecting protein gene expression include immunoassays, such as the enzyme linked immunosorbent assay (ELISA) and the radioimmunoassay (RIA). Suitable assay labels are known in the art and include enzyme labels, such as, glucose oxidase; radioisotopes luminescent labels, such as luminol; and fluorescent labels, such as fluorescein and rhodamine, and biotin.

Alpha-fetoprotein fusion proteins of the invention can also be detected in vivo by imaging. Labels or markers for in vivo imaging of protein include those detectable by X-radiography, nuclear magnetic resonance (NMR) or electron spin relaxation (ESR). For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the alpha-fetoprotein fusion protein by labeling of nutrients given to a cell line expressing the alpha-fetoprotein fusion protein of the invention.

An alpha-fetoprotein fusion protein which has been labeled with an appropriate detectable imaging moiety, such as a radioisotope, a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁸mTc. The labeled alpha-fetoprotein fusion protein will then preferentially accumulate at locations in the body (e.g., organs, cells, extracellular spaces or matrices) where one or more receptors, ligands or substrates (corresponding to that of the therapeutic protein used to make the alpha-fetoprotein fusion protein of the invention) are located. Alternatively, in the case where the alpha-fetoprotein fusion protein comprises at least a fragment or variant of a therapeutic antibody, the labeled alpha-fetoprotein fusion protein will then preferentially accumulate at the locations in the body (e.g., organs, cells, extracellular spaces or matrices) where the polypeptides/epitopes corresponding to those bound by the therapeutic antibody (used to make the alpha-fetoprotein fusion protein of the invention) are located. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds. (Masson Publishing Inc., 1982). The protocols described therein could easily be modified by one of skill in the art for use with the alpha-fetoprotein fusion proteins of the invention.

In one embodiment, the invention provides a method for the specific delivery of alpha-fetoprotein fusion proteins of the invention to cells by administering alpha-fetoprotein fusion proteins of the invention (e.g., polypeptides encoded by polynucleotides encoding alpha-fetoprotein fusion proteins of the invention and/or antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering alpha-fetoprotein fusion proteins of the invention in association with toxins or cytotoxic prodrugs.

By “toxin” is meant one or more compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, PA toxin (anthrax), ricin, abrin, Pseudomonas exotoxin A, diphtheria toxin, saponin, tuberculosis toxin, momordin, gelonin, pokeweed antiviral protein, alpha-sarcin and cholera toxin. “Toxin” also includes a cytostatic or cytocidal agent, a therapeutic agent or a radioactive metal ion, e.g., alpha-emitters such as, for example, ²¹³Bi, or other radioisotopes, luminescent labels, such as luminol; and fluorescent labels, such as fluorescein and rhodamine, and biotin. In a specific embodiment, the invention provides a method for the specific destruction of cells (e.g., the destruction of tumor cells) by administering polypeptides of the invention or antibodies of the invention in association with the radioisotope ⁹⁰Y. In another specific embodiment, the invention provides a method for the specific destruction of cells (e.g., the destruction of tumor cells) by administering polypeptides of the invention or antibodies of the invention in association with the radioisotope ¹¹¹In. In a further specific embodiment, the invention provides a method for the specific destruction of cells (e.g., the destruction of tumor cells) by administering polypeptides of the invention or antibodies of the invention in association with the radioisotope ¹¹³I.

Techniques known in the art may be applied to lable polypeptides of the invention. Such techniques include, but are not limited to, the use of bifunctional conjugating agents (U.S. Pat. Nos. 5,756,065; 5,714,631; 5,696,239; 5,652,361; 5,505,931; 5,489,425; 5,435,990; 5,428,139; 5,342,604; 5,274;119; 4,994,560; and 5,808,003).

The alpha-fetoprotein fusion proteins of the invention are useful for diagnosis, treatment, prevention and/or prognosis of various disorders in mammals, preferably humans. Such disorders include, but are not limited to, those described herein under the section heading “Biological Activities,” below.

Thus, the invention provides a diagnostic method of a disorder which comprises: (a) assaying the expression level of a certain polypeptide in cells or body fluid of an individual using an alpha-fetoprotein fusion protein of the invention; and (b) comparing the assayed polypeptide expression level with a standard polypeptide expression level, whereby an increase or decrease in the assayed polypeptide expression level compared to the standard expression level is indicative of a disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Moreover, alpha-fetoprotein fusion proteins of the invention can be used to treat or prevent diseases or conditions such as, for example, neural disorders, immune system disorders, muscular disorders, reproductive disorders, gastrointestinal disorders, pulmonary disorders, cardiovascular disorders, renal disorders, proliferative disorders, and/or cancerous diseases and conditions. For example, patients can be administered a polypeptide of the invention in an effort to replace absent or decreased levels of the polypeptide (e.g., insulin), to supplement absent or decreased levels of a different polypeptide (e.g., hemoglobin S for hemoglobin B, SOD, catalase, DNA repair proteins), to inhibit the activity of a polypeptide (e.g., an oncogene or tumor supressor), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth inhibition, enhancement of the immune response to proliferative cells or tissues).

In particular, alpha-fetoprotein fusion proteins comprising of at least a fragment or variant of a therapeutic antibody can also be used to treat disease (as described supra, and elsewhere herein). For example, administration of an alpha-fetoprotein fusion protein comprising of at least a fragment or variant of a therapeutic antibody can bind, and/or neutralize the polypeptide to which the therapeutic antibody used to make the alpha-fetoprotein fusion protein specifically binds, and/or reduce overproduction of the polypeptide to which the therapeutic antibody used to make the alpha-fetoprotein fusion protein specifically binds. Similarly, administration of an alpha-fetoprotein fusion protein comprising of at least a fragment or variant of a therapeutic antibody can activate the polypeptide to which the therapeutic antibody used to make the alpha-fetoprotein fusion protein specifically binds, by binding to the polypeptide bound to a membrane (receptor).

At the very least, the alpha-fetoprotein fusion proteins of the invention of the invention can be used as molecular weight markers on SDS-PAGE gels or on molecular sieve gel filtration columns using methods well known to those of skill in the art. Alpha-fetoprotein fusion proteins of the invention can also be used to raise antibodies, which in turn may be used to measure protein expression of the therapeutic protein, alpha-fetoprotein, and/or the alpha-fetoprotein fusion protein of the invention from a recombinant cell, as a way of assessing transformation of the host cell, or in a biological sample. Moreover, the alpha-fetoprotein fusion proteins of the invention can be used to test the biological activities described herein.

VIII. Diagnostic Assays

The compounds of the invention are useful for diagnosis, treatment, prevention and/or prognosis of various disorders in mammals, preferably humans. Such disorders include, but are not limited to, those described under the section headings “Immune Activity,” “Blood Related Disorders,” “Hyperproliferative Disorders,” “Renal Disorders,” “Cardiovascular Disorders,” “Respiratory Disorders,” “Anti-Angiogenesis Activity,” “Diseases at the Cellular Level,” “Wound Healing and Epithelial Cell Proliferation,” “Neural Activity and Neurological Diseases,” “Endocrine Disorders,” “Reproductive System Disorders,” “Infectious Disease,” “Regeneration,” and/or “Gastrointestinal Disorders,” infra.

For a number of disorders, substantially altered (increased or decreased) levels of gene expression can be detected in tissues, cells or bodily fluids (e.g., sera, plasma, urine, semen, synovial fluid or spinal fluid) taken from an individual having such a disorder, relative to a “standard” gene expression level, that is, the expression level in tissues or bodily fluids from an individual not having the disorder. Thus, the invention provides a diagnostic method useful during diagnosis of a disorder, which involves measuring the expression level of the gene encoding a polypeptide in tissues, cells or body fluid from an individual and comparing the measured gene expression level with a standard gene expression level, whereby an increase or decrease in the gene expression level(s) compared to the standard is indicative of a disorder. These diagnostic assays may be performed in vivo or in vitro, such as, for example, on blood samples, biopsy tissue or autopsy tissue.

The invention is also useful as a prognostic indicator, whereby patients exhibiting enhanced or depressed gene expression will experience a worse clinical outcome

By “assaying the expression level of the gene encoding a polypeptide” is intended qualitatively or quantitatively measuring or estimating the level of a particular polypeptide or the level of the mRNA encoding the polypeptide of the invention in a first biological sample either directly (e.g., by determining or estimating absolute protein level or mRNA level) or relatively (e.g., by comparing to the polypeptide level or mRNA level in a second biological sample). Preferably, the polypeptide expression level or mRNA level in the first biological sample is measured or estimated and compared to a standard polypeptide level or mRNA level, the standard being taken from a second biological sample obtained from an individual not having the disorder or being determined by averaging levels from a population of individuals not having the disorder. As will be appreciated in the art, once a standard polypeptide level or mRNA level is known, it can be used repeatedly as a standard for comparison.

By “biological sample” is intended any biological sample obtained from an individual, cell line, tissue culture, or other source containing polypeptides of the invention (including portions thereof) or mRNA. As indicated, biological samples include body fluids (such as sera, plasma, urine, synovial fluid and spinal fluid) and tissue sources found to express the full length or fragments thereof of a polypeptide or mRNA. Methods for obtaining tissue biopsies and body fluids from mammals are well known in the art. Where the biological sample is to include mRNA, a tissue biopsy is the preferred source.

Total cellular RNA can be isolated from a biological sample using any suitable technique such as the single-step guanidinium-thiocyanate-phenol-chloroform method described in Chomczynski and Sacchi, Anal. Biochem., 162:156-159 (1987). Levels of mRNA encoding the polypeptides of the invention are then assayed using any appropriate method. These include Northern blot analysis, SI nuclease mapping, the polymerase chain reaction (PCR), reverse transcription in combination with the polymerase chain reaction (RT-PCR), and reverse transcription in combination with the ligase chain reaction (RT-LCR).

The invention also relates to diagnostic assays such as quantitative and diagnostic assays for detecting levels of polypeptides that bind to, are bound by, or associate with alpha-fetoprotein fusion proteins of the invention, in a biological sample (e.g., cells and tissues), including determination of normal and abnormal levels of polypeptides. Thus, for instance, a diagnostic assay in accordance with the invention for detecting abnormal expression of polypeptides that bind to, are bound by, or associate with alpha-fetoprotein fusion proteins compared to normal control tissue samples may be used to detect the presence of tumors. Assay techniques that can be used to determine levels of a polypeptide that bind to, are bound by, or associate with alpha-fetoprotein fusion proteins of the invention in a sample derived from a host are well-known to those of skill in the art. Such assay methods include radioimmunoassays, competitive-binding assays, Western Blot analysis and ELISA assays. Assaying polypeptide levels in a biological sample can occur using any art-known method.

Assaying polypeptide levels in a biological sample can occur using a variety of techniques. For example, polypeptide expression in tissues can be studied with classical immunohistological methods (Jalkanen et al., J. Cell. Biol., 101:976-985 (1985); Jalkanen et al., J. Cell. Biol., 105:3087-3096 (1987)). Other methods useful for detecting polypeptide gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein, rhodamine, and biotin.

The tissue or cell type to be analyzed will generally include those which are known, or suspected, to express the gene of interest (such as, for example, cancer). The protein isolation methods employed herein may, for example, be such as those described in Harlow and Lane (Harlow et al., “Antibodies: A Laboratory Manual” (Cold Spring Harbor Laboratory Press, Cold Spring Harbor, N.Y., 1988). The isolated cells can be derived from cell culture or from a patient. The analysis of cells taken from culture may be a necessary step in the assessment of cells that could be used as part of a cell-based gene therapy technique or, alternatively, to test the effect of compounds on the expression of the gene.

For example, alpha-fetoprotein fusion proteins may be used to quantitatively or qualitatively detect the presence of polypeptides that bind to, are bound by, or associate with alpha-fetoprotein fusion proteins of the present invention. This can be accomplished, for example, by immunofluorescence techniques employing a fluorescently labeled alpha-fetoprotein fusion protein coupled with light microscopic, flow cytometric, or fluorimetric detection.

In a preferred embodiment, alpha-fetoprotein fusion proteins comprising at least a fragment or variant of an antibody that specifically binds at least a therapeutic protein disclosed herein or otherwise known in the art may be used to quantitatively or qualitatively detect the presence of gene products or conserved variants or peptide fragments thereof. This can be accomplished, for example, by immunofluorescence techniques employing a fluorescently labeled antibody coupled with light microscopic, flow cytometric, or fluorimetric detection.

The alpha-fetoprotein fusion proteins of the present invention may, additionally, be employed histologically, as in immunofluorescence, immunoelectron microscopy or non-immunological assays, for in situ detection of polypeptides that bind to, are bound by, or associate with an alpha-fetoprotein fusion protein of the present invention. In situ detection may be accomplished by removing a histological specimen from a patient, and applying thereto a labeled antibody or polypeptide of the invention. The alpha-fetoprotein fusion proteins are preferably applied by overlaying the labeled alpha-fetoprotein fusion proteins onto a biological sample. Through the use of such a procedure, it is possible to determine not only the presence of the polypeptides that bind to, are bound by, or associate with alpha-fetoprotein fusion proteins, but also its distribution in the examined tissue. Using the invention, those of ordinary skill will readily perceive that any of a wide variety of histological methods (such as staining procedures) can be modified to achieve such in situ detection.

Immunoassays and non-immunoassays that detect polypeptides that bind to, are bound by, or associate with alpha-fetoprotein fusion proteins will typically comprise incubating a sample, such as a biological fluid, a tissue extract, freshly harvested cells, or lysates of cells which have been incubated in cell culture, in the presence of a detectably labeled antibody capable of binding gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled alpha-fetoprotein fusion protein of the invention. The solid phase support may then be washed with the buffer a second time to remove unbound antibody or polypeptide. Optionally the antibody is subsequently labeled. The amount of bound label on solid support may then be detected by conventional means.

By “solid phase support or carrier” is intended any support capable of binding a polypeptide (e.g., an alpha-fetoprotein fusion protein, or polypeptide that binds, is bound by, or associates with an alpha-fetoprotein fusion protein of the invention). Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the invention. The support material may have virtually any possible structural configuration so long as the coupled molecule is capable of binding to a polypeptide. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of alpha-fetoprotein fusion protein may be determined according to well known methods. Those skilled in the art will be able to determine operative and optimal assay conditions for each determination by employing routine experimentation.

In addition to assaying polypeptide levels in a biological sample obtained from an individual, polypeptide can also be detected in vivo by imaging. For example, in one embodiment of the invention, alpha-fetoprotein fusion proteins of the invention are used to image diseased or neoplastic cells. Labels or markers for in vivo imaging of alpha-fetoprotein fusion proteins of the invention include those detectable by X-radiography, NMR, MR1, CAT-scans or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the alpha-fetoprotein fusion protein by labeling of nutrients of a cell line (or bacterial or yeast strain) engineered.

Additionally, alpha-fetoprotein fusion proteins of the invention whose presence can be detected, can be administered. For example, alpha-fetoprotein fusion proteins of the invention labeled with a radio-opaque or other appropriate compound can be administered and visualized in vivo, as discussed, above for labeled antibodies. Further, such polypeptides can be utilized for in vitro diagnostic procedures.

A polypeptide-specific antibody or antibody fragment which has been labeled with an appropriate detectable imaging moiety, such as a radioisotope, a radio-opaque substance, or a material detectable by nuclear magnetic resonance, is introduced (for example, parenterally, subcutaneously or intraperitoneally) into the mammal to be examined for a disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled alpha-fetoprotein fusion protein will then preferentially accumulate at the locations in the body which contain a polypeptide or other substance that binds to, is bound by or associates with an alpha-fetoprotein fusion protein of the invention. In vivo tumor imaging is described in S. W. Burchiel et al., “Immunopharmacokinetics of Radiolabeled Antibodies and Their Fragments” (Chapter 13 in Tumor Imaging. The Radiochemical Detection of Cancer, S. W. Burchiel and B. A. Rhodes, eds. (Masson Publishing Inc., 1982).

One of the ways in which an alpha-fetoprotein fusion protein of the invention can be detectably labeled is by linking the same to a reporter enzyme and using the linked product in an enzyme immunoassay (EIA) (Voller, A., “The Enzyme Linked Immunosorbent Assay (ELISA)”, Diagnostic Horizons 2:1-7, Microbiological Associates Quarterly Publication, Walkersville, Md., 1978); Voller et al., J. Clin. Pathol., 31:507-520 (1978); Butler, J. E., Meth. Enzymol., 73:482-523 (1981); Maggio, E. (ed.), Enzyme Immunoassay (CRC Press, Boca Raton, Fla., 1980); Ishikawa, E. et al., (eds.), Enzyme Immunoassay (Kgaku Shoin, Tokyo, 1981)). The reporter enzyme which is bound to the antibody will react with an appropriate substrate, preferably a chromogenic substrate, in such a manner as to produce a chemical moiety which can be detected, for example, by spectrophotometric, fluorimetric or by visual means. Reporter enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucoseb-phosphate dehydrogenase, glucoamylase and acetylcholinesterase. Additionally, the detection can be accomplished by colorimetric methods which employ a chromogenic substrate for the reporter enzyme. Detection may also be accomplished by visual comparison of the extent of enzymatic reaction of a substrate in comparison with similarly prepared standards.

Alpha-fetoprotein fusion proteins may also be radiolabelled and used in any of a variety of other immunoassays. For example, by radioactively labeling the alpha-fetoprotein fusion proteins, it is possible to the use the alpha-fetoprotein fusion proteins in a radioimmunoassay (RIA) (Weintraub, B., Principles of Radioimmunoassays, Seventh Training Course on Radioligand Assay Techniques (The Endocrine Society, March, 1986). The radioactive isotope can be detected by means including, but not limited to, a gamma counter, a scintillation counter, or autoradiography.

Additionally, chelator molecules, are known in the art and can be used to label the Alpha-fetoprotein fusion proteins. Chelator molecules may be attached Alpha-fetoprotein fusion proteins of the invention to facilitate labeling the protein with metal ions including radionuclides or fluorescent labels (Subramanian, R. and Meares, C. F., “Bifunctional Chelating Agents for Radiometal-labeled monoclonal Antibodies,” in Cancer Imaging with Radiolabeled Antibodies, D. M. Goldenberg, Ed. (Kluwer Academic Publications, Boston, 1990); Saji, H., “Targeted delivery of radiolabeled imaging and therapeutic agents: bifunctional radiopharmaceuticals.” Crit. Rev. Ther. Drug Carrier Syst., 16:209-244 (1999); Srivastava S. C. and Mease R. C., “Progress in research on ligands, nuclides and techniques for labeling monoclonal antibodies.” Int. J. Rad. Appl. Instrum. B., 18:589-603 (1991); and Liu, S. and Edwards, D. S., “Bifunctional chelators for therapeutic lanthamide radiopharmaceuticals.” Bioconjug. Chem., 12:7-34 (2001).) Any chelator which can be covalently bound to said Alpha-fetoprotein fusion proteins may be used according to the invention. The chelator may further comprise a linker moiety that connects the chelating moiety to the Alpha-fetoprotein fusion protein.

In one embodiment, the Alpha-fetoprotein fusion protein of the invention is attached to an acyclic chelator such as diethylene triamine-N,N,N′,N″,N″-pentaacetic acid (DPTA), analogues of DPTA, or derivatives of DPTA. As non-limiting examples, the chelator may be 2-(p-isothiocyanatobenzyl)-6-methyldiethylenetriaminepentaacetic acid (1B4M-DPTA, also known as MX-DTPA), 2-methyl-6-(rho-nitrobenzyl)-1,4,7-tr-iazaheptane-N,N,N′,N″,N″-pentaacetic acid (nitro-IB4M-DTPA or nitro-MX-DTPA); 2-p-isothiocyanatobenzyl)-cyclohexyldiethylenetriaminepen-taacetic acid (CHX-DTPA), or N-[2-amino-3-rho-nitrophenyl)propyl]-trans-cyclohexane-1,2-diamine-N,N′,N″-pentaacetic acid (nitro-CHX-A-DTPA).

In another embodiment, the Alpha-fetoprotein fusion protein of the invention is attached to an acyclic terpyridine chelator such as 6,6″-bis[[N,N,N″,N″-tetra(carboxymethyl)amino]methyl]-4′-(3-amino-4-methoxyphenyl)-2,2′:6′,2″-terpyridine (TMT-amine).

In specific embodiments, the macrocyclic chelator which is attached to the Alpha-fetoprotein fusion protein of the invention is 1,4,7,10-tetraazacyclododecane-N,N′,N″,N′″-tetraacetic acid (DOTA). In other specific embodiments, the DOTA is attached to the Alpha-fetoprotein fusion protein of the invention via a linker molecule. Examples of linker molecules useful for conjugating DOTA to a polypeptide are commonly known in the art (DeNardo et al., Clin. Cancer Res., 4(10):2483-90, 1998; Peterson et al., Bioconjug. Chem., 10(4):553-7, 1999; and Zimmerman et al., Nucl. Med. Biol., 26(8):943-50 (1999). In addition, U.S. Pat. Nos. 5,652,361 and 5,756,065 disclose chelating agents that may be conjugated to antibodies and methods for making and using them. Though U.S. Pat. Nos. 5,652,361 and 5,756,065 focus on conjugating chelating agents to antibodies, one skilled in the art could readily adapt the method disclosed therein to conjugate chelating agents to other polypeptides.

Bifunctional chelators based on macrocyclic ligands in which conjugation is via an activated arm, or functional group, attached to the carbon backbone of the ligand can be employed as described by Moi et al., J. Amer. Chem. Soc., 49:2639 (1989) (2-p-nitrobenzyl-1,4,7,10-tetraaza-cyclododecane-N,N′,N″,N′″-tetraacetic acid); Deshpande et al, J. Nucl. Med., 31:473 (1990); Ruser et al., Bioconj. Chem., 1:345 (1990); Broan et al., J. C. S. Chem. Comm., 23:1739 (1990); and Anderson et al., J. Nucl. Med., 36:850 (1995).

In one embodiment, a macrocyclic chelator, such as polyazamacrocyclic chelators, optionally containing one or more carboxy, amino, hydroxamate, phosphonate, or phosphate groups, are attached to the Alpha-fetoprotein fusion protein of the invention. In another embodiment, the chelator is a chelator selected from the group consisting of DOTA, analogues of DOTA, and derivatives of DOTA.

In one embodiment, suitable chelator molecules that may be attached to the Alpha-fetoprotein fusion protein of the invention include DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), and THT (4′-3-amino-4-methoxy-phenyl)-6,6″-bis(-N′,N′-dicarboxymethyl-N-methylhydrazino)-2,2′:6′,2″-terpyridine), and analogs and derivatives thereof. See Ohmono et al., J. Med. Chem., 35: 157-162 (1992); Kung et al., J. Nucl. Med., 25: 326-332 (1984); Jurisson et al., Chem. Rev., 93:1137-1156 (1993); and U.S. Pat. No. 5,367,080. Other suitable chelators include chelating agents disclosed in U.S. Pat. Nos. 4,647,447; 4,687,659; 4,885,363; EP-A-71564; WO89/00557; and EP-A-232751.

In another embodiment, suitable macrocyclic carboxylic acid chelators which can be used in the invention include 1,4,7,10-tetraazacyclododecane-N,N′,N″,N′″-tetraacetic acid (DOTA); 1,4,8,12-traaeacyclopentadecane-N,N′,N″N′″-tetraacetic acid (15N4); 1,4,7-triazacyclononane-N,N′,N″-triacetic acid (9N3); 1,5,9-triazacyclododecane-N,N′,N″-triacetic acid (12N3); and 6-bromoacetamido-benzyl-1,4,8,11-tetraazacyclotetradecane-N,N′,N″,N′″-tetraacetic acid (BAT).

A preferred chelator that can be attached to the Alpha-fetoprotein Fusion protein of the invention is alpha-(5-isothiocyanato-2-methoxyphenyl)-1,-4,7,10-tetraazacyclododecane-1,4,7,10-tetraacetic acid, which is also known as MeO-DOTA-NCS. A salt or ester of alpha-(5-isothiocyanato-2-met-hoxyphenyl)-1,4,7,10-tetraazacyclododecane-1,4,7,10-tetraacetic acid may also be used.

Alpha-fetoprotein fusion proteins of the invention to which chelators such as those described are covalently attached may be labeled (via the coordination site of the chelator) with radionuclides that are suitable for therapeutic, diagnostic, or both therapeutic and diagnostic purposes. Examples of appropriate metals include Ag, At, Au, Bi, Cu, Ga, Ho, In, Lu, Pb, Pd, Pm, Pr, Rb, Re, Rh, Sc, Sr, Tc, Tl, Y, and Yb. Examples of the radionuclide used for diagnostic purposes are Fe, Gd, ¹¹¹In, ⁶⁷Ga, or ⁶⁸Ga. In another embodiment, the radionuclide used for diagnostic purposes is ¹¹¹n, or ⁶⁷Ga. Examples of the radionuclide used for therapeutic purposes are ¹⁶⁶Ho, ¹⁶⁵Dy, ⁹⁰Y, ¹¹⁵mIn, ⁵²Fe, or ⁷²Ga. In one embodiment, the radionuclide used for diagnostic purposes is ¹⁶⁶Ho or ⁹⁰Y. Examples of the radionuclides used for both therapeutic and diagnostic purposes include ¹⁵³Sm, ¹⁷⁷Lu, ¹⁵⁹Gd, ¹⁷⁵Yb, or ⁴⁷Sc. In one embodiment, the radionuclide is ¹⁵³SM, ¹⁷⁷Lu, ¹⁷⁵Yb, or ¹⁵⁹Gd.

Preferred metal radionuclides include ⁹⁰Y, ⁹⁹mTc, ¹¹¹In, ⁴⁷Sc, ⁶⁷Ga, ⁵¹Cr, ⁷⁷ mSn, ⁶⁷Cu, ¹⁶⁷Tm, ⁹⁷Ru, ¹⁸⁸Re, ¹⁷⁷Lu, ¹⁹⁹Au, ⁴⁷Sc, ⁶⁷Ga, ⁵¹Cr, ¹⁷⁷m, ⁶⁷Cu, ¹⁶⁷Tm, ⁹⁵Ru, ¹⁷⁷Lu, ¹⁹⁹Au, ²⁰³Pb and ¹⁴¹Ce.

In a particular embodiment, Alpha-fetoprotein fusion proteins of the invention to which chelators are covalently attached may be labeled with a metal ion selected from the group consisting of ⁹⁰Y, ¹¹¹In, ¹⁷⁷Lu, ¹⁶⁶Ho, ²¹⁵Bi, and ²²⁵Ac.

Moreover, Remitting radionuclides, such as 99mTc, ¹¹¹In, ⁶⁷Ga, and ¹⁶⁹Yb have been approved or under investigation for diagnostic imaging, while beta-emitters, such as ⁶⁷Cu, ¹¹¹Ag, ¹⁸⁶Re, and ⁹⁰Y are useful for the applications in tumor therapy. Also other useful radionuclides include gamma-emitters, such as ⁹⁹mTc, ¹¹¹In, ⁶⁷Ga, and ¹⁶⁹Y, and beta-emitters, such as ⁶⁷Cu, ¹¹¹Ag, ¹⁸⁶Re, ¹⁸⁶Re and ⁹⁰Y, as well as other radionuclides of interest such as ²¹¹At, ²¹²Bi, ¹⁷⁷Lu, ⁸⁶Rb, ¹⁰⁵Rh, ¹⁵³Sm, ¹⁹⁸Au, ¹⁴⁹Pm, ⁸⁵Sr, ¹⁴²Pr, ²¹⁴Pb, ¹⁰⁹Pd, ¹⁶⁶Ho, ²⁰⁸Tl, ⁴⁴Sc. Alpha-fetoprotein fusion proteins of the invention to which chelators are covalently attached may be labeled with the radionuclides described above.

In another embodiment, Alpha-fetoprotein fusion proteins of the invention to which chelators are covalently attached may be labeled with paramagnetic metal ions including ions of transition and lanthamide metal, such as metals having atomic numbers of 21-29, 42, 43, 44, or 57-71, in particular ions of Cr, V, Mn, Fe, Co, Ni, Cu, La, Ce, Pr, Nd, Pm, Sm, Eu, Gd, Th, Dy, Ho, Er, Tm, Yb, and Lu. The paramagnetic metals used in compositions for magnetic resonance imaging include the elements having atomic numbers of 22 to 29, 42, 44 and 58-70.

In another embodiment, Alpha-fetoprotein fusion proteins of the invention to which chelators are covalently attached may be labeled with fluorescent metal ions including lanthamides, in particular La, Ce, Pr, Nd, Pm, Sm, Eu (e.g., ¹⁵²Eu), Gd, Th, Dy, Ho, Er, Tm, Yb, and Lu.

In another embodiment, Alpha-fetoprotein fusion proteins of the invention to which chelators are covalently attached may be labeled with heavy metal-containing reporters may include atoms of Mo, Bi, Si, and W.

It is also possible to label the alpha-fetoprotein fusion proteins with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, ophthaldehyde and fluorescamine. The alpha-fetoprotein fusion protein can also be detectably labeled using fluorescence emitting metals such as ¹⁵²Eu, or others of the lanthamide series. These metals can be attached to the antibody using such metal chelating groups as diethylenetriaminepentacetic acid (DTPA) or ethylenediaminetetraacetic acid (EDTA).

The alpha-fetoprotein fusion proteins can also can be detectably labeled by coupling it to a chemiluminescent compound. The presence of the chemiluminescent-tagged alpha-fetoprotein fusion protein is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label alpha-fetoprotein fusion proteins of the invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

IX. Transgenic Organisms

Transgenic organisms that express the alpha-fetoprotein fusion proteins of the invention are also included in the invention. Transgenic organisms are genetically modified organisms into which recombinant, exogenous or cloned genetic material has been transferred. Such genetic material is often referred to as a transgene. The nucleic acid sequence of the transgene may include one or more transcriptional regulatory sequences and other nucleic acid sequences such as introns, that may be necessary for optimal expression and secretion of the encoded protein. The transgene may be designed to direct the expression of the encoded protein in a manner that facilitates its recovery from the organism or from a product produced by the organism, e.g. from the milk, blood, urine, eggs, hair or seeds of the organism. The transgene may consist of nucleic acid sequences derived from the genome of the same species or of a different species than the species of the target animal. The transgene may be integrated either at a locus of a genome where that particular nucleic acid sequence is not otherwise normally found or at the normal locus for the transgene.

The term “germ cell line transgenic organism” refers to a transgenic organism in which the genetic alteration or genetic information was introduced into a germ line cell, thereby conferring the ability of the transgenic organism to transfer the genetic information to offspring. If such offspring in fact possess some or all of that alteration or genetic information, then they too are transgenic organisms. The alteration or genetic information may be foreign to the species of organism to which the recipient belongs, foreign only to the particular individual recipient, or may be genetic information already possessed by the recipient. In the last case, the altered or introduced gene may be expressed differently than the native gene.

A transgenic organism may be a transgenic animal or a transgenic plant. Transgenic animals can be produced by a variety of different methods including transfection, electroporation, microinjection, gene targeting in embryonic stem cells and recombinant viral and retroviral infection (U.S. Pat. No. 4,736,866; U.S. Pat. No. 5,602,307; Mullins et al., Hypertension, 22(4):630-633 (1993); Brenin et al., Surg. Oncol., 6(2)99-110 (1997); Tuan (ed.), Recombinant Gene Expression Protocols, Methods in Molecular Biology, No. 62 (Humana Press, 1997). The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307.

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor I (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al., Genetics, 143(4):1753-1760 (1996)); or, are capable of generating a fully human antibody response (McCarthy, The Lancet, 349(9049):405 (1997)).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (Kim et al., Mol. Reprod. Dev., 46(4):515-526 (1997); Houdebine, Reprod. Nutr. Dev., 35(6):609-617 (1995); Petters, Reprod. Fertil. Dev., 6(5):643-645 (1994); Schnieke et al., Science, 278(5346):2130-2133 (1997); and Amoah, J. Animal Science, 75(2):578-585 (1997)).

To direct the secretion of the transgene-encoded protein of the invention into the milk of transgenic mammals, it may be put under the control of a promoter that is preferentially activated in mammary epithelial cells. Promoters that control the genes encoding milk proteins are preferred, for example the promoter for casein, beta lactoglobulin, whey acid protein, or lactalbumin (DiTullio, BioTechnology, 10:74-77 (1992); Clark et al., BioTechnology, 7:487-492 (1989); Gorton et al., BioTechnology, 5:1183-1187 (1987); and Soulier et al., FEBS Letts., 297:13 (1992)). The transgenic mammals of choice would produce large volumes of milk and have long lactating periods, for example goats, cows, camels or sheep.

An alpha-fetoprotein fusion protein of the invention can also be expressed in a transgenic plant, e.g. a plant in which the DNA transgene is inserted into the nuclear or plastidic genome. Plant transformation procedures used to introduce foreign nucleic acids into plant cells or protoplasts are known in the art. See Methods in Enzymology, Vol. 153 (“Recombinant DNA Part D”), Wu and Grossman Eds., (Academic Press, 1987); and European Patent Application No. EP 693554 A1. Methods for generation of genetically engineered plants are further described in U.S. Pat. No. 5,283,184, U.S. Pat. No. 5,482,852, and European Patent Application No EP 693 554 A1.

X. Pharmaceutical or Therapeutic Compositions

The alpha-fetoprotein fusion proteins of the invention or formulations thereof may be administered by any conventional method including parenteral (e.g. subcutaneous or intramuscular) injection or intravenous infusion, orally, rectally, parenterally, intracistemally, intravaginally, intraperitoneally, topically (as by powders, ointments, gels, drops or transdermal patch), otically, ocularly, bucally, pulmonarily (e.g., as an oral or nasal spray or as an aerosol dispersion). The treatment may consist of a single dose or a plurality of doses over a period of time.

Pharmaceutical formulations of the AFP fusion proteins may include aerosol formulations. Suitable aerosol formulations may include aqueous, propellant-based or nonpropellant-based formulations for pulmonary delivery, nasal delivery, or both, in which essentially every inhaled particle contains the AFP fusion protein. Suitable aerosol formulations of the AFP fusion protein may include dry powder aerosol formulations (e.g., dry powder, propellant-based aerosol formulations). The AFP fusion protein may be present in the aerosol formulation at any suitable concentration or concentration range. For aqueous aerosol formulations, the AFP fusion protein may be present, for example, at a concentration range of about 0.05 mg/mL, 0.1 mg/mL, 2 mg/mL, 40 mg/mL, or 100 mg/mL, to about 600 mg/mL. about 0.05 mg/g, 0.1 mg/g, 2 mg/g, 40 mg/g, or 100 mg/g, to about 990 mg/g. Aerosol formulations of the AFP fusion proteins may provide effective delivery of the AFP fusion protein to appropriate areas of the lung cavities, nasal cavities, or both. In some embodiments, aerosol formulations may deliver an effective concentration of the AFP fusion protein to the lung in less than 120 seconds (preferably less than 60 seconds, more preferably less than 30 seconds, most preferably less than 15 seconds).

A. Pharmaceutical Carriers

While it is possible for an alpha-fetoprotein fusion protein of the invention to be administered alone, it is preferable to present it as a pharmaceutical formulation, together with one or more acceptable carriers. The carrier(s) must be “acceptable” in the sense of being compatible with the alpha-fetoprotein fusion protein and not deleterious to the recipients thereof. Typically, the carriers will be water or saline which will be sterile and pyrogen free. Alpha-fetoprotein fusion proteins of the invention are particularly well suited to formulation in aqueous carriers such as sterile pyrogen free water, saline or other isotonic solutions because of their extended shelf-life in solution. For instance, pharmaceutical compositions of the invention may be formulated well in advance in aqueous form, for instance, weeks or months or longer time periods before being dispensed.

Generally, the formulations are prepared by contacting the alpha-fetoprotein fusion protein and/or polynucleotide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably comprises minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as alpha-fetoprotein, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The alpha-fetoprotein fusion protein is typically formulated in such vehicles at a concentration of about 0.1 mg/mL to 100 mg/ml, or about 1 mg/mL to about 10 mg/mL, at a pH of about 3 to 8. It will be understood that the use of certain of the foregoing excipients, carriers, or stabilizers will result in the formation of polypeptide salts.

For example, formulations containing the alpha-fetoprotein fusion protein may be prepared taking into account the extended shelf-life of the alpha-fetoprotein fusion protein in aqueous formulations. As discussed above, the shelf-life of many of these therapeutic proteins are markedly increased or prolonged after fusion to alpha-fetoprotein.

In instances where aerosol administration is appropriate, the alpha-fetoprotein fusion proteins of the invention can be formulated as aerosols using standard procedures. The term “aerosol” includes any gas-borne suspended phase of an alpha-fetoprotein fusion protein of the instant invention which is capable of being inhaled into the bronchioles or nasal passages, and includes dry powder and aqueous aerososl, and pulmonary and nasal aerosols. Specifically, aerosol includes a gas-borne suspension of droplets of an alpha-fetoprotein fusion protein of the instant invention, as may be produced in a metered dose inhaler or nebulizer, or in a mist sprayer. Aerosol also includes a dry powder composition of a compound of the invention suspended in air or other carrier gas, which may be delivered by insufflation from an inhaler device, for example. See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al., Pharmacol. Toxicol. Methods, 27:143-159 (1992).

The formulations of the invention are also typically non-immunogenic, in part, because of the use of the components of the alpha-fetoprotein fusion protein being derived from the proper species. For instance, for human use, both the therapeutic protein and alpha-fetoprotein portions of the alpha-fetoprotein fusion protein will typically be human. In some cases, wherein either component is non human-derived, that component may be humanized by substitution of key amino acids so that specific epitopes appear to the human immune system to be human in nature rather than foreign.

B. Exemplary Dosage Forms

The formulations may conveniently be presented in unit dosage form and may be prepared by any of the methods well known in the art of pharmacy. Such methods include the step of bringing into association the alpha-fetoprotein fusion protein with the carrier that constitutes one or more accessory ingredients. In general the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation appropriate for the intended recipient; and aqueous and non-aqueous sterile suspensions which may include suspending agents and thickening agents. The formulations may be presented in unit-dose or multi-dose containers, for example sealed ampules, vials or syringes, and may be stored in a freeze-dried (lyophilised) condition requiring only the addition of the sterile liquid carrier, for example water for injections, immediately prior to use. Extemporaneous injection solutions and suspensions may be prepared from sterile powders. Dosage formulations may contain the therapeutic protein portion at a lower molar concentration or lower dosage compared to the non-fused standard formulation for the therapeutic protein given the extended serum hAFP-life exhibited by many of the alpha-fetoprotein fusion proteins of the invention.

Formulations or compositions of the invention may be packaged together with, or included in a kit with, instructions or a package insert referring to the extended shelf-life of the alpha-fetoprotein fusion protein component. For instance, such instructions or package inserts may address recommended storage conditions, such as time, temperature and light, taking into account the extended or prolonged shelf-life of the alpha-fetoprotein fusion proteins of the invention. Such instructions or package inserts may also address the particular advantages of the alpha-fetoprotein fusion proteins of the inventions, such as the ease of storage for formulations that may require use in the field, outside of controlled hospital, clinic or office conditions. As described above, formulations of the invention may be in aqueous form and may be stored under less than ideal circumstances without significant loss of therapeutic activity.

Alpha-fetoprotein fusion proteins of the invention can also be included in nutraceuticals. For instance, certain alpha-fetoprotein fusion proteins of the invention may be administered in natural products, including milk or milk product obtained from a transgenic mammal which expresses alpha-fetoprotein fusion protein. Such compositions can also include plant or plant products obtained from a transgenic plant which expresses the alpha-fetoprotein fusion protein. The alpha-fetoprotein fusion protein can also be provided in powder or tablet form, with or without other known additives, carriers, fillers and diluents. Exemplary nutraceuticals are described in Scott Hegenhart, Food Product Design, December 1993.

The invention also provides methods of treatment and/or prevention of diseases or disorders (such as, for example, any one or more of the diseases or disorders disclosed herein) by administration to a subject of an effective amount of an alpha-fetoprotein fusion protein of the invention or a polynucleotide encoding an alpha-fetoprotein fusion protein of the invention (“alpha-fetoprotein fusion polynucleotide”) in a pharmaceutically acceptable carrier.

The alpha-fetoprotein fusion protein and/or polynucleotide will be formulated and dosed in a fashion consistent with good medical practice, taking into account the clinical condition of the individual patient (especially the side effects of treatment with the alpha-fetoprotein fusion protein and/or polynucleotide alone), the site of delivery, the method of administration, the scheduling of administration, and other factors known to practitioners. The “effective amount” for purposes herein is thus determined by such considerations.

As a general proposition, the total pharmaceutically effective amount of the alpha-fetoprotein fusion protein administered parenterally per dose will be in the range of about 1 ug/kg/day to 10 mg/kg/day of patient body weight, although, as noted above, this will be subject to therapeutic discretion. In other embodiments, this dose is at least about 0.01 mg/kg/day, and for humans between about 0.01 and about 1 mg/kg/day. If given continuously, the alpha-fetoprotein fusion protein is typically administered at a dose rate of about 1 ug/kg/hour to about 50 ug/kg/hour, either by multiple (for example, 14) injections per day or by continuous subcutaneous infusions, for example, using a mini-pump. An intravenous bag solution may also be employed. The length of treatment needed to observe changes and the interval following treatment for responses to occur appears to vary depending on the desired effect.

“Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Alpha-fetoprotein fusion proteins and/or polynucleotides of the invention are also suitably administered by sustained-release systems. Examples of sustained-release alpha-fetoprotein fusion proteins and/or polynucleotides are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. Additional examples of sustained-release alpha-fetoprotein fusion proteins and/or polynucleotides include suitable polymeric materials (such as, for example, semi-permeable polymer matrices in the form of shaped articles, e.g., films, or mirocapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers, 22:547-556 (1983)), poly(2-hydroxyethyl methacrylate) (Langer et al., J. Biomed. Mater. Res., 15:167-277 (1981), and Langer, Chem. Tech., 12:98-105 (1982)), ethylene vinyl acetate (Langer et al., Id.) or poly-D-(−)-3-hydroxybutyric acid (EP 133,988).

Sustained-release alpha-fetoprotein fusion proteins and/or polynucleotides also include liposomally entrapped alpha-fetoprotein fusion proteins and/or polynucleotides of the invention (see generally, Langer, Science, 249:1527-1533 (1990); Treat et al., in Liposomes in the Therapy of Infectious Disease and Cancer, Lopez-Berestein and Fidler (eds.), pp. 317-327 and 353-365 (Liss, N.Y., 1989). Liposomes comprising the alpha-fetoprotein fusion protein and/or polynucleotide are prepared by methods known per se: DE 3,218,121; Epstein et al., Proc. Natl. Acad. Sci. (USA), 82:3688-3692 (1985); Hwang et al., Proc. Natl. Acad. Sci. (USA), 77:40304034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapeutic.

In yet an additional embodiment, the alpha-fetoprotein fusion proteins and/or polynucleotides of the invention are delivered by way of a pump (see Langer, supra; Sefton, CRC Crit. Ref. Biomed. Eng., 14:201 (1987); Buchwald et al., Surgery, 88:507 (1980); Saudek et al., N. Engl. J. Med., 321:574 (1989)).

Other controlled release systems are discussed in the review by Langer (Science 249:1527-1533 (1990)).

For parenteral administration, in one embodiment, the alpha-fetoprotein fusion protein and/or polynucleotide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the therapeutic.

Any pharmaceutical used for therapeutic administration can be sterile. Sterility is readily accomplished by filtration through sterile filtration membranes (e.g., 0.2 micron membranes). Alpha-fetoprotein fusion proteins and/or polynucleotides generally are placed into a container having a sterile access port, for example, an intravenous solution bag or vial having a stopper pierceable by a hypodermic injection needle.

Alpha-fetoprotein fusion proteins and/or polynucleotides ordinarily will be stored in unit or multi-dose containers, for example, sealed ampoules or vials, as an aqueous solution or as a lyophilized formulation for reconstitution. As an example of a lyophilized formulation, 10-mi vials are filled with 5 ml of sterile-filtered 1% (w/v) aqueous alpha-fetoprotein fusion protein and/or polynucleotide solution, and the resulting mixture is lyophilized. The infusion solution is prepared by reconstituting the lyophilized alpha-fetoprotein fusion protein and/or polynucleotide using bacteriostatic Water-for-Injection.

The invention also provides a pharmaceutical pack or kit comprising one or more containers filled with one or more of the ingredients of the alpha-fetoprotein fusion proteins and/or polynucleotides of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the alpha-fetoprotein fusion proteins and/or polynucleotides may be employed in conjunction with other therapeutic compounds.

C. Adjuvants

The alpha-fetoprotein fusion proteins and/or polynucleotides of the invention may be administered alone or in combination with adjuvants. Adjuvants that may be administered with the alpha-fetoprotein fusion proteins and/or polynucleotides of the invention include, but are not limited to, cytokines and/or interleukins (such as IL2, IL3, IL4, IL5, IL6, IL7, IL8, IL-9, IL10, IL-11, IL12, IL13, IL-14, IL15, IIL16, IL-17, IL-18, IL-19, IL-20, IL-21, anti-CD40, CD40L, IFN-gamma, TNF-alpha, IL-1alpha, IL-1beta), Lipid A, including monophosphoryl lipid A, bacterial products, endotoxins, cholesterol, fatty acids, aliphatic amines, paraffinic and vegetable oils, threonyl derivative, and muramyl dipeptide, alum, alum plus deoxycholate (ImmunoAg), MTP-PE (Biocine Corp.), QS21 (Genentech, Inc.), BCG (e.g., THERACYS®), MPL and nonviable preparations of Corynebacterium parvum. In a specific embodiment, alpha-fetoprotein fusion proteins and/or polynucleotides of the invention are administered in combination with alum. In another specific embodiment, alpha-fetoprotein fusion proteins and/or polynucleotides of the invention are administered in combination with QS-21. Further adjuvants that may be administered with the alpha-fetoprotein fusion proteins and/or polynucleotides of the invention include, but are not limited to, Monophosphoryl lipid immunomodulator, AdjuVax 100a, QS-21, QS-18, CRL1005, Aluminum salts, MF-59, and Virosomal adjuvant technology.

D. Vaccines

Vaccines that may be administered with the alpha-fetoprotein fusion proteins and/or polynucleotides of the invention include any antigen capable of eliciting an immune response. Exemplary vaccines include, but are not limited to, vaccines directed toward protection against MMR (measles, mumps, rubella), polio, varicella, tetanus/diptheria, hepatitis A, hepatitis B, Haemophilus influenzae B, whooping cough, pneumonia, influenza, Lyme's Disease, rotavirus, cholera, yellow fever, Japanese encephalitis, poliomyelitis, rabies, typhoid fever, pertussis, PA-toxin (e.g., anthrax), Human Immunodeficiency Virus (HIV-1 and HIV-2), Avian Flu antigen (e.g., H5N1), cancer, Severe Acute Respiratory Syndrome (SARS), and tuberculosis. Useful antigens include but are not limited to viral, prion, bacterial, parasitic, mycotic, etc. antigens.

Glycosylation of AFP can be exploited to improve immunogenicity, and therefore efficacy, of AFP-fusion proteins to be used as vaccines. When produced so that the glycosylation pattern is non-human in nature, either by chemical or enzymatic modification or by production in hosts that produce glycosylation patterns that vary from the AFP standard (hosts include but are not exclusive to yeasts like Saccharomyces cerevisiae and Pichia pastoris), increased antigenicity is observed (J. Immunol., 175(11):7496-503 (Dec. 1, 2005)). This makes AFP a uniquely superior vaccine platform.

Combinations may be administered either concomitantly, e.g., as an admixture, separately but simultaneously or concurrently; or sequentially. In addition, as used herein “combination administration” includes compounds which are attached to the AFP protein at either the C or N terminus (e.g., an antigen at the C terminus and an adjuvant at the N terminus; or two different antibodies useful in cancer therapy, one at the C terminus and one at the N terminus of AFP). This also includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

E. Combination Compositions Comprising the AFP Fusion Proteins

The alpha-fetoprotein fusion proteins and/or polynucleotides of the invention may be administered alone or in combination with other therapeutic agents. Alpha-fetoprotein fusion protein and/or polynucleotide agents that may be administered in combination with the alpha-fetoprotein fusion proteins and/or polynucleotides of the invention, include but not limited to, chemotherapeutic agents, antibiotics, steroidal and non-steroidal anti-inflammatories, conventional immunotherapeutic agents, and/or therapeutic treatments described below. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

1. Anticoagulant, Thrombolytic and/or Antiplatelet Drugs

In one embodiment, the alpha-fetoprotein fusion proteins and/or polynucleotides of the invention are administered in combination with an anticoagulant. Anticoagulants that may be administered with the compositions of the invention include, but are not limited to, heparin, low molecular weight heparin, warfarin sodium (e.g., COUMADIN®), dicumarol, 4-hydroxycoumarin, anisindione (e.g., MIRADON™), acenocoumarol (e.g., nicoumalone, SINTHROME™), indan-1,3-dione, phenprocoumon (e.g., MARCUMAR™), ethyl biscoumacetate (e.g., TROMEXAN™), and aspirin. In a specific embodiment, compositions of the invention are administered in combination with heparin and/or warfarin. In another specific embodiment, compositions of the invention are administered in combination with warfarin. In another specific embodiment, compositions of the invention are administered in combination with warfarin and aspirin. In another specific embodiment, compositions of the invention are administered in combination with heparin. In another specific embodiment, compositions of the invention are administered in combination with heparin and aspirin.

In another embodiment, the alpha-fetoprotein fusion proteins and/or polynucleotides of the invention are administered in combination with thrombolytic drugs. Thrombolytic drugs that may be administered with the compositions of the invention include, but are not limited to, plasminogen, lys-plasminogen, alpha2-antiplasmin, streptokinae (e.g., KABWNASE™), antiresplace (e.g., EMINASE™), tissue plasminogen activator (t-PA, altevase, ACTIVASE™), urokinase (e.g., ABBOKINASE™), sauruplase, (Prourokinase, single chain urohinase), and aminocaproic acid (e.g., AMICAR™). In a specific embodiment, compositions of the invention are administered in combination with tissue plasminogen activator and aspirin.

In another embodiment, the alpha-fetoprotein fusion proteins and/or polynucleotides of the invention are administered in combination with antiplatelet drugs. Antiplatelet drugs that may be administered with the compositions of the invention include, but are not limited to, aspirin, dipyridamole (e.g., PERSANTINE™), and ticlopidine (e.g., TICLID™).

In specific embodiments, the use of anti-coagulants, thrombolytic and/or antiplatelet drugs in combination with alpha-fetoprotein fusion proteins and/or polynucleotides of the invention is contemplated for the prevention, diagnosis, and/or treatment of thrombosis, arterial thrombosis, venous thrombosis, thromboembolism, pulmonary embolism, atherosclerosis, myocardial infarction, transient ischemic attack, unstable angina. In specific embodiments, the use of anticoagulants, thrombolytic drugs and/or antiplatelet drugs in combination with alpha-fetoprotein fusion proteins and/or polynucleotides of the invention is contemplated for the prevention of occlusion of saphenous grafts, for reducing the risk of periprocedural thrombosis as might accompany angioplasty procedures, for reducing the risk of stroke in patients with atrial fibrillation including nonrheumatic atrial fibrillation, for reducing the risk of embolism associated with mechanical heart valves and or mitral valves disease. Other uses for the therapeutics of the invention, alone or in combination with antiplatelet, anticoagulant, and/or thrombolytic drugs, include, but are not limited to, the prevention of occlusions in extracorporeal devices (e.g., intravascular canulas, vascular access shunts in hemodialysis patients, hemodialysis machines, and cardiopulmonary bypass machines).

2. Antiretroviral Agents

In certain embodiments, alpha-fetoprotein fusion proteins and/or polynucleotides of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs). NRTIs that may be administered in combination with the alpha-fetoprotein fusion proteins and/or polynucleotides of the invention, include, but are not limited to, RETROVIR™ (zidovudine/AZT), VIDEX™ (didanosinelddl), HIVID™ (zalcitabine/ddC), ZERIT™ (stavudine/d4T), EPIVIR™ (lamivudine/3TC), and COMBIVIR™ (zidovudine/lamivudine). NNRTIs that may be administered in combination with the alpha-fetoprotein fusion proteins and/or polynucleotides of the invention, include, but are not limited to, VIRAMUNE™ (nevirapine), RESCRIPTOR™ (delavirdine), and SUSTIVA™ (efavirenz). Protease inhibitors that may be administered in combination with the alpha-fetoprotein fusion proteins and/or polynucleotides of the invention, include, but are not limited to, CRIXIVAN™ (indinavir), NORVIR™ (ritonavir), INVIRASE™ (saquinavir), and VIRACEPT™ (nelfinavir). In a specific embodiment, antiretroviral agents, nucleoside reverse transcriptase inhibitors, non-nucleoside reverse transcriptase inhibitors, and/or protease inhibitors may be used in any combination with alpha-fetoprotein fusion proteins and/or polynucleotides of the invention to treat AIDS and/or to prevent or treat HIV infection.

Additional NRTIs include LODENOSINE™ (F-ddA; an acid-stable adenosine NRTI; Triangle/Abbott; COVIRACIL™ (emtricitabine/FTC; structurally related to lamivudine (3TC) but with 3- to 10-fold greater activity in vitro; Triangle/Abbott); dOTC (BCH-10652, also structurally related to lamivudine but retains activity against a substantial proportion of lanivudine-resistant isolates; Biochem Pharma); Adefovir (refused approval for anti-HIV therapy by FDA; Gilead Sciences); PREVEON® (Adefovir Dipivoxil, the active prodrug of adefovir; its active form is PMEA-pp); TENOFOVIR™ (bis-POC PMPA, a PMPA prodrug; Gilead); DAPD/DXG (active metabolite of DAPD; Triangle/Abbott); D-D4FC (related to 3TC, with activity against AZT/3TC-resistant virus); GW420867.times. (Glaxo Wellcome); ZIAGEN™ (abacavir/159U89; Glaxo Wellcome Inc.); CS-87 (3′ azido-2′,3′-dideoxyuridine; WO 99/66936); and S-acyl-2-thioethyl (SATE)-bearing prodrug forms of .beta.-L-FD4C and P-L-FddC (WO 98/17281).

Additional NNRTIs include COACTINON™ (Emivirine/MKC442, potent NNRTI of the HEPT class; Triangle/Abbott); CAPRAVIRINE™ (AG-1549/S-1153, a next generation NNRTI with activity against viruses containing the K103N mutation; Agouron); PNU-142721 (has 20- to 50-fold greater activity than its predecessor delavirdine and is active against K103N mutants; Pharmacia & Upjohn); DPC-961 and DPC-963 (second-generation derivatives of efavirenz, designed to be active against viruses with the K103N mutation; DuPont); GW420867X (has 25-fold greater activity than HBY097 and is active against K103N mutants; Glaxo Wellcome); CALANOLIDE A (naturally occurring agent from the latex tree; active against viruses containing either or both the Y181C and K103N mutations); and Propolis (WO 99/49830).

Additional protease inhibitors include LOPINAVIR™ (ABT378/r; Abbott Laboratories); BMS-232632 (an azapeptide; Bristol-Myres Squibb); TIPRANAVIR™ (PNU-140690, a non-peptic dihydropyrone; Pharmacia & Upjohn); PD-178390 (a nonpeptidic dihydropyrone; Parke-Davis); BMS 232632 (an azapeptide; Bristol-Myers Squibb); L-756,423 (an indinavir analog; Merck); DMP450 (a cyclic urea compound; Avid & DuPont); AG-1776 (a peptidomimetic with in vitro activity against protease inhibitor-resistant viruses; Agouron); VX-175/GW433908 (phosphate prodrug of amprenavir; Vertex & Glaxo Welcome); CGP61755 (Ciba); and AGENERASE™ (amprenavir; Glaxo Wellcome Inc.).

Additional antiretroviral agents include fusion inhibitors/gp41 binders. Fusion inhibitors/gp41 binders include T-20 (a peptide from residues 643-678 of the HIV gp41 transmembrane protein ectodomain which binds to gp41 in its resting state and prevents transformation to the fusogenic state; Trimeris) and T-1249 (a second-generation fusion inhibitor; Trimeris).

Additional antiretroviral agents include fusion inhibitors/chemokine receptor antagonists. Fusion inhibitors/chemokine receptor antagonists include CXCR4 antagonists such as AMD 3100 (a bicyclam), SDF-1 and its analogs, and ALX404C (a cationic peptide), T22 (an 18 amino acid peptide; Trimeris) and the T22 analogs T134 and T140; CCR5 antagonists such as RANTES (9-68), AOP-RANTES, NNY-RANTES, and TAK-779; and CCR5/CXCR4 antagonists such as NSC 651016 (a distamycin analog). Also included are CCR2B, CCR3, and CCR6 antagonists. Chemokine receptor agonists such as RANTES, SDF-1, MEP-1 alpha, MIP-1beta, etc., may also inhibit fusion.

Additional antiretroviral agents include integrase inhibitors. Integrase inhibitors include dicaffeoylquinic (DFQA) acids; L-chicoric acid (a dicaffeoyltartaric (DCTA) acid); quinalizarin (QLC) and related anthraquinones; ZINTEVIR™ (AR 177, an oligonucleotide that probably acts at cell surface rather than being a true integrase inhibitor; Arondex); and naphthols such as those disclosed in WO 98/50347.

Additional antiretroviral agents include hydroxyurea-like compounds such as BCX-34 (a purine nucleoside phosphorylase inhibitor; Biocryst); ribonucleotide reductase inhibitors such as DIDOX™ (Molecules for Health); inosine monophosphate dehydrogenase (IMPDH) inhibitors such as VX-497 (Vertex); and mycopholic acids such as CellCept (mycophenolate mofetil; Roche).

Additional antiretroviral agents include inhibitors of viral integrase, inhibitors of viral genome nuclear translocation such as arylene bis(methylketone) compounds; inhibitors of HIV entry such as AOP-RANTES, NNY-RANTES, RANTES-IgG fusion protein, soluble complexes of RANTES and glycosaminoglycans (GAG), and AMD-3100; nucleocapsid zinc finger inhibitors such as dithiane compounds; targets of HIV Tat and Rev; and pharmacoenhancers such as ABT-378.

Other antiretroviral therapies and adjunct therapies include cytokines and lymphokines such as MIP-1 alpha, MIP-1beta, SDF-1 alpha, IL-2, PROLEUKIN™ (aldesleukin/L2-7001; Chiron), IL4, IL-10, IL-12, and IL-13; interferons such as IFN-alpha2a, IFN-alpha2b, or IFN-beta; antagonists of TNFs, NFkappaB, GM-CSF, M-CSF, and IL-10; agents that modulate immune activation such as cyclosporin and prednisone; vaccines such as Remune™ (HIV Immunogen), APL 400-003 (Apollon), recombinant gp120 and fragments, bivalent (B/E) recombinant envelope glycoprotein, rgp120CM235, MN rgp120, SF-2 rgp120, gp120/soluble CD4 complex, Delta JR-FL protein, branched synthetic peptide derived from discontinuous gp120 C3/C4 domain, fusion-competent immunogens, and Gag, Pol, Nef, and Tat vaccines; gene-based therapies such as genetic suppressor elements (GSEs; WO 98/54366), and intrakines (genetically modified CC chemokines targetted to the ER to block surface expression of newly synthesized CCR5 (Yang et al., PNAS, 94:11567-72 (1997); Chen et al., Nat. Med., 3:1110-16 (1997)); antibodies such as the anti-CXCR4 antibody 12G5, the anti-CCR5 antibodies 2D7, 5C7, PA8, PA9, PA10, PA11, PA12, and PA 14, the anti-CD4 antibodies Q4120 and RPA-T4, the anti-CCR3 antibody 7B11, the anti-gp120 antibodies 17b, 48d, 447-52D, 257-D, 268-D and 50.1, anti-Tat antibodies, anti-TNF-alpha antibodies, and monoclonal antibody 33A; aryl hydrocarbon (AH) receptor agonists and antagonists such as TCDD, 3,3′,4,4′,5-pentachlorobiphenyl, 3,3′,4,4′-tetrachlorobiphenyl, and alpha-naphthoflavone (WO 98/30213); and antioxidants such as gamma-L-glutamyl-L-cysteine ethyl ester (gamma-GCE; WO 99/56764).

In a further embodiment, the alpha-fetoprotein fusion proteins and/or polynucleotides of the invention are administered in combination with an antiviral agent. Antiviral agents that may be administered with the alpha-fetoprotein fusion proteins and/or polynucleotides of the invention include, but are not limited to, acyclovir, ribavirin, amantadine, remantidine, maxamine, or thymAFPasin. Specifically, interferon alpha-fetoprotein fusion protein can be administered in combination with any of these agents. Moreover, interferon alpha alpha-fetoprotein fusion protein can also be administered with any of these agents, and preferably, interferon alpha 2a or 2b alpha-fetoprotein fusion protein can be administered with any of these agents. Furthermore, interferon beta alpha-fetoprotein fusion protein can also be administered with any of these agents. Additionally, any of the IFN hybrids alpha-fetoprotein fusion proteins can be administered in combination with any of these agents.

In a most preferred embodiment, interferon alpha-fetoprotein fusion protein is administered in combination with ribavirin. In a further preferred embodiment, interferon alpha alpha-fetoprotein fusion protein is administered in combination with ribavirin. In a further preferred embodiment, interferon alpha 2a alpha-fetoprotein fusion protein is administered in combination with ribavirin. In a further preferred embodiment, interferon alpha 2b alpha-fetoprotein fusion protein is administered in combination with ribavirin. In a further preferred embodiment, interferon beta alpha-fetoprotein fusion protein is administered in combination with ribavirin. In a further preferred embodiment, hybrid interferon alpha-fetoprotein fusion protein is administered in combination with ribavirin.

3. Anti-Infection Agents

In other embodiments, alpha-fetoprotein fusion proteins and/or polynucleotides of the invention may be administered in combination with anti-opportunistic infection agents. Anti-opportunistic agents that may be administered in combination with the alpha-fetoprotein fusion proteins and/or polynucleotides of the invention, include, but are not limited to, TRIMETHOPRIM-SULFAMETHOXAZOLE™, DAPSONE™, PENTAMIDINE™, ATOVAQUONE™, ISONIAZID™, RIFAMPIN™, PYRAZINAMIDE™, ETHAMBUTOL™, RIFABUTIN™, CLARfTHROMYCIN™, AZITHROMYC™, GANCICLOVIR™, FOSCARNE™, CIDOFOVIR™, FLUCONAZOLE™, ITRACONAZOLE™, KETOCONAZOLE™, ACYCLOVIR™, FAMCICOLVIR™, PYRINMETHAMINE™, LEUCOVORIN™, NEUPOGEN™ (filgrastim/G-CSF), and LEUKINE™ (sargramostim/GM-CSF). In a specific embodiment, alpha-fetoprotein fusion proteins and/or polynucleotides of the invention are used in any combination with TRIMETHOPRIM-SULFAMETHOXAZOLE™, DAPSONE™, PENTAMIDINE™, and/or ATOVAQUONE™ to prophylactically treat or prevent an opportunistic Pneumocystis carinii pneumonia infection. In another specific embodiment, alpha-fetoprotein fusion proteins and/or polynucleotides of the invention are used in any combination with ISONIAZID™, RIFAMPIN™, PYRAZINAMIDE™, and/or ETHAMBUTOL™ to prophylactically treat or prevent an opportunistic Mycobacterium avium complex infection. In another specific embodiment, alpha-fetoprotein fusion proteins and/or polynucleotides of the invention are used in any combination with RIFABUTIN™, CLARITHROMYCIN™, and/or AZITHROMYCIN™ to prophylactically treat or prevent an opportunistic Mycobacterium tuberculosis infection. In another specific embodiment, alpha-fetoprotein fusion proteins and/or polynucleotides of the invention are used in any combination with GANCICLOVIR™, FOSCARNET™, and/or CIDOFOVIR™ to prophylactically treat or prevent an opportunistic cytomegalovirus infection. In another specific embodiment, alpha-fetoprotein fusion proteins and/or polynucleotides of the invention are used in any combination with FLUCONAZOLE™, IRCONAZOLE™, and/or KETOCONAZOLE™ to prophylactically treat or prevent an opportunistic fungal infection. In another specific embodiment, alpha-fetoprotein fusion proteins and/or polynucleotides of the invention are used in any combination with ACYCLOVIR™ and/or FAMCICOLVIR™ to prophylactically treat or prevent an opportunistic herpes simplex virus type I and/or type II infection. In another specific embodiment, alpha-fetoprotein fusion proteins and/or polynucleotides of the invention are used in any combination with PYRIMETHAMINE™ and/or LEUCOVORIN™ to prophylactically treat or prevent an opportunistic Toxoplasma gondil infection. In another specific embodiment, alpha-fetoprotein fusion proteins and/or polynucleotides of the invention are used in any combination with LEUCOVORIN™ and/or NEUPOGEN™ to prophylactically treat or prevent an opportunistic bacterial infection.

4. Antibiotics

In a further embodiment, the alpha-fetoprotein fusion proteins and/or polynucleotides of the invention are administered in combination with an antibiotic agent. Antibiotic agents that may be administered with the alpha-fetoprotein fusion proteins and/or polynucleotides of the invention include, but are not limited to, amoxicillin, beta-lactamases, aminoglycosides, beta-lactam (glycopeptide), beta-lactamases, Clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethopfim, trimethoprim-sulfamethoxazole, and vancomycin.

5. Immunostimulant and/or Immunosuppressive Agents

In other embodiments, the alpha-fetoprotein fusion proteins and/or polynucleotides of the invention are administered in combination with immunestimulants. Immunostimulants that may be administered in combination with the alpha-fetoprotein fusion proteins and/or polynucleotides of the invention include, but are not limited to, levamisole (e.g., ERGAMISOL™), isoprinosine (e.g. INOSIPLEX™), interferons (e.g. interferon alpha), and interleukins (e.g., IL-2).

In other embodiments, alpha-fetoprotein fusion proteins and/or polynucleotides of the invention are administered in combination with immunosuppressive agents. Immunosuppressive agents that may be administered in combination with the alpha-fetoprotein fusion proteins and/or polynucleotides of the invention include, but are not limited to, steroids, cyclosporine, cyclosporine analogs, cyclophosphamide methylprednisone, prednisone, azathioprine, FK-506, 115deoxyspergualin, and other immunosuppressive agents that act by suppressing the function of responding T cells. Other immunosuppressive agents that may be administered in combination with the alpha-fetoprotein fusion proteins and/or polynucleotides of the invention include, but are not limited to, prednisolone, methotrexate, thalidomide, methoxsalen, rapamycin, leflunomide, mizoribine (BREDININ™), brequinar, deoxyspergualin, and azaspirane (SKF 105685), ORTHOCLONE OKT® 3 (muromonab-CD3), SANDIMMUNE™, NEORAL™, SANGDYA™ (cyclosporine), PROGRAF® (FK506, tacrolimus), CELLCEPT® (mycophenolate motefil, of which the active metabolite is mycophenolic acid), IMURAN™ (azathioprine), glucocorticosteroids, adrenocortical steroids such as DELTASONE™ (prednisone) and HYDELTRASOLT™(prednisolone), FOLEX™ and MEXATE™ (methotrxate), OXSORALEN-ULTRA™ (methoxsalen) and RAPAMUNE™ (sirolimus). In a specific embodiment, immunosuppressants may be used to prevent rejection of organ or bone marrow transplantation.

In an additional embodiment, alpha-fetoprotein fusion proteins and/or polynucleotides of the invention are administered alone or in combination with one or more intravenous immune globulin preparations. Intravenous immune globulin preparations that may be administered with the alpha-fetoprotein fusion proteins and/or polynucleotides of the invention include, but not limited to, GAMMAR™, IVEEGAM™, SANDOGLOBULIN™, GAMMAGARD S/D™, ATGAM™ (antithymocyte glubulin), and GAMIMune™. In a specific embodiment, alpha-fetoprotein fusion proteins and/or polynucleotides of the invention are administered in combination with intravenous immune globulin preparations in transplantation therapy (e.g., bone marrow transplant).

6. Cancer Therapies

In another embodiment, the alpha-fetoprotein fusion proteins and/or polynucleotides of the invention are administered alone or as part of a combination therapy, either in vivo to patients or in vitro to cells, for the treatment of cancer. In a specific embodiment, the alpha-fetoprotein fusion proteins, particularly IL-2-alpha-fetoprotein fusions, are administered repeatedly during passive immunotherapy for cancer, such as adoptive cell transfer therapy for metastatic melanoma as described in Dudley et al. (Science Express, 19 Sep. 2002., at www.scienceexpress.org).

7. Anti-Inflammatory Agents

In certain embodiments, the alpha-fetoprotein fusion proteins and/or polynucleotides of the invention are administered alone or in combination with an anti-inflammatory agent. Anti-inflammatory agents that may be administered with the alpha-fetoprotein fusion proteins and/or polynucleotides of the invention include, but are not limited to, corticosteroids (e.g. betamethasone, budesonide, cortisone, dexamethasone, hydrocortisone, methylprednisolone, prednisolone, prednisone, and triamcinolone), nonsteroidal anti-inflammatory drugs (e.g., diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic acid, and tolmetin.), as well as antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

8. Angiogenesis Agents

In an additional embodiment, the compositions of the invention are administered alone or in combination with an anti-angiogenic agent. Anti-angiogenic agents that may be administered with the compositions of the invention include, but are not limited to, Angiostatin (Entremed, Rockville, Md.), Troponin-1 (Boston Life Sciences, Boston, Mass.), anti-Invasive Factor, retinoic acid and derivatives thereof, paclitaxel (Taxol), Suramin, Tissue Inhibitor of Metalloproteinase-1, Tissue Inhibitor of Metalloproteinase-2, VEGI, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (MV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include, but are not limited to, platelet factor 4; protamine sulphate; sulphated chitin derivatives (prepared from queen crab shells) (Murata et al., Cancer Res., 51:22-26, (1991)); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5(4-pyridinyl)-2(3H)-oxazolone; Methotrexate; Mitoxantrone; Heparin; Interferons; 2 Macroglobulin-serum; ChIlMP-3 (Pavloff et al., J. Bio. Chem., 267:17321-17326, (1992)); Chymostatin (Tomkinson et al., Biochem J, 286:475-480, (1992)); Cyclodextrin Tetradecasulfate; Eponemycin; Camptothecin; Fumagillin (Ingber et al., Nature, 348:555-557 (1990)); Gold Sodium Thiomalate (“GST”; Matsubara and Ziff, J. Clin. Invest., 79:1440-1446, (1987)); anticollagenase-serum; alpha2-antiplasmin (Holmes et al., J. Biol. Chem., 262(4):1659-1664, (1987)); Bisantrene (National Cancer Institute); Lobenzarit disodium (N-(2)-carboxyphenyl-4-ch-loroanthronilic acid disodium or “CCA”; (Takeuchi et al., Agents Actions, 36:312-316, (1992)); and metalloproteinase inhibitors such as BB94.

Additional anti-angiogenic factors that may also be utilized within the context of the present invention include Thalidomide, (Celgene, Warren, N.J.); Angiostatic steroid; AGM-1470 (H. Brem and J. Folkman, J. Pediatr. Surg., 28:445-51 (1993)); an integrin alpha v beta 3 antagonist (C. Storgard et al., J. Clin. Invest., 103:47-54 (1999)); carboxynaminolmidazole; Carboxyamidotriazole (CA) (National Cancer Institute, Bethesda, Md.); Conbretastatin A4 (CA4P) (OXiGENE, Boston, Mass.); Squalamine (Magainin Pharmaceuticals, Plymouth Meeting, Pa.); TNP470, (Tap Pharmaceuticals, Deerfield, Ill.); ZD-0101 AstraZeneca (London, UK); APRA (CT2584); Benefin, Byrostatin-1 (SC339555); CGP41251 (PKC 412); CM101; Dexrazoxane (ICRF187); DMXAA; Endostatin; Flavopridiol; Genestein; GTE; ImmTher; Iressa (ZD1839); Octreotide (Somatostatin); Panretin; Penacillamine; Photopoint; PI-88; Prinomastat (AG-3340) Purlytin; Suradista (FCE26644); Tamoxifen (Nolvadex); Tazarotene; Tetrathiomolybdate; Xeloda (Capecitabine); and 5-Fluorouracil.

Anti-angiogenic agents that may be administered in combination with the compounds of the invention may work through a variety of mechanisms including, but not limited to, inhibiting proteolysis of the extracellular matrix, blocking the function of endothelial cell-extracellular matrix adhesion molecules, by antagonizing the function of angiogenesis inducers such as growth factors, and inhibiting integrin receptors expressed on proliferating endothelial cells. Examples of anti-angiogenic inhibitors that interfere with extracellular matrix proteolysis and which may be administered in combination with the compositions of the invention include, but are not limited to, AG-3340 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), and Metastat (Aeterna, St-Foy, Quebec). Examples of anti-angiogenic inhibitors that act by blocking the function of endothelial cell-extracellular matrix adhesion molecules and which may be administered in combination with the compositions of the invention include, but are not limited to, EMD-121974 (Merck KcgaA Darmstadt, Germany) and Vitaxin (Ixsys, La Jolla, Calif./Medimmune, Gaithersburg, Md.). Examples of anti-angiogenic agents that act by directly antagonizing or inhibiting angiogenesis inducers and which may be administered in combination with the compositions of the invention include, but are not limited to, Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), and SU-6668 (Sugen). Other anti-angiogenic agents act to indirectly inhibit angiogenesis. Examples of indirect inhibitors of angiogenesis which may be administered in combination with the compositions of the invention include, but are not limited to, IM-862 (Cytran, Kirkland, Wash.), Interferon-alpha, 11LZ2 (Roche, Nutley, N.J.), and Pentosan polysulfate (Georgetown University, Washington D.C.).

In particular embodiments, the use of compositions of the invention in combination with anti-angiogenic agents is contemplated for the treatment, prevention, and/or amelioration of an autoimmune disease, such as for example, an autoimmune disease described herein.

In a particular embodiment, the use of compositions of the invention in combination with anti-angiogenic agents is contemplated for the treatment, prevention, and/or amelioration of arthritis. In a more particular embodiment, the use of compositions of the invention in combination with anti-angiogenic agents is contemplated for the treatment, prevention, and/or amelioration of rheumatoid arthritis.

In another embodiment, the polynucleotides encoding a polypeptide of the invention are administered in combination with an angiogenic protein, or polynucleotides encoding an angiogenic protein. Examples of angiogenic proteins that may be administered with the compositions of the invention include, but are not limited to, acidic and basic fibroblast growth factors, VEGF-1, VEGF-2, VEGF-3, epidermal growth factor alpha and beta, platelet-derived endothelial cell growth factor, platelet-derived growth factor, tumor necrosis factor alpha, hepatocyte growth factor, insulin-like growth factor, colony stimulating factor, macrophage colony stimulating factor, granulocyte/macrophage colony stimulating factor, and nitric oxide synthase.

9. Chemotherapeutic Agents

In additional embodiments, compositions of the invention are administered in combination with a chemotherapeutic agent. Chemotherapeutic agents that may be administered with the alpha-fetoprotein fusion proteins and/or polynucleotides of the invention include, but are not limited to alkylating agents such as nitrogen mustards (for example, Mechlorethamine, cyclophosphamide, Cyclophosphamide Ifosfamide, Melphalan (L-sarcolysin), and Chlorambucil), ethylenimines and methylmelamines (for example, Hexamethylmelamine and Thiotepa), alkyl sulfonates (for example, Busulfan), nitrosoureas (for example, Carmustine (BCNU), Lomustine (CCNU), Semustine (methyl-CCNU), and Streptozocin (streptozotocin)), triazenes (for example, Dacarbazine (DTIC; dimethyltriazenoimidazolecarbo-xamide)), folic acid analogs (for example, Methotrexate (amethopterin)), pyrimidine analogs (for example, Fluorouacil (5-fluorouracil; 5-FU), Floxuridine (fluorodeoxyuridine; FudR), and Cytarabine (cytosine arabinoside)), purine analogs and related inhibitors (for example, Mercaptopurine (6-mercaptopurine; 6-MP), Thioguanine (6-thioguanine; TG), and Pentostatin (2′-deoxycoformycin)), vinca alkaloids (for example, Vinblastine (VLB, vinblastine sulfate)) and Vincristine (vincristine sulfate)), epipodophyllotoxins (for example, Etoposide and Teniposide), antibiotics (for example, Dactinomycin (actinomycin D), Daunorubicin (daunomycin; rubidomycin), Doxorubicin, Bleomycin, Plicamycin (mithramycin), and Mitomycin (mitomycin C), enzymes (for example, L-Asparaginase), biological response modifiers (for example, Interferon-alpha and interferon-alpha-2b), platinum coordination compounds (for example, Cisplatin (cis-DDP) and Carboplatin), anthracenedione (Mitoxantrone), substituted ureas (for example, Hydroxyurea), methylhydrazine derivatives (for example, Procarbazine (N-methylhydrazine; MIH), adrenocorticosteroids (for example, Prednisone), progestins (for example, Hydroxyprogesterone caproate, Medroxyprogesterone, Medroxyprogesterone acetate, and Megestrol acetate), estrogens (for example, Diethylstilbestrol (DES), Diethylstilbestrol diphosphate, Estradiol, and Ethinyl estradiol), antiestrogens (for example, Tamoxifen), androgens (Testosterone proprionate, and Fluoxymesterone), antiandrogens (for example, Flutamide), gonadotropin-releasing hormone analogs (for example, Leuprolide), other hormones and hormone analogs (for example, methyltestosterone, estramustine, estramustine phosphate sodium, chlorotrianisene, and testolactone), and others (for example, dicarbazine, glutamic acid, and mitotane).

In one embodiment, the compositions of the invention are administered in combination with one or more of the following drugs: infliximab (Remicade®, Centocor, Inc.), Trocade® (Roche, RO-32-3555), Leflunomide (Arava®, Hoechst Marion Roussel), Kinere™ (an IL-1 Receptor antagonist also known as Anakinra®, Amgen, Inc.).

In a specific embodiment, compositions of the invention are administered in combination with CHOP (cyclophosphamide, doxorubicin, vincristine, and prednisone) or combination of one or more of the components of CHOP. In one embodiment, the compositions of the invention are administered in combination with anti-CD20 antibodies, human monoclonal anti-CD20 antibodies. In another embodiment, the compositions of the invention are administered in combination with anti-CD20 antibodies and CHOP, or anti-CD20 antibodies and any combination of one or more of the components of CHOP, particularly cyclophosphamide and/or prednisone. In a specific embodiment, compositions of the invention are administered in combination with Rituximab. In a further embodiment, compositions of the invention are administered with Rituximab and CHOP, or Rituximab and any combination of one or more of the components of CHOP, particularly cyclophosphamide and/or prednisone. In a specific embodiment, compositions of the invention are administered in combination with tositumomab. In a further embodiment, compositions of the invention are administered with tositumomab and CHOP, or tositumomab and any combination of one or more of the components of CHOP, particularly cyclophosphamide and/or prednisone. The anti-CD20 antibodies may optionally be associated with radioisotopes, toxins or cytotoxic prodrugs.

In another specific embodiment, the compositions of the invention are administered in combination Zevalin™. In a further embodiment, compositions of the invention are administered with Zevalin™ and CHOP, or Zevalin™ and any combination of one or more of the components of CHOP, particularly cyclophosphamide and/or prednisone. Zevalin™ may be associated with one or more radisotopes. Particularly preferred isotopes are ⁹⁰Y and ¹¹¹In.

In an additional embodiment, the alpha-fetoprotein fusion proteins and/or polynucleotides of the invention are administered in combination with cytokines. Cytokines that may be administered with the alpha-fetoprotein fusion proteins and/or polynucleotides of the invention include, but are not limited to, IL2, IL3, IL4, IL5, IL6, IL7, IL10, IL12, IL13, IL15, anti-CD40, CD40L, IFN-gamma and TNF-alpha. In another embodiment, alpha-fetoprotein fusion proteins and/or polynucleotides of the invention may be administered with any interleukin, including, but not limited to, IL-1alpha, IL-1beta, IL-2, IL-3, ILA4, IL-5, IL-6, IL-7, L8, IL-9, IL-10, IL-11, IL-12, IL-13, IL-14, L115, IIL16, IL-17, IL-18, IL-19, IL-20, and IL-21.

In one embodiment, the alpha-fetoprotein fusion proteins and/or polynucleotides of the invention are administered in combination with members of the TNF family. TNF, TNF-related or TNF-like molecules that may be administered with the alpha-fetoprotein fusion proteins and/or polynucleotides of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-IBBL, DcR3, OX40L, TNF-gamma (WO 96/14328), AIM4-(WO 97/33899), endokine-alpha (WO 98/07880), OPG, and neutrokine-alpha (WO 98/18921, OX40, and nerve growth factor (NGF), and soluble forms of Fas, CD30, CD27, CD40 and 4-IBB, TR2 (WO 96/34095), DR3 (International Publication No. WO 97/33904), DR4 (WO 98/32856), TR5 (WO 98/30693), TRANK, TR9 (WO 98/56892), TR10 (WO 98/54202), 312C2 (WO 98/06842), and TR12, and soluble forms CD154, CD70, and CD153.

In an additional embodiment, the alpha-fetoprotein fusion proteins and/or polynucleotides of the invention are administered in combination with angiogenic proteins. Angiogenic proteins that may be administered with the alpha-fetoprotein fusion proteins and/or polynucleotides of the invention include, but are not limited to, Glioma Derived Growth Factor (GDGF), as disclosed in European Patent Number EP-399816; Platelet Derived Growth Factor-A (PDGF-A), as disclosed in European Patent Number EP-682110; Platelet Derived Growth Factor-B (PDGF-B), as disclosed in European Patent Number EP-282317; Placental Growth Factor (PIGF), as disclosed in International Publication Number WO 92/06194; Placental Growth Factor-2 (PIGF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in WO 98/02543; Vascular Endothelial Growth Factor-D (VEGF-D), as disclosed in WO 98/07832; and Vascular Endothelial Growth Factor-E (VEGF-E), as disclosed in German Patent Number DE19639601.

In an additional embodiment, the alpha-fetoprotein fusion proteins and/or polynucleotides of the invention are administered in combination with Fibroblast Growth Factors. Fibroblast Growth Factors that may be administered with the alpha-fetoprotein fusion proteins and/or polynucleotides of the invention include, but are not limited to, FGF-1, FGF-2, FGF-3, FGF4, FGF-5, FGF-6, FGF-7, FGF-8, FGF-9, FGF-10, FGF-11, FGF-12, FGF-13, FGF-14, and FGF-15.

10. Hematopoietic Growth Factors

In an additional embodiment, the alpha-fetoprotein fusion proteins and/or polynucleotides of the invention are administered in combination with hematopoietic growth factors. Hematopoietic growth factors that may be administered with the alpha-fetoprotein fusion proteins and/or polynucleotides of the invention include, but are not limited to, granulocyte macrophage colony stimulating factor (GM-CSF) (sargranostim, LEUKINE™, PROKINE™), granulocyte colony stimulating factor (G-CSF) (filgrastim, NEUPOGEN™), macrophage colony stimulating factor (M-CSF, CSF-1) erythropoietin (epoetin AFPa, EPOGEN™, PROCRIT™), stem cell factor (SCF, c-kit ligand, steel factor), megakaryocyte colony stimulating factor, PDCY321 (a GMCSF/IL-3 fusion protein), interleukins, especially any one or more of IL-1 through IL-12, interferon-gamma, or thrombopoietin.

In certain embodiments, alpha-fetoprotein fusion proteins and/or polynucleotides of the present invention are administered in combination with adrenergic blockers, such as, for example, acebutolol, atenolol, betaxolol, bisoprolol, carteolol, labetalol, metoprolol, nadolol, oxprenolol, penbutolol, pindolol, propranolol, sotalol, and timolol.

In another embodiment, the alpha-fetoprotein fusion proteins and/or polynucleotides of the invention are administered in combination with an antiarrhythmic drug (e.g., adenosine, amidoarone, bretylium, digitalis, digoxin, digitoxin, diliazem, disopyramide, esmolol, flecamide, lidocaine, mexiletine, moricizine, phenyloin, procainamide, N-acetyl procainamide, propafenone, propranolol, quinidine, sotalol, tocamide, and verapamil).

In another embodiment, the alpha-fetoprotein fusion proteins and/or polynucleotides of the invention are administered in combination with diuretic agents, such as carbonic anhydrase-inhibiting agents (e.g., acetazolamide, dichlorphenamide, and methazolamide), osmotic diuretics (e.g., glycerin, isosorbide, mannitol, and urea), diuretics that inhibit Na⁺-K⁺-2Cl⁻ symport (e.g., furosemide, bumetamide, azosemide, piretamide, tripamide, ethacrynic acid, muzolimine, and torsemide), thiazide and thiazide-like diuretics (e.g., bendroflumethiazide, benzthiazide, chlorothiazide, hydrochlorothiazide, hydroflumethiazide, methyclothiazide, polythiazide, trichormethiazide, chlorthalidone, indapamide, metolazone, and quinethazone), potassium sparing diuretics (e.g., amiloride and triamterene), and mineralcorticoid receptor antagonists (e.g., spironolactone, canrenone, and potassium canrenoate).

In one embodiment, the alpha-fetoprotein fusion proteins and/or polynucleotides of the invention are administered in combination with treatments for endocrine and/or hormone imbalance disorders. Treatments for endocrine and/or hormone imbalance disorders include, but are not limited to, ¹²⁷I, radioactive isotopes of iodine such as ¹³¹I and ¹²³I; recombinant growth hormone, such as HUMATROPE™ (recombinant somatropin); growth hormone analogs such as PROTROPIN™ (somatrem); dopamine agonists such as PARLODEL™ (bromocriptine); somatostatin analogs such as SANDOSTATIN™ (octreotide); gonadotropin preparations such as PREGNYL™, A.P.L.Tm and PROFASI™ (chorionic gonadotropin (CG)), PERGONAL™ (menotropins), and METRODIN™ (urofollitropin (uFSH)); synthetic human gonadotropin releasing hormone preparations such as FACTREL™ and LUTREPULSE™ (gonadorelin hydrochloride); synthetic gonadotropin agonists such as LUPRONM (leuprolide acetate), SUPPRELIN™ (histrelin acetate), SYNAREL™ (nafarelin acetate), and ZOLADEX'M (goserelin acetate); synthetic preparations of thyrotropin-releasing hormone such as RELEFACT TRH™ and THYPINONE™ (protirelin); recombinant human TSH such as THYROGEN™; synthetic preparations of the sodium salts of the natural isomers of thyroid hormones such as L-T₄, SYNTIROID™ and LEVOTHROID™ (levothyroxine sodium), L-T₃, CYTOMEL™ and TRIOSTAT™ (liothyroine sodium), and THYROLART (liotrix); antithyroid compounds such as 6-n-propylthiouracil (propylthiouracil), 1-methyl-2-mercaptoimidazole and TAPAZOLE™ (methimazole), NEO-MERCAZOLE™ (carbimazole); beta-adrenergic receptor antagonists such as propranolol and esmolol; Ca² channel blockers; dexamethasone and iodinated radiological contrast agents such as TELEPAQUE™ (iopanoic acid) and ORAGRAFIN™ (sodium ipodate).

Additional treatments for endocrine and/or hormone imbalance disorders include, but are not limited to, estrogens or congugated estrogens such as ESTRACE™ (estradiol), ESTINYL™ (ethinyl estradiol), PREMARIN™, ESTRATAB™, ORTHO-EST™, OGEN™ and estropipate (estrone), ESTROVIS™ (quinestrol), ESTRADERM™ (estradiol), DELESTROGEN™ and VALERGEN™ (estradiol valerate), DEPO-ESTRADIOL CYPIONATE™ and ESTROJECT LA™ (estradiol cypionate); antiestrogens such as NOLVADEX™ (tamoxifen), SEROPHENE™ and CLOMID™ (clomiphene); progestins such as DURALUTIN™ (hydroxyprogesterone caproate), MPA™ and DEPO-PROVERA™ (medroxyprogesterone acetate), PROVERA™ and CYCRIN™ (MPA), MEGACE™ (megestrol acetate), NORLUTIN™ (norethindrone), and NORLUTATE™ and AYGESTIN™ (norethindrone acetate); progesterone implants such as NORPLANT SYSTEM™ (subdermal implants of norgestrel); antiprogestins such as RU 486™ (mifepristone); hormonal contraceptives such as ENOVID™ (norethynodrel plus mestranol), PROGESTASERT™ (intrauterine device that releases progesterone), LOESTRIN™, BREVICON™, MODICON™, GENORA™, NELONA™, NORINYL™, OVACON-35™ and OVACON-50 ™(ethinyl estradiovnorethindrone), LEVLEN™, NORDETTE™, TR1-LEVLEN™ and TRIPHASIL-21 ™ (ethinyl estradiol/levonorgestrel) LO/OVRAL™ and OVRAL™ (ethinyl estradiol/norgestrel), DEMULEN™ (ethinyl estradiol/ethynodiol diacetate), NORINYL™, ORTHO-NOVUM™, NORETHIN™, GENORA™, and NELOVA™ (norethindrone/mestranol), DESOGEN™ and ORTHO-CEPT™ (ethinyl estradiol/desogestrel), ORTHO-CYCLEN™ and ORTHO-TRICYCLENT™ (ethinyl estradiol/norgestimate), MICRONOR™ and NOR-QD™ (norethindrone), and OVRETTE™ (norgestrel).

Additional treatments for endocrine and/or hormone imbalance disorders include, but are not limited to, testosterone esters such as methenolone acetate and testosterone undecanoate; parenteral and oral androgens such as TESTOJECT-50™ (testosterone), TESTEX™ (testosterone propionate), DELATESTRYLM (testosterone enanthate), DEPO-TESTOSTERONE™ (testosterone cypionate), DANOCRINE™ (danazol), HALOTESTIN™ (fluoxymesterone), ORETON METHYL™, TESTRED™ and VIRILONTr (methyltestosterone), and OXANDRIN™ (oxandrolone); testosterone transdermal systems such as TESTODERM™; androgen receptor antagonist and 5-alpha-reductase inhibitors such as ANDROCUR™ (cyproterone acetate), EULEXN™ (flutamide), and PROSCAR™ (finasteride); adrenocorticotropic hormone preparations such as CORTROSYN™ (cosyntropin); adrenocortical steroids and their synthetic analogs such as ACLOVATE™ (alclometasone dipropionate), CYCLOCORT™ (amcinonide), BECLOVENT™ and VANCERIL™ (beclomethasone dipropionate), CELESTONE™ (betamethasone), BENISONE™ and UTICOR™ (betamnethasone benzoate), DIPROSONE™ (betarnethasone dipropionate), CELESTONE PHOSPHATE™ (betamethasone sodium phosphate), CELESTONE SOLUSPAN™ (betamethasone sodium phosphate and acetate), BETA-VAL™ and VALISONE™ (betamethasone valerate), TEMOVATE™ (clobetasol propionate), CLODERM™ (clocortolone pivalate), CORTEF™ and HYDROCORTONE™ (cortisol (hydrocortisone)), HYDROCORTONE ACETATE™ (cortisol (hydrocortisone) acetate), LOCOID™ (cortisol (hydrocortisone) butyrate), HYDROCORTONE PHOSPHATE™ (cortisol (hydrocortisone) sodium phosphate), A-HYDROCOR™ and SOLU CORTEF™ (cortisol (hydrocortisone) sodium succinate), WESTCOR™ (cortisol (hydrocortisone) valerate), CORTISONE ACETATE™ (cortisone acetate), DESOWEN™ and TRIDESILON™ (desonide), TOPICORT™ (desoximetasone), DECADRON™ (dexamethasone), DECADRON LA™ (dexamethasone acetate), DECADRON PHOSPHATE™ and HEXADROL PHOSPHATE™ (dexamethasone sodium phosphate), FLORONE™ and MAXIFLOR™ (diflorasone diacetate), FLORINEF ACETATE™ (fludrocortisone acetate), AEROBID™ and NASALIDE™ (flunisolide), FLUONID™ and SYNALAR™ (fluocinolone acetonide), LIDEXTh (fluocinonide), FLUOR-OP™ and FML™ (fluorometholone), CORDRAN™ (flurandrenolide), HALOG™ (halcinonide), HMS LIZUIFILM™ (medrysone), MEDROL™ (methylprednisolone), DEPO-MEDROL™ and MEDROL ACETATE™ (methylprednisone acetate), A-METHAPRED™ and SOLUMEDROL™ (methylprednisolone sodium succinate), ELOCON™ (mometasone furoate), HALDRONE™ (pararnethasone acetate), DELTA-CORTEF™ (prednisolone), ECONOPRED™ (prednisolone acetate), HYDELTRASOL™ (prednisolone sodium phosphate), HYDELTRA-T.B.A™ (prednisolone tebutate), DELTASONE™ (prednisone), ARISTOCORT™ and KENACORT™ (triamcinolone), KENALOG™ (triamcinolone acetonide), ARISTOCOR™ and KENACORT DIACETATE™ (triamcinolone diacetate), and ARISTOSPAN™ (triamcinolone hexacetonide); inhibitors of biosynthesis and action of adrenocortical steroids such as CYTADREN™ (aminoglutethimide), NIZORAL™ (ketoconazole), MODRASTANE™ (trilostane), and METOPIRONE™ (metyrapone); bovine, porcine or human insulin or mixtures thereof; insulin analogs; recombinant human insulin such as HUMULIN™ and NOVOLIN™; oral hypoglycemic agents such as ORAMIDE™ and ORINASE™ (tolbutamide), DIABINESE™ (chlorpropamide), TOLAMIDE™ and TOLINASE™ (tolazamide), DYMELOR™ (acetohexamide), glibenclamide, MICRONASE™, DIBETA™ and GLYNASE™ (glyburide), GLUCOTROL™ (glipizide), and DIAMICRON™ (gliclazide), GLUCOPHAGE™ (metformin), ciglitazone, pioglitazone, and alpha-glucosidase inhibitors; bovine or porcine glucagon; somatostatins such as SANDOSTATIN™ (octreotide); and diazoxides such as PROGLYCEM™ (diazoxide).

In one embodiment, the alpha-fetoprotein fusion proteins and/or polynucleotides of the invention are administered in combination with treatments for uterine motility disorders. Treatments for uterine motility disorders include, but are not limited to, estrogen drugs such as conjugated estrogens (e.g., PREMARIN® and ESTRATAB®), estradiols (e.g., CLIMARA® and ALORA®), estropipate, and chlorotrianisene; progestin drugs (e.g., AMEN® (medroxyprogesterone), MICRONOR® (norethidrone acetate), PROMETRIUM® progesterone, and megestrol acetate); and estrogen/progesterone combination therapies such as, for example, conjugated estrogens/medroxyprogesterone (e.g., PREMPRO™ and PREMPHASE®) and norethindrone acetate/ethinyl estsradiol (e.g., FEMHR™).

In an additional embodiment, the alpha-fetoprotein fusion proteins and/or polynucleotides of the invention are administered in combination with drugs effective in treating iron deficiency and hypochromic anemias, including but not limited to, ferrous sulfate (iron sulfate, FEOSOL™), ferrous fumarate (e.g., FEOSTA™), ferrous gluconate (e.g., FERGON™), polysaccharide-iron complex (e.g., NIFEREX™), iron dextran injection (e.g., INFED™), cupric sulfate, pyroxidine, riboflavin, Vitamin B₁₂, cyancobalamin injection (e.g., REDISOL™, RUBRAMIN PC™), hydroxocobalamin, folic acid (e.g., FOLVITE™), leucovorin (folinic acid, 5-CHOH4PteGlu, citrovorum factor) or WELLCOVORIN (Calcium salt of leucovorin), transfernin or ferritin.

In certain embodiments, the alpha-fetoprotein fusion proteins and/or polynucleotides of the invention are administered in combination with agents used to treat psychiatric disorders. Psychiatric drugs that may be administered with the alpha-fetoprotein fusion proteins and/or polynucleotides of the invention include, but are not limited to, antipsychotic agents (e.g., chlorpromazine, chlorprothixene, clozapine, fluphenazine, haloperidol, loxapine, mesoridazine, molindone, olanzapine, perphenazine, pimozide, quetiapine, risperidone, thioridazine, thiothixene, trifluoperazine, and triflupromazine), antimanic agents (e.g., carbamazepine, divalproex sodium, lithium carbonate, and lithium citrate), antidepressants (e.g., amitriptyline, amoxapine, bupropion, citalopram, clomipramine, desipramine, doxepin, fluvoxamine, fluoxetine, imipramine, isocarboxazid, maprotiline, mirtazapine, nefazodone, nortriptyline, paroxetine, phenelzine, protriptyline, sertraline, tranylcypromine, trazodone, trimipramine, and venlafaxine), antianxiety agents (e.g., alprazolam, buspirone, chlordiazepoxide, clorazepate, diazepam, halazepam, lorazepam, oxazepam, and prazepam), and stimulants (e.g., d-amphetamine, methylphenidate, and pemoline).

In other embodiments, the alpha-fetoprotein fusion proteins and/or polynucleotides of the invention are administered in combination with agents used to treat neurological disorders. Neurological agents that may be administered with the alpha-fetoprotein fusion proteins and/or polynucleotides of the invention include, but are not limited to, antiepileptic agents (e.g., carbamazepine, clonazepam, ethosuximide, phenobarbital, phenyloin, primidone, valproic acid, divalproex sodium, felbamate, gabapentin, lamotrigine, levetiracetam, oxcarbazepine, tiagabine, topiramate, zonisamide, diazepam, lorazepam, and clonazepam), antiparkinsonian agents (e.g., levodopa/carbidopa, selegiline, amantidine, bromocriptine, pergolide, ropinirole, pramipexole, benztropine; biperiden; ethopropazine; procyclidine; trihexyphenidyl, tolcapone), and ALS therapeutics (e.g. riluzole).

In another embodiment, alpha-fetoprotein fusion proteins and/or polynucleotides of the invention are administered in combination with vasodilating agents and/or calcium channel blocking agents. Vasodilating agents that may be administered with the alpha-fetoprotein fusion proteins and/or polynucleotides of the invention include, but are not limited to, Angiotensin Converting Enzyme (ACE) inhibitors (e.g., papaverine, isoxsuprine, benazepril, captopril, cilazapril, enalapril, enalaprilat, fosinopril, lisinopril, moexipril, perindopril, quinapril, ramipril, spirapril, trandolapril, and nylidrin), and nitrates (e.g., isosorbide dinitrate, isosorbide mononitrate, and nitroglycerin). Examples of calcium channel blocking agents that may be administered in combination with the alpha-fetoprotein fusion proteins and/or polynucleotides of the invention include, but are not limited to amlodipine, bepridil, diltiazem, felodipine, flunarizine, isradipine, nicardipine, nifedipine, nimodipine, and verapamil.

In certain embodiments, the alpha-fetoprotein fusion proteins and/or polynucleotides of the invention are administered in combination with treatments for gastrointestinal disorders. Treatments for gastrointestinal disorders that may be administered with the alpha-fetoprotein fusion protein and/or polynucleotide of the invention include, but are not limited to, H₂ histamine receptor antagonists (e.g., TAGAME™ (cimetidine), ZANTAC™ (ranitidine), PEPCID™ (famotidine), and AXID™ (nizatidine)); inhibitors of H⁺, K^{+ATPase} (e.g., PREVACID™ (lansoprazole) and PRILOSEC™ (omeprazole)); Bismuth compounds (e.g., PEPTO-BISMOL™ (bismuth subsalicylate) and DE-NOL: (bismuth subcitrate)); various antacids; sucrAFPate; prostaglandin analogs (e.g. CYTOTEC™ (misoprostol)); muscarinic cholinergic antagonists; laxatives (e.g., surfactant laxatives, stimulant laxatives, saline and osmotic laxatives); antidiarrheal agents (e.g., LOMOTILC (diphenoxylate), MOTOFEN™ (diphenoxin), and IMODIUM™ (loperamide hydrochloride)), synthetic analogs of somatostatin such as SANDOSTATIN™ (octreotide), antiemetic agents (e.g., ZOFRAN™ (ondansetron), KYTRIL™ (granisetron hydrochloride), tropisetron, dolasetron, metoclopramide, chlorpromazine, perphenazine, prochlorperazine, promethazine, thiethylperazine, triflupromazine, domperidone, haloperidol, droperidol, trimethobenzamide, dexamethasone, methylprednisolone, dronabinol, and nabilone); D2 antagonists (e.g., metoclopramide, trimethobenzamide and chlorpromazine); bile salts; chenodeoxycholic acid; ursodeoxycholic acid; and pancreatic enzyme preparations such as pancreatin and pancrelipase.

In additional embodiments, the alpha-fetoprotein fusion proteins and/or polynucleotides of the invention are administered in combination with other therapeutic or prophylactic regimens, such as, for example, radiation therapy.

The invention also provides a pharmaceutical pack or kit comprising one or more containers filled with one or more of the ingredients of the pharmaceutical compositions comprising alpha-fetoprotein fusion proteins of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

11. Additional Agents for Treating and/or Preventing Metabolic Disorders and Related Conditions

The alpha-fetoprotein fusion proteins may be administered, either alone as an active agent or in combination with one or more additional active agents, for the treatment and/or prevention of metabolic disorders and related conditions, including but not limited to hyperglycemia, IGT (impaired glucose tolerance), insulin resistance syndromes, syndrome X, Type 1 diabetes, Type 2 diabetes, hyperlipidemia, dyslipidemia, hypertriglyceridemia, hyperlipoproteinemia, hypercholesterolemia, arteriosclerosis including atherosclerosis, glucagonomas, acute pancreatitis, cardiovascular diseases, hypertension, cardiac hypertrophy, gastrointestinal disorders, obesity, diabetes as a consequence of obesity, and diabetic dyslipidemia. The additional active agents may be administered before, concurrently with, or after administering the alpha-fetoprotein fusion protein. In a further aspect of the invention the present compounds are combined with diet and/or exercise.

In some embodiments, the alpha-fetoprotein fusion proteins are administered in combination with one or more further active agents in any suitable ratios. Further active agents may include but are not limited to antidiabetic agents, antihyperlipidemic agents, antiobesity agents, antihypertensive agents and agents for the treatment of complications resulting from or associated with diabetes.

Suitable antidiabetic agents may include insulin, insulin analogues and derivatives such as those disclosed in EP 792 290 (Novo Nordisk A/S), e.g. N^{ε}B29-tetradecanoyl des (B30) human insulin, EP 214 826 and EP 705 275 (Novo Nordisk A/S), e.g. Asp^{B28} human insulin, U.S. Pat. No. 5,504,188 (Eli Lilly), e.g. Lys^{B28}Pro^{B29} human insulin, EP 368 187 (Aventis), e.g. Lantus, which are all incorporated herein by reference, GLP-1 derivatives such as those disclosed in WO 98/08871 (Novo Nordisk A/S), which is incorporated herein by reference. Insulin analogues may include insulin fusion proteins as described herein and as described in WO 93/15199 and WO 01/79271 (Aventis Molecules), which are incorporated herein by reference.

The alpha-fetoprotein fusion protein may be administered in combination with additional agents (e.g., hypoglycaemic agents) which may include imidazolines, sulphonylureas (e.g., glibenclamide or glyburide), biguamides (e.g., metformin), meglitinides (e.g., repaglinide or nateglinide), oxadiazolidinediones, thiazolidinediones (e.g., troglitazone, ciglitazone, piogitazone, rosiglitazone, isaglitazone, darglitazone, englitazone, CS-011/C1-1037 or T174 or the compounds disclosed in WO 97/41097, WO 97/41119, WO 97/41220, WO00/41121 and WO 98/45292 (Dr. Reddy's Research Foundation)), glucosidase inhibitors, glucagon antagonists, GLP-1 agonists, agents acting on the ATP-dependent potassium channel of the β-cells (e.g., potassium channel openers such as those disclosed in WO 97/26265, WO 99/03861 and WO 00/37474 (Novo Nordisk A/S)) which are incorporated herein by reference, or nateglinide or potassium channel blockers (e.g., tolbutamide, chlorpropamide, tolazamide, glibenclamide, glyburide, glipizide, glicazide, BTS-67582, repaglinide or nateglinide), insulin sensitizers (e.g., G1262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 63192, WO 00/63193 (Dr. Reddy's Research Foundation) and WO 00/23425, WO 00/23415, WO 00/23441, WO 00/23445, WO 00/23417, WO 00/23416, WO 00/63153, WO 00/63196, WO 00/63209, WO 63190 and WO 00/63189 (Novo Nordisk A/S)), DPP-IV (dipeptidyl peptidase-IV) inhibitors, PTPase inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, PPAR (peroxisome proliferator-activated receptor) and RXR (retinoid X receptor) agonists such as ALRT-268, LG-1268 or LG-1069.

In other embodiments, the alpha-fetoprotein fusion proteions may be administered in combination with an insulin sensitizer such as G1262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 63192, WO 00/63193 (Dr. Reddy's Research Foundation) and WO 00/23425, WO 00/23415, WO 00/23441, WO 00/23445, WO 00/23417, WO 00/23416, WO 00/63153, WO 00/63196, WO 00/63209, WO 63190 and WO 00/63189 (Novo Nordisk A/S). Additional agents may include α-glucosidase inhibitors (e.g., voglibose, emiglitate, miglitol or acarbose).

In further embodiments, the alpha-fetoprotein fusion proteions may be administered in combination with an antihyperlipidemic agent or antilipidemic agent (e.g., cholestyramine, colestipol, clofibrate, gemfibrozil, lovastatin, pravastatin, simvastatin, probucol or dextrothyroxine).

In further embodiments, the alpha-fetoprotein fusion proteions may be administered in combination with one or more antiobesity agents or appetite regulating agents. Such agents may include CART (cocaine amphetamine regulated transcript) agonists, NPY (neuropeptide Y) antagonists, MC4 (melanocortin 4) agonists, orexin antagonists, TNF (tumor necrosis factor) modulators, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxeline, seroxal or citalopram, serotonin and noradrenaline re-uptake inhibitors, 5HT (serotonin) agonists, bombesin agonists, galanin antagonists, growth hormone (growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators or TR β agonists. Exemplary antiobesity agent may include leptin, dexamphetamine. amphetamine, fenfluramine, dexfenfluramine, sibutramine, orlistat, mazindol, and phentermine.

In further embodiments, the alpha-fetoprotein fusion proteions may be administered in combination with one or more antihypertensive agents, which may include β-blockers such as alprenolol, atenolol, timolol, pindolol, propanolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, ditiazem and verapamil, and alpha.-blockers such as doxazosin, urapidil, prazosin and terazosin.

XI. Gene Therapy

Constructs encoding alpha-fetoprotein fusion proteins of the invention can be used as a part of a gene therapy protocol to deliver therapeutically effective doses of the alpha-fetoprotein fusion protein. A preferred approach for in vivo introduction of nucleic acid into a cell is by use of a viral vector comprising nucleic acid encoding an alpha-fetoprotein fusion protein of the invention. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous nucleic acid molecules encoding alpha-fetoprotein fusion proteins in vivo. These vectors provide efficient delivery of nucleic acids into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D., Blood, 76:271 (1990)). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.), Sections 9.10-9.14 (Greene Publishing Associates, 1989), and other standard laboratory manuals.

Another viral gene delivery system useful in the present invention uses adenovirus-derived vectors. The genome of an adenovirus can be manipulated such that it encodes and expresses a gene product of interest but is inactivated in terms of its ability to replicate in a normal lytic viral life cycle. See, for example, Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al., (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g. retroviral DNA). Moreover, the carrying capacity of the adenoviral genome for foreign DNA is large (up to 8 kilobases) relative to other gene delivery vectors (Berkner et al., cited supra; Haj-Ahmand et al., J. Virol., 57:267 (1986)).

In another embodiment, non-viral gene delivery systems of the invention rely on endocytic pathways for the uptake of the subject nucleotide molecule by the targeted cell. Exemplary gene delivery systems of this type include liposomal derived systems, poly-lysine conjugates, and artificial viral envelopes. In a representative embodiment, a nucleic acid molecule encoding an alpha-fetoprotein fusion protein of the invention can be entrapped in liposomes bearing positive charges on their surface (e.g., lipofectins) and (optionally) which are tagged with antibodies against cell surface antigens of the target tissue (Mizuno et al., No Shinkei Geka, 20:547-551 (1992); WO 91/06309; Japanese patent application 1047381; and European patent publication EP-A-43075).

Gene delivery systems for a gene encoding an alpha-fetoprotein fusion protein of the invention can be introduced into a patient by any of a number of methods. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by Stereotactic injection (e.g. Chen et al., PNAS, 91:3054-3057 (1994)). The pharmaceutical preparation of the gene therapy construct can consist essentially of the gene delivery system in an acceptable diluent, or can comprise a slow release matrix in which the gene delivery vehicle is imbedded. Where the alpha-fetoprotein fusion protein can be produced intact from recombinant cells, e.g. retroviral vectors, the pharmaceutical preparation can comprise one or more cells which produce the alpha-fetoprotein fusion protein.

A. Additional Gene Therapy Methods

Also encompassed by the invention are gene therapy methods for treating or preventing disorders, diseases and conditions. The gene therapy methods relate to the introduction of nucleic acid (DNA, RNA and antisense DNA or RNA) sequences into an animal to achieve expression of an alpha-fetoprotein fusion protein of the invention. This method requires a polynucleotide which codes for an alpha-fetoprotein fusion protein of the invention operatively linked to a promoter and any other genetic elements necessary for the expression of the fusion protein by the target tissue. Such gene therapy and delivery techniques are known in the art, see, for example, WO 90/11092.

Thus, for example, cells from a patient may be engineered with a polynucleotide (DNA or RNA) comprising a promoter operably linked to a polynucleotide encoding an alpha-fetoprotein fusion protein of the invention ex vivo, with the engineered cells then being provided to a patient to be treated with the fusion protein of the invention. Such methods are well-known in the art (Belldegrun et al., J. Natl. Cancer Inst., 85: 207-216 (1993); Ferrantini et al., Cancer Research, 53:1107-1112 (1993); Ferrantini et al., J. Immunology, 153: 4604-4615 (1994); Kaido et al., Int. J. Cancer, 60:221-229 (1995); Ogura et al., Cancer Research, 50:5102-5106 (1990); Santodonato et al., Human Gene Therapy, 7:1-10 (1996); Santodonato et al., Gene Therapy, 4:1246-1255 (1997); and Zhang et al., Cancer Gene Therapy, 3: 31-38 (1996)). In one embodiment, the cells which are engineered are arterial cells. The arterial cells may be reintroduced into the patient through direct injection to the artery, the tissues surrounding the artery, or through catheter injection.

As discussed in more detail below, the polynucleotide constructs can be delivered by any method that delivers injectable materials to the cells of an animal, such as, injection into the interstitial space of tissues (heart, muscle, skin, lung, liver, and the like). The polynucleotide constructs may be delivered in a pharmaceutically acceptable liquid or aqueous carrier.

In one embodiment, polynucleotides encoding the alpha-fetoprotein fusion proteins of the present invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, polynucleotides encoding the alpha-fetoprotein fusion proteins of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859.

The polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, PMSG and pSVL available from Pharmacia; and pEFIIN5, pcDNA3.1, and pRc/CMV2 available from Invitrogen. Other suitable vectors will be readily apparent to the skilled artisan.

Any strong promoter known to those skilled in the art can be used for driving the expression of the polynucleotide sequence. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the alpha-fetoprotein promoter; the ApoAl promoter; human globin promoters; viral thymidine kinase promoters, such as the Herpes Simplex thymidine kinase promoter; retroviral LTRs; the b-actin promoter; and human growth hormone promoters. The promoter also may be the native promoter for the gene corresponding to the therapeutic protein portion of the alpha-fetoprotein fusion proteins of the invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art. The constructs may also be delivered with delivery vehicles such as viral sequences, viral particles, liposome formulations, lipofectin, precipitating agents, etc. Such methods of delivery are known in the art.

In certain embodiments, the polynucleotide constructs are complexed in a liposome preparation. Liposomal preparations for use in the invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA, 84:7413-7416 (1987)); mRNA (Malone et al., Proc. Natl. Acad. Sci. USA, 86:6077-6081 (1989)); and purified transcription factors (Debs et al., J. Biol. Chem., 265:10189-10192 (1990), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA, 84:7413-7416 (1987)). Other commercially available liposomes include transfectace (DDAB/DOPE) and DOTAP/DOPE (Boehringer).

Other cationic liposomes can be prepared from readily available materials using techniques well known in the art. See WO 90/11092 for a description of the synthesis of DOTAP (1,2-bis(oleoyloxy)-3-(trimet-hylammonio)propane) liposomes. Preparation of DOTMA liposomes is explained in the literature (Felgner et al., Proc. Natl. Acad. Sci. USA, 84:7413-7417 (1987). Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15 degrees celcius. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SU,s being preferred. The various liposome-nucleic acid complexes are prepared using methods well known in the art. See Straubinger et al., Methods of Immunology, 101:512-527 (1983). For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta, 394:483 (1975); Wilson et al., Cell, 17:77 (1979)); ether injection (Deamer, D. and Bangham, A., Biochim. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch, H. and Strittmatter, P., Proc. Natl. Acad. Sci. USA, 76:145 (1979)); and reverse-phase evaporation (REV) (Fraley et al., J. Biol. Chem., 255:10431 (1980); Szoka, F. and Papahadjopoulos, D., Proc. Natl. Acad. Sci. USA, 75:145 (1978); Schaefer-Ridder et al., Science, 215:166 (1982)).

Generally, the ratio of DNA to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

U.S. Pat. No. 5,676,954 reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and WO 94/9469 provide cationic lipids for use in transfecting DNA into cells and mammals. U.S. Pat. Nos. 5,589,466, 5,693,622, 5,580,859, 5,703,055, and WO 94/9469 provide methods for delivering DNA-cationic lipid complexes to mammals.

In certain embodiments, cells are engineered, ex vivo or in vivo, using a retroviral particle containing RNA which comprises a sequence encoding an alpha-fetoprotein fusion protein of the present invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PAl 2, T19-14X, VT-19-17-H2, RCRE, RCRIP, GP+E-86, GP+envAm12, and DAN cell lines as described in Miller, Human Gene Therapy, 1:5-14 (1990). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include polynucleotide encoding an alpha-fetoprotein fusion protein of the invention. Such retroviral vector particles then may be employed, to transduce eukaryotic cells, either in vitro or in vivo. The transduced eukaryotic cells will express a fusion protein of the invention.

In certain other embodiments, cells are engineered, ex vivo or in vivo, with polynucleotide contained in an adenovirus vector. Adenovirus can be manipulated such that it encodes and expresses fusion protein of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz et al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in human cancer were uniformly negative (Green et al., Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the invention.

Preferably, the adenoviruses used in the invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol., 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the invention will include all the sequences necessary for DNA replication, encapsidation, and host-cell integration. The polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells, either ex vivo or in vivo. The transduced cells will contain the polynucleotide construct integrated into its genome, and will express a fusion protein of the invention.

Another method of gene therapy involves operably associating heterologous control regions and endogenous polynucleotide sequences (e.g. encoding a polypeptide of the invention) via homologous recombination (U.S. Pat. No. 5,641,670; WO 96/29411; WO 94/12650; Koller et al., Proc. Natl. Acad. Sci. USA, 86:8932-8935 (1989); and Zijlstra et al., Nature, 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotide encoding an alpha-fetoprotein fusion protein of the present invention may contain a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science, 243:375 (1989)).

A preferred method of local administration is by direct injection. Preferably, an alpha-fetoprotein fusion protein of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include fusion proteins of the present invention complexed to a targeted delivery vehicle of the present invention. Suitable delivery vehicles for use with systemic administration comprise liposomes comprising ligands for targeting the vehicle to a particular site. In specific embodiments, suitable delivery vehicles for use with systemic administration comprise liposomes comprising alpha-fetoprotein fusion proteins of the invention for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (Stribling et al., Proc. Natl. Acad. Sci. USA, 189:11277-11281, 1992). Oral delivery can be performed by complexing a polynucleotide construct of the invention to a carrier capable of withstanding degradation by digestive enzymes in the gut of an animal. Examples of such carriers, include plastic capsules or tablets, such as those known in the art. Topical delivery can be performed by mixing a polynucleotide construct of the invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Alpha-fetoprotein fusion proteins of the invention can be administered to any animal, preferably to mammals and birds. Preferred mammals include humans, dogs, cats, mice, rats, rabbits sheep, cattle, horses and pigs, with humans being particularly preferred.

XII. Use of the Fusion Proteins of the Invention in Assays for Biological and/or Immune Activity

A. Biological Activity

Alpha-fetoprotein fusion proteins and/or polynucleotides encoding alpha-fetoprotein fusion proteins of the invention can be used in assays to test for one or more biological activities. If an alpha-fetoprotein fusion protein and/or polynucleotide exhibits an activity in a particular assay, it is likely that the therapeutic protein corresponding to the fusion protein may be involved in the diseases associated with the biological activity. Thus, the fusion protein could be used to treat the associated disease.

In preferred embodiments, the invention encompasses a method of treating a disease or disorder comprising administering to a patient in which such treatment, prevention or amelioration is desired an alpha-fetoprotein fusion protein of the invention that comprises a therapeutic protein portion corresponding to a therapeutic protein in an amount effective to treat, prevent or ameliorate the disease or disorder.

In a further preferred embodiment, the invention encompasses a method of treating a disease or disorder comprising administering to a patient in which such treatment, prevention or amelioration is desired an alpha-fetoprotein fusion protein of the invention that comprises a therapeutic protein portion corresponding to the therapeutic protein for which the indications noted below are related in an amount effective to treat, prevent or ameliorate the disease or disorder.

In preferred embodiments, fusion proteins of the invention may be used in the diagnosis, prognosis, prevention and/or treatment of diseases and/or disorders relating to diseases and disorders of the endocrine system (see, for example, “Endocrine Disorders” section below), the nervous system, the immune system (see, for example, “Immune Activity” section below), respiratory system (see, for example, “Respiratory Disorders” section below), cardiovascular system (see, for example, “Cardiovascular Disorders” section below), reproductive system (see, for example, “Reproductive System Disorders” section below) digestive system (see, for example, “Gastrointestinal Disorders” section below), diseases and/or disorders relating to cell proliferation (see, for example, “Hyperproliferative Disorders” section below), and/or diseases or disorders relating to the blood (see, for example, “Blood-Related Disorders” section below).

In certain embodiments, an alpha-fetoprotein fusion protein of the invention may be used to diagnose and/or prognose diseases and/or disorders associated with the tissue(s) in which the gene corresponding to the therapeutic protein portion of the fusion protein of the invention is expressed.

Thus, fusion proteins of the invention and polynucleotides encoding alpha-fetoprotein fusion proteins of the invention are useful in the diagnosis, detection and/or treatment of diseases and/or disorders associated with activities that include, but are not limited to, prohonnone activation, neurotransmitter activity, cellular signaling, cellular proliferation, cellular differentiation, and cell migration. More generally, fusion proteins of the invention and polynucleotides encoding alpha-fetoprotein fusion proteins of the invention may be useful for the diagnosis, prognosis, prevention and/or treatment of diseases and/or disorders associated with the following systems.

B. Immune Activity

Alpha-fetoprotein fusion proteins of the invention and polynucleotides encoding alpha-fetoprotein fusion proteins of the invention may be useful in treating, preventing, diagnosing and/or prognosing diseases, disorders, and/or conditions of the immune system, by, for example, activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune diseases, disorders, and/or conditions may be genetic, somatic, such as cancer and some autoimmune diseases, acquired (e.g., by chemotherapy or toxins), or infectious. Moreover, fusion proteins of the invention and/or polynucleotides encoding alpha-fetoprotein fusion proteins of the invention can be used as a marker or detector of a particular immune system disease or disorder.

In another embodiment, a fusion protein of the invention and/or polynucleotide encoding an alpha-fetoprotein fusion protein of the invention, may be used to treat diseases and disorders of the immune system and/or to inhibit or enhance an immune response generated by cells associated with the tissue(s) in which the polypeptide of the invention is expressed.

Alpha-fetoprotein fusion proteins of the invention and/or polynucleotides encoding alpha-fetoprotein fusion proteins of the invention may be useful in treating, preventing, diagnosing, and/or prognosing immunodeficiencies, including both congenital and acquired immunodeficiencies. Examples of B cell immunodeficiencies in which immunoglobulin levels B cell function and/or B cell numbers are decreased include: X-linked agammaglobulinemia (Bruton's disease), X-linked infantile agammaglobulinemia, X-linked immunodeficiency with hyper IgM, non X-linked immunodeficiency with hyper IgM, X-linked lymphoproliferative syndrome (XLP), agammaglobulinemia including congenital and acquired agammaglobulinemia, adult onset agammaglobulinemia, late-onset agammaglobulinemia, dysgammaglobulinemia, hypogammaglobulinemia, unspecified hypogammaglobulinemia, recessive agammaglobulinemia (Swiss type), Selective IgM deficiency, selective IgA deficiency, selective IgG subclass deficiencies, IgG subclass deficiency (with or without IgA deficiency), Ig deficiency with increased IgM, IgG and IgA deficiency with increased IgM, antibody deficiency with normal or elevated Igs, Ig heavy chain deletions, kappa chain deficiency, B cell lymphoproliferative disorder (BLPD), common variable immunodeficiency (CVID), common variable immunodeficiency (CVI) (acquired), and transient hypogammaglobulinemia of infancy.

In specific embodiments, ataxia-telangiectasia or conditions associated with ataxia-telangiectasia are treated, prevented, diagnosed, and/or prognosing using the, fusion proteins of the invention and/or polynucleotides encoding alpha-fetoprotein fusion proteins of the invention.

Examples of congenital immunodeficiencies in which T cell and/or B cell function and/or number is decreased include, but are not limited to: DiGeorge anomaly, severe combined immunodeficiencies (SCID) (including, but not limited to, X-linked SCID, autosomal recessive SCID, adenosine deaminase deficiency, purine nucleoside phosphorylase (PNP) deficiency, Class II MHC deficiency (Bare lymphocyte syndrome), Wiskott-Aldrich syndrome, and ataxia telangiectasia), thymic hypoplasia, third and fourth pharyngeal pouch syndrome, 22q11.2 deletion, chronic mucocutaneous candidiasis, natural killer cell deficiency (NK), idiopathic CD4+T-lymphocytopenia, immunodeficiency with predominant T cell defect (unspecified), and unspecified immunodeficiency of cell mediated immunity.

In specific embodiments, DiGeorge anomaly or conditions associated with DiGeorge anomaly are treated, prevented, diagnosed, and/or prognosed using fusion proteins of the invention and/or polynucleotides encoding alpha-fetoprotein fusion proteins of the invention.

Other immunodeficiencies that may be treated, prevented, diagnosed, and/or prognosed using fusion proteins of the invention and/or polynucleotides encoding alpha-fetoprotein fusion proteins of the invention, include, but are not limited to, chronic granulomatous disease, Chediak-Higashi syndrome, myeloperoxidase deficiency, leukocyte glucose-6-phosphate dehydrogenase deficiency, X-linked lymphoproliferative syndrome (XLP), leukocyte adhesion deficiency, complement component deficiencies (including C1, C2, C3, C4, C5, C6, C7, C8 and/or C9 deficiencies), reticular dysgenesis, thymic alymphoplasia-aplasia, immunodeficiency with thymoma, severe congenital leukopenia, dysplasia with immunodeficiency, neonatal neutropenia, short limbed dwarfism, and Nezelof syndrome-combined immunodeficiency with Igs.

In a preferred embodiment, the immunodeficiencies and/or conditions associated with the immunodeficiencies recited above are treated, prevented, diagnosed and/or prognosed using fusion proteins of the invention and/or polynucleotides encoding alpha-fetoprotein fusion proteins of the invention.

In a preferred embodiment fusion proteins of the invention and/or polynucleotides encoding alpha-fetoprotein fusion proteins of the invention could be used as an agent to boost immunoresponsiveness among immunodeficient individuals. In specific embodiments, fusion proteins of the invention and/or polynucleotides encoding alpha-fetoprotein fusion proteins of the invention could be used as an agent to boost immunoresponsiveness among B cell and/or T cell immunodeficient individuals.

The alpha-fetoprotein fusion proteins of the invention and/or polynucleotides encoding alpha-fetoprotein fusion proteins of the invention may be useful in treating, preventing, diagnosing and/or prognosing autoimmune disorders. Many autoimmune disorders result from inappropriate recognition of self as foreign material by immune cells. This inappropriate recognition results in an immune response leading to the destruction of the host tissue. Therefore, the administration of fusion proteins of the invention and/or polynucleotides encoding alpha-fetoprotein fusion proteins of the invention that can inhibit an immune response, particularly the proliferation, differentiation, or chemotaxis of T-cells, may be an effective therapy in preventing autoimmune disorders.

Autoimmune diseases or disorders that may be treated, prevented, diagnosed and/or prognosed by fusion proteins of the invention and/or polynucleotides encoding alpha-fetoprotein fusion proteins of the invention include, but are not limited to, one or more of the following: systemic lupus erythematosus, rheumatoid arthritis, ankylosing spondylitis, multiple sclerosis, autoimmune thyroiditis, Hashimoto's thyroiditis, autoimmune hemolytic anemia, hemolytic anemia, thrombocytopenia, autoimmune thrombocytopenia purpura, autoimmune neonatal thrombocytopenia, idiopathic thrombocytopenia purpura, purpura (e.g., Henloch-Scoenlein purpura), autoimmunocytopenia, Goodpasture's syndrome, Pemphigus vulgaris, myasthenia gravis, Grave's disease (hyperthyroidism), and insulin-resistant diabetes mellitus.

Additional disorders that are likely to have an autoimmune component that may be treated, prevented, and/or diagnosed with the alpha-fetoprotein fusion proteins of the invention and/or polynucleotides encoding alpha-fetoprotein fusion proteins of the invention include, but are not limited to, type II collagen-induced arthritis, antiphospholipid syndrome, dermatitis, allergic encephalomyelitis, myocarditis, relapsing polychondritis, rheumatic heart disease, neuritis, uveitis ophthalmia, polyendocrinopathies, Reiter's Disease, Stiff-Man Syndrome, autoimmune pulmonary inflammation, autism, Guillain-Barre Syndrome, insulin dependent diabetes mellitus, and autoimmune inflammatory eye disorders.

Additional disorders that are likely to have an autoimmune component that may be treated, prevented, diagnosed and/or prognosed with the alpha-fetoprotein fusion proteins of the invention and/or polynucleotides encoding alpha-fetoprotein fusion proteins of the invention include, but are not limited to, scleroderma with anti-collagen antibodies (often characterized, e.g., by nucleolar and other nuclear antibodies), mixed connective tissue disease (often characterized, e.g., by antibodies to extractable nuclear antigens (e.g., ribonucleoprotein)), polymyositis (often characterized, e.g., by nonhistone ANA), pernicious anemia (often characterized, e.g., by antiparietal cell, microsomes, and intrinsic factor antibodies), idiopathic Addison's disease (often characterized, e.g., by humoral and cell-mediated adrenal cytotoxicity, infertility (often characterized, e.g., by antispermatozoal antibodies), glomerulonephritis (often characterized, e.g., by glomerular basement membrane antibodies or immune complexes), bullous pemphigoid (often characterized, e.g., by IgG and complement in basement membrane), Sjogren's syndrome (often characterized, e.g., by multiple tissue antibodies, and/or a specific nonhistone ANA (SS-B)), diabetes mellitus (often characterized, e.g., by cell-mediated and humoral islet cell antibodies), and adrenergic drug resistance (including adrenergic drug resistance with asthma or cystic fibrosis) (often characterized, e.g., by beta-adrenergic receptor antibodies).

Additional disorders that may have an autoimmune component that may be treated, prevented, diagnosed and/or prognosed with the alpha-fetoprotein fusion proteins of the invention and/or polynucleotides encoding alpha-fetoprotein fusion proteins of the invention include, but are not limited to, chronic active hepatitis (often characterized, e.g., by smooth muscle antibodies), primary biliary cirrhosis (often characterized, e.g., by mitochondria antibodies), other endocrine gland failure (often characterized, e.g., by specific tissue antibodies in some cases), vitiligo (often characterized, e.g., by melanocyte antibodies), vasculitis (often characterized, e.g., by Ig and complement in vessel walls and/or low serum complement), post-MI (often characterized, e.g., by myocardial antibodies), cardiotomy syndrome (often characterized, e.g., by myocardial antibodies), urticaria (often characterized, e.g., by IgG and IgM antibodies to IgE), atopic demmatitis (often characterized, e.g., by IgG and IgM antibodies to IgE), asthma (often characterized, e.g., by IgG and IgM antibodies to IgE), and many other inflammatory, granulomatous, degenerative, and atrophic disorders.

In a preferred embodiment, the autoimmune diseases and disorders and/or conditions associated with the diseases and disorders recited above are treated, prevented, diagnosed and/or prognosed using for example, fusion proteins of the invention and/or polynucleotides encoding alpha-fetoprotein fusion proteins of the invention. In a specific preferred embodiment, rheumatoid arthritis is treated, prevented, and/or diagnosed using fusion proteins of the invention and/or polynucleotides encoding alpha-fetoprotein fusion proteins of the invention.

In another specific preferred embodiment, systemic lupus erythematosus is treated, prevented, and/or diagnosed using fusion proteins of the invention and/or polynucleotides encoding alpha-fetoprotein fusion proteins of the invention. In another specific preferred embodiment, idiopathic thrombocytopenia purpura is treated, prevented, and/or diagnosed using fusion proteins of the invention and/or polynucleotides encoding alpha-fetoprotein fusion proteins of the invention.

In another specific preferred embodiment IgA nephropathy is treated, prevented, and/or diagnosed using fusion proteins of the invention and/or polynucleotides encoding alpha-fetoprotein fusion proteins of the invention.

In a preferred embodiment, the autoimmune diseases and disorders and/or conditions associated with the diseases and disorders recited above are treated, prevented, diagnosed and/or prognosed using fusion proteins of the invention and/or polynucleotides encoding alpha-fetoprotein fusion proteins of the invention.

In preferred embodiments, fusion proteins of the invention and/or polynucleotides encoding alpha-fetoprotein fusion proteins of the invention are used as a immunosuppressive agent(s).

Alpha-fetoprotein fusion proteins of the invention and/or polynucleotides encoding alpha-fetoprotein fusion proteins of the invention may be useful in treating, preventing, prognosing, and/or diagnosing diseases, disorders, and/or conditions of hematopoietic cells. Alpha-fetoprotein fusion proteins of the invention and/or polynucleotides encoding alpha-fetoprotein fusion proteins of the invention could be used to increase differentiation and proliferation of hematopoietic cells, including the pluripotent stem cells, in an effort to treat or prevent those diseases, disorders, and/or conditions associated with a decrease in certain (or many) types hematopoietic cells, including but not limited to, leukopenia, neutropenia, anemia, and thrombocytopenia. Alternatively, fusion proteins of the invention and/or polynucleotides encoding alpha-fetoprotein fusion proteins of the invention could be used to increase differentiation and proliferation of hematopoietic cells, including the pluripotent stem cells, in an effort to treat or prevent those diseases, disorders, and/or conditions associated with an increase in certain (or many) types of hematopoietic cells, including but not limited to, histiocytosis.

Allergic reactions and conditions, such as asthma (particularly allergic asthma) or other respiratory problems, may also be treated, prevented, diagnosed and/or prognosed using fusion proteins of the invention and/or polynucleotides encoding alpha-fetoprotein fusion proteins of the invention. Moreover, these molecules can be used to treat, prevent, prognose, and/or diagnose anaphylaxis, hypersensitivity to an antigenic molecule, or blood group incompatibility.

Additionally, fusion proteins of the invention and/or polynucleotides encoding alpha-fetoprotein fusion proteins of the invention, may be used to treat, prevent, diagnose and/or prognose IgE-mediated allergic reactions. Such allergic reactions include, but are not limited to, asthma, rhinitis, and eczema. In specific embodiments, fusion proteins of the invention and/or polynucleotides encoding alpha-fetoprotein fusion proteins of the invention may be used to modulate IgE concentrations in vitro or in vivo.

Moreover, fusion proteins of the invention and/or polynucleotides encoding alpha-fetoprotein fusion proteins of the invention have uses in the diagnosis, prognosis, prevention, and/or treatment of inflammatory conditions. For example, since fusion proteins of the invention and/or polynucleotides encoding alpha-fetoprotein fusion proteins of the invention may inhibit the activation, proliferation and/or differentiation of cells involved in an inflammatory response, these molecules can be used to prevent and/or treat chronic and acute inflammatory conditions. Such inflammatory conditions include, but are not limited to, for example, inflammation associated with infection (e.g., septic shock, sepsis, or systemic inflammatory response syndrome), ischemia-reperfusion injury, endotoxin lethality, complement-mediated hyperacute rejection, nephritis, cytokine or chemokine induced lung injury, inflammatory bowel disease, Crohn's disease, over production of cytokines (e.g., TNF or IL-1.), respiratory disorders (e.g., asthma and allergy); gastrointestinal disorders (e.g., inflammatory bowel disease); cancers (e.g., gastric, ovarian, lung, bladder, liver, and breast); CNS disorders (e.g., multiple sclerosis; ischemic brain injury and/or stroke, traumatic brain injury, neurodegenerative disorders (e.g., Parkinson's disease and Alzheimer's disease); AIDS-related dementia; and prion disease); cardiovascular disorders (e.g., atherosclerosis, myocarditis, cardiovascular disease, and cardiopulmonary bypass complications); as well as many additional diseases, conditions, and disorders that are characterized by inflammation (e.g., hepatitis, rheumatoid arthritis, gout, trauma, pancreatitis, sarcoidosis, dermatitis, renal ischemia-reperfusion injury, Grave's disease, systemic lupus erythematosus, diabetes mellitus, and allogenic transplant rejection).

Because inflammation is a fundamental defense mechanism, inflammatory disorders can effect virtually any tissue of the body. Accordingly, fusion proteins of the invention and/or polynucleotides encoding alpha-fetoprotein fusion proteins of the invention, have uses in the treatment of tissue-specific inflammatory disorders, including, but not limited to, adrenalitis, alveolitis, angiocholecystitis, appendicitis, balanitis, blepharitis, bronchitis, bursitis, carditis, cellulitis, cervicitis, cholecystitis, chorditis, cochlitis, colitis, conjunctivitis, cystitis, dermatitis, diverticulitis, encephalitis, endocarditis, esophagitis, eustachitis, fibrositis, folliculitis, gastritis, gastroenteritis, gingivitis, glossitis, hepatosplenitis, keratitis, labyrinthitis, laryngitis, lymphangitis, mastitis, media otitis, meningitis, metritis, mucitis, myocarditis, myosititis, myringitis, nephritis, neuritis, orchitis, osteochondritis, otitis, pericarditis, peritendonitis, peritonitis, pharyngitis, phlebitis, poliomyelitis, prostatitis, pulpitis, retinitis, rhinitis, salpingitis, scleritis, sclerochoroiditis, scrotitis, sinusitis, spondylitis, steatitis, stomatitis, synovitis, syringitis, tendonitis, tonsillitis, urethritis, and vaginitis.

In specific embodiments, fusion proteins of the invention and/or polynucleotides encoding alpha-fetoprotein fusion proteins of the invention, are useful to diagnose, prognose, prevent, and/or treat organ transplant rejections and grafl-versus-host disease. Organ rejection occurs by host immune cell destruction of the transplanted tissue through an immune response. Similarly, an immune response is also involved in GVHD, but, in this case, the foreign transplanted immune cells destroy the host tissues. Polypeptides, antibodies, or polynucleotides of the invention, and/or agonists or antagonists thereof, that inhibit an immune response, particularly the activation, proliferation, differentiation, or chemotaxis of T-cells, may be an effective therapy in preventing organ rejection or GVHD. In specific embodiments, fusion proteins of the invention and/or polynucleotides encoding alpha-fetoprotein fusion proteins of the invention, that inhibit an immune response, particularly the activation, proliferation, differentiation, or chemotaxis of T-cells, may be an effective therapy in preventing experimental allergic and hyperacute xenografR rejection.

In other embodiments, fusion proteins of the invention and/or polynucleotides encoding alpha-fetoprotein fusion proteins of the invention, are useful to diagnose, prognose, prevent, and/or treat immune complex diseases, including, but not limited to, serum sickness, post streptococcal glomerulonephritis, polyarteritis nodosa, and immune complex-induced vasculitis.

Alpha-fetoprotein fusion proteins of the invention and/or polynucleotides encoding alpha-fetoprotein fusion proteins of the invention can be used to treat, detect, and/or prevent infectious agents. For example, by increasing the immune response, particularly increasing the proliferation activation and/or differentiation of B and/or T cells, infectious diseases may be treated, detected, and/or prevented. The immune response may be increased by either enhancing an existing immune response, or by initiating a new immune response. Alternatively, fusion proteins of the invention and/or polynucleotides encoding alpha-fetoprotein fusion proteins of the invention may also directly inhibit the infectious agent (refer to section of application listing infectious agents, etc), without necessarily eliciting an immune response.

In another embodiment, alpha-fetoprotein fusion proteins of the invention and/or polynucleotides encoding alpha-fetoprotein fusion proteins of the invention are used as a vaccine adjuvant that enhances immune responsiveness to an antigen. In a specific embodiment, alpha-fetoprotein fusion proteins of the invention and/or polynucleotides encoding alpha-fetoprotein fusion proteins of the invention are used as an adjuvant to enhance tumor-specific immune responses.

In another specific embodiment, alpha-fetoprotein fusion proteins of the invention and/or polynucleotides encoding alpha-fetoprotein fusion proteins of the invention are used as an adjuvant to enhance anti-viral immune responses. Anti-viral immune responses that may be enhanced using the compositions of the invention as an adjuvant, include virus and virus associated diseases or symptoms described herein or otherwise known in the art. In specific embodiments, the compositions of the invention are used as an adjuvant to enhance an immune response to a virus, disease, or symptom selected from the group consisting of: AIDS, meningitis, Dengue, EBV, and hepatitis (e.g., hepatitis B). In another specific embodiment, the compositions of the invention are used as an adjuvant to enhance an immune response to a virus, disease, or symptom selected from the group consisting of: HIV/AIDS, herpes, respiratory syncytial virus, Dengue, rotavirus, Japanese B encephalitis, influenza A and B, parainfluenza, measles, cytomegalovirus, rabies, Junin, Chikungunya, Rift Valley Fever, herpes simplex, and yellow fever.

In another specific embodiment, alpha-fetoprotein fusion proteins of the invention and/or polynucleotides encoding alpha-fetoprotein fusion proteins of the invention are used as an adjuvant to enhance anti-bacterial or anti-fungal immune responses. Anti-bacterial or anti-fungal immune responses that may be enhanced using the compositions of the invention as an adjuvant, include bacteria or fungus and bacteria or fungus associated diseases or symptoms described herein or otherwise known in the art. In specific embodiments, the compositions of the invention are used as an adjuvant to enhance an immune response to a bacteria or fungus, disease, or symptom selected from the group consisting of: tetanus, Diphtheria, botulism, and meningitis type B.

In another specific embodiment, the compositions of the invention are used as an adjuvant to enhance an immune response to a bacteria or fungus, disease, or symptom selected from the group consisting of: Vibrio cholerae, Mycobacterium leprae, Salmonella typhi, Salmonella paratyphi, Meisseria meningitidis, Streptococcus pneumoniae, Group B streptococcus, Shigella spp., Enterotoxigenic Escherichia coli, Enterohemorrhagic E. coli, and Borrelia burgdorferi.

In another specific embodiment, alpha-fetoprotein fusion proteins of the invention and/or polynucleotides encoding alpha-fetoprotein fusion proteins of the invention are used as an adjuvant to enhance anti-parasitic immune responses. Anti-parasitic immune responses that may be enhanced using the compositions of the invention as an adjuvant, include parasite and parasite associated diseases or symptoms described herein or otherwise known in the art. In specific embodiments, the compositions of the invention are used as an adjuvant to enhance an immune response to a parasite. In another specific embodiment, the compositions of the invention are used as an adjuvant to enhance an immune response to Plasmodium (malaria) or Leishmania.

In another specific embodiment, alpha-fetoprotein fusion proteins of the invention and/or polynucleotides encoding alpha-fetoprotein fusion proteins of the invention may also be employed to treat infectious diseases including silicosis, sarcoidosis, and idiopathic pulmonary fibrosis; for example, by preventing the recruitment and activation of mononuclear phagocytes.

In another specific embodiment, alpha-fetoprotein fusion proteins of the invention and/or polynucleotides encoding alpha-fetoprotein fusion proteins of the invention are used as an antigen for the generation of antibodies to inhibit or enhance immune mediated responses against polypeptides of the invention.

In one embodiment, alpha-fetoprotein fusion proteins of the invention and/or polynucleotides encoding alpha-fetoprotein fusion proteins of the invention are administered to an animal (e.g., mouse, rat, rabbit, hamster, guinea pig, pigs, micro-pig, chicken, camel, goat, horse, cow, sheep, dog, cat, non-human primate, and human, most preferably human) to boost the immune system to produce increased quantities of one or more antibodies (e.g., IgG, IgA, IgM, and IgE), to induce higher affinity antibody production and immunoglobulin class switching (e.g., IgG, IgA, IgM, and IgE), and/or to increase an immune response.

In another specific embodiment, alpha-fetoprotein fusion proteins of the invention and/or polynucleotides encoding alpha-fetoprotein fusion proteins of the invention are used as a stimulator of B cell responsiveness to pathogens.

In another specific embodiment, alpha-fetoprotein fusion proteins of the invention and/or polynucleotides encoding alpha-fetoprotein fusion proteins of the invention are used as an activator of T cells.

In another specific embodiment, alpha-fetoprotein fusion proteins of the invention and/or polynucleotides encoding alpha-fetoprotein fusion proteins of the invention are used as an agent that elevates the immune status of an individual prior to their receipt of immunosuppressive therapies.

In another specific embodiment, alpha-fetoprotein fusion proteins of the invention and/or polynucleotides encoding alpha-fetoprotein fusion proteins of the invention are used as an agent to induce higher affinity antibodies.

In another specific embodiment, alpha-fetoprotein fusion proteins of the invention and/or polynucleotides encoding alpha-fetoprotein fusion proteins of the invention are used as an agent to increase serum immunoglobulin concentrations.

In another specific embodiment, alpha-fetoprotein fusion proteins of the invention and/or polynucleotides encoding alpha-fetoprotein fusion proteins of the invention are used as an agent to accelerate recovery of immunocompromised individuals.

In another specific embodiment, alpha-fetoprotein fusion proteins of the invention and/or polynucleotides encoding alpha-fetoprotein fusion proteins of the invention are used as an agent to boost immunoresponsiveness among aged populations and/or neonates.

In another specific embodiment, alpha-fetoprotein fusion proteins of the invention and/or polynucleotides encoding alpha-fetoprotein fusion proteins of the invention are used as an immune system enhancer prior to, during, or after bone marrow transplant and/or other transplants (e.g., allogeneic or xenogeneic organ transplantation). With respect to transplantation, compositions of the invention may be administered prior to, concomitant with, and/or after transplantation. In a specific embodiment, compositions of the invention are administered after transplantation, prior to the beginning of recovery of T-cell populations. In another specific embodiment, compositions of the invention are first administered after transplantation after the beginning of recovery of T cell populations, but prior to full recovery of B cell populations.

In another specific embodiment, alpha-fetoprotein fusion proteins of the invention and/or polynucleotides encoding alpha-fetoprotein fusion proteins of the invention are used as an agent to boost immunoresponsiveness among individuals having an acquired loss of B cell function. Conditions resulting in an acquired loss of B cell function that may be ameliorated or treated by administering the alpha-fetoprotein fusion proteins of the invention and/or polynucleotides encoding alpha-fetoprotein fusion proteins of the invention, include, but are not limited to, HIV Infection, herpes, AIDS, bone marrow transplant, and B cell chronic lymphocytic leukemia (CLL).

In another specific embodiment, alpha-fetoprotein fusion proteins of the invention and/or polynucleotides encoding alpha-fetoprotein fusion proteins of the invention are used as an agent to boost immunoresponsiveness among individuals having a temporary immune deficiency. Conditions resulting in a temporary immune deficiency that may be ameliorated or treated by administering the alpha-fetoprotein fusion proteins of the invention and/or polynucleotides encoding alpha-fetoprotein fusion proteins of the invention, include, but are not limited to, recovery from viral infections (e.g., influenza), conditions associated with malnutrition, recovery from infectious mononucleosis, or conditions associated with stress, recovery from measles, recovery from blood transfusion, and recovery from surgery.

In another specific embodiment, alpha-fetoprotein fusion proteins of the invention and/or polynucleotides encoding alpha-fetoprotein fusion proteins of the invention are used as a regulator of antigen presentation by monocytes, dendritic cells, and/or B-cells. In one embodiment, alpha-fetoprotein fusion proteins of the invention and/or polynucleotides encoding alpha-fetoprotein fusion proteins of the invention enhance antigen presentation or antagonize antigen presentation in vitro or in vivo. Moreover, in related embodiments, this enhancement or antagonism of antigen presentation may be useful as an anti-tumor treatment or to modulate the immune system.

In another specific embodiment, alpha-fetoprotein fusion proteins of the invention and/or polynucleotides encoding alpha-fetoprotein fusion proteins of the invention are used as an agent to direct an individual's immune system towards development of a humoral response (i.e. TH2) as opposed to a TH I cellular response.

In another specific embodiment, alpha-fetoprotein fusion proteins of the invention and/or polynucleotides encoding alpha-fetoprotein fusion proteins of the invention are used as a means to induce tumor proliferation and thus make it more susceptible to anti-neoplastic agents. For example, multiple myeloma is a slowly dividing disease and is thus refractory to virtually all anti-neoplastic regimens. If these cells were forced to proliferate more rapidly their susceptibility profile would likely change.

In another specific embodiment, alpha-fetoprotein fusion proteins of the invention and/or polynucleotides encoding alpha-fetoprotein fusion proteins of the invention are used as a stimulator of B cell production in pathologies such as AIDS, chronic lymphocyte disorder and/or Common Variable Immunodeficiency.

In another specific embodiment, alpha-fetoprotein fusion proteins of the invention and/or polynucleotides encoding alpha-fetoprotein fusion proteins of the invention are used as a therapy for generation and/or regeneration of lymphoid tissues following surgery, trauma or genetic defect. In another specific embodiment, alpha-fetoprotein fusion proteins of the invention and/or polynucleotides encoding alpha-fetoprotein fusion proteins of the invention are used in the pretreatment of bone marrow samples prior to transplant.

In another specific embodiment, alpha-fetoprotein fusion proteins of the invention and/or polynucleotides encoding alpha-fetoprotein fusion proteins of the invention are used as a gene-based therapy for genetically inherited disorders resulting in immuno-incompetence/immunodeficiency such as observed among SCID patients.

In another specific embodiment, alpha-fetoprotein fusion proteins of the invention and/or polynucleotides encoding alpha-fetoprotein fusion proteins of the invention are used as a means of activating monocytes/macrophages to defend against parasitic diseases that effect monocytes such as Leishmania.

In another specific embodiment, alpha-fetoprotein fusion proteins of the invention and/or polynucleotides encoding alpha-fetoprotein fusion proteins of the invention are used as a means of regulating secreted cytokines that are elicited by polypeptides of the invention.

In another embodiment, alpha-fetoprotein fusion proteins of the invention and/or polynucleotides encoding alpha-fetoprotein fusion proteins of the invention are used in one or more of the applications described herein, as they may apply to veterinary medicine.

In another specific embodiment, alpha-fetoprotein fusion proteins of the invention and/or polynucleotides encoding alpha-fetoprotein fusion proteins of the invention are used as a means of blocking various aspects of immune responses to foreign agents or self. Examples of diseases or conditions in which blocking of certain aspects of immune responses may be desired include autoimmune disorders such as lupus, and arthritis, as well as immunoresponsiveness to skin allergies, inflammation, bowel disease, injury and diseases/disorders associated with pathogens.

In another specific embodiment, alpha-fetoprotein fusion proteins of the invention and/or polynucleotides encoding alpha-fetoprotein fusion proteins of the invention are used as a therapy for preventing the B cell proliferation and Ig secretion associated with autoimmune diseases such as idiopathic thrombocytopenic purpura, systemic lupus erythematosus and multiple sclerosis.

In another specific embodiment, polypeptides, antibodies, polynucleotides and/or agonists or antagonists of the present fusion proteins of the invention and/or polynucleotides encoding alpha-fetoprotein fusion proteins of the invention are used as a inhibitor of B and/or T cell migration in endothelial cells. This activity disrupts tissue architecture or cognate responses and is useful, for example in disrupting immune responses, and blocking sepsis.

In another specific embodiment, alpha-fetoprotein fusion proteins of the invention and/or polynucleotides encoding alpha-fetoprotein fusion proteins of the invention are used as a therapy for chronic hypergammaglobulinemia evident in such diseases as monoclonal gammopathy of undetermined significance (MGUS), Waldenstrom's disease, related idiopathic monoclonal gammopathies, and plasmacytomas.

In another specific embodiment, alpha-fetoprotein fusion proteins of the invention and/or polynucleotides encoding alpha-fetoprotein fusion proteins of the invention may be employed for instance to inhibit polypeptide chemotaxis and activation of macrophages and their precursors, and of neutrophils, basophils, B lymphocytes and some T-cell subsets, e.g., activated and CD8 cytotoxic T cells and natural killer cells, in certain autoimmune and chronic inflammatory and infective diseases. Examples of autoimmune diseases are described herein and include multiple sclerosis, and insulin-dependent diabetes.

The alpha-fetoprotein fusion proteins of the invention and/or polynucleotides encoding alpha-fetoprotein fusion proteins of the invention may also be employed to treat idiopathic hyper-eosinophilic syndrome by, for example, preventing eosinophil production and migration.

In another specific embodiment, alpha-fetoprotein fusion proteins of the invention and/or polynucleotides encoding alpha-fetoprotein fusion proteins of the invention are used to enhance or inhibit complement mediated cell lysis.

In another specific embodiment, alpha-fetoprotein fusion proteins of the invention and/or polynucleotides encoding alpha-fetoprotein fusion proteins of the invention are used to enhance or inhibit antibody dependent cellular cytotoxicity.

In another specific embodiment, alpha-fetoprotein fusion proteins of the invention and/or polynucleotides encoding alpha-fetoprotein fusion proteins of the invention may also be employed for treating atherosclerosis, for example, by preventing monocyte infiltration in the artery wall.

In another specific embodiment, alpha-fetoprotein fusion proteins of the invention and/or polynucleotides encoding alpha-fetoprotein fusion proteins of the invention may be employed to treat adult respiratory distress syndrome (ARDS).

In another specific embodiment, alpha-fetoprotein fusion proteins of the invention and/or polynucleotides encoding alpha-fetoprotein fusion proteins of the invention may be useful for stimulating wound and tissue repair, stimulating angiogenesis, and/or stimulating the repair of vascular or lymphatic diseases or disorders. Additionally, fusion proteins of the invention and/or polynucleotides encoding alpha-fetoprotein fusion proteins of the invention may be used to stimulate the regeneration of mucosal surfaces.

In a specific embodiment, alpha-fetoprotein fusion proteins of the invention and/or polynucleotides encoding alpha-fetoprotein fusion proteins of the invention are used to diagnose, prognose, treat, and/or prevent a disorder characterized by primary or acquired immunodeficiency, deficient serum immunoglobulin production, recurrent infections, and/or immune system dysfunction. Moreover, fusion proteins of the invention and/or polynucleotides encoding alpha-fetoprotein fusion proteins of the invention may be used to treat or prevent infections of the joints, bones, skin, and/or parotid glands, blood-borne infections (e.g., sepsis, meningitis, septic arthritis, and/or osteomyelitis), autoimmune diseases (e.g., those disclosed herein), inflammatory disorders, and malignancies, and/or any disease or disorder or condition associated with these infections, diseases, disorders and/or malignancies) including, but not limited to, CVID, other primary immune deficiencies, HIV disease, CLL, recurrent bronchitis, sinusitis, otitis media, conjunctivitis, pneumonia, hepatitis, meningitis, herpes zoster (e.g., severe herpes zoster), and/or pneumocystis camii. Other diseases and disorders that may be prevented, diagnosed, prognosed, and/or treated with fusion proteins of the invention and/or polynucleotides encoding alpha-fetoprotein fusion proteins of the invention include, but are not limited to, HIV infection, HTLV-BLV infection, lymphopenia, phagocyte bactericidal dysfunction anemia, thrombocytopenia, and hemoglobinuria.

In another embodiment, alpha-fetoprotein fusion proteins of the invention and/or polynucleotides encoding alpha-fetoprotein fusion proteins of the invention are used to treat, and/or diagnose an individual having common variable immunodeficiency disease (“CVID”; also known as “acquired agammaglobulinemia” and “acquired hypogammaglobulinemia”) or a subset of this disease.

In a specific embodiment, alpha-fetoprotein fusion proteins of the invention and/or polynucleotides encoding alpha-fetoprotein fusion proteins of the invention may be used to diagnose, prognose, prevent, and/or treat cancers or neoplasms including immune cell or immune tissue-related cancers or neoplasms. Examples of cancers or neoplasms that may be prevented, diagnosed, or treated by fusion proteins of the invention and/or polynucleotides encoding alpha-fetoprotein fusion proteins of the invention include, but are not limited to, acute myelogenous leukemia, chronic myelogenous leukemia, Hodgkin's disease, non-Hodgkin's lymphoma, acute lymphocytic anemia (ALL) Chronic lymphocyte leukemia, plasmacytomas, multiple myeloma, Burkin's lymphoma, EBV-transformed diseases, and/or diseases and disorders described in the section entitled “Hyperproliferative Disorders” elsewhere herein.

In another specific embodiment, alpha-fetoprotein fusion proteins of the invention and/or polynucleotides encoding alpha-fetoprotein fusion proteins of the invention are used as a therapy for decreasing cellular proliferation of Large B-cell Lymphomas.

In another specific embodiment, alpha-fetoprotein fusion proteins of the invention and/or polynucleotides encoding alpha-fetoprotein fusion proteins of the invention are used as a means of decreasing the involvement of B cells and Ig associated with Chronic Myelogenous Leukemia.

In specific embodiments, the compositions of the invention are used as an agent to boost immunoresponsiveness among B cell immunodeficient individuals, such as, for example, an individual who has undergone a partial or complete splenectomy.

C. Blood-Related Disorders

The alpha-fetoprotein fusion proteins of the invention and/or polynucleotides encoding alpha-fetoprotein fusion proteins of the invention may be used to modulate hemostatic (the stopping of bleeding) or thrombolytic (clot dissolving) activity. For example, by increasing hemostatic or thrombolytic activity, fusion proteins of the invention and/or polynucleotides encoding alpha-fetoprotein fusion proteins of the invention could be used to treat or prevent blood coagulation diseases, disorders, and/or conditions (e.g., afibrinogenemia, factor deficiencies, hemophilia), blood platelet diseases, disorders, and/or conditions (e.g., thrombocytopenia), or wounds resulting from trauma, surgery, or other causes. Alternatively, fusion proteins of the invention and/or polynucleotides encoding alpha-fetoprotein fusion proteins of the invention that can decrease hemostatic or thrombolytic activity could be used to inhibit or dissolve clotting. These molecules could be important in the treatment or prevention of heart attacks (infarction), strokes, or scarring.

In specific embodiments, the alpha-fetoprotein fusion proteins of the invention and/or polynucleotides encoding alpha-fetoprotein fusion proteins of the invention may be used to prevent, diagnose, prognose, and/or treat thrombosis, arterial thrombosis, venous thrombosis, thromboembolism, pulmonary embolism, atherosclerosis, myocardial infarction, transient ischemic attack, unstable angina. In specific embodiments, the alpha-fetoprotein fusion proteins of the invention and/or polynucleotides encoding alpha-fetoprotein fusion proteins of the invention may be used for the prevention of occulsion of saphenous grafts, for reducing the risk of periprocedural thrombosis as might accompany angioplasty procedures, for reducing the risk of stroke in patients with atrial fibrillation including nonrheumatic atrial fibrillation, for reducing the risk of embolism associated with mechanical heart valves and or mitral valves disease. Other uses for the alpha-fetoprotein fusion proteins of the invention and/or polynucleotides encoding alpha-fetoprotein fusion proteins of the invention, include, but are not limited to, the prevention of occlusions in extrcorporeal devices (e.g., intravascular canulas, vascular access shunts in hemodialysis patients, hemodialysis machines, and cardiopulmonary bypass machines).

In another embodiment, alpha-fetoprotein fusion proteins of the invention and/or polynucleotides encoding alpha-fetoprotein fusion proteins of the invention, may be used to prevent, diagnose, prognose, and/or treat diseases and disorders of the blood and/or blood forming organs associated with the tissue(s) in which the polypeptide of the invention is expressed.

The fusion proteins of the invention and/or polynucleotides encoding alpha-fetoprotein fusion proteins of the invention may be used to modulate hematopoietic activity (the formation of blood cells). For example, the alpha-fetoprotein fusion proteins of the invention and/or polynucleotides encoding alpha-fetoprotein fusion proteins of the invention may be used to increase the quantity of all or subsets of blood cells, such as, for example, erythrocytes, lymphocytes (B or T cells), myeloid cells (e.g., basophils, eosinophils, neutrophils, mast cells, macrophages) and platelets. The ability to decrease the quantity of blood cells or subsets of blood cells may be useful in the prevention, detection, diagnosis and/or treatment of anemias and leukopenias described below. Alternatively, the alpha-fetoprotein fusion proteins of the invention and/or polynucleotides encoding alpha-fetoprotein fusion proteins of the invention may be used to decrease the quantity of all or subsets of blood cells, such as, for example, erythrocytes, lymphocytes (B or T cells), myeloid cells (e.g., basophils, eosinophils, neutrophils, mast cells, macrophages) and platelets. The ability to decrease the quantity of blood cells or subsets of blood cells may be useful in the prevention, detection, diagnosis and/or treatment of leukocytoses, such as, for example eosinophilia.

The fusion proteins of the invention and/or polynucleotides encoding alpha-fetoprotein fusion proteins of the invention may be used to prevent, treat, or diagnose blood dyscrasia.

Anemias are conditions in which the number of red blood cells or amount of hemoglobin (the protein that carries oxygen) in them is below normal. Anemia may be caused by excessive bleeding, decreased red blood cell production, or increased red blood cell destruction (hemolysis). The alpha-fetoprotein fusion proteins of the invention and/or polynucleotides encoding alpha-fetoprotein fusion proteins of the invention may be useful in treating, preventing, and/or diagnosing anemias. Anemias that may be treated prevented or diagnosed by the alpha-fetoprotein fusion proteins of the invention and/or polynucleotides encoding alpha-fetoprotein fusion proteins of the invention include iron deficiency anemia, hypochromic anemia, microcytic anemia, chlorosis, hereditary sideroblastic anemia, idiopathic acquired sideroblastic anemia, red cell aplasia, megaloblastic anemia (e.g., pemicious anemia, (vitamin B12 deficiency) and folic acid deficiency anemia), aplastic anemia, hemolytic anemias (e.g., autoimmune helolytic anemia, microangiopathic hemolytic anemia, and paroxysmal noctumal hemoglobinuria). The alpha-fetoprotein fusion proteins of the invention and/or polynucleotides encoding alpha-fetoprotein fusion proteins of the invention may be useful in treating, preventing, and/or diagnosing anemias associated with diseases including but not limited to, anemias associated with systemic lupus erythematosus, cancers, lymphomas, chronic renal disease, and enlarged spleens. The alpha-fetoprotein fusion proteins of the invention and/or polynucleotides encoding alpha-fetoprotein fusion proteins of the invention may be useful in treating, preventing, and/or diagnosing anemias arising from drug treatments such as anemias associated with methyldopa, dapsone, and/or sulfadrugs. Additionally, fusion proteins of the invention and/or polynucleotides encoding alpha-fetoprotein fusion proteins of the invention may be useful in treating, preventing, and/or diagnosing anemias associated with abnormal red blood cell architecture including but not limited to, hereditary spherocytosis, hereditary elliptocytosis, glucose-6-phosphate dehydrogenase deficiency, and sickle cell anemia.

The alpha-fetoprotein fusion proteins of the invention and/or polynucleotides encoding alpha-fetoprotein fusion proteins of the invention may be useful in treating, preventing, and/or diagnosing hemoglobin abnormalities, (e.g., those associated with sickle cell anemia, hemoglobin C disease, hemoglobin S-C disease, and hemoglobin E disease). Additionally, the alpha-fetoprotein fusion proteins of the invention and/or polynucleotides encoding alpha-fetoprotein fusion proteins of the invention may be useful in diagnosing, prognosing, preventing, and/or treating thalassemias, including, but not limited to, major and minor forms of alpha-thalassemia and beta-thalassemia.

In another embodiment, the alpha-fetoprotein fusion proteins of the invention and/or polynucleotides encoding alpha-fetoprotein fusion proteins of the invention may be useful in diagnosing, prognosing, preventing, and/or treating bleeding disorders including, but not limited to, thrombocytopenia (e.g., idiopathic thrombocytopenic purpura, and thrombotic thrombocytopenic purpura), Von Willebrand's disease, hereditary platelet disorders (e.g., storage pool disease such as Chediak-Higashi and Hermansky-Pudlak syndromes, thromboxane A2 dysfunction, thromboasthenia, and Bemard-Soulier syndrome), hemolytic-uremic syndrome, hemophelias such as hemophelia A or Factor VII deficiency and Christmas disease or Factor IX deficiency, Hereditary Hemorhhagic Telangiectsia, also known as RenduOsler-Weber syndrome, allergic purpura (Henoch Schonlein purpura) and disseminated intravascular coagulation.

The effect of the alpha-fetoprotein fusion proteins of the invention and/or polynucleotides encoding alpha-fetoprotein fusion proteins of the invention on the clotting time of blood may be monitored using any of the clotting tests known in the art including, but not limited to, whole blood partial thromboplastin time (PTT), the activated partial thromboplastin time (aPT-), the activated clotting time (ACT), the recalcified activated clotting time, or the Lee-White Clotting time.

Several diseases and a variety of drugs can cause platelet dysfunction. Thus, in a specific embodiment, the alpha-fetoprotein fusion proteins of the invention and/or polynucleotides encoding alpha-fetoprotein fusion proteins of the invention may be useful in diagnosing, prognosing, preventing, and/or treating acquired platelet dysfunction such as platelet dysfunction accompanying kidney failure, leukemia, multiple myeloma, cirrhosis of the liver, and systemic lupus erythematosus as well as platelet dysfunction associated with drug treatments, including treatment with aspirin, ticlopidine, nonsteroidal anti-inflammatory drugs (used for arthritis, pain, and sprains), and penicillin in high doses.

In another embodiment, the alpha-fetoprotein fusion proteins of the invention and/or polynucleotides encoding alpha-fetoprotein fusion proteins of the invention may be useful in diagnosing, prognosing, preventing, and/or treating diseases and disorders characterized by or associated with increased or decreased numbers of white blood cells. Leukopenia occurs when the number of white blood cells decreases below normal. Leukopenias include, but are not limited to, neutropenia and lymphocytopenia. An increase in the number of white blood cells compared to normal is known as leukocytosis. The body generates increased numbers of white blood cells during infection. Thus, leukocytosis may simply be a normal physiological parameter that reflects infection. Alternatively, leukocytosis may be an indicator of injury or other disease such as cancer. Leokocytoses, include but are not limited to, eosinophilia, and accumulations of macrophages. In specific embodiments, the alpha-fetoprotein fusion proteins of the invention and/or polynucleotides encoding alpha-fetoprotein fusion proteins of the invention may be useful in diagnosing, prognosing, preventing, and/or treating leukopenia. In other specific embodiments, the alpha-fetoprotein fusion proteins of the invention and/or polynucleotides encoding alpha-fetoprotein fusion proteins of the invention may be useful in diagnosing, prognosing, preventing, and/or treating leukocytosis.

Leukopenia may be a generalized decreased in all types of white blood cells, or may be a specific depletion of particular types of white blood cells. Thus, in specific embodiments, the alpha-fetoprotein fusion proteins of the invention and/or polynucleotides encoding alpha-fetoprotein fusion proteins of the invention may be useful in diagnosing, prognosing, preventing, and/or treating decreases in neutrophil numbers, known as neutropenia. Neutropenias that may be diagnosed, prognosed, prevented, and/or treated by the alpha-fetoprotein fusion proteins of the invention and/or polynucleotides encoding alpha-fetoprotein fusion proteins of the invention include, but are not limited to, infantile genetic agranulocytosis, familial neutropenia, cyclic neutropenia, neutropenias resulting from or associated with dietary deficiencies (e.g., vitamin B 12 deficiency or folic acid deficiency), neutropenias resulting from or associated with drug treatments (e.g., antibiotic regimens such as penicillin treatment, sulfonamide treatment, anticoagulant treatment, anticonvulsant drugs, anti-thyroid drugs, and cancer chemotherapy), and neutropenias resulting from increased neutrophil destruction that may occur in association with some bacterial or viral infections, allergic disorders, autoimmune diseases, conditions in which an individual has an enlarged spleen (e.g., Felty syndrome, malaria and sarcoidosis), and some drug treatment regimens.

The alpha-fetoprotein fusion proteins of the invention and/or polynucleotides encoding alpha-fetoprotein fusion proteins of the invention may be useful in diagnosing, prognosing, preventing, and/or treating lymphocytopenias (decreased numbers of B and/or T lymphocytes), including, but not limited to, lymphocytopenias resulting from or associated with stress, drug treatments (e.g., drug treatment with corticosteroids, cancer chemotherapies, and/or radiation therapies), AIDS infection and/or other diseases such as, for example, cancer, rheumatoid arthritis, systemic lupus erythematosus, chronic infections, some viral infections and/or hereditary disorders (e.g., DiGeorge syndrome, Wiskott-Aldrich Syndrome, severe combined immunodeficiency, ataxia telangiectsia).

The alpha-fetoprotein fusion proteins of the invention and/or polynucleotides encoding alpha-fetoprotein fusion proteins of the invention may be useful in diagnosing, prognosing, preventing, and/or treating diseases and disorders associated with macrophage numbers and/or macrophage function including, but not limited to, Gaucher's disease, Niemann-Pick disease, Letterer-Siwe disease and Hand-Schuller-Christian disease.

In another embodiment, the alpha-fetoprotein fusion proteins of the invention and/or polynucleotides encoding alpha-fetoprotein fusion proteins of the invention may be useful in diagnosing, prognosing, preventing, and/or treating diseases and disorders associated with eosinophil numbers and/or eosinophil function including, but not limited to, idiopathic hypereosinophilic syndrome, eosinophilia-myalgia syndrome, and Hand-Schuller-Christian disease.

In yet another embodiment, the alpha-fetoprotein fusion proteins of the invention and/or polynucleotides encoding alpha-fetoprotein fusion proteins of the invention may be useful in diagnosing, prognosing, preventing, and/or treating leukemias and lymphomas including, but not limited to, acute lymphocytic (lymphpblastic) leukemia (ALL), acute mycloid (myelocytic, myelogenous, myeloblastic, or myelomonocytic) leukemia, chronic lymphocytic leukemia (e.g., B cell leukemias, T cell leukemias, Sezary syndrome, and Hairy cell leukenia), chronic myelocytic (myeloid, myelogenous, or granulocytic) leukemia, Hodgkin's lymphoma, non-hodgkin's lymphoma, Burkitt's lymphoma, and mycosis fungoides.

In other embodiments, the alpha-fetoprotein fusion proteins of the invention and/or polynucleotides encoding alpha-fetoprotein fusion proteins of the invention may be useful in diagnosing, prognosing, preventing, and/or treating diseases and disorders of plasma cells including, but not limited to, plasma cell dyscrasias, monoclonal gammaopathies, monoclonal gammopathies of undetermined significance, multiple myeloma, macroglobulinemia, Waldenstrom's macroglobulinemia, cryoglobulinemia, and Raynaud's phenomenon.

In other embodiments, the alpha-fetoprotein fusion proteins of the invention and/or polynucleotides encoding alpha-fetoprotein fusion proteins of the invention may be useful in treating, preventing, and/or diagnosing myeloproliferative disorders, including but not limited to, polycythemia vera, relative polycythemia, secondary polycythemia, myelofibrosis, acute myelofibrosis, agnogenic myelod metaplasia, thrombocythemia, (including both primary and seconday thrombocythemia) and chronic myelocytic leukemia.

In other embodiments, the alpha-fetoprotein fusion proteins of the invention and/or polynucleotides encoding alpha-fetoprotein fusion proteins of the invention may be useful as a treatment prior to surgery, to increase blood cell production.

In other embodiments, the alpha-fetoprotein fusion proteins of the invention and/or polynucleotides encoding alpha-fetoprotein fusion proteins of the invention may be useful as an agent to enhance the migration, phagocytosis, superoxide production, antibody dependent cellular cytotoxicity of neutrophils, eosionophils and macrophages.

In other embodiments, the alpha-fetoprotein fusion proteins of the invention and/or polynucleotides encoding alpha-fetoprotein fusion proteins of the invention may be useful as an agent to increase the number of stem cells in circulation prior to stem cells pheresis. In another specific embodiment, the alpha-fetoprotein fusion proteins of the invention and/or polynucleotides encoding alpha-fetoprotein fusion proteins of the invention may be useful as an agent to increase the number of stem cells in circulation prior to platelet pheresis.

In other embodiments, the alpha-fetoprotein fusion proteins of the invention and/or polynucleotides encoding alpha-fetoprotein fusion proteins of the invention may be useful as an agent to increase cytokine production.

In other embodiments, the alpha-fetoprotein fusion proteins of the invention and/or polynucleotides encoding alpha-fetoprotein fusion proteins of the invention may be useful in preventing, diagnosing, and/or treating primary hematopoietic disorders.

D. Hyperproliferative Disorders

In certain embodiments, fusion proteins of the invention and/or polynucleotides encoding alpha-fetoprotein fusion proteins of the invention can be used to treat or detect hyperproliferative disorders, including neoplasms. Alpha-fetoprotein fusion proteins of the invention and/or polynucleotides encoding alpha-fetoprotein fusion proteins of the invention may inhibit the proliferation of the disorder through direct or indirect interactions. Alternatively, fusion proteins of the invention and/or polynucleotides encoding alpha-fetoprotein fusion proteins of the invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by fusion proteins of the invention and/or polynucleotides encoding alpha-fetoprotein fusion proteins of the invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvis, skin, soft tissue, spleen, thorax, and urogenital tract.

Similarly, other hyperproliferative disorders can also be treated or detected by fusion proteins of the invention and/or polynucleotides encoding alpha-fetoprotein fusion proteins of the invention. Examples of such hyperproliferative disorders include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple MyelomalPlasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Normelanoma Skin Cancer, Non-Small Cell Lung Cancer, Occult Primary Metastatic Squamous Neck Cancer, Oropharyngeal Cancer, Osteo-/Malignant Fibrous Sarcoma, OsteosarcomaWMalignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In another preferred embodiment, alpha-fetoprotein fusion proteins of the invention and/or polynucleotides encoding alpha-fetoprotein fusion proteins of the invention are used to diagnose, prognose, prevent, and/or treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79.)

Hyperplasia is a form of controlled cell proliferation, involving an increase in cell number in a tissue or organ, without significant alteration in structure or function. Hyperplastic disorders which can be diagnosed, prognosed, prevented, and/or treated with fusion proteins of the invention and/or polynucleotides encoding alpha-fetoprotein fusion proteins of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be diagnosed, prognosed, prevented, and/or treated with fusion proteins of the invention and/or polynucleotides encoding alpha-fetoprotein fusion proteins of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be diagnosed, prognosed, prevented, and/or treated with fusion proteins of the invention and/or polynucleotides encoding alpha-fetoprotein fusion proteins of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be diagnosed, prognosed, prevented, and/or treated with fusion proteins of the invention and/or polynucleotides encoding alpha-fetoprotein fusion proteins of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In another embodiment, alpha-fetoprotein fusion proteins of the invention and/or polynucleotides encoding alpha-fetoprotein fusion proteins of the invention, may be used to diagnose and/or prognose disorders associated with the tissue(s) in which the polypeptide of the invention is expressed.

In another embodiment, alpha-fetoprotein fusion proteins of the invention and/or polynucleotides encoding alpha-fetoprotein fusion proteins of the invention conjugated to a toxin or a radioactive isotope, as described herein, may be used to treat cancers and neoplasms, including, but not limited to, those described herein. In a further preferred embodiment, alpha-fetoprotein fusion proteins of the invention and/or polynucleotides encoding alpha-fetoprotein fusion proteins of the invention conjugated to a toxin or a radioactive isotope, as described herein, may be used to treat acute myelogenous leukemia.

Additionally, fusion proteins of the invention and/or polynucleotides encoding alpha-fetoprotein fusion proteins of the invention may affect apoptosis, and therefore, would be useful in treating a number of diseases associated with increased cell survival or the inhibition of apoptosis. For example, diseases associated with increased cell survival or the inhibition of apoptosis that could be diagnosed, prognosed, prevented, and/or treated by polynucleotides, polypeptides, and/or agonists or antagonists of the invention, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection.

In preferred embodiments, fusion proteins of the invention and/or polynucleotides encoding alpha-fetoprotein fusion proteins of the invention are used to inhibit growth, progression, and/or metastasis of cancers, in particular those listed above.

Additional diseases or conditions associated with increased cell survival that could be diagnosed, prognosed, prevented, and/or treated by fusion proteins of the invention and/or polynucleotides encoding alpha-fetoprotein fusion proteins of the invention, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

Diseases associated with increased apoptosis that could be diagnosed, prognosed, prevented, and/or treated by fusion proteins of the invention and/or polynucleotides encoding alpha-fetoprotein fusion proteins of the invention, include AIDS; neurodegenerative disorders (such as Alzheimer's disease, Parkinson's disease, amyotrophic lateral sclerosis, retinitis pigmentosa, cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemialreperfusion injury, cholestosis (bile duct injury) and liver cancer); toxin-induced liver disease (such as that caused by alcohol), septic shock, cachexia and anorexia.

Hyperproliferative diseases and/or disorders that could be diagnosed, prognosed, prevented, and/or treated by fusion proteins of the invention and/or polynucleotides encoding alpha-fetoprotein fusion proteins of the invention, include, but are not limited to, neoplasms located in the liver, abdomen, bone, breast, digestive system, pancreas, peritoneum, endocrine glands (adrenal, parathyroid, pituitary, testicles, ovary, thymus, thyroid), eye, head and neck, nervous system (central and peripheral), lymphatic system, pelvis, skin, soft tissue, spleen, thorax, and urogenital tract.

Similarly, other hyperproliferative disorders can also be diagnosed, prognosed, prevented, and/or treated by fusion proteins of the invention and/or polynucleotides encoding alpha-fetoprotein fusion proteins of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above.

Another preferred embodiment utilizes polynucleotides encoding alpha-fetoprotein fusion proteins of the invention to inhibit aberrant cellular division, by gene therapy using the present invention, and/or protein fusions or fragments thereof. Thus, the present invention provides a method for treating cell proliferative disorders by inserting into an abnormally proliferating cell a polynucleotide encoding an alpha-fetoprotein fusion protein of the present invention, wherein said polynucleotide represses said expression.

Another embodiment of the present invention provides a method of treating cell-proliferative disorders in individuals comprising administration of one or more active gene copies of the present invention to an abnormally proliferating cell or cells. In a preferred embodiment, polynucleotides of the present invention is a DNA construct comprising a recombinant expression vector effective in expressing a DNA sequence encoding said polynucleotides. In another preferred embodiment of the present invention, the DNA construct encoding the fusion protein of the present invention is inserted into cells to be treated utilizing a retrovirus, or more preferably an adenoviral vector (See G J. Nabel, et. al., PNAS 1999 96: 324-326, which is hereby incorporated by reference). In a most preferred embodiment, the viral vector is defective and will not transform non-proliferating cells, only proliferating cells. Moreover, in a preferred embodiment, the polynucleotides of the present invention inserted into proliferating cells either alone, or in combination with or fused to other polynucleotides, can then be modulated via an external stimulus (i.e. magnetic, specific small molecule, chemical, or drug administration, etc.), which acts upon the promoter upstream of said polynucleotides to induce expression of the encoded protein product. As such the beneficial therapeutic affect of the present invention may be expressly modulated (i.e. to increase, decrease, or inhibit expression of the present invention) based upon said external stimulus.

Polynucleotides of the present invention may be useful in repressing expression of oncogenic genes or antigens. By “repressing expression of the oncogenic genes” is intended the suppression of the transcription of the gene, the degradation of the gene transcript (pre-message RNA), the inhibition of splicing, the destruction of the messenger RNA, the prevention of the post-translational modifications of the protein, the destruction of the protein, or the inhibition of the normal function of the protein.

For local administration to abnormally proliferating cells, polynucleotides of the present invention may be administered by any method known to those of skill in the art including, but not limited to transfection, electroporation, microinjection of cells, or in vehicles such as liposomes, lipofectin, or as naked polynucleotides, or any other method described throughout the specification. The polynucleotide of the present invention may be delivered by known gene delivery systems such as, but not limited to, retroviral vectors (Gilboa, J. Virology 44:845 (1982); Hocke, Nature 320:275 (1986); Wilson, et al., Proc. Natl. Acad. Sci. U.S.A. 85:3014), vaccinia virus system (Chakrabarty et al., Mol. Cell. Biol. 5:3403 (1985) or other efficient DNA delivery systems (Yates et al., Nature 313:812 (1985)) known to those skilled in the art. These references are exemplary only and are hereby incorporated by reference. To specifically deliver or transfect cells which are abnormally proliferating and spare non-dividing cells, it is preferable to utilize a retrovirus, or adenoviral (as described in the art and elsewhere herein) delivery system known to those of skill in the art. Since host DNA replication is required for retroviral DNA to integrate and the retrovirus will be unable to self replicate due to the lack of the retrovirus genes needed for its life cycle. Utilizing such a retroviral delivery system for polynucleotides of the present invention will target said gene and constructs to abnormally proliferating cells and will spare the non-dividing normal cells.

The polynucleotides of the present invention may be delivered directly to cell proliferative disorder/disease sites in internal organs, body cavities and the like by use of imaging devices used to guide an injecting needle directly to the disease site. The polynucleotides of the present invention may also be administered to disease sites at the time of surgical intervention.

By “cell proliferative disease” is meant any human or animal disease or disorder, affecting any one or any combination of organs, cavities, or body parts, which is characterized by single or multiple local abnormal proliferations of cells, groups of cells, or tissues, whether benign or malignant.

Any amount of the polynucleotides of the present invention may be administered as long as it has a biologically inhibiting effect on the proliferation of the treated cells. Moreover, it is possible to administer more than one of the polynucleotide of the present invention simultaneously to the same site. By “biologically inhibiting” is meant partial or total growth inhibition as well as decreases in the rate of proliferation or growth of the cells. The biologically inhibitory dose may be determined by assessing the effects of the polynucleotides of the present invention on target malignant or abnormally proliferating cell growth in tissue culture, tumor growth in animals and cell cultures, or any other method known to one of ordinary skill in the art.

Moreover, fusion proteins of the invention and/or polynucleotides encoding alpha-fetoprotein fusion proteins of the invention of the present invention are useful in inhibiting the angiogenesis of proliferative cells or tissues, either alone, as a protein fusion, or in combination with other polypeptides directly or indirectly, as described elsewhere herein. In a most preferred embodiment, the anti-angiogenesis effect may be achieved indirectly, for example, through the inhibition of hematopoietic, tumor-specific cells, such as tumor-associated macrophages (See Joseph I B, et al. J Natl Cancer Inst, 90(21):1648-53 (1998), which is hereby incorporated by reference).

Alpha-fetoprotein fusion proteins of the invention and/or polynucleotides encoding alpha-fetoprotein fusion proteins of the invention may be useful in inhibiting proliferative cells or tissues through the induction of apoptosis. These fusion proteins and/or polynucleotides may act either directly, or indirectly to induce apoptosis of proliferative cells and tissues, for example in the activation of a death-domain receptor, such as tumor necrosis factor (TNF) receptor-1, CD95 (Fas/APO-1), TNF-receptor-related apoptosis-mediated protein (TRAMP) and TNF-related apoptosis-inducing ligand (TRAIL) receptor-1 and -2 (See Schulze-Osthoff K, et. al., Eur J Biochem 254(3):439-59 (1998), which is hereby incorporated by reference). Moreover, in another preferred embodiment of the present invention, these fusion proteins and/or polynucleotides may induce apoptosis through other mechanisms, such as in the activation of other proteins which will activate apoptosis, or through stimulating the expression of these proteins, either alone or in combination with small molecule drugs or adjuvants, such as apoptonin, galectins, thioredoxins, anti-inflammatory proteins (See for example, Mutat Res 400(1-2):447-55 (1998), Med Hypotheses.50(5):423-33 (1998), Chem Biol Interact. April 24; 111-112:23-34 (1998), J Mol Med. 76(6):402-12 (1998), Int J Tissue React; 20(1):3-15 (1998), which are all hereby incorporated by reference).

Alpha-fetoprotein fusion proteins of the invention and/or polynucleotides encoding alpha-fetoprotein fusion proteins of the invention are useful in inhibiting the metastasis of proliferative cells or tissues. Inhibition may occur as a direct result of administering these alpha-fetoprotein fusion proteins and/or polynucleotides, or indirectly, such as activating the expression of proteins known to inhibit metastasis, for example alpha 4 integrins, (See, e.g., Curr Top Microbiol Immunol 1998; 231:12541, which is hereby incorporated by reference). Such therapeutic affects of the present invention may be achieved either alone, or in combination with small molecule drugs or adjuvants.

In another embodiment, the invention provides a method of delivering compositions containing the alpha-fetoprotein fusion proteins of the invention and/or polynucleotides encoding alpha-fetoprotein fusion proteins of the invention to targeted cells expressing the a polypeptide bound by, that binds to, or associates with an alpha-fetoprotein fusion protein of the invention. Alpha-fetoprotein fusion proteins of the invention may be associated with heterologous polypeptides, heterologous nucleic acids, toxins, or prodrugs via hydrophobic, hydrophilic, ionic and/or covalent interactions.

Alpha-fetoprotein fusion proteins of the invention are useful in enhancing the immunogenicity and/or antigenicity of proliferating cells or tissues, either directly, such as would occur if the alpha-fetoprotein fusion proteins of the invention ‘vaccinated’ the immune response to respond to proliferative antigens and immunogens, or indirectly, such as in activating the expression of proteins known to enhance the immune response (e.g. chemokines), to said antigens and immunogens.

E. Renal Disorders

Alpha-fetoprotein fusion proteins of the invention and/or polynucleotides encoding alpha-fetoprotein fusion proteins of the invention, may be used to treat, prevent, diagnose, and/or prognose disorders of the renal system. Renal disorders which can be diagnosed, prognosed, prevented, and/or treated with compositions of the invention include, but are not limited to, kidney failure, nephritis, blood vessel disorders of kidney, metabolic and congenital kidney disorders, urinary disorders of the kidney, autoimmune disorders, sclerosis and necrosis, electrolyte imbalance, and kidney cancers.

Kidney diseases which can be diagnosed, prognosed, prevented, and/or treated with compositions of the invention include, but are not limited to, acute kidney failure, chronic kidney failure, atheroembolic renal failure, end-stage renal disease, inflammatory diseases of the kidney (e.g., acute glomerulonephritis, postinfectious glomerulonephritis, rapidly progressive glomerulonephritis, nephrotic syndrome, membranous glomerulonephritis, familial nephrotic syndrome, membranoproliferative glomerulonephritis I and II, mesangial proliferative glomerulonephritis, chronic glomerulonephritis, acute tubulointerstitial nephritis, chronic tubulointerstitial nephritis, acute post-streptococcal glomerulonephritis (PSGN), pyelonephritis, lupus nephritis, chronic nephritis, interstitial nephritis, and post-streptococcal glomerulonephritis), blood vessel disorders of the kidneys (e.g., kidney infarction, atheroembolic kidney disease, cortical necrosis, malignant nephrosclerosis, renal vein thrombosis, renal underperfusion, renal retinopathy, renal ischemia-reperfusion, renal artery embolism, and renal artery stenosis), and kidney disorders resulting form urinary tract disease (e.g., pyelonephritis, hydronephrosis, urolithiasis (renal lithiasis, nephrolithiasis), reflux nephropathy, urinary tract infections, urinary retention, and acute or chronic unilateral obstructive uropathy.)

In addition, compositions of the invention can be used to diagnose, prognose, prevent, and/or treat metabolic and congenital disorders of the kidney (e.g., uremia, renal amyloidosis, renal osteodystrophy, renal tubular acidosis, renal glycosuria, nephrogenic diabetes insipidus, cystinuria, Fanconi's syndrome, renal fibrocystic osteosis (renal rickets), Hartnup disease, Bartter's syndrome, Liddle's syndrome, polycystic kidney disease, medullary cystic disease, medullary sponge kidney, Alport's syndrome, nail-patella syndrome, congenital nephrotic syndrome, CRUSH syndrome, horseshoe kidney, diabetic nephropathy, nephrogenic diabetes insipidus, analgesic nephropathy, kidney stones, and membranous nephropathy), and autoimmune disorders of the kidney (e.g., systemic lupus erythematosus (SLE), Goodpasture syndrome, IgA nephropathy, and IgM mesangial proliferative glomerulonephritis).

Compositions of the invention can also be used to diagnose, prognose, prevent, and/or treat sclerotic or necrotic disorders of the kidney (e.g., glomerulosclerosis, diabetic nephropathy, focal segmental glomerulosclerosis (FSGS), necrotizing glomerulonephritis, and renal papillary necrosis), cancers of the kidney (e.g., nephroma, hypernephroma, nephroblastoma, renal cell cancer, transitional cell cancer, renal adenocarcinoma, squamous cell cancer, and Wilm's tumor), and electrolyte imbalances (e.g., nephrocalcinosis, pyuria, edema, hydronephritis, proteinuria, hyponatremia, hypematremia, hypokalemia, hyperkalemia, hypocalcemia, hypercalcemia, hypophosphatemia, and hyperphosphatemia).

Compositions of the invention may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Compositions of the invention may be administered as part of a therapeutic, described in more detail below. Methods of delivering polynucleotides of the invention are described in more detail herein.

F. Cardiovascular Disorders

Alpha-fetoprotein fusion proteins of the invention and/or polynucleotides encoding alpha-fetoprotein fusion proteins of the invention, may be used to treat, prevent, diagnose, and/or prognose cardiovascular disorders, including, but not limited to, peripheral artery disease, such as limb ischemia.

Cardiovascular disorders include, but are not limited to, cardiovascular abnormalities, such as arterio-arterial fistula, arteriovenous fistula, cerebral arteriovenous mAFPormations, congenital heart defects, pulmonary atresia, and Scimitar Syndrome. Congenital heart defects include, but are not limited to, aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular disorders also include, but are not limited to, heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include, but are not limited to, sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve diseases include, but are not limited to, aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include, but are not limited to, alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include, but are not limited to, coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular disorders, diabetic angiopathies, diabetic retinopathy, embolisms, thrombosis, erythromelaigia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include, but are not limited to, dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include, but are not limited to, arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular disorders include, but are not limited to, carotid artery diseases, cerebral amyloid angiopathy, cerebral aneurysm, cerebral anoxia, cerebral arteriosclerosis, cerebral arteriovenous mAFPormation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subelavian steal syndrome, periventricular leukomalacia, vascular headache, cluster headache, migraine, and vertebrobasilar insufficiency.

Embolisms include, but are not limited to, air embolisms, amniotic fluid embolisms, cholesterol embolisms, blue toe syndrome, fat embolisms, pulmonary embolisms, and thromoboembolisms. Thrombosis include, but are not limited to, coronary thrombosis, hepatic vein thrombosis, retinal vein occlusion, carotid artery thrombosis, sinus thrombosis, Wallenberg's syndrome, and thrombophlebitis.

Ischemic disorders include, but are not limited to, cerebral ischemia, ischemic colitis, compartment syndromes, anterior compartment syndrome, myocardial ischemia, reperfusion injuries, and peripheral limb ischemia. Vasculitis includes, but is not limited to, aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

Alpha-fetoprotein fusion proteins of the invention and/or polynucleotides encoding alpha-fetoprotein fusion proteins of the invention may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Methods of delivering polynucleotides are described in more detail herein.

G. Respiratory Disorders

Alpha-fetoprotein fusion proteins of the invention and/or polynucleotides encoding alpha-fetoprotein fusion proteins of the invention may be used to treat, prevent, diagnose, and/or prognose diseases and/or disorders of the respiratory system.

Diseases and disorders of the respiratory system include, but are not limited to, nasal vestibulitis, nonallergic rhinitis (e.g., acute rhinitis, chronic rhinitis, atrophic rhinitis, vasomotor rhinitis), nasal polyps, and sinusitis, juvenile angiofibromas, cancer of the nose and juvenile papillomas, vocal cord polyps, nodules (singer's nodules), contact ulcers, vocal cord paralysis, laryngoceles, pharyngitis (e.g., viral and bacterial), tonsillitis, tonsillar cellulitis, parapharyngeal abscess, laryngitis, laryngoceles, and throat cancers (e.g., cancer of the nasopharynx, tonsil cancer, larynx cancer), lung cancer (e.g., squamous cell carcinoma, small cell (oat cell) carcinoma, large cell carcinoma, and adenocarcinoma), allergic disorders (eosinophilic pneumonia, hypersensitivity pneumonitis (e.g., extrinsic allergic alveolitis, allergic interstitial pneumonitis, organic dust pneumoconiosis, allergic bronchopulmonary aspergillosis, asthma, Wegener's granulomatosis (granulomatous vasculitis), Goodpasture's syndrome)), pneumonia (e.g., bacterial pneumonia (e.g., Streptococcus pneumoniae (pneumoncoccal pneumonia), Staphylococcus aureus (staphylococcal pneumonia), Gram-negative bacterial pneumonia (caused by, e.g., Klebsiella and Pseudomas spp.), Mycoplasma pneumoniae pneumonia, Hemophilus influenzae pneumonia, Legionella pneumophila (Legionnaires' disease), and Chlamydia psittaci (Psittacosis)), and viral pneumonia (e.g., influenza, chickenpox (varicella).

Additional diseases and disorders of the respiratory system include, but are not limited to bronchiolitis, polio (poliomyelitis), croup, respiratory syncytial viral infection, mumps, erythema infectiosum (fifth disease), roseola infantum, progressive rubella panencephalitis, german measles, and subacute sclerosing panencephalitis), fungal pneumonia (e.g., Histoplasmosis, Coccidioidomycosis, Blastomycosis, fungal infections in people with severely suppressed immune systems (e.g., cryptococcosis, caused by Cryptococcus neoformans; aspergillosis, caused by Aspergillus spp.; candidiasis, caused by Candida; and mucormycosis)), Pneumocystis carinji (pneumocystis pneumonia), atypical pneumonias (e.g., Mycoplasma and Chlamydia spp.), opportunistic infection pneumonia, nosocomial pneumonia, chemical pneumonitis, and aspiration pneumonia, pleural disorders (e.g., pleurisy, pleural effusion, and pneumothorax (e.g., simple spontaneous pneumothorax, complicated spontaneous pneumothorax, tension pneumothorax)), obstructive airway diseases (e.g., asthma, chronic obstructive pulmonary disease (COPD), emphysema, chronic or acute bronchitis), occupational lung diseases (e.g., silicosis, black lung (coal workers' pneumoconiosis), asbestosis, berylliosis, occupational asthsma, byssinosis, and benign pneumoconioses), Infiltrative Lung Disease (e.g., pulmonary fibrosis (e.g., fibrosing alveolitis, usual interstitial pneumonia), idiopathic pulmonary fibrosis, desquamative interstitial pneumonia, lymphoid interstitial pneumonia, histiocytosis X (e.g., Letterer-Siwe disease, Hand-Schuller Christian disease, eosinophilic granuloma), idiopathic pulmonary hemosiderosis, sarcoidosis and pulmonary alveolar proteinosis), Acute respiratory distress syndrome (also called, e.g., adult respiratory distress syndrome), edema, pulmonary embolism, bronchitis (e.g., viral, bacterial), bronchiectasis, atelectasis, lung abscess (caused by, e.g., Staphylococcus aureus or Legionella pneumophila), and cystic fibrosis.

H. Anti-Angiogenesis Activity

The naturally occurring balance between endogenous stimulators and inhibitors of angiogenesis is one in which inhibitory influences predominate. Rastinejad et al. Cell 56:345-355 (1989). In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye disorders, and psoriasis. See, e.g., reviews by Moses et al., Biotech. 9:630-634 (1991); Follanan et al., N. Engl. J. Med., 333:1757-1763 (1995); Auerbach et al., J. Microvasc. Res. 29:401-411 (1985); Follanan, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases or disorders associated with neovascularization by administration of fusion proteins of the invention and/or polynucleotides encoding alpha-fetoprotein fusion proteins of the invention. Malignant and metastatic conditions which can be treated with the polynucleotides and polypeptides, or agonists or antagonists of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)). Thus, the present invention provides a method of treating an angiogenesis-related disease and/or disorder, comprising administering to an individual in need thereof a therapeutically effective amount of an alpha-fetoprotein fusion protein of the invention and/or polynucleotides encoding an alpha-fetoprotein fusion protein of the invention. For example, fusion proteins of the invention and/or polynucleotides encoding alpha-fetoprotein fusion proteins of the invention may be utilized in a variety of additional methods in order to therapeutically treat a cancer or tumor. Cancers which may be treated with fusion proteins of the invention and/or polynucleotides encoding alpha-fetoprotein fusion proteins of the invention include, but are not limited to solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer; colorectal cancer; advanced malignancies; and blood born tumors such as leukemias. For example, fusion proteins of the invention and/or polynucleotides encoding alpha-fetoprotein fusion proteins of the invention may be delivered topically, in order to treat cancers such as skin cancer, head and neck tumors, breast tumors, and Kaposi's sarcoma.

Within yet other aspects, fusion proteins of the invention and/or polynucleotides encoding alpha-fetoprotein fusion proteins of the invention may be utilized to treat superficial forms of bladder cancer by, for example, intravesical administration. Alpha-fetoprotein fusion proteins of the invention and/or polynucleotides encoding alpha-fetoprotein fusion proteins of the invention may be delivered directly into the tumor, or near the tumor site, via injection or a catheter. Of course, as the artisan of ordinary skill will appreciate, the appropriate mode of administration will vary according to the cancer to be treated. Other modes of delivery are discussed herein.

Alpha-fetoprotein fusion proteins of the invention and/or polynucleotides encoding alpha-fetoprotein fusion proteins of the invention may be useful in treating other disorders, besides cancers, which involve angiogenesis. These disorders include, but are not limited to: benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous mAFPormations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within one aspect of the present invention methods are provided for treating hypertrophic scars and keloids, comprising the step of administering alpha-fetoprotein fusion proteins of the invention and/or polynucleotides encoding alpha-fetoprotein fusion proteins of the invention to a hypertrophic scar or keloid.

Within one embodiment of the present invention fusion proteins of the invention and/or polynucleotides encoding alpha-fetoprotein fusion proteins of the invention are directly injected into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development. As noted above, the present invention also provides methods for treating neovascular diseases of the eye, including for example, corneal neovascularization, neovascular glaucoma, proliferative diabetic retinopathy, retrolental fibroplasia and macular degeneration.

Moreover, Ocular disorders associated with neovascularization which can be treated with the alpha-fetoprotein fusion proteins of the invention and/or polynucleotides encoding alpha-fetoprotein fusion proteins of the invention include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Thus, within one aspect of the present invention methods are provided for treating neovascular diseases of the eye such as corneal neovascularization (including corneal graft neovascularization), comprising the step of administering to a patient a therapeutically effective amount of a compound (e.g., fusion proteins of the invention and/or polynucleotides encoding alpha-fetoprotein fusion proteins of the invention) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of disorders can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within particularly preferred embodiments of the invention, may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. Within preferred embodiments, the anti-angiogenic composition is prepared with a muco-adhesive polymer which binds to cornea. Within further embodiments, the anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the compounds described above may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating neovascular glaucoma, comprising the step of administering to a patient a therapeutically effective amount of an alpha-fetoprotein fusion protein of the invention and/or polynucleotides encoding an alpha-fetoprotein fusion protein of the invention to the eye, such that the formation of blood vessels is inhibited. In one embodiment, the compound may be administered topically to the eye in order to treat early forms of neovascular glaucoma. Within other embodiments, the compound may be implanted by injection into the region of the anterior chamber angle. Within other embodiments, the compound may also be placed in any location such that the compound is continuously released into the aqueous humor. Within another aspect of the present invention, methods are provided for treating proliferative diabetic retinopathy, comprising the step of administering to a patient a therapeutically effective amount of an alpha-fetoprotein fusion protein of the invention and/or polynucleotides encoding an alpha-fetoprotein fusion protein of the invention to the eyes, such that the formation of blood vessels is inhibited.

Within particularly preferred embodiments of the invention, proliferative diabetic retinopathy may be treated by injection into the aqueous humor or the vitreous to increase the local concentration of the polynucleotide, polypeptide, antagonist and/or agonist in the retina. Preferably, this treatment should be initiated prior to the acquisition of severe disease requiring photocoagulation.

Within another aspect of the present invention, methods are provided for treating retrolental fibroplasia, comprising the step of administering to a patient a therapeutically effective amount of an alpha-fetoprotein fusion protein of the invention and/or polynucleotides encoding an alpha-fetoprotein fusion protein of the invention to the eye, such that the formation of blood vessels is inhibited. The compound may be administered topically, via intravitreous injection and/or via intraocular implants.

Additionally, disorders which can be treated with fusion proteins of the invention and/or polynucleotides encoding alpha-fetoprotein fusion proteins of the invention include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, disorders and/or states, which can be treated, prevented, diagnosed, and/or prognosed with the alpha-fetoprotein fusion proteins of the invention and/or polynucleotides encoding alpha-fetoprotein fusion proteins of the invention of the invention include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous mAFPormations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

In one aspect of the birth control method, an amount of the compound sufficient to block embryo implantation is administered before or after intercourse and fertilization have occurred, thus providing an effective method of birth control, possibly a “morning after” method. Alpha-fetoprotein fusion proteins of the invention and/or polynucleotides encoding alpha-fetoprotein fusion proteins of the invention may also be used in controlling menstruation or administered as either a peritoneal lavage fluid or for peritoneal implantation in the treatment of endometriosis.

Alpha-fetoprotein fusion proteins of the invention and/or polynucleotides encoding alpha-fetoprotein fusion proteins of the invention may be incorporated into surgical sutures in order to prevent stitch granulomas.

Alpha-fetoprotein fusion proteins of the invention and/or polynucleotides encoding alpha-fetoprotein fusion proteins of the invention may be utilized in a wide variety of surgical procedures. For example, within one aspect of the present invention a compositions (in the form of, for example, a spray or film) may be utilized to coat or spray an area prior to removal of a tumor, in order to isolate normal surrounding tissues from malignant tissue, and/or to prevent the spread of disease to surrounding tissues. Within other aspects of the present invention, compositions (e.g., in the form of a spray) may be delivered via endoscopic procedures in order to coat tumors, or inhibit angiogenesis in a desired locale. Within yet other aspects of the present invention, surgical meshes which have been coated with anti-angiogenic compositions of the present invention may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

Within further aspects of the invention, methods are provided for treating tumor excision sites, comprising administering alpha-fetoprotein fusion proteins of the invention and/or polynucleotides encoding alpha-fetoprotein fusion proteins of the invention to the resection margins of a tumor subsequent to excision, such that the local recurrence of cancer and the formation of new blood vessels at the site is inhibited. Within one embodiment of the invention, the anti-angiogenic compound is administered directly to the tumor excision site (e.g., applied by swabbing, brushing or otherwise coating the resection margins of the tumor with the anti-angiogenic compound). Alternatively, the anti-angiogenic compounds may be incorporated into known surgical pastes prior to administration. Within particularly preferred embodiments of the invention, the anti-angiogenic compounds are applied after hepatic resections for malignancy, and after neurosurgical operations.

Within one aspect of the invention, fusion proteins of the invention and/or polynucleotides encoding alpha-fetoprotein fusion proteins of the invention may be administered to the resection margin of a wide variety of tumors, including for example, breast, colon, brain and hepatic tumors. For example, within one embodiment of the invention, anti-angiogenic compounds may be administered to the site of a neurological tumor subsequent to excision, such that the formation of new blood vessels at the site are inhibited.

The alpha-fetoprotein fusion proteins of the invention and/or polynucleotides encoding alpha-fetoprotein fusion proteins of the invention may also be administered along with other anti-angiogenic factors. Representative examples of other anti-angiogenic factors include: Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VD) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the invention. Representative examples include platelet factor 4; protamine sulphate; sulphated chitin derivatives (prepared from queen crab shells), (Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChDMP-3 (Pavioff et al., J. Bio. Chem. 267:17321-17326, (1992)); Chymostatin (Tomkinson et al., Biochem J. 286:475-480, (1992)); Cyclodextrin Tetradecasulfate; Eponemycin; Camptothecin; Fumagillin (Ingber et al., Nature 348:555-557, 1990); Gold Sodium Thiomalate (“GST”; Matsubara and Ziff, J. Clin. Invest. 79:1440-1446, (1987)); anticollagenase-serum; alpha2-antiplasmin (Holmes et al., J. Biol. Chem. 262(4):1659-1664, (1987)); Bisantrene (National Cancer Institute); Lobenzarit disodium (N-2)-carboxyphenyl-4-ch-loroanthronilic acid disodium or “CCA”; Takeuchi et al., Agents Actions 36:312-316, (1992)); Thalidomide; Angostatic steroid; AGM-1470; carboxynaminolmidazole; and metalloproteinase inhibitors such as BB94.

I. Diseases at the Cellular Level

Diseases associated with increased cell survival or the inhibition of apoptosis that could be treated, prevented, diagnosed, and/or prognosed using fusion proteins of the invention and/or polynucleotides encoding alpha-fetoprotein fusion proteins of the invention, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection.

In preferred embodiments, fusion proteins of the invention and/or polynucleotides encoding alpha-fetoprotein fusion proteins of the invention are used to inhibit growth, progression, and/or metasis of cancers, in particular those listed above.

Additional diseases or conditions associated with increased cell survival that could be treated or detected by fusion proteins of the invention and/or polynucleotides encoding alpha-fetoprotein fusion proteins of the invention include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated, prevented, diagnosed, and/or prognesed using fusion proteins of the invention and/or polynucleotides encoding alpha-fetoprotein fusion proteins of the invention, include, but are not limited to, AIDS; neurodegenerative disorders (such as Alzheimer's disease, Parkinson's disease, Amyotrophic lateral sclerosis, Retinitis pigmentosa, Cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemiatreperfusion injury, cholestosis (bile duct injury) and liver cancer); toxin-induced liver disease (such as that caused by alcohol), septic shock, cachexia and anorexia.

J. Wound Healing and Enithelial Cell Proliferation

In accordance with yet a further aspect of the present invention, there is provided a process for utilizing fusion proteins of the invention and/or polynucleotides encoding alpha-fetoprotein fusion proteins of the invention, for therapeutic purposes, for example, to stimulate epithelial cell proliferation and basal keratinocytes for the purpose of wound healing, and to stimulate hair follicle production and healing of dermal wounds. Alpha-fetoprotein fusion proteins of the invention and/or polynucleotides encoding alpha-fetoprotein fusion proteins of the invention, may be clinically useful in stimulating wound healing including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from heat exposure or chemicals, and other abnormal wound healing conditions such as uremia, malnutrition, vitamin deficiencies and complications associated with systemic treatment with steroids, radiation therapy and antineoplastic drugs and antimetabolites. Alpha-fetoprotein fusion proteins of the invention and/or polynucleotides encoding alpha-fetoprotein fusion proteins of the invention, could be used to promote dermal reestablishment subsequent to dermal loss

Alpha-fetoprotein fusion proteins of the invention and/or polynucleotides encoding alpha-fetoprotein fusion proteins of the invention, could be used to increase the adherence of skin grafts to a wound bed and to stimulate re-epithelialization from the wound bed. The following are types of grafts that fusion proteins of the invention and/or polynucleotides encoding alpha-fetoprotein fusion proteins of the invention, could be used to increase adherence to a wound bed: autografts, artificial skin, allografts, autodermic graft, autoepdermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft Alpha-fetoprotein fusion proteins of the invention and/or polynucleotides encoding alpha-fetoprotein fusion proteins of the invention, can be used to promote skin strength and to improve the appearance of aged skin.

It is believed that fusion proteins of the invention and/or polynucleotides encoding alpha-fetoprotein fusion proteins of the invention, will also produce changes in hepatocyte proliferation, and epithelial cell proliferation in the lung, breast, pancreas, stomach, small intestine, and large intestine. Alpha-fetoprotein fusion proteins of the invention and/or polynucleotides encoding alpha-fetoprotein fusion proteins of the invention, could promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. Alpha-fetoprotein fusion proteins of the invention and/or polynucleotides encoding alpha-fetoprotein fusion proteins of the invention, may promote proliferation of endothelial cells, keratinocytes, and basal keratinocytes.

Alpha-fetoprotein fusion proteins of the invention and/or polynucleotides encoding alpha-fetoprotein fusion proteins of the invention, could also be used to reduce the side effects of gut toxicity that result from radiation, chemotherapy treatments or viral infections. Alpha-fetoprotein fusion proteins of the invention and/or polynucleotides encoding alpha-fetoprotein fusion proteins of the invention, may have a cytoprotective effect on the small intestine mucosa. Alpha-fetoprotein fusion proteins of the invention and/or polynucleotides encoding alpha-fetoprotein fusion proteins of the invention, may also stimulate healing of mucositis (mouth ulcers) that result from chemotherapy and viral infections.

Alpha-fetoprotein fusion proteins of the invention and/or polynucleotides encoding alpha-fetoprotein fusion proteins of the invention, could further be used in full regeneration of skin in full and partial thickness skin defects, including burns, (i.e., repopulation of hair follicles, sweat glands, and sebaceous glands), treatment of other skin defects such as psoriasis. Alpha-fetoprotein fusion proteins of the invention and/or polynucleotides encoding alpha-fetoprotein fusion proteins of the invention, could be used to treat epidermolysis bullosa, a defect in adherence of the epidermis to the underlying dermis which results in frequent, open and painful blisters by accelerating reepithelialization of these lesions. Alpha-fetoprotein fusion proteins of the invention and/or polynucleotides encoding alpha-fetoprotein fusion proteins of the invention, could also be used to treat gastric and doudenal ulcers and help heal by scar formation of the mucosal lining and regeneration of glandular mucosa and duodenal mucosal lining more rapidly. Inflammatory bowel diseases, such as Crohn's disease and ulcerative colitis, are diseases which result in destruction of the mucosal surface of the small or large intestine, respectively. Thus, fusion proteins of the invention and/or polynucleotides encoding alpha-fetoprotein fusion proteins of the invention, could be used to promote the resurfacing of the mucosal surface to aid more rapid healing and to prevent progression of inflammatory bowel disease. Treatment with fusion proteins of the invention and/or polynucleotides encoding alpha-fetoprotein fusion proteins of the invention, is expected to have a significant effect on the production of mucus throughout the gastrointestinal tract and could be used to protect the intestinal mucosa from injurious substances that are ingested or following surgery. Alpha-fetoprotein fusion proteins of the invention and/or polynucleotides encoding alpha-fetoprotein fusion proteins of the invention, could be used to treat diseases associate with the under expression.

Moreover, fusion proteins of the invention and/or polynucleotides encoding alpha-fetoprotein fusion proteins of the invention, could be used to prevent and heal damage to the lungs due to various pathological states. Alpha-fetoprotein fusion proteins of the invention and/or polynucleotides encoding alpha-fetoprotein fusion proteins of the invention, which could stimulate proliferation and differentiation and promote the repair of alveoli and brochiolar epithelium to prevent or treat acute or chronic lung damage. For example, emphysema, which results in the progressive loss of aveoli, and inhalation injuries, i.e., resulting from smoke inhalation and burns, that cause necrosis of the bronchiolar epithelium and alveoli could be effectively treated using polynucleotides or polypeptides, agonists or antagonists of the present invention. Also fusion proteins of the invention and/or polynucleotides encoding alpha-fetoprotein fusion proteins of the invention, could be used to stimulate the proliferation of and differentiation of type II pneumocytes, which may help treat or prevent disease such as hyaline membrane diseases, such as infant respiratory distress syndrome and bronchopulmonary displasia, in premature infants.

Alpha-fetoprotein fusion proteins of the invention and/or polynucleotides encoding alpha-fetoprotein fusion proteins of the invention, could stimulate the proliferation and differentiation of hepatocytes and, thus, could be used to alleviate or treat liver diseases and pathologies such as fulminant liver failure caused by cirrhosis, liver damage caused by viral hepatitis and toxic substances (i.e., acetaminophen, carbon tetraholoride and other hepatotoxins known in the art).

In addition, fusion proteins of the invention and/or polynucleotides encoding alpha-fetoprotein fusion proteins of the invention, could be used treat or prevent the onset of diabetes mellitus. In patients with newly diagnosed Types I and II diabetes, where some islet cell function remains, fusion proteins of the invention and/or polynucleotides encoding alpha-fetoprotein fusion proteins of the invention, could be used to maintain the islet function so as to alleviate, delay or prevent permanent manifestation of the disease. Also, fusion proteins of the invention and/or polynucleotides encoding alpha-fetoprotein fusion proteins of the invention, could be used as an auxiliary in islet cell transplantation to improve or promote islet cell function.

K. Neural Activity and Neurological Diseases

The alpha-fetoprotein fusion proteins of the invention and/or polynucleotides encoding alpha-fetoprotein fusion proteins of the invention may be used for the diagnosis and/or treatment of diseases, disorders, damage or injury of the brain and/or nervous system. Nervous system disorders that can be treated with the compositions of the invention (e.g., fusion proteins of the invention and/or polynucleotides encoding alpha-fetoprotein fusion proteins of the invention), include, but are not limited to, nervous system injuries, and diseases or disorders which result in either a disconnection of axons, a diminution or degeneration of neurons, or demyelination. Nervous system lesions which may be treated in a patient (including human and non-human mammalian patients) according to the methods of the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, or syphilis; (5) degenerative lesions, in which a portion of the nervous system is destroyed or injured as a result of a degenerative process including but not limited to, degeneration associated with Parkinson's disease, Alzheimer's disease, Huntington's chorea, or amyotrophic lateral sclerosis (ALS); (6) lesions associated with nutritional diseases or disorders, in which a portion of the nervous system is destroyed or injured by a nutritional disorder or disorder of metabolism including, but not limited to, vitamin B12 deficiency, folic acid deficiency, Wemicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In one embodiment, the alpha-fetoprotein fusion proteins of the invention and/or polynucleotides encoding alpha-fetoprotein fusion proteins of the invention are used to protect neural cells from the damaging effects of hypoxia. In a further preferred embodiment, the alpha-fetoprotein fusion proteins of the invention and/or polynucleotides encoding alpha-fetoprotein fusion proteins of the invention are used to protect neural cells from the damaging effects of cerebral hypoxia. According to this embodiment, the compositions of the invention are used to treat or prevent neural cell injury associated with cerebral hypoxia. In one non-exclusive aspect of this embodiment, the alpha-fetoprotein fusion proteins of the invention and/or polynucleotides encoding alpha-fetoprotein fusion proteins of the invention, are used to treat or prevent neural cell injury associated with cerebral ischemia. In another non-exclusive aspect of this embodiment, the alpha-fetoprotein fusion proteins of the invention and/or polynucleotides encoding alpha-fetoprotein fusion proteins of the invention are used to treat or prevent neural cell injury associated with cerebral infarction.

In another preferred embodiment, alpha-fetoprotein fusion proteins of the invention and/or polynucleotides encoding alpha-fetoprotein fusion proteins of the invention are used to treat or prevent neural cell injury associated with a stroke. In a specific embodiment, alpha-fetoprotein fusion proteins of the invention and/or polynucleotides encoding alpha-fetoprotein fusion proteins of the invention are used to treat or prevent cerebral neural cell injury associated with a stroke.

In another preferred embodiment, alpha-fetoprotein fusion proteins of the invention and/or polynucleotides encoding alpha-fetoprotein fusion proteins of the invention are used to treat or prevent neural cell injury associated with a heart attack. In a specific embodiment, alpha-fetoprotein fusion proteins of the invention and/or polynucleotides encoding alpha-fetoprotein fusion proteins of the invention are used to treat or prevent cerebral neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture either in the presence or absence of hypoxia or hypoxic conditions; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, in Zhang et al., Proc Natl Acad Sci USA 97:363742 (2000) or in Arakawa et al., J. Neurosci., 10:3507-15 (1990); increased sprouting of neurons may be detected by methods known in the art, such as, for example, the methods set forth in Pestronk et al., Exp. Neurol., 70:65-82 (1980), or Brown et al., Ann. Rev. Neurosci, 4:1742 (1981); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron disorders that may be treated according to the invention include, but are not limited to, disorders such as infarction, infection, exposure to toxin, trauma, surgical damage, degenerative disease or malignancy that may affect motor neurons as well as other components of the nervous system, as well as disorders that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

Further, fusion proteins of the invention and/or polynucleotides encoding alpha-fetoprotein fusion proteins of the invention may play a role in neuronal survival; synapse formation; conductance; neural differentiation, etc. Thus, compositions of the invention (including fusion proteins of the invention and/or polynucleotides encoding alpha-fetoprotein fusion proteins of the invention) may be used to diagnose and/or treat or prevent diseases or disorders associated with these roles, including, but not limited to, learning and/or cognition disorders. The compositions of the invention may also be useful in the treatment or prevention of neurodegenerative disease states and/or behavioural disorders. Such neurodegenerative disease states and/or behavioral disorders include, but are not limited to, Alzheimer's Disease, Parkinson's Disease, Huntington's Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, compositions of the invention may also play a role in the treatment, prevention and/or detection of developmental disorders associated with the developing embryo, or sexually-linked disorders.

Additionally, fusion proteins of the invention and/or polynucleotides encoding alpha-fetoprotein fusion proteins of the invention, may be useful in protecting neural cells from diseases, damage, disorders, or injury, associated with cerebrovascular disorders including, but not limited to, carotid artery diseases (e.g., carotid artery thrombosis, carotid stenosis, or Moyamoya Disease), cerebral amyloid angiopathy, cerebral aneurysm, cerebral anoxia, cerebral arteriosclerosis, cerebral arteriovenous mAFPormations, cerebral artery diseases, cerebral embolism and thrombosis (e.g., carotid artery thrombosis, sinus thrombosis, or Wallenberg's Syndrome), cerebral hemorrhage (e.g., epidural or subdural hematoma, or subarachnoid hemorrhage), cerebral infarction, cerebral ischemia (e.g., transient cerebral ischemia, Subclavian Steal Syndrome, or vertebrobasilar insufficiency), vascular dementia (e.g., multi-infarct), leukomalacia, periventricular, and vascular headache (e.g., cluster headache or migraines).

In accordance with yet a further aspect of the present invention, there is provided a process for utilizing fusion proteins of the invention and/or polynucleotides encoding alpha-fetoprotein fusion proteins of the invention, for therapeutic purposes, for example, to stimulate neurological cell proliferation and/or differentiation. Therefore, fusion proteins of the invention and/or polynucleotides encoding alpha-fetoprotein fusion proteins of the invention may be used to treat and/or detect neurologic diseases. Moreover, fusion proteins of the invention and/or polynucleotides encoding alpha-fetoprotein fusion proteins of the invention, can be used as a marker or detector of a particular nervous system disease or disorder.

Examples of neurologic diseases which can be treated or detected with fusion proteins of the invention and/or polynucleotides encoding alpha-fetoprotein fusion proteins of the invention include, brain diseases, such as metabolic brain diseases which includes phenylketonuria such as matemal phenylketonuria, pyruvate carboxylase deficiency, pyruvate dehydrogenase complex deficiency, Wemicke's Encephalopathy, brain edema, brain neoplasms such as cerebellar neoplasms which include infratentorial neoplasms, cerebral ventricle neoplasms such as choroid plexus neoplasms, hypothalamic neoplasms, supratentorial neoplasms, canavan disease, cerebellar diseases such as cerebellar ataxia which include spinocerebellar degeneration such as ataxia telangiectasia, cerebellar dyssynergia, Friederich's Ataxia, Machado-Joseph Disease, olivopontocerebellar atrophy, cerebellar neoplasms such as infratentorial neoplasms, diffuse cerebral sclerosis such as encephalitis periaxialis, globoid cell leukodystrophy, metachromatic leukodystrophy and subacute sclerosing panencephalitis.

Additional neurologic diseases which can be treated or detected with fusion proteins of the invention and/or polynucleotides encoding alpha-fetoprotein fusion proteins of the invention include cerebrovascular disorders (such as carotid artery diseases which include carotid artery thrombosis, carotid stenosis and Moyamoya Disease), cerebral amyloid angiopathy, cerebral aneurysm, cerebral anoxia, cerebral arteriosclerosis, cerebral arteriovenous mAFPormations, cerebral artery diseases, cerebral embolism and thrombosis such as carotid artery thrombosis, sinus thrombosis and Wallenberg's Syndrome, cerebral hemorrhage such as epidural hematoma, subdural hematoma and subarachnoid hemorrhage, cerebral infarction, cerebral ischemia such as transient cerebral ischemia, Subclavian Steal Syndrome and vertebrobasilar insufficiency, vascular dementia such as multi-infarct dementia, periventricular leukomalacia, vascular headache such as cluster headache and migraine.

Additional neurologic diseases which can be treated or detected with fusion proteins of the invention and/or polynucleotides encoding alpha-fetoprotein fusion proteins of the invention include dementia such as AIDS Dementia Complex, presenile dementia such as Alzheimer's Disease and Creutzfeldt-Jakob Syndrome, senile dementia such as Alzheimer's Disease and progressive supranuclear palsy, vascular dementia such as multi-infarct dementia, encephalitis which include encephalitis periaxialis, viral encephalitis such as epidemic encephalitis, Japanese Encephalitis, St. Louis Encephalitis, tick-borne encephalitis and West Nile Fever, acute disseminated encephalomyelitis, meningoencephalitis such as uveomeningoencephalitic syndrome, Postencephalitic Parkinson Disease and subacute sclerosing panencephalitis, encephalomalacia such as periventricular leukomalacia, epilepsy such as generalized epilepsy which includes infantile spasms, absence epilepsy, myoclonic epilepsy which includes MERRF Syndrome, tonic-clonic epilepsy, partial epilepsy such as complex partial epilepsy, frontal lobe epilepsy and temporal lobe epilepsy, post-traumatic epilepsy, status epilepticus such as Epilepsia Partialis Continua, and Hallervorden-Spatz Syndrome.

Additional neurologic diseases which can be treated or detected with fusion proteins of the invention and/or polynucleotides encoding alpha-fetoprotein fusion proteins of the invention include hydrocephalus such as Dandy-Walker Syndrome and normal pressure hydrocephalus, hypothalamic diseases such as hypothalamic neoplasms, cerebral malaria, narcolepsy which includes cataplexy, bulbar poliomyelitis, cerebri pseudotumor, Rett Syndrome, Reye's Syndrome, thalamic diseases, cerebral toxoplasmosis, intracranial tuberculoma and Zellweger Syndrome, central nervous system infections such as AIDS Dementia Complex, Brain Abscess, subdural empyema, encephalomyelitis such as Equine Encephalomyelitis, Venezuelan Equine Encephalomyelitis, Necrotizing Hemorrhagic Encephalomyelitis, Visna, and cerebral malaria.

Additional neurologic diseases which can be treated or detected with fusion proteins of the invention and/or polynucleotides encoding alpha-fetoprotein fusion proteins of the invention include meningitis such as arachnoiditis, aseptic meningtitis such as viral meningtitis which includes lymphocytic choriomeningitis, Bacterial meningtitis which includes Haemophilus Meningtitis, Listeria Meningtitis, Meningococcal Meningtitis such as Waterhouse-Friderichsen Syndrome, Pneumococcal Meningtitis and meningeal tuberculosis, fungal meningitis such as Cryptococcal Meningtitis, subdural effusion, meningoencephalitis such as uvemeningoencephalitic syndrome, myelitis such as transverse myelitis, neurosyphilis such as tabes dorsalis, poliomyelitis which includes bulbar poliomyelitis and postpoliomyelitis syndrome, prion diseases (such as Creutzfeldt-Jakob Syndrome, Bovine Spongiform Encephalopathy, Gerstmann-Straussler Syndrome, Kuru, Scrapie), and cerebral toxoplasmosis.

Additional neurologic diseases which can be treated or detected with fusion proteins of the invention and/or polynucleotides encoding alpha-fetoprotein fusion proteins of the invention include central nervous system neoplasms such as brain neoplasms that include cerebellar neoplasms such as infratentorial neoplasms, cerebral ventricle neoplasms such as choroid plexus neoplasms, hypothalamic neoplasms and supratentorial neoplasms, meningeal neoplasms, spinal cord neoplasms which include epidural neoplasms, demyelinating diseases such as Canavan Diseases, diffuse cerebral sceloris which includes adrenoleukodystrophy, encephalitis periaxialis, globoid cell leukodystrophy, diffuse cerebral sclerosis such as metachromatic leukodystrophy, allergic encephalomyelitis, necrotizing hemorrhagic encephalomyelitis, progressive multifocal leukoencephalopathy, multiple sclerosis, central pontine myelinolysis, transverse myelitis, neuromyelitis optica, Scrapie, Swayback, Chronic Fatigue Syndrome, Visna, High Pressure Nervous Syndrome, Meningism, spinal cord diseases such as amyotonia congenita, amyotrophic lateral sclerosis, spinal muscular atrophy such as Werdnig-Hoffmann Disease, spinal cord compression, spinal cord neoplasms such as epidural neoplasms, syringomyelia, Tabes Dorsalis, Stiff-Man Syndrome, mental retardation such as Angelman Syndrome, Cri-du-Chat Syndrome, De Lange's Syndrome, Down Syndrome, Gangliosidoses such as gangliosidoses G(MI), Sandhoff Disease, Tay-Sachs Disease, Hartnup Disease, homocystinuria, Laurence-Moon-Biedl Syndrome, Lesch-Nyhan Syndrome, Maple Syrup Urine Disease, mucolipidosis such as fucosidosis, neuronal ceroid-lipofuscinosis, oculocerebrorenal syndrome, phenylketonuria such as matemal phenylketonuria, Prader-Willi Syndrome, Rett Syndrome, Rubinstein-Taybi Syndrome, Tuberous Sclerosis, WAGR Syndrome, nervous system abnormalities such as holoprosencephaly, neural tube defects such as anencephaly which includes hydrangencephaly, Amold-Chairi Deformity, encephalocele, meningocele, meningomyelocele, spinal dysraphism such as spina bifida cystica and spina bifida occulta.

Additional neurologic diseases which can be treated or detected with fusion proteins of the invention and/or polynucleotides encoding alpha-fetoprotein fusion proteins of the invention include hereditary motor and sensory neuropathies which include Charcot-Marie Disease, Hereditary optic atrophy, Refsum's Disease, hereditary spastic paraplegia, Werdnig-Hoffmann Disease, Hereditary Sensory and Autonomic Neuropathies such as Congenital Analgesia and Familial Dysautonomia, Neurologic manifestations (such as agnosia that include Gerstmann's Syndrome, Amnesia such as retrograde amnesia, apraxia, neurogenic bladder, cataplexy, communicative disorders such as hearing disorders that includes deafness, partial hearing loss, loudness recruitment and tinnitus, language disorders such as aphasia which include agraphia, anomia, broca aphasia, and Wemicke Aphasia, Dyslexia such as Acquired Dyslexia, language development disorders, speech disorders such as aphasia which includes anomia, broca aphasia and Wemicke Aphasia, articulation disorders, communicative disorders such as speech disorders which include dysarthria, echolalia, mutism and stuttering, voice disorders such as aphonia and hoarseness, decerebrate state, delirium, fasciculation, hallucinations, meningism, movement disorders such as angelman syndrome, ataxia, athetosis, chorea, dystonia, hypokinesia, muscle hypotonia, myoclonus, tic, torticollis and tremor, muscle hypertonia such as muscle rigidity such as stiff-man syndrome, muscle spasticity, paralysis such as facial paralysis which includes Herpes Zoster Oticus, Gastroparesis, Hemiplegia, opthalmoplegia such as diplopia, Duane's Syndrome, Horner's Syndrome, Chronic progressive external opthalmoplegia such as Keams Syndrome, Bulbar Paralysis, Tropical Spastic Paraparesis, Paraplegia such as Brown-Sequard Syndrome, quadriplegia, respiratory paralysis and vocal cord paralysis, paresis, phantom limb, taste disorders such as ageusia and dysgeusia, vision disorders such as amblyopia, blindness, color vision defects, diplopia, hemianopsia, scotoma and subnormal vision, sleep disorders such as hypersomnia which includes Kleine-Levin Syndrome, insomnia, and somnambulism, spasm such as trismus, unconsciousness such as coma, persistent vegetative state and syncope and vertigo, neuromuscular diseases such as amyotonia congenita, amyotrophic lateral sclerosis, Lambert-Eaton Myasthenic Syndrome, motor neuron disease, muscular atrophy such as spinal muscular atrophy, Charcot-Marie Disease and Werdnig-Hoffmann Disease, Postpoliomyelitis Syndrome, Muscular Dystrophy, Myasthenia Gravis, Myotonia Atrophica, Myotonia Confenita, Nemaline Myopathy, Familial Periodic Paralysis, Multiplex Paramyloclonus, Tropical Spastic Paraparesis and Stiff-Man Syndrome, peripheral nervous system diseases such as acrodynia, amyloid neuropathies, autonomic nervous system diseases such as Adie's Syndrome, Barre-Lieou Syndrome, Familial Dysautonomia, Horner's Syndrome, Reflex Sympathetic Dystrophy and Shy-Drager Syndrome, Cranial Nerve Diseases such as Acoustic Nerve Diseases such as Acoustic Neuroma which includes Neurofibromatosis 2, Facial Nerve Diseases such as Facial Neuralgia, Melkersson-Rosenthal Syndrome, ocular motility disorders which includes amblyopia, nystagmus, oculomotor nerve paralysis, opthalmoplegia such as Duane's Syndrome, Horner's Syndrome, Chronic Progressive External Opthalmoplegia which includes Keams Syndrome, Strabismus such as Esotropia and Exotropia, Oculomotor Nerve Paralysis, Optic Nerve Diseases such as Optic Atrophy which includes Hereditary Optic Atrophy, Optic Disk Drusen, Optic Neuritis such as Neuromyelitis Optica, Papilledema, Trigeminal Neuralgia, Vocal Cord Paralysis, Demyelinating Diseases such as Neuromyelitis Optica and Swayback, and Diabetic neuropathies such as diabetic foot. 107571 Additional neurologic diseases which can be treated or detected with fusion proteins of the invention and/or polynucleotides encoding alpha-fetoprotein fusion proteins of the invention include nerve compression syndromes such as carpal tunnel syndrome, tarsal tunnel syndrome, thoracic outlet syndrome such as cervical rib syndrome, ulnar nerve compression syndrome, neuralgia such as causalgia, cervico-brachial neuralgia, facial neuralgia and trigeminal neuralgia, neuritis such as experimental allergic neuritis, optic neuritis, polyneuritis, polyradiculoneuritis and radiculities such as polyradiculitis, hereditary motor and sensory neuropathies such as Charcot-Marie Disease, Hereditary Optic Atrophy, Refsum's Disease, Hereditary Spastic Paraplegia and Werdnig-Hoffmann Disease, Hereditary Sensory and Autonomic Neuropathies which include Congenital Analgesia and Familial Dysautonomia, POEMS Syndrome, Sciatica, Gustatory Sweating and Tetany).

L. Endocrine Disorders

Alpha-fetoprotein fusion proteins of the invention and/or polynucleotides encoding alpha-fetoprotein fusion proteins of the invention, may be used to treat, prevent, diagnose, and/or prognose disorders and/or diseases related to hormone imbalance, and/or disorders or diseases of the endocrine system.

Hormones secreted by the glands of the endocrine system control physical growth, sexual function, metabolism, and other functions. Disorders may be classified in two ways: disturbances in the production of hormones, and the inability of tissues to respond to hormones. The etiology of these hormone imbalance or endocrine system diseases, disorders or conditions may be genetic, somatic, such as cancer and some autoimmune diseases, acquired (e.g., by chemotherapy, injury or toxins), or infectious. Moreover, fusion proteins of the invention and/or polynucleotides encoding alpha-fetoprotein fusion proteins of the invention can be used as a marker or detector of a particular disease or disorder related to the endocrine system and/or hormone imbalance.

Endocrine system and/or hormone imbalance and/or diseases encompass disorders of uterine motility including, but not limited to: complications with pregnancy and labor (e.g., pre-term labor, post-term pregnancy, spontaneous abortion, and slow or stopped labor); and disorders and/or diseases of the menstrual cycle (e.g., dysmenorrhea and endometriosis).

Endocrine system and/or hormone imbalance disorders and/or diseases include disorders and/or diseases of the pancreas, such as, for example, diabetes mellitus, diabetes insipidus, congenital pancreatic agenesis, pheochromocytoma-islet cell tumor syndrome; disorders and/or diseases of the adrenal glands such as, for example, Addison's Disease, corticosteroid deficiency, virilizing disease, hirsutism, Cushing's Syndrome, hyperaldosteronism, pheochromocytoma; disorders and/or diseases of the pituitary gland, such as, for example, hyperpituitarism, hypopituitarism, pituitary dwarfism, pituitary adenoma, panhypopituitarism, acromegaly, gigantism; disorders and/or diseases of the thyroid, including but not limited to, hyperthyroidism, hypothyroidism, Plummer's disease, Graves' disease (toxic diffuse goiter), toxic nodular goiter, thyroiditis (Hashimoto's thyroiditis, subacute granulomatous thyroiditis, and silent lymphocytic thyroiditis), Pendred's syndrome, myxedema, cretinism, thyrotoxicosis, thyroid hormone coupling defect, thymic aplasia, Hurthle cell tumours of the thyroid, thyroid cancer, thyroid carcinoma, Medullary thyroid carcinoma; disorders and/or diseases of the parathyroid, such as, for example, hyperparathyroidism, hypoparathyroidism; disorders and/or diseases of the hypothalamus.

In addition, endocrine system and/or hormone imbalance disorders and/or diseases may also include disorders and/or diseases of the testes or ovaries, including cancer. Other disorders and/or diseases of the testes or ovaries further include, for example, ovarian cancer, polycystic ovary syndrome, Klinefelter's syndrome, vanishing testes syndrome (bilateral anorchia), congenital absence of Leydig's cells, cryptorchidism, Noonan's syndrome, myotonic dystrophy, capillary haemangioma of the testis (benign), neoplasias of the testis and neo-testis.

Moreover, endocrine system and/or hormone imbalance disorders and/or diseases may also include disorders and/or diseases such as, for example, polyglandular deficiency syndromes, pheochromocytoma, neuroblastoma, multiple Endocrine neoplasia, and disorders and/or cancers of endocrine tissues.

In another embodiment, alpha-fetoprotein fusion proteins of the invention and/or polynucleotides encoding alpha-fetoprotein fusion proteins of the invention, may be used to diagnose, prognose, prevent, and/or treat endocrine diseases and/or disorders associated with the tissue(s) in which the therapeutic protein corresponding to the therapeutic protein portion of the alpha-fetoprotein protein of the invention is expressed.

M. Reproductive System Disorders

The alpha-fetoprotein fusion proteins of the invention and/or polynucleotides encoding alpha-fetoprotein fusion proteins of the invention may be used for the diagnosis, treatment, or prevention of diseases and/or disorders of the reproductive system. Reproductive system disorders that can be treated by the compositions of the invention, include, but are not limited to, reproductive system injuries, infections, neoplastic disorders, congenital defects, and diseases or disorders which result in infertility, complications with pregnancy, labor, or parturition, and postpartum difficulties.

Reproductive system disorders and/or diseases include diseases and/or disorders of the testes, including testicular atrophy, testicular feminization, cryptorchism (unilateral and bilateral), anorchia, ectopic testis, epididymitis and orchitis (typically resulting from infections such as, for example, gonorrhea, mumps, tuberculosis, and syphilis), testicular torsion, vasitis nodosa, germ cell tumors (e.g., seminomas, embryonal cell carcinomas, teratocarcinomas, choriocarcinomas, yolk sac tumors, and teratomas), stromal tumors (e.g., Leydig cell tumors), hydrocele, hematocele, varicocele, spermatocele, inguinal hernia, and disorders of sperm production (e.g., immotile cilia syndrome, aspermia, asthenozoospermia, azoospermia, oligospermia, and teratozoospermia).

Reproductive system disorders also include disorders of the prostate gland, such as acute non-bacterial prostatitis, chronic non-bacterial prostatitis, acute bacterial prostatitis, chronic bacterial prostatitis, prostatodystonia, prostatosis, granulomatous prostatitis, malacoplakia, benign prostatic hypertrophy or hyperplasia, and prostate neoplastic disorders, including adenocarcinomas, transitional cell carcinomas, ductal carcinomas, and squamous cell carcinomas.

Additionally, the compositions of the invention may be useful in the diagnosis, treatment, and/or prevention of disorders or diseases of the penis and urethra, including inflammatory disorders, such as balanoposthitis, balanitis xerotica obliterans, phimosis, paraphimosis, syphilis, herpes simplex virus, gonorrhea, non-gonococcal urethritis, chlamydia, mycoplasma, trichomonas, HIV, AIDS, Reiter's syndrome, condyloma acuminatum, condyloma latum, and pearly penile papules; urethral abnormalities, such as hypospadias, epispadias, and phimosis; premalignant lesions, including Erythroplasia of Queyrat, Bowen's disease, Bowenoid paplosis, giant condyloma of Buscke-Lowenstein, and varrucous carcinoma; penile cancers, including squamous cell carcinomas, carcinoma in situ, verrucous carcinoma, and disseminated penile carcinoma; urethral neoplastic disorders, including penile urethral carcinoma, bulbomembranous urethral carcinoma, and prostatic urethral carcinoma; and erectile disorders, such as priapism, Peyronie's disease, erectile dysfunction, and impotence.

Moreover, diseases and/or disorders of the vas deferens include vasculititis and CBAVD (congenital bilateral absence of the vas deferens); additionally, the alpha-fetoprotein fusion proteins of the invention and/or polynucleotides encoding alpha-fetoprotein fusion proteins of the invention may be used in the diagnosis, treatment, and/or prevention of diseases and/or disorders of the seminal vesicles, including hydatid disease, congenital chloride diarrhea, and polycystic kidney disease.

Other disorders and/or diseases of the male reproductive system include, for example, Klinefelter's syndrome, Young's syndrome, premature ejaculation, diabetes mellitus, cystic fibrosis, Kartagener's syndrome, high fever, multiple sclerosis, and gynecomastia.

Further, the polynucleotides, fusion proteins of the invention and/or polynucleotides encoding alpha-fetoprotein fusion proteins of the invention may be used in the diagnosis, treatment, and/or prevention of diseases and/or disorders of the vagina and vulva, including bacterial vaginosis, candida vaginitis, herpes simplex virus, chancroid, granuloma inguinale, lymphogranuloma venereum, scabies, human papillomavirus, vaginal trauma, vulvar trauma, adenosis, chlamydia vaginitis, gonorrhea, trichomonas vaginitis, condyloma acuminatum, syphilis, molluscum contagiosum, atrophic vaginitis, Paget's disease, lichen sclerosus, lichen planus, vulvodynia, toxic shock syndrome, vaginismus, vulvovaginitis, vulvar vestibulitis, and neoplastic disorders, such as squamous cell hyperplasia, clear cell carcinoma, basal cell carcinoma, melanomas, cancer of Bartholin's gland, and vulvar intraepithelial neoplasia.

Disorders and/or diseases of the uterus include dysmenorrhea, retroverted uterus, endometriosis, fibroids, adenomyosis, anovulatory bleeding, amenorrhea, Cushing's syndrome, hydatidiform moles, Asherman's syndrome, premature menopause, precocious puberty, uterine polyps, dysfunctional uterine bleeding (e.g., due to aberrant hormonal signals), and neoplastic disorders, such as adenocarcinomas, keiomyosarcomas, and sarcomas. Additionally, the alpha-fetoprotein fusion proteins of the invention and/or polynucleotides encoding alpha-fetoprotein fusion proteins of the invention may be useful as a marker or detector of, as well as in the diagnosis, treatment, and/or prevention of congenital uterine abnormalities, such as bicomuate uterus, septate uterus, simple unicomuate uterus, unicomuate uterus with a noncavitary rudimentary hom, unicomuate uterus with a non-communicating cavitary rudimentary hom, unicomuate uterus with a communicating cavitary hom, arcuate uterus, uterine didelfus, and T-shaped uterus.

Ovarian diseases and/or disorders include anovulation, polycystic ovary syndrome (Stein-Leventhal syndrome), ovarian cysts, ovarian hypofunction, ovarian insensitivity to gonadotropins, ovarian overproduction of androgens, fight ovarian vein syndrome, amenorrhea, hirutism, and ovarian cancer (including, but not limited to, primary and secondary cancerous growth, Sertoli-Leydig tumors, endometriod carcinoma of the ovary, ovarian papillary serous adenocarcinoma, ovarian mucinous adenocarcinoma, and Ovarian Krukenberg tumors).

Cervical diseases and/or disorders include cervicitis, chronic cervicitis, mucopurulent cervicitis, cervical dysplasia, cervical polyps, Nabothian cysts, cervical erosion, cervical incompetence, and cervical neoplasms (including, for example, cervical carcinoma, squamous metaplasia, squamous cell carcinoma, adenosquamous cell neoplasia, and columnar cell neoplasia).

Additionally, diseases and/or disorders of the reproductive system include disorders and/or diseases of pregnancy, including miscarriage and stillbirth, such as early abortion, late abortion, spontaneous abortion, induced abortion, therapeutic abortion, threatened abortion, missed abortion, incomplete abortion, complete abortion, habitual abortion, missed abortion, and septic abortion; ectopic pregnancy, anemia, Rh incompatibility, vaginal bleeding during pregnancy, gestational diabetes, intrauterine growth retardation, polyhydramnios, HELLP syndrome, abruptio placentae, placenta previa, hyperemesis, preeclampsia, eclampsia, herpes gestationis, and urticaria of pregnancy. Additionally, the alpha-fetoprotein fusion proteins of the invention and/or polynucleotides encoding alpha-fetoprotein fusion proteins of the invention may be used in the diagnosis, treatment, and/or prevention of diseases that can complicate pregnancy, including heart disease, heart failure, rheumatic heart disease, congenital heart disease, mitral valve prolapse, high blood pressure, anemia, kidney disease, infectious disease (e.g., rubella, cytomegalovirus, toxoplasmosis, infectious hepatitis, chlamydia, HIV, AIDS, and genital herpes), diabetes mellitus, Graves' disease, thyroiditis, hypothyroidism, Hashimoto's thyroiditis, chronic active hepatitis, cirrhosis of the liver, primary biliary cirrhosis, asthma, systemic lupus eryematosis, rheumatoid arthritis, myasthenia gravis, idiopathic thrombocytopenic purpura, appendicitis, ovarian cysts, gallbladder disorders, and obstruction of the intestine.

Complications associated with labor and parturition include premature rupture of the membranes, pre-term labor, post-term pregnancy, postmaturity, labor that progresses too slowly, fetal distress (e.g., abnormal heart rate (fetal or matemal), breathing problems, and abnormal fetal position), shoulder dystocia, prolapsed umbilical cord, amniotic fluid embolism, and aberrant uterine bleeding.

Further, diseases and/or disorders of the postdelivery period, including endometritis, myometritis, parametritis, peritonitis, pelvic thrombophlebitis, pulmonary embolism, endotoxemia, pyelonephritis, saphenous thrombophlebitis, mastitis, cystitis, postpartum hemorrhage, and inverted uterus.

Other disorders and/or diseases of the female reproductive system that may be diagnosed, treated, and/or prevented by the alpha-fetoprotein fusion proteins of the invention and/or polynucleotides encoding alpha-fetoprotein fusion proteins of the invention include, for example, Tumer's syndrome, pseudohermaphroditism, premenstrual syndrome, pelvic inflammatory disease, pelvic congestion (vascular engorgement), frigidity, anorgasmia, dyspareunia, ruptured fallopian tube, and Mittelschmerz.

N. Infectious Disease

Alpha-fetoprotein fusion proteins of the invention and/or polynucleotides encoding alpha-fetoprotein fusion proteins of the invention can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, fusion proteins of the invention and/or polynucleotides encoding alpha-fetoprotein fusion proteins of the invention may also directly inhibit the infectious agent, without necessarily eliciting an immune response.

Viruses are one example of an infectious agent that can cause disease or symptoms that can be treated or detected by alpha-fetoprotein fusion proteins of the invention and/or polynucleotides encoding alpha-fetoprotein fusion proteins of the invention. Examples of viruses, include, but are not limited to Examples of viruses, include, but are not limited to the following DNA and RNA viruses and viral families: Arbovirus, Adenoviridae, Arenaviridae, Arterivirus, Bimaviridae, Bunyaviridae, Caliciviridae, Circoviridae, Coronaviridae, Dengue, EBV, HIV, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza A, Influenza B, and parainfluenza), Papiloma virus, Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, respiratory syncytial virus, encephalitis, eye infections (e.g., conjunctivitis, keratitis), chronic fatigue syndrome, hepatitis (A, B, C, E, Chronic Active, Delta), Japanese B encephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. Alpha-fetoprotein fusion proteins of the invention and/or polynucleotides encoding alpha-fetoprotein fusion proteins of the invention, can be used to treat or detect any of these symptoms or diseases. In specific embodiments, fusion proteins of the invention and/or polynucleotides encoding alpha-fetoprotein fusion proteins of the invention are used to treat: meningitis, Dengue, EBV, and/or hepatitis (e.g., hepatitis B). In an additional specific embodiment fusion proteins of the invention and/or polynucleotides encoding alpha-fetoprotein fusion proteins of the invention are used to treat patients nonresponsive to one or more other commercially available hepatitis vaccines. In a further specific embodiment fusion proteins of the invention and/or polynucleotides encoding alpha-fetoprotein fusion proteins of the invention are used to treat AIDS.

Similarly, bacterial and fungal agents that can cause disease or symptoms and that can be treated or detected by alpha-fetoprotein fusion proteins of the invention and/or polynucleotides encoding alpha-fetoprotein fusion proteins of the invention include, but not limited to, the following Gram-Negative and Gram-positive bacteria, bacterial families, and fungi: Actinomyces (e.g., Norcardia), Acinetobacter, Cryptococcus neoformans, Aspergillus, Bacillaceae (e.g., Bacillus anthrasis), Bacteroides (e.g., Bacteroides fragilis), Blastomycosis, Bordetella, Borrelia (e.g., Borrelia burgdorferi), Brucella, Candidia, Campylobacter, Chlamydia, Clostridium (e.g., Clostridium botulinum, Clostridium dificile, Clostridium perfringens, Clostridium tetani), Coccidioides, Corynebacterium (e.g., Corynebacterium diptheriae), Cryptococcus, Dermatocycoses, E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Salmonella typhi), Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Neisseriaceae (e.g., Neisseria gonorrhea, Neisseria meningitidis), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Staphylococcus (e.g., Staphylococcus aureus), Meningiococcus, Pneumococcus and Streptococcus (e.g., Streptococcus pneumoniae and Groups A, B, and C Streptococci), and Ureaplasmas. These bacterial, parasitic, and fungal families can cause diseases or symptoms, including, but not limited to: antibiotic-resistant infections, bacteremia, endocarditis, septicemia, eye infections (e.g., conjunctivitis), uveitis, tuberculosis, gingivitis, bacterial diarrhea, opportunistic infections (e.g., AIDS related infections), paronychia, prosthesis-related infections, dental caries, Reiter's Disease, respiratory tract infections, such as Whooping Cough or Empyema, sepsis, Lyme Disease, Cat-Scratch Disease, dysentery, paratyphoid fever, food poisoning, Legionella disease, chronic and acute inflammation, erythema, yeast infections, typhoid, pneumonia, gonorrhea, meningitis (e.g., mengitis types A and B), chiamydia, syphillis, diphtheria, leprosy, brucellosis, peptic ulcers, anthrax, spontaneous abortions, birth defects, pneumonia, lung infections, ear infections, deafness, blindness, lethargy, malaise, vomiting, chronic diarrhea, Crohn's disease, colitis, vaginosis, sterility, pelvic inflammatory diseases, candidiasis, paratuberculosis, tuberculosis, lupus, botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections, noscomial infections. Alpha-fetoprotein fusion proteins of the invention and/or polynucleotides encoding alpha-fetoprotein fusion proteins of the invention, can be used to treat or detect any of these symptoms or diseases. In specific embodiments, fusion proteins of the invention and/or polynucleotides encoding alpha-fetoprotein fusion proteins of the invention are used to treat: tetanus, diptheria, botulism, and/or meningitis type B.

Moreover, parasitic agents causing disease or symptoms that can be treated, prevented, and/or diagnosed by fusion proteins of the invention and/or polynucleotides encoding alpha-fetoprotein fusion proteins of the invention include, but not limited to, the following families or class: Amebiasis, Babesiosis, Coccidiosis, Cryptosporidiosis, Dientamoebiasis, Dourine, Ectoparasitic, Giardias, Helminthiasis, Leishmaniasis, Schistisoma, Theileriasis, Toxoplasmosis, Trypanosomiasis, and Trichomonas and Sporozoans (e.g., Plasmodium virax, Plasmodium falciparium, Plasmodium malariae and Plasmodium ovale). These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), malaria, pregnancy complications, and toxoplasmosis. Alpha-fetoprotein fusion proteins of the invention and/or polynucleotides encoding alpha-fetoprotein fusion proteins of the invention, can be used to treat, prevent, and/or diagnose any of these symptoms or diseases. In specific embodiments, fusion proteins of the invention and/or polynucleotides encoding alpha-fetoprotein fusion proteins of the invention are used to treat, prevent, and/or diagnose malaria.

Alpha-fetoprotein fusion proteins of the invention and/or polynucleotides encoding alpha-fetoprotein fusion proteins of the invention could either be by administering an effective amount of an alpha-fetoprotein fusion protein of the invention to the patient, or by removing cells from the patient, supplying the cells with a polynucleotide of the present invention, and returning the engineered cells to the patient (ex vivo therapy). Moreover, the alpha-fetoprotein fusion proteins of the invention and/or polynucleotides encoding alpha-fetoprotein fusion proteins of the invention can be used as an antigen in a vaccine to raise an immune response against infectious disease.

O. Regeneration

Alpha-fetoprotein fusion proteins of the invention and/or polynucleotides encoding alpha-fetoprotein fusion proteins of the invention can be used to differentiate, proliferate, and attract cells, leading to the regeneration of tissues. (See, Science 276:59-87 (1997)). The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, fusion proteins of the invention and/or polynucleotides encoding alpha-fetoprotein fusion proteins of the invention, may increase regeneration of tissues difficult to heal. For example, increased tendon/ligament regeneration would quicken recovery time after damage. Alpha-fetoprotein fusion proteins of the invention and/or polynucleotides encoding alpha-fetoprotein fusion proteins of the invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using fusion proteins of the invention and/or polynucleotides encoding alpha-fetoprotein fusion proteins of the invention, to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the alpha-fetoprotein fusion proteins of the invention and/or polynucleotides encoding alpha-fetoprotein fusion proteins of the invention.

P. Gastrointestinal Disorders

Alpha-fetoprotein fusion proteins of the invention and/or polynucleotides encoding alpha-fetoprotein fusion proteins of the invention, may be used to treat, prevent, diagnose, and/or prognose gastrointestinal disorders, including inflammatory diseases and/or conditions, infections, cancers (e.g., intestinal neoplasms (carcinoid tumor of the small intestine, non-Hodgkin's lymphoma of the small intestine, small bowl lymphoma)), and ulcers, such as peptic ulcers.

Gastrointestinal disorders include dysphagia, odynophagia, inflammation of the esophagus, peptic esophagitis, gastric reflux, submucosal fibrosis and structuring, Mallory-Weiss lesions, leiomyomas, lipomas, epidermal cancers, adeoncarcinomas, gastric retention disorders, gastroenteritis, gastric atrophy, gastric/stomach cancers, polyps of the stomach, autoimmune disorders such as pernicious anemia, pyloric stenosis, gastritis (bacterial, viral, eosinophilic, stress-induced, chronic erosive, atrophic, plasma cell, and Menetrier's), and peritoneal diseases (e.g., chyloperioneum, hemoperitoneum, mesenteric cyst, mesenteric lymphadenitis, mesenteric vascular occlusion, panniculitis, neoplasms, peritonitis, pneumoperitoneum, bubphrenic abscess,).

Gastrointestinal disorders also include disorders associated with the small intestine, such as malabsorption syndromes, distension, irritable bowel syndrome, sugar intolerance, celiac disease, duodenal ulcers, duodenitis, tropical sprue, Whipple's disease, intestinal lymphangiectasia, Crohn's disease, appendicitis, obstructions of the ileum, Meckel's diverticulum, multiple diverticula, failure of complete rotation of the small and large intestine, lymphoma, and bacterial and parasitic diseases (such as Traveler's diarrhea, typhoid and paratyphoid, cholera, infection by Roundworms (Ascariasis lumbricoides), Hookworms (Ancylostoma duodenale), Threadworms (Enterobius vermicularis), Tapeworms (Taenia saginata, Echinococcus granulosus, Diphyllobothrium spp., and T. solium).

Liver diseases and/or disorders include intrahepatic cholestasis (alagille syndrome, biliary liver cirrhosis), fatty liver (alcoholic fatty liver, reye syndrome), hepatic vein thrombosis, hepatolentricular degeneration, hepatomegaly, hepatopulmonary syndrome, hepatorenal syndrome, portal hypertension (esophageal and gastric varices), liver abscess (amebic liver abscess), liver cirrhosis (alcoholic, biliary and experimental), alcoholic liver diseases (fatty liver, hepatitis, cirrhosis), parasitic (hepatic echinococcosis, fascioliasis, amebic liver abscess), jaundice (hemolytic, hepatocellular, and cholestatic), cholestasis, portal hypertension, liver enlargement, ascites, hepatitis (alcoholic hepatitis, animal hepatitis, chronic hepatitis (autoimmune, hepatitis B, hepatitis C, hepatitis D, drug induced), toxic hepatitis, viral human hepatitis (hepatitis A, hepatitis B, hepatitis C, hepatitis D, hepatitis E), Wilson's disease, granulomatous hepatitis, secondary biliary cirrhosis, hepatic encephalopathy, portal hypertension, varices, hepatic encephalopathy, primary biliary cirrhosis, primary sclerosing cholangitis, hepatocellular adenoma, hemangiomas, bile stones, liver failure (hepatic encephalopathy, acute liver failure), and liver neoplasms (angiomyolipoma, calcified liver metastases, cystic liver metastases, epithelial tumors, fibrolamellar hepatocarcinoma, focal nodular hyperplasia, hepatic adenoma, hepatobiliary cystadenoma, hepatoblastoma, hepatocellular carcinoma, hepatoma, liver cancer, liver hemangioendothelioma, mesenchymal hamartoma, mesenchymal tumors of liver, nodular regenerative hyperplasia, benign liver tumors (Hepatic cysts [Simple cysts, Polycystic liver disease, Hepatobiliary cystadenoma, Choledochal cyst], Mesenchymal tumors [Mesenchymal hamartoma, Infantile hemangioendothelioma, Hemangioma, Peliosis hepatis, Lipomas, Inflammatory pseudotumor, Miscellaneous], Epithelial tumors [Bile duct epithelium (Bile duct hamartoma, Bile duct adenoma), Hepatocyte (Adenoma, Focal nodular hyperplasia, Nodular regenerative hyperplasia)], malignant liver tumors [hepatocellular, hepatoblastoma, hepatocellular carcinoma, cholangiocellular, cholangiocarcinoma, cystadenocarcinoma, tumors of blood vessels, angiosarcoma, Karposi's sarcoma, hemangioendothelioma, other tumors, embryonal sarcoma, fibrosarcoma, leiomyosarcoma, rhabdomyosarcoma, carcinosarcoma, teratoma, carcinoid, squamous carcinoma, primary lymphoma]), peliosis hepatis, erythrohepatic porphyria, hepatic porphyria (acute intermittent porphyria, porphyria cutanea tarda), Zellweger syndrome).

Pancreatic diseases and/or disorders include acute pancreatitis, chronic pancreatitis (acute necrotizing pancreatitis, alcoholic pancreatitis), neoplasms (adenocarcinoma of the pancreas, cystadenocarcinoma, insulinoma, gastrinoma, and glucagonoma, cystic neoplasms, islet-cell tumors, pancreoblastoma), and other pancreatic diseases (e.g., cystic fibrosis, cyst (pancreatic pseudocyst, pancreatic fistula, insufficiency)).

Gallbladder diseases include gallstones (cholelithiasis and choledocholithiasis), postcholecystectomy syndrome, diverticulosis of the gallbladder, acute cholecystitis, chronic cholecystitis, bile duct tumors, and mucocele.

Diseases and/or disorders of the large intestine include antibiotic-associated colitis, diverticulitis, ulcerative colitis, acquired megacolon, abscesses, fungal and bacterial infections, anorectal disorders (e.g., fissures, hemorrhoids), colonic diseases (colitis, colonic neoplasms [colon cancer, adenomatous colon polyps (e.g., villous adenoma), colon carcinoma, colorectal cancer], colonic diverticulitis, colonic diverticulosis, megacolon [Hirschsprung disease, toxic megacolon]; sigmnoid diseases [proctocolitis, sigmoin neoplasms]), constipation, Crohn's disease, diarrhea (infantile diarrhea, dysentery), duodenal diseases (duodenal neoplasms, duodenal obstruction, duodenal ulcer, duodenitis), enteritis (enterocolitis), HIV enteropathy, ileal diseases (ileal neoplasms, ileitis), immunoproliferative small intestinal disease, inflammatory bowel disease (ulcerative colitis, Crohn's disease), intestinal atresia, parasitic diseases (anisakiasis, balantidiasis, blastocystis infections, cryptosporidiosis, dientamoebiasis, amebic dysentery, giardiasis), intestinal fistula (rectal fistula), intestinal neoplasms (cecal neoplasms, colonic neoplasms, duodenal neoplasms, ileal neoplasms, intestinal polyps, jejunal neoplasms, rectal neoplasms), intestinal obstruction (afferent loop syndrome, duodenal obstruction, impacted feces, intestinal pseudo-obstruction [cecal volvulus], intussusception), intestinal perforation, intestinal polyps (colonic polyps, gardner syndrome, peutz-jeghers syndrome), jejunal diseases jejunal neoplasms), malabsorption syndromes (blind loop syndrome, celiac disease, lactose intolerance, short bowl syndrome, tropical sprue, whipple's disease), mesenteric vascular occlusion, pneumatosis cystoides intestinalis, protein-losing enteropathies (intestinal lymphagiectasis), rectal diseases (anus diseases, fecal incontinence, hemorrhoids, proctitis, rectal fistula, rectal prolapse, rectocele), peptic ulcer (duodenal ulcer, peptic esophagitis, hemorrhage, perforation, stomach ulcer, Zollinger-Ellison syndrome), postgastrectomy syndromes (dumping syndrome), stomach diseases (e.g., achlorhydria, duodenogastric reflux (bile reflux), gastric antral vascular ectasia, gastric fistula, gastric outlet obstruction, gastritis (atrophic or hypertrophic), gastroparesis, stomach dilatation, stomach diverticulum, stomach neoplasms (gastric cancer, gastric polyps, gastric adenocarcinoma, hyperplastic gastric polyp), stomach rupture, stomach ulcer, stomach volvulus), tuberculosis, visceroptosis, vomiting (e.g., hematemesis, hyperemesis gravidarum, postoperative nausea and vomiting) and hemorrhagic colitis.

Further diseases and/or disorders of the gastrointestinal system include biliary tract diseases, such as, gastroschisis, fistula (e.g., biliary fistula, esophageal fistula, gastric fistula, intestinal fistula, pancreatic fistula), neoplasms (e.g., biliary tract neoplasms, esophageal neoplasms, such as adenocarcinoma of the esophagus, esophageal squamous cell carcinoma, gastrointestinal neoplasms, pancreatic neoplasms, such as adenocarcinoma of the pancreas, mucinous cystic neoplasm of the pancreas, pancreatic cystic neoplasms, pancreatoblastoma, and peritoneal neoplasms), esophageal disease (e.g., bullous diseases, candidiasis, glycogenic acanthosis, ulceration, barrett esophagus varices, atresia, cyst, diverticulum (e.g., Zenker's diverticulum), fistula (e.g., tracheoesophageal fistula), motility disorders (e.g., CREST syndrome, deglutition disorders, achalasia, spasm, gastroesophageal reflux), neoplasms, perforation (e.g., Boerhaave syndrome, Mallory-Weiss syndrome), stenosis, esophagitis, diaphragmatic hemia (e.g., hiatal hemia); gastrointestinal diseases, such as, gastroenteritis (e.g., cholera morbus, norwalk virus infection), hemorrhage (e.g., hematemesis, melena, peptic ulcer hemorrhage), stomach neoplasms (gastric cancer, gastric polyps, gastric adenocarcinoma, stomach cancer)), hemia (e.g., congenital diaphragmatic hemia, femoral hemia, inguinal hemia, obturator hemia, umbilical hemia, ventral hemia), and intestinal diseases (e.g., cecal diseases (appendicitis, cecal neoplasms)).

Q. Chemotaxis

Alpha-fetoprotein fusion proteins of the invention and/or polynucleotides encoding alpha-fetoprotein fusion proteins of the invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

Alpha-fetoprotein fusion proteins of the invention and/or polynucleotides encoding alpha-fetoprotein fusion proteins of the invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that fusion proteins of the invention and/or polynucleotides encoding alpha-fetoprotein fusion proteins of the invention may inhibit chemotactic activity. These molecules could also be used to treat disorders. Thus, fusion proteins of the invention and/or polynucleotides encoding alpha-fetoprotein fusion proteins of the invention could be used as an inhibitor of chemotaxis.

R. Binding Activity

Alpha-fetoprotein fusion proteins of the invention may be used to screen for molecules that bind to the therapeutic protein portion of the fusion protein or for molecules to which the therapeutic protein portion of the fusion protein binds. The binding of the fusion protein and the molecule may activate (agonist), increase, inhibit (antagonist), or decrease activity of the fusion protein or the molecule bound. Examples of such molecules include antibodies, oligonucleotides, proteins (e.g., receptors), or small molecules.

Preferably, the molecule is closely related to the natural ligand of the therapeutic protein portion of the fusion protein of the invention, e.g., a fragment of the ligand, or a natural substrate, a ligand, a structural or functional mimetic. (See, Coligan et al., Current Protocols in Immunology 1(2):Chapter 5 (1991)). Similarly, the molecule can be closely related to the natural receptor to which the therapeutic protein portion of an alpha-fetoprotein fusion protein of the invention binds, or at least, a fragment of the receptor capable of being bound by the therapeutic protein portion of an alpha-fetoprotein fusion protein of the invention (e.g., active site). In either case, the molecule can be rationally designed using known techniques.

Preferably, the screening for these molecules involves producing appropriate cells which express the alpha-fetoprotein fusion proteins of the invention. Preferred cells include cells from mammals, yeast, Drosophila, or E. coli.

The assay may simply test binding of a candidate compound to an alpha-fetoprotein fusion protein of the invention, wherein binding is detected by a label, or in an assay involving competition with a labeled competitor. Further, the assay may test whether the candidate compound results in a signal generated by binding to the fusion protein.

Alternatively, the assay can be carried out using cell-free preparations, fusion protein/molecule affixed to a solid support, chemical libraries, or natural product mixtures. The assay may also simply comprise the steps of mixing a candidate compound with a solution containing an alpha-fetoprotein fusion protein, measuring fusion protein/molecule activity or binding, and comparing the fusion protein/molecule activity or binding to a standard.

Preferably, an ELISA assay can measure fusion protein level or activity in a sample (e.g., biological sample) using a monoclonal or polyclonal antibody. The antibody can measure fusion protein level or activity by either binding, directly or indirectly, to the alpha-fetoprotein fusion protein or by competing with the alpha-fetoprotein fusion protein for a substrate.

Additionally, the receptor to which a therapeutic protein portion of an alpha-fetoprotein fusion protein of the invention binds can be identified by numerous methods known to those of skill in the art, for example, ligand panning and FACS sorting (Coligan, et al., Current Protocols in Immun., 1(2), Chapter 5, (1991)). For example, in cases wherein the therapeutic protein portion of the fusion protein corresponds to FGF, expression cloning may be employed wherein polyadenylated RNA is prepared from a cell responsive to the alpha-fetoprotein fusion protein, for example, NIH3T3 cells which are known to contain multiple receptors for the FGF family proteins, and SC-3 cells, and a cDNA library created from this RNA is divided into pools and used to transfect COS cells or other cells that are not responsive to the alpha-fetoprotein fusion protein. Transfected cells which are grown on glass slides are exposed to the alpha-fetoprotein fusion protein of the present invention, after they have been labeled. The alpha-fetoprotein fusion proteins can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, a labeled alpha-fetoprotein fusion protein can be photoaffinity linked with cell membrane or extract preparations that express the receptor molecule for the Therapeutic protein component of an alpha-fetoprotein fusion protein of the invention, the linked material may be resolved by PAGE analysis and exposed to X-ray film. The labeled complex containing the receptors of the fusion protein can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling, and/or codon-shuffling (collectively referred to as “DNA shuffling”) may be employed to modulate the activities of the fusion protein, and/or therapeutic protein portion or alpha-fetoprotein component of an alpha-fetoprotein fusion protein of the present invention, thereby effectively generating agonists and antagonists of an alpha-fetoprotein fusion protein of the present invention. In one embodiment, alteration of polynucleotides encoding alpha-fetoprotein fusion proteins of the invention and thus, the alpha-fetoprotein fusion proteins encoded thereby, may be achieved by DNA shuffling. DNA shuffling involves the assembly of two or more DNA segments into a desired molecule by homologous, or site-specific, recombination. In another embodiment, polynucleotides encoding alpha-fetoprotein fusion proteins of the invention and thus, the alpha-fetoprotein fusion proteins encoded thereby, may be altered by being subjected to random mutagenesis by error-prone PCR, random nucleotide insertion or other methods prior to recombination. In another embodiment, one or more components, motifs, sections, parts, domains, fragments, etc., of an alpha-fetoprotein fusion protein of the present invention may be recombined with one or more components, motifs, sections, parts, domains, fragments, etc. of one or more heterologous molecules. In preferred embodiments, the heterologous molecules are family members. In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic(dpp), 60A, OP-2, dorsalin, growth differentiation factors (GDFs), nodal, MIS, inhibin-alpha, TGF-beta1, TGF-beta2, TGF-beta3, TGF-beta5, and glial-derived neurotrophic factor (GDNF).

Other preferred fragments are biologically active fragments of the therapeutic protein portion and/or alpha-fetoprotein component of the alpha-fetoprotein fusion proteins of the present invention. Biologically active fragments are those exhibiting activity similar, but not necessarily identical, to an activity of a therapeutic protein portion and/or alpha-fetoprotein component of the alpha-fetoprotein fusion proteins of the present invention. The biological activity of the fragments may include an improved desired activity, or a decreased undesirable activity.

Additionally, this invention provides a method of screening compounds to identify those which modulate the action of an alpha-fetoprotein fusion protein of the present invention. An example of such an assay comprises combining a mammalian fibroblast cell, an alpha-fetoprotein fusion protein of the present invention, and the compound to be screened and 3[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of ³[H] thymidine in each case. The amount of fibroblast cell proliferation is measured by liquid scintillation chromatography which measures the incorporation of ³[H] thymidine. Both agonist and antagonist compounds may be identified by this procedure.

In another method, a mammalian cell or membrane preparation expressing a receptor for the therapeutic protein component of a fusion protein of the invention is incubated with a labeled fusion protein of the present invention in the presence of the compound. The ability of the compound to enhance or block this interaction could then be measured. Alternatively, the response of a known second messenger system following interaction of a compound to be screened and the receptor is measured and the ability of the compound to bind to the receptor and elicit a second messenger response is measured to determine if the compound is a potential fusion protein. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat disease or to bring about a particular result in a patient (e.g., blood vessel growth) by activating or inhibiting the fusion protein/molecule. Moreover, the assays can discover agents which may inhibit or enhance the production of the alpha-fetoprotein fusion proteins of the invention from suitably manipulated cells or tissues.

Therefore, the invention includes a method of identifying compounds which bind to an alpha-fetoprotein fusion protein of the invention comprising the steps of: (a) incubating a candidate binding compound with an alpha-fetoprotein fusion protein of the present invention; and (b) determining if binding has occurred. Moreover, the invention includes a method of identifying agonists/antagonists comprising the steps of: (a) incubating a candidate compound with an alpha-fetoprotein fusion protein of the present invention, (b) assaying a biological activity, and (b) determining if a biological activity of the fusion protein has been altered.

S. Targeted Delivery

In another embodiment, the invention provides a method of delivering compositions to targeted cells expressing a receptor for a component of an alpha-fetoprotein fusion protein of the invention.

As discussed herein, fusion proteins of the invention may be associated with heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering fusion proteins of the invention (including antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering an alpha-fetoprotein fusion protein of the invention (e.g., polypeptides of the invention or antibodies of the invention) in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, PA toxin (anthrax), ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

T. Drug Screening

Further contemplated is the use of the alpha-fetoprotein fusion proteins of the present invention, or the polynucleotides encoding these fusion proteins, to screen for molecules which modify the activities of the alpha-fetoprotein fusion protein of the present invention or proteins corresponding to the therapeutic protein portion of the alpha-fetoprotein fusion protein. Such a method would include contacting the fusion protein with a selected compound(s) suspected of having antagonist or agonist activity, and assaying the activity of the fusion protein following binding.

This invention is particularly useful for screening therapeutic compounds by using the alpha-fetoprotein fusion proteins of the present invention, or binding fragments thereof, in any of a variety of drug screening techniques. The alpha-fetoprotein fusion protein employed in such a test may be affixed to a solid support, expressed on a cell surface, free in solution, or located intracellularly. One method of drug screening utilizes eukaryotic or prokaryotic host cells which are stably transformed with recombinant nucleic acids expressing the alpha-fetoprotein fusion protein. Drugs are screened against such transformed cells or supernatants obtained from culturing such cells, in competitive binding assays. One may measure, for example, the formulation of complexes between the agent being tested and an alpha-fetoprotein fusion protein of the present invention.

Thus, the present invention provides methods of screening for drugs or any other agents which affect activities mediated by the alpha-fetoprotein fusion proteins of the present invention. These methods comprise contacting such an agent with an alpha-fetoprotein fusion protein of the present invention or a fragment thereof and assaying for the presence of a complex between the agent and the alpha-fetoprotein fusion protein or a fragment thereof, by methods well known in the art. In such a competitive binding assay, the agents to screen are typically labeled. Following incubation, free agent is separated from that present in bound form, and the amount of free or uncomplexed label is a measure of the ability of a particular agent to bind to the alpha-fetoprotein fusion protein of the present invention.

Another technique for drug screening provides high throughput screening for compounds having suitable binding affinity to an alpha-fetoprotein fusion protein of the present invention, and is described in great detail in European Patent Application 84/03564, published on Sep. 13, 1984, which is incorporated herein by reference herein. Briefly stated, large numbers of different small peptide test compounds are synthesized on a solid substrate, such as plastic pins or some other surface. The peptide test compounds are reacted with an alpha-fetoprotein fusion protein of the present invention and washed. Bound peptides are then detected by methods well known in the art. Purified alpha-fetoprotein fusion protein may be coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies capable of binding an alpha-fetoprotein fusion protein of the present invention specifically compete with a test compound for binding to the alpha-fetoprotein fusion protein or fragments thereof. In this manner, the antibodies are used to detect the presence of any peptide which shares one or more antigenic epitopes with an alpha-fetoprotein fusion protein of the invention.

1. Binding Peptides and Other Molecules

The invention also encompasses screening methods for identifying polypeptides and nonpolypeptides that bind alpha-fetoprotein fusion proteins of the invention, and the binding molecules identified thereby. These binding molecules are useful, for example, as agonists and antagonists of the alpha-fetoprotein fusion proteins of the invention. Such agonists and antagonists can be used, in accordance with the invention, in the therapeutic embodiments described in detail, below.

This method comprises the steps of. contacting an alpha-fetoprotein fusion protein of the invention with a plurality of molecules; and identifying a molecule that binds the alpha-fetoprotein fusion protein.

The step of contacting the alpha-fetoprotein fusion protein of the invention with the plurality of molecules may be effected in a number of ways. For example, one may contemplate immobilizing the alpha-fetoprotein fusion protein on a solid support and bringing a solution of the plurality of molecules in contact with the immobilized polypeptides. Such a procedure would be akin to an affinity chromatographic process, with the affinity matrix being comprised of the immobilized alpha-fetoprotein fusion protein of the invention. The molecules having a selective affinity for the alpha-fetoprotein fusion protein can then be purified by affinity selection. The nature of the solid support, process for attachment of the alpha-fetoprotein fusion protein to the solid support, solvent, and conditions of the affinity isolation or selection are largely conventional and well known to those of ordinary skill in the art.

Alternatively, one may also separate a plurality of polypeptides into substantially separate fractions comprising a subset of or individual polypeptides. For instance, one can separate the plurality of polypeptides by gel electrophoresis, column chromatography, or like method known to those of ordinary skill for the separation of polypeptides. The individual polypeptides can also be produced by a transformed host cell in such a way as to be expressed on or about its outer surface (e.g., a recombinant phage). Individual isolates can then be “probed” by an alpha-fetoprotein fusion protein of the invention, optionally in the presence of an inducer should one be required for expression, to determine if any selective affinity interaction takes place between the alpha-fetoprotein fusion protein and the individual clone. Prior to contacting the alpha-fetoprotein fusion protein with each fraction comprising individual polypeptides, the polypeptides could first be transferred to a solid support for additional convenience. Such a solid support may simply be a piece of filter membrane, such as one made of nitrocellulose or nylon. In this manner, positive clones could be identified from a collection of transformed host cells of an expression library, which harbor a DNA construct encoding a polypeptide having a selective affinity for an alpha-fetoprotein fusion protein of the invention. Furthermore, the amino acid sequence of the polypeptide having a selective affinity for an alpha-fetoprotein fusion protein of the invention can be determined directly by conventional means or the coding sequence of the DNA encoding the polypeptide can frequently be determined more conveniently. The primary sequence can then be deduced from the corresponding DNA sequence. If the amino acid sequence is to be determined from the polypeptide itself, one may use microsequencing techniques. The sequencing technique may include mass spectroscopy.

In certain situations, it may be desirable to wash away any unbound polypeptides from a mixture of an alpha-fetoprotein fusion protein of the invention and the plurality of polypeptides prior to attempting to determine or to detect the presence of a selective affinity interaction. Such a wash step may be particularly desirable when the alpha-fetoprotein fusion protein of the invention or the plurality of polypeptides are bound to a solid support.

The plurality of molecules provided according to this method may be provided by way of diversity libraries, such as random or combinatorial peptide or nonpeptide libraries which can be screened for molecules that specifically bind an alpha-fetoprotein fusion protein of the invention. Many libraries are known in the art that can be used, e.g., chemically synthesized libraries, recombinant (e.g., phage display libraries), and in vitro translation-based libraries. Examples of chemically synthesized libraries are described in Fodor et al., Science 251:767-773 (1991); Houghten et al., Nature 354:84-86 (1991); Lam et al., Nature 354:82-84 (1991); Medynski, Bio/technology 12:709-710 (1994); Gallop et al., J. Medicinal Chemistry 37(9):1233-1251 (1994); Ohlmeyer et al., Proc. Natl. Acad. Sci. USA 90:10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); PCT Publication No. WO 93/20242; and Brenner and Lerner, Proc. Natl. Acad. Sci. USA 89:5381-5383 (1992).

Examples of phage display libraries are described in Scott et al., Science 249:386-390 (1990); Devlin et al., Science, 249:404-406 (1990); Christian et al., 1992, J. Mol. Biol. 227:711-718 1992); Lenstra, J. Immunol. Meth. 152:149-157 (1992); Kay et al., Gene 128:59-65 (1993); and PCT Publication No. WO 94/18318 dated Aug. 18, 1994.

In vitro translation-based libraries include but are not limited to those described in PCT Publication No. WO 91/05058 dated Apr. 18, 1991; and Mattheakis et al., Proc. Natl. Acad. Sci. USA 91:9022-9026 (1994).

By way of examples of nonpeptide libraries, a benzodiazepine library (see e.g., Bunin et al., Proc. Natl. Acad. Sci. USA 91:47084712 (1994)) can be adapted for use. Peptoid libraries (Simon et al., Proc. Natl. Acad. Sci. USA 89:9367-9371 (1992)) can also be used. Another example of a library that can be used, in which the amide functionalities in peptides have been permethylated to generate a chemically transformed combinatorial library, is described by Ostresh et al. (Proc. Natl. Acad. Sci. USA 91:11138-11142 (1994)).

The variety of non-peptide libraries that are useful in the present invention is great. For example, Ecker and Crooke (Bio/Technology 13:351-360 (1995) list benzodiazepines, hydantoins, piperazinediones, biphenyls, sugar analogs, beta-mercaptoketones, arylacetic acids, acylpiperidines, benzopyrans, cubanes, xanthines, aminimides, and oxazolones as among the chemical species that form the basis of various libraries.

Non-peptide libraries can be classified broadly into two types: decorated monomers and oligomers. Decorated monomer libraries employ a relatively simple scaffold structure upon which a variety functional groups is added. Often the scaffold will be a molecule with a known useful pharmacological activity. For example, the scaffold might be the benzodiazepine structure.

Non-peptide oligomer libraries utilize a large number of monomers that are assembled together in ways that create new shapes that depend on the order of the monomers. Among the monomer units that have been used are carbamates, pyrrolinones, and morpholinos. Peptoids, peptide-like oligomers in which the side chain is attached to the alpha amino group rather than the alpha carbon, form the basis of another version of non-peptide oligomer libraries. The first non-peptide oligomer libraries utilized a single type of monomer and thus contained a repeating backbone. Recent libraries have utilized more than one monomer, giving the libraries added flexibility.

Screening the libraries can be accomplished by any of a variety of commonly known methods. See, e.g., the following references, which disclose screening of peptide libraries: Parmley et al., Adv. Exp. Med. Biol. 251:215-218 (1989); Scott et al., Science 249:386-390(1990); Fowlkes et al., BioTechniques 13:422-427 (1992); Oldenburg et al., Proc. Natl. Acad. Sci. USA 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. USA 89:6988-6992 (1992); Ellington et al., Nature 355:850-852 (1992); U.S. Pat. No. 5,096,815, U.S. Pat. No. 5,223,409, and U.S. Pat. No. 5,198,346, all to Ladner et al.; Rebar et al., Science 263:671-673 (1993); and PCT Publication No. WO 94/18318.

In a specific embodiment, screening to identify a molecule that binds an alpha-fetoprotein fusion protein of the invention can be carried out by contacting the library members with an alpha-fetoprotein fusion protein of the invention immobilized on a solid phase and harvesting those library members that bind to the alpha-fetoprotein fusion protein. Examples of such screening methods, termed “panning” techniques are described by way of example in Parmley et al., Gene 73:305-318 (1988); Fowlkes et al., BioTechniques 13:422-427 (1992); PCT Publication No. WO 94/18318; and in references cited herein.

In another embodiment, the two-hybrid system for selecting interacting proteins in yeast (Fields et al., Nature 340:245-246 (1989); Chien et al., Proc. Natl. Acad. Sci. USA 88:9578-9582 (1991) can be used to identify molecules that specifically bind to polypeptides of the invention.

Where the binding molecule is a polypeptide, the polypeptide can be conveniently selected from any peptide library, including random peptide libraries, combinatorial peptide libraries, or biased peptide libraries. The term “biased” is used herein to mean that the method of generating the library is manipulated so as to restrict one or more parameters that govern the diversity of the resulting collection of molecules, in this case peptides.

Thus, a truly random peptide library would generate a collection of peptides in which the probability of finding a particular amino acid at a given position of the peptide is the same for all 20 amino acids. A bias can be introduced into the library, however, by specifying, for example, that a lysine occur every fifth amino acid or that positions 4, 8, and 9 of a decapeptide library be fixed to include only arginine. Clearly, many types of biases can be contemplated, and the present invention is not restricted to any particular bias. Furthermore, the present invention contemplates specific types of peptide libraries, such as phage displayed peptide libraries and those that utilize a DNA construct comprising a lambda phage vector with a DNA insert.

As mentioned above, in the case of a binding molecule that is a polypeptide, the polypeptide may have about 6 to less than about 60 amino acid residues, preferably about 6 to about 10 amino acid residues, and most preferably, about 6 to about 22 amino acids. In another embodiment, a binding polypeptide has in the range of 15-100 amino acids, or 20-50 amino acids.

The selected binding polypeptide can be obtained by chemical synthesis or recombinant expression.

U. Other Activities

An alpha-fetoprotein fusion protein of the invention and/or polynucleotide encoding an alpha-fetoprotein fusion protein of the invention, may be employed in treatment for stimulating re-vascularization of ischemic tissues due to various disease conditions such as thrombosis, arteriosclerosis, and other cardiovascular conditions. The alpha-fetoprotein fusion proteins of the invention and/or polynucleotides encoding alpha-fetoprotein fusion proteins of the invention may also be employed to stimulate angiogenesis and limb regeneration, as discussed above.

An alpha-fetoprotein fusion protein of the invention and/or polynucleotide encoding an alpha-fetoprotein fusion protein of the invention may also be employed for treating wounds due to injuries, burns, post-operative tissue repair, and ulcers since they are mitogenic to various cells of different origins, such as fibroblast cells and skeletal muscle cells, and therefore, facilitate the repair or replacement of damaged or diseased tissue.

An alpha-fetoprotein fusion protein of the invention and/or polynucleotide encoding an alpha-fetoprotein fusion protein of the invention may also be employed stimulate neuronal growth and to treat and prevent neuronal damage which occurs in certain neuronal disorders or neuro-degenerative conditions such as Alzheimer's disease, Parkinson's disease, and AIDS-related complex. An alpha-fetoprotein fusion protein of the invention and/or polynucleotide encoding an alpha-fetoprotein fusion protein of the invention may have the ability to stimulate chondrocyte growth, therefore, they may be employed to enhance bone and periodontal regeneration and aid in tissue transplants or bone grafts.

An alpha-fetoprotein fusion protein of the invention and/or polynucleotide encoding an alpha-fetoprotein fusion protein of the invention may be also be employed to prevent skin aging due to sunburn by stimulating keratinocyte growth.

An alpha-fetoprotein fusion protein of the invention and/or polynucleotide encoding an alpha-fetoprotein fusion protein of the invention may also be employed for preventing hair loss. Along the same lines, an alpha-fetoprotein fusion protein of the invention and/or polynucleotide encoding an alpha-fetoprotein fusion protein of the invention may be employed to stimulate growth and differentiation of hematopoietic cells and bone marrow cells when used in combination with other cytokines.

An alpha-fetoprotein fusion protein of the invention and/or polynucleotide encoding an alpha-fetoprotein fusion protein of the invention may also be employed to maintain organs before transplantation or for supporting cell culture of primary tissues. An alpha-fetoprotein fusion protein of the invention and/or polynucleotide encoding an alpha-fetoprotein fusion protein of the invention may also be employed for inducing tissue of mesodermal origin to differentiate in early embryos.

An alpha-fetoprotein fusion protein of the invention and/or polynucleotide encoding an alpha-fetoprotein fusion protein of the invention may also increase or decrease the differentiation or proliferation of embryonic stem cells, besides, as discussed above, hematopoietic lineage.

An alpha-fetoprotein fusion protein of the invention and/or polynucleotide encoding an alpha-fetoprotein fusion protein of the invention may also be used to modulate mammalian characteristics, such as body height, weight, hair color, eye color, skin, percentage of adipose tissue, pigmentation, size, and shape (e.g., cosmetic surgery). Similarly, an alpha-fetoprotein fusion protein of the invention and/or polynucleotide encoding an alpha-fetoprotein fusion protein of the invention may be used to modulate mammalian metabolism affecting catabolism, anabolism, processing, utilization, and storage of energy.

An alpha-fetoprotein fusion protein of the invention and/or polynucleotide encoding an alpha-fetoprotein fusion protein of the invention may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

An alpha-fetoprotein fusion protein of the invention and/or polynucleotide encoding an alpha-fetoprotein fusion protein of the invention may also be used as a food additive or preservative, such as to increase or decrease storage capabilities, fat content, lipid, protein, carbohydrate, vitamins, minerals, cofactors or other nutritional components.

The above-recited applications have uses in a wide variety of hosts. Such hosts include, but are not limited to, human, murine, rabbit, goat, guinea pig, camel, horse, mouse, rat, hamster, pig, micro-pig, chicken, goat, cow, sheep, dog, cat, non-human primate, and human. In specific embodiments, the host is a mouse, rabbit, goat, guinea pig, chicken, rat, hamster, pig, sheep, dog or cat. In preferred embodiments, the host is a mammal. In most preferred embodiments, the host is a human.

The following examples are given to illustrate the invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

Without further description, it is believed that one of ordinary skill in the art can, using the preceding description and the following illustrative examples, make and utilize the alterations detected in the present invention and practice the claimed methods. The following prophetic (examples 1-7) and working (examples 8-10) examples therefore, specifically point out preferred embodiments of the present invention, and are not to be construed as limiting in any way the remainder of the disclosure.

### EXAMPLES

General Cloning Techniques

The methods conventionally used in molecular biology, such as the preparative extractions of plasmid DNA, the centrifugation of plasmid DNA in caesium chloride gradient, electrophoresis on agarose or acrylamide gels, purification of DNA fragments by electroclution, extractions of proteins with phenol or phenol-chloroform, DNA precipitation in saline medium with ethanol or isopropanol, transformation in Escherichia coli, and the like are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

The restriction enzymes can be provided by New England Biolabs (Biolabs), Bethesda Research Laboratories (BRL) or Amersham and can be used according to the recommendations of the suppliers.

The pBR322 and pUC type plasmids and the phages of the M13 series are of commercial origin (Bethesda Research Laboratories).

For the ligations, the DNA fragments can be separated according to their size by electrophoresis on agarose or acrylamide gels, extracted with phenol or with a phenol/chloroform mixture, precipitated with ethanol, and then incubated in the presence of phage T4 DNA ligase (Biolabs) according to the recommendations of the manufacturer.

The filling of the protruding 5′ ends can be carried out by the Klenow fragment of DNA polymerase I of E. coli (Biolabs) according to the specifications of the supplier. The destruction of the protruding 3′ ends can be carried out in the presence of phage T4 DNA polymerase (Biolabs) used according to the recommendations of the manufacturer. The destruction of the protruding 5′ ends can be carried out by a controlled treatment with S1 nuclease.

Site-directed mutagenesis in vitro with synthetic oligodeoxynucleotides can be carried out according to the method developed by Taylor et al. [Nucleic Acids Res. 13 (1985) 8749-8764] using the kit distributed by Amersham.

The enzymatic amplification of DNA fragments by the so-called PCR technique [Polymerase-catalyzed Chain Reaction, Saiki R. K. et al., Science 230 (1985) 1350-1354; Mullis K. B. and Faloona F. A., Meth. Enzym. 155 (1987) 335-350] can be carried out-using a “DNA thermal cycler” (Perkin Elmer Cetus) according to the specifications of the manufacturer.

The verification of the nucleotide sequences can be carried out by the method developed by Sanger et al. [Proc. Natl. Acad. Sci. U.S.A., 74 (1977) 5463-5467] using the kit distributed by Amersham.

The transformations of K. lactis with DNA from the plasmids for expression of the proteins of the present invention can be carried out by any technique known to persons skilled in the art, and of which an example is given in the text.

Except where otherwise stated, the bacterial strains envisioned are E. coli MC1060 (lacIPOZYA, X74, galU, galK, strAr), or E. coli TG1 (lac, proA,B, supE, thi, hsdD5/FtraD36, proA+B+, lacIq, lacZ, M15).

The yeast strains belong to the budding yeasts and more particularly to yeasts of the genus Kluyveromyces. The K. lactis MW98-8C (a, uraA, arg, lys, K⁺, pKDlo) and K. lactis CBS 293.91 strain may be used; a sample of the MW98-8C strain was deposited on 16 Scp. 1988 at Centraalbureau voor Schimmelkulturen (CBS) at Baarn (the Netherlands) where it was registered under the number CBS 579.88. Examples 1-7 are prophetic, and do not describe actual experimental data and results.

### Example 1

Coupling at the C-Terminus, N-Terminus, or Both the C- and N-Termini of Alpha-Fetoprotein

A plasmid containing a restriction fragment encoding AFPa-fetoprotein (AFP) can be used for cloning a biologically active peptide to couple in translational phase at the C-terminus of AFP. In another embodiment, the biologically active peptide may be present more than once in the chimera.

In a specific embodiment, the combined techniques of site-directed mutagenesis and PCR amplification make it possible to construct hybrid genes encoding a chimeric protein resulting from the translational coupling between a signal peptide, a sequence including the biologically active peptide and the mature form of AFP or one of its molecular variants. In a still more specific embodiment, the biologically active peptide may be present more than once in the chimera.

The combined techniques of site-directed mutagenesis and PCR amplification described in Examples 1 and 2 make it possible to construct hybrid genes encoding a chimeric protein resulting from the translational coupling between the mature form of AFP, or one of its molecular variants, and a biologically active peptide coupled to the N- and C-terminal ends of AFP. The peptide at the C-terminal end can be the same as or different from the peptide at teh N-terminal end of AFP. In a still more specific embodiment, the biologically active peptide may be present more than once in the chimera.

In a first example, an antigen for a vaccine is coupled to one terminus of AFP. Any antigen that elicits and immune response can be utilized in the compositions of the invention. Exemplary vaccine antigens include, but are not limited to, bacterial antigens, mycotic antigens, prion antigens, parasites, PA-toxin (e.g., anthrax), Human Immunodeficiency Virus (HIV-1 and HIV-2), Avian Flu antigen (e.g., H₅N₁), hepatitis, herpes, cancer, Severe Acute Respiratory Syndrome (SARS). Other exemplary vaccine antigens are described herein.

In a second example, an immunoadjuvant is coupled to the other end of AFP. Examples of adjuvants useful in vaccines include, but are not limited to, cytokines such as IL-2, Lipid A, including monophosphoryl lipid A, bacterial products, endotoxins, cholesterol, fatty acids, aliphatic amines, paraffinic and vegetable oils, threonyl derivative, and muramyl dipeptide.

### Example 2

Transformation in Yeast

A. Expression Plasmids

The chimeric proteins of the preceding examples can be expressed in yeasts using functional, regulatable or constitutive promoters such as, for example, those present in the plasmids pYG105 (LAC4 promoter of Kluyveromyces lactis), pYG106 (PGK promoter of Saccharomyces cerevisiae), pYG536 (PHO5 promoter of S. cerevisiae), or hybrid promoters such as those described in Patent Application EP 361 991.

B. Transformation of the Yeasts

The transformation of the yeasts belonging to the genus Kluyveromyces, and in particular the strains MW98-8C and CBS 293.91 of K. lactis can be carried out for example by the technique for treating whole cells with lithium acetate [Ito H. et al., J. Bacteriol. 153 (1983) 163-168], adapted as follows. The growth of the cells is carried out at 28° C. in 50 ml of YPD medium, with stirring and up to an optical density of 600 nm (OD600) of between 0.6 and 0.8; the cells can be harvested by centrifugation at low speed, washed in a sterile solution of TE (10 mM Tris HCl pH 7.4; 1 mM EDTA), resuspended in 3-4 ml of lithium acetate (0.1M in TE) to obtain a cellular density of about 2×108 cells/ml, and then incubated at 30° C. for 1 hour with moderate stirring. Aliquots of 0.1 ml of the resulting suspension of competent cells can be incubated at 30° C. for 1 hour in the presence of DNA and at a final concentration of 35% polyethylene glycol (PEG4000, Sigma). After a heat shock of 5 minutes at 42° C., the cells can be washed twice, resuspended in 0.2 ml of sterile water, and incubated for 16 hours at 28° C. in 2 ml of YPD medium to permit the phenotypic expression of the gene for resistance to G418 expressed under the control of the Pkl promoter (cf. EP 361 991); 200 μl of the cellular suspension can then be then plated on selective YPD dishes (G418, 200 μg/ml). The dishes can be incubated at 28° C. and the transformants should appear after 2 to 3 days of cell growth.

C. Secretion of the Chimeras

After selection on rich medium supplemented with G418, the recombinant clones can be tested for their capacity to secrete the mature form of the AFP chimeric proteins. Few clones can be incubated in YPD or YPL medium at 28° C. The cellular supernatants can be recovered by centrifugation when the cells reach the stationary growth phase, optionally concentrated 10 times by precipitation for 30 minutes at −20° C. in a final concentration of 60% ethanol, and then tested after electrophoresis on an 8.5% SDS-PAGE gel, either directly by staining the gel with coomassie blue, or after immunoblotting using primary antibodies directed against the biologically active part or a rabbit polyclonal serum directed against AFP. During the experiments for immunological detection, the nitrocellulose filter is first incubated in the presence of specific primary antibodies, washed several times, incubated in the presence of goat antibodies directed against the primary antibodies, and then incubated in the presence of an avidin-peroxidase complex using the “ABC kit” distributed by Vectastain (Biosys S. A., Compiegne, France). The immunological reaction is then revealed by the addition of 3,3′-diamino benzidine tetrahydrochloride (Prolabo) in the presence of hydrogen peroxide, according to the recommendations of the manufacturer.

### Example 3

Chimeras Derived from the Von Willebrand Factor

E.3.1. Fragments Antagonizing the Binding of vWF to the Platelets

E.3.1.1. Thr470-Val713 Residues of vWF

The plasmid pET-8c52K contains a fragment of the vWF cDNA encoding residues 445 to 733 of human vWF and therefore includes several crucial determinants of the interaction between vWF and the platelets on the one hand, and certain elements of the basal membrane and the sub-endothelial tissue on the other, and especially the peptides G10 and D5 which antagonize the interaction between vWF and GPIb [Mori H. et al., J. Biol. Chem. 263 (1988) 17901-17904]. This peptide sequence is identical to the corresponding sequence described by Titani et al. [Biochemistry 25, (1986) 3171-3184]. The amplification of these genetic determinants can be carried out using the plasmid pET-8c52K, for example by the PCR amplification technique, using as primer oligodeoxynucleotides encoding contiguous residues localized on either side of the sequence to be amplified. The amplified fragments can then be cloned into vectors of the M13 type for their verification by sequencing using either the universal primers situated on either side of the multiple cloning site, or oligodeoxynucleotides specific for the amplified region of the vWF gene of which the sequence of several isomorphs is known [Sadler J. E. et al., Proc. Natl. Acad. Sci. 82 (1985) 6394-6398; Verweij C. L. et al., EMBO J. 5 (1986) 1839-1847; Shelton-Inloes B. B. et al., Biochemistry 25 (1986) 3164-3171; Bonthron D. et al., Nucleic Acids Res. 17 (1986) 7125-7127]. Thus, the PCR amplification of the plasmid pET-8c52K with the oligodeoxynucleotides 5′-CCCGGGATCCCTTAGGCTTAACCTGTGAAGCCTGC-3′ and 5′-CCCGGGATCCAAGCTTAGACTTGTGCCATGTCG-3′ generates an MstII-HindIII restriction fragment including the Thr470 to Val713 residues of vWF.

The ligation of this fragment to a restriction fragment corresponding to the entire gene encoding AFP can generate a restriction fragment containing a hybrid gene encoding a chimeric protein of the AFP-PEPTIDE type. This restriction fragment can then be cloned in the productive orientation and into a suitable restriction site of a plasmid, which generates an expression plasmid.

E.3.1.2. Molecular Variants:

In another embodiment, the binding site of vWF is a peptide including the Thr470 to Asp498 residues of the mature vWF. This sequence including the peptide G10 (Cys474-Pro488) described by Mori et al. [J. Biol. Chem. 263 (1988) 17901-17904] and is capable of antagonizing the interaction of human vWF with the GP1b of the human platelets. The sequence corresponding to the peptide G10 is first included in an MstII-HindIII restriction fragment, for example by PCR amplification of the plasmid pET-8c52K with the oligodeoxynucleotides Sq 1969 and 5′-CCCGGGATCCAAGCTTAGTCCTCCACATACAG-3′, which generates an MstII-HindIII restriction fragment including the peptide G10, and whose sequence is: 5′-CCTTAGGCTTAACCTGTGAAGCCTGCCAGGAGCCGGGAGGCCTGGTGGTGCCTCC CA CAGATGCCCCGGTGAGCCCC-ACCACTCTGTATGTGGAGGACTAAGCTT-3′. The ligation of this fragment to a restriction fragment corresponding to the entire gene encoding AFP will generate a restriction fragment containing a hybrid gene encoding a chimeric protein of the AFP-PEPTIDE type. This restriction fragment can then be cloned in the productive orientation into a restriction site of a plasmid to generate an expression plasmid.

In another embodiment, the site for binding of vWF to GP1b can be directly designed with the aid of synthetic oligodeoxynucleotides, and for example the oligodeoxynucleotides 5′-TTAGGCCTCTGTGACCTTGCCCCTGAAGCCCCTCCTCCTACTCTGCCCCCCTAAGC TT A-3′ and 5′-GATCTAAGCTTAGGGGGGCAGAGTAGGAGGAGGGCCTTCAGGGGCAAGGTCACA G AGGCC-3′. These oligodeoxynucleotides form, by pairing, a MstII-BgIII restriction fragment including the MstII-HindIII fragment corresponding to the peptide D5 defined by the Leu694 to Pro708 residues of vWF. The ligation of the MstII-HindIII fragment to a restriction fragment corresponding to the entire gene encoding AFP generates a restriction fragment containing a hybrid gene encoding a chimeric protein of the AFP-PEPTIDE type. This restriction fragment can be cloned in the productive orientation into a restriction site of a plasmid to generate an expression plasmid.

Useful variants of the expression plasmid can be deleted by site-directed mutagenesis between the peptides GI 0 and G5, for example sites for binding to collagen, and/or to heparin, and/or to botrocetin, and/or to sulphatides and/or to ristocetin.

In other embodiments, the use of combined techniques of site-directed mutagenesis and PCR amplification makes it possible to generate at will variants of a restriction fragment containing a hybrid gene encoding a chimeric protein of the AFP-PEPTIDE but deleted of one or more sites for binding to sulphatides and/or to botrocetin and/or to heparin and/or to collagen, and/or substituted by any residue involved in the vWF-associated emergence of IIB type pathologies.

In other useful variants of an expression plasmid comprising a hybrid gene encoding a chimeric protein of the AFP-PEPTIDE type, mutations can be introduced, for example by site-directed mutagenesis, to replace or suppress all or part of the set of cysteincs present at positions 471, 474, 509 and 695 of the human vWF. Specific examples are the plasmids p5E and p7E in which the cysteins present at positions 471 and 474, on the one hand, and at positions 471, 474, 509 and 695, on the other hand, have been respectively replaced by glycine residues. The PCR amplification of these plasmids with the oligodeoxynucleotides Sq2149 (5′-CCCGGGATCCCTTAGGCTTAACCGGTGAAGCCGGC-3′ (SEQ ID NO:28), the MstII site is underlined) and Sq2029 makes it possible to generate MstII-HindIII restriction fragments including the Thr470 to Val713 residues of the natural vWF with the exception that at least the cystein residues at positions 471 and 474 were mutated to glycine residues. The ligation of these fragments to a restriction fragment corresponding to the entire gene encoding AFP can generate a restriction fragment containing a hybrid gene encoding a chimeric protein of the AFP-PEPTIDE type. These restriction fragments can be cloned in the productive orientation into a restriction site of a plasmid to generate an expression plasmid.

Other particularly useful mutations affect at least one residue involved in vWF-associated type IIB pathologies (increase in the intrinsic affinity of vWF for GP1b), such as the residues Arg543, Arg545, Trp550, Val551, Val553, Pro574 or Arg578 for example. The genetic recombination techniques in vitro also make it possible to introduce at will one or more additional residues into the sequence of vWF and for example a supernumerary methionine between positions Asp539 and Glu542.

E.3.2. Fragments Antagonizing the Binding of vWF to the Sub-Endothelium

In a specific embodiment, the sites for binding of vWF to the components of the sub-endothelial tissue, and for example collagen, are generated by PCR amplification of the plasmid pET-8c52K, for example with the oligodeoxynucleotides Sq2258 (5′-GGATCCTTAGGGCTGTGCAGCAGGCTACTGGACCTGGTC-3′ and Sq2259 (5′-GAATTCAAGCTTAACAGAGGTAGCTAA-CGATCTCGTCCC-3′, which generates an MstII-HindIII restriction fragment encoding the Cys509 to Cys695 residues of the natural vWF. Deletion molecular variants or modified variants are also generated which contain any desired combination between the sites for binding of vWF to the sulphatides and/or to botrocetin and/or to heparin and/or to collagen and/or any residue responsible for a modification of the affinity of vWF for GP1b (vWF-associated type II pathologies). In another embodiment, the domain capable of binding to collagen may also come from the vWF fragment which is between the residues 911 and 1114 and described by Pareti et al. [J. Biol. Chem. (1987) 262: 13835-13841]. The ligation of these fragments to a restriction fragment corresponding to the entire gene encoding AFP can generate a restriction fragment containing a hybrid gene encoding a chimeric protein of the AFP-PEPTIDE type. These restriction fragments can be cloned in the productive orientation into a restriction site of a plasmid pYG105 to generate an expression plasmid.

E.3.3. Purification and Molecular Characterization of the Chimeras Between AFP and vWF

The chimeras present in the culture supernatants transformed, for example with the expression plasmids according to Examples E.3.1. and E.3.2., can be characterized by means of antibodies specific for the AFP part and for the vWF part. The results will demonstrate whether the yeast K. lactis is capable of secreting chimeric proteins between AFP and a fragment of vWF, and whether these chimeras are immunologically reactive. It may also be desirable to purify some of these chimeras. The culture can then centrifuged (10,000 g, 30 min), the supernatant can be passed through a 0.22 mm filter (Millipore) and then concentrated by ultrafiltration (Amicon) using a membrane whose discrimination threshold is situated at 30 kDa. The concentrate obtained is then dialysed against a Tris-HCl solution (5 mM pH 8) and then purified on a column, e.g., affinity chromatography or ion-exchange chromatography. After elution of the column, the fractions containing the protein are pooled, dialysed against water and freeze-dried before characterization. The essentially monomeric character of the chimeric proteins between AFP and vWF can also be confirmed by their elution profile on a TSK 3000 column [Toyo Soda Company, equilibrated with a cacodylate solution (pH 7) containing 0.2M Na2 SO4].

### Example 4

Chimeras Derived from Urokinase

E.4.1. Constructs

A fragment corresponding to the amino-terminal fragment of urokinase (ATF: EGF-like domain+ringle domain) can be obtained from the corresponding messenger RNA of cells of certain human carcinoma, for example using the RT-PCR kit distributed by Pharmacia. This fragment can then be ligated to a restriction fragment comprising the full length AFP gene. The cloning in the productive orientation of a restriction fragment comprising a chimeric protein of AFP-urokinase into a restriction site of a plasmid generates an expression plasmid.

E.4.2. Secretion of the Hybrids

After selection on rich medium supplemented with G418, the recombinant clones can be tested for their capacity to secrete the mature form of the chimeric proteins AFP-UK. A few clones corresponding to the strain K. lactis CBS 293.91, which is transformed with the expression plasmids according to Example E.4.1., can be incubated in selective complete liquid medium at 28° C. The cellular supernatants can then be tested after electrophoresis on an 8.5% acrylamide gel, either directly by staining of the gel with coomassie blue, or after immunoblotting using as primary antibodies a rabbit polyclonal serum directed against AFPa-fetoprotein or against human urokinase. The results can demonstrate whether the hybrid proteins AFP-UK1→46 and AFP-UK1→135 are particularly well secreted by the yeast Kluyveromyces.

E.4.3 Purification of the Chimeras Between AFP and Urokinase

After centrifugation of a culture of a strain transformed with the expression plasmids according to Example E.4.1., the culture supernatant can be passed through a 0.22 mm filter (Millipore) and then concentrated by ultrafiltration (Amicon) using a membrane whose discrimination threshold is situated at 30 kDa. The concentrate obtained can then be adjusted to 50 mM Tris-HCl starlting with a stock solution of 1M Tris-HCl (pH 7), and then loaded in 20 ml fractions onto an anion-exchange column (3 ml) (D-Zephyr, Sepracor) equilibrated in the same buffer. The chimeric protein (AFP-UK1→46 or AFP-UK1→135) can then be eluted from the column by a gradient (0 to 1M) of NaCl. The fractions containing the chimeric protein can then be pooled and dialysed against a 50 mM Tris-HCl solution (pII 6) and reloaded onto a D-Zephyr column equilibrated in the same buffer. After elution of the column, the fractions comprising the protein can be pooled, dialysed against water and freeze-dried before characterization of their biological activity and especially with respect to their ability to displace urokinase from its cellular receptor.

### Example 5

Chimeras Derived from G-CSF

E.5.1. Constructs

E.5.1.1. Coupling at the C-Terminus of AFP.

An MstII-HindIII restriction fragment including the mature form of human G-CSF can be generated, for example according to the following strategy: a KpnI-HindIII restriction fragment is first obtained by the enzymatic PCR amplification technique using the oligodeoxynucleotides Sq2291 (5′-CAAGGATCCAAGCTTCAGGGCTGCGCAAGGTGGCGTAG-3′ and Sq2292 (5′-CGGGGTACCTTAGGCTTAACCCCCCTG-GGCCCTGCCAGC-3′ as primer on the plasmid BBG13 serving as template. The plasmid BBG13 contains the gene encoding the B form (174 amino acids) of mature human G-CSF, which is obtained from British Bio-technology Limited, Oxford, England. The enzymatic amplification product of about 550 nucleotides can then be digested with the restriction enzymes KpnI and HindIII and cloned into the vector pUC19 cut with the same enzymes, which generates the recombinant plasmid pYG1255. This plasmid can then be the source of an MstII-HindIII restriction fragment which makes it possible to fuse G-CSF immediately downstream of AFP (chimera AFP-G.CSF).

It may also be desirable to insert a peptide linker between the AFP part and G-CSF, for example to permit a better functional presentation of the transducing part.

The ligation of a restriction fragment comprising the full length AFP to the MstII-HindIII fragment of the plasmid pYG1255 makes it possible to generate a restriction fragment which encodes a chimeric protein in which the B form of the mature G-CSF is positioned by genetic coupling in translational phase at the C-terminus of the AFP molecule (AFP-G.CSF).

An identical restriction fragment may also be easily generated and which encodes a chimeric protein in which the B form of the mature G-CSF is positioned by genetic coupling in translational phase at the C-terminus of the AFP molecule and a specific peptide linker. For example, this linker consists of 4 glycine residues (chimera AFP-Gly4-G.CSF).

The restriction fragment is cloned in the productive orientation and into a restriction site of an expression plasmid, which generates an expression plasmid.

E.5.1.2. Coupling at the N-Terminus of AFP

In a specific embodiment, the combined techniques of site-directed mutagenesis and PCR amplification make it possible to construct hybrid genes encoding a chimeric protein resulting from the translational coupling between a signal peptide (and for example the prepro region of AFP), a sequence including a gene having a G-CSF activity, and the mature form of AFP or one of its molecular variants (cf. chimera of panel B, FIG. 1). These hybrid genes are preferably bordered in 5′ of the translational initiator ATG and in 3′ of the translational stop codon by HindIII restriction sites. For example the oligodeoxynucleotide Sq2369 (5′-GTTCTACGCCACCTTGCGCAGCCCGGTGGAGGCGGTGATGCACACAAGAGTGAG GTTGCTCATCGG-3′ (SEQ ID NO:35) the residues underlined (optional) correspond in this particular chimera to a peptide linker composed of 4 glycine residues) makes it possible, by site-directed mutagenesis, to put in translational phase the mature form of the human G-CSF of the plasmid BBG13 immediately upstream of the mature form of AFP, which generates the intermediate plasmid A. Likewise, the use of the oligodeoxynucleotide Sq2338 [5′-CAGGGAGCTGGCAGGGCCCAGGGGGGTTCGACGAAACACACCCCTGGAATAAGC CGAGCT-3′ (non-coding strand), the nucleotides complementary to the nucleotides encoding the first N-terminal residues of the mature form of the human G-CSF are underlined] makes it possible, by site-directed mutagenesis, to couple in translational reading phase the prepro region of AFP immediately upstream of the mature form of the human G-CSF, which generates the intermediate plasmid B. A HindIII fragment encoding a chimeric protein of the PEPTIDE-AFP type can then be generated by combining the restriction fragment (joining prepro region of AFP+N-terminal fragment of the mature G-CSF) with the restriction fragment of the plasmid [joining mature G-CSF-(glycine)×4-mature AFP].

E.5.2. Secretion of the Hybrids.

After selection on rich medium supplemented with G418, the recombinant clones can be tested for their capacity to secrete the mature form of the chimeric proteins between AFP and G-CSF. A few clones corresponding to the strain K. lactis CBS 293.91 transformed with the chimeric protein expression plasmids can be incubated in selective complete liquid medium at 28° C. The cellular supernatants can then be tested after electrophoresis on an 8.5% acrylamide gel, either directly by staining the gel with coomassie blue, or after immunoblotting using as primary antibodies rabbit polyclonal antibodies directed against the human G-CSF or a rabbit polyclonal serum directed against AFPa-fetoprotein. The results will demonstrate whether the hybrid protein AFP-G.CSF is recognized both by antibodies directed against AFP and human G-CSF.

E.5.3. Purification and Molecular Characterization of the Chimeras Between AFP and G-CSF.

After centrifugation of a culture of the CBS 293.91 strain transformed with the expression plasmids according to Example E.5.1., the culture supernatant can be passed through a 0.22 mm filter (Millipore) and then concentrated by ultrafiltration (Amicon) using a membrane whose discrimination threshold is situated at 30 kDa. The concentrate obtained can then be adjusted to 50 mM Tris-HCl from a 1M stock solution of Tris-HCl (pH 6), and then loaded in 20 ml fractions onto an ion-exchange column (5 ml) (Q Fast Flow, Pharmacia) equilibrated in the same buffer. The chimeric protein can then be eluted from the column by a gradient (0 to 1M) of NaCl. The fractions containing the chimeric protein can then be pooled and dialysed against a 50 mM Tris-HCl solution (pH 6) and reloaded onto a Q Fast Flow column (1 ml) equilibrated in the same buffer. After elution of the column, the fractions containing the protein can be pooled, dialysed against water and freeze-dried before characterization.

### Example 6

Chimeras Derived from an Immunoglobulin

E.6.1. Constructs

An Fv′ fragment can be constructed by genetic engineering techniques, and which encodes the variable fragments of the heavy and light chains of an immunoglobulin (Ig), linked to each other by a linker peptide [Bird et al., Science (1988) 242: 423; Huston et al., (1988) Proc. Natl. Acad. Sci. 85: 5879]. Schematically, the variable regions (about 120 residues) of the heavy and light chains of a given Ig can be cloned from the messenger RNA of the corresponding hybridoma, for example using the RT-PCR kit distributed by Pharmacia (Mouse ScFv module). In a second stage, the variable regions are genetically coupled by genetic engineering via a synthetic linkage peptide and for example the linker (GGGGS)×3. The ligation of a restriction fragment comprising the full AFP gene makes it possible to generate a restriction fragment which encodes a chimeric protein in which the AFP molecule is genetically coupled to the Fv′ fragment (chimera AFP-Fv′). The cloning in the productive orientation of the restriction fragment into the restriction site of a plasmid generates an expression plasmid.

E.6.2. Secretion of the Hybrids

After selection on rich medium supplemented with G418, the recombinant clones can be tested for their capacity to secrete the mature form of the chimeric protein AFP-Fv′. A few clones corresponding to the strain K. lactis CBS 293.91 transformed with the expression plasmids (AFP-Fv′) can be incubated in selective complete liquid medium at 28° C. The cellular supernatants can then be tested after electrophoresis on an 8.5% acrylamide gel, either directly by staining of the gel with coomassie blue, or after immunoblotting using as primary antibodies a rabbit polyclonal serum directed against AFP, or directly incubated with biotinylated antibodies directed against the immunoglobulins of murine origin.

### Example 7

Biological Activity of the Chimeras

E.7.1. Biological Activity In Vitro.

E.7.1.1. Chimeras Between AFP and vWF.

The antagonistic activity of the products is determined by measuring the dose-dependent inhibition of the agglutination of human platelets fixed with paraformaldehyde according to the method described by Prior et al. [Bio/Technology (1992) 10: 66]. The measurements are carried out in an aggregameter (PAP-4, Bio Data, Horsham, Pa., U.S.A.) which records the variations over time of the optical transmission, with stirring, at 37° C. in the presence of vWF, of botrocetin (8.2 mg/ml) and of the test product at various dilutions (concentrations). For each measurement, 400 ml (8×107 platelets) of a suspension of human platelets stabilized with paraformaldehyde (0.5%, and then resuspended in [NaCl (137 mM); MgCl2 (1 mM); NaH₂ PO₄ (0.36 mM); NaHCO₃ (10 mM); KCl (2.7 mM); glucose (5.6 mM); AFP (3.5 mg/ml); HEPES buffer (10 mM, pH 7.35)] can be preincubated at 37° C. in a cylindrical tank (8.75×50 mm, Wellcome Distriwell, 159 rue Nationale, Paris) of the aggregameter for 4 min and then supplemented with 30 ml of the solution of the test product at various dilutions in apyrogenic formulation vehicle [mannitol (50 g/l); citric acid (192 mg/l); L-lysine monohydrochloride (182.6 mg/l); NaCl (88 mg/l); pH adjusted to 3.5 by addition of NaOH (1M)], or formulation vehicle alone (control assay). The resulting suspension can then be incubated for 1 min at 37° C. and 12.5 ml of human vWF [American Bioproducts, Parsippany, N.J.; U.S.A.; 11% von Willebrand activity measured according to the recommendations for the use of PAP-4 (Platelet Aggregation Profilers) with the aid of platelets fixed with formaldehyde (2×105 platelets/ml), human plasma containing 0 to 100% vWF and ristocetin (10 mg/ml, cf. p. 36-45: vW Program™] are added and incubated at 37° C. for 1 min before adding 12.5 ml of botrocetin solution [purified from freeze-dried venom of Bothrops jararaca (Sigma) according to the procedure described by Sugimoto et al., Biochemistry (1991) 266: 18172]. The recording of the reading of the transmission as a function of time is then carried out for 2 min with stirring by means of a magnetic bar (Wellcome Distriwell) placed in the tank and with a magnetic stirring of 1,100 rpm provided by the aggregameter.

The mean variation of the optical transmission (n3 5 for each dilution) over time is therefore a measurement of the platelet agglutination due to the presence of vWF and botrocetin, in the absence or in the presence of variable concentrations of the test product. From such recordings, the % inhibition of the platelet agglutination due to each concentration of product is then determined and the straight line giving the % inhibition as a function of the reciprocal of the product dilution in log-log scale is plotted. The IC50 (or concentration of product causing 50% inhibition of the agglutination) is then determined on this straight line. The results are expected to show that some of the AFP-vWF chimeras of the present invention are better antagonists of platelet agglutination than the product RG12986 described by Prior et al. [Bio/Technology (1992) 10: 66] and included in the assays as standard value. Identical tests for the inhibition of the agglutination of human platelets in the presence of vWF of pig plasma (Sigma) makes it possible, furthermore, to demonstrate that some of the hybrids of the present invention, and especially some type IIB variants, are very good antagonists of platelet agglutination in the absence of botrocetin-type cofactors. The botrocetin-independent antagonism of these specific chimeras can also be demonstrated according to the procedure initially described by Ware et al. [Proc. Natl. Acad. Sci. (1991) 88: 2946] by displacing the monoclonal antibody 125 I-LJ-IB1 (10 mg/ml), a competitive inhibitor of the binding of vWF to the platelet GIb [Handa M. et al., (1986) J. Biol. Chem. 261: 12579] after 30 mim of incubation at 22° C. in the presence of fresh platelets (108 platelets/ml).

E.7.1.2. Chimeras Between AFP and G-CSF

The purified chimeras can be tested for their capacity to permit the in vitro proliferation of the IL3-dependant murine line NFS60, by measuring the incorporation of tritiated thymidine essentially according to the procedure described by Tsuchiya et al. [Proc. Natl. Acad. Sci. (1986) 83 7633]. For each chimera, the measurements can be carried out between 3 and 6 times in a three-point test (three dilutions of the product) in a zone or the relation between the quantity of active product and incorporation of labelled thymidine (Amersham) is linear. In each microtitre plate, the activity of a reference product consisting of recombinant human G-CSF expressed in mammalian cells is also systematically incorporated.

E.7.2. Biological Activity In Vivo

The activity of stimulation of the AFP-G-CSF chimeras on granulopoiesis in vivo can be tested after subcutaneous injection in rats (Sprague-Dawley/CD, 250-300 g, 8-9 weeks) and compared to that of the reference G-CSF expressed using mammalian cells. Each product can be injected subcutaneously into the dorso-scapular region at the rate of 100 ml for 7 consecutive days, (D1-D7). 500 ml of blood can be collected on days D-6, D2 (before the 2nd injection). D5 (before the 5th injection) and D8, and a blood count can be performed. The following examples describe actual experimental data and results, and are not prophetic.

### Example 8

Cloning of AFP, Generation of an Engineered Mammalian Expression Cassette, and Description of Strategy for Generation of Fusion Proteins at the N-, C-Terminus or Both.

First, a PCR product was generated that allowed replacement of the natural AFP signal peptide with a preferred signal peptide called SPCON2 (MRPTWAWWLFLV LLLALWAPARG). Cloning was performed in a way that other signal peptides can be substituted where desired to improve production or alter the N-terminal cleavage site.

The N-terminal segment of the vector, including the SPCON2 signal peptide through the NheI site, was generated by combining, denaturing, and renaturing two PCR products, PCR1 and PCR2. PCR1 was generated using Oligo #1 (TGCGCCCTACCTGGGCCTGGT GGCTGTTCCTGGTGCTGCTGCTGGCACTGT) and Oligo #2 (TTCATTCCTATGCAAGG TGCCGCGGGCTGGAGCCCACAGTGCCAGCAGCAGCA) as template with Oligo #3 (5′) (CTAGAGGATCCGCCACCATGCGCCCTACCTGGGCCTGGT) and Oligo #4 (3′) (CTATTCCATATTCATTCCTATGCAAGGT) as primers. PCR2 was generated using Oligo #'s 1 and 2 as template with Oligo #5 (5′) (AGGATCCGCCACCATGCGCC CTACCTGGGCCTGGT) and Oligo #6 (3′) (CTAGCTATTCCATATTCATTCCTAT GCAAGGT) as primers. A fraction of the reannealled PCR products will result in an insert that has NheI-compatible sticky ends at both ends, but renders the cloning NheI site uncuttable and thus the NheI site engineered into the ORF unique.

This fragment was ligated into the NheI site of the plasmid pcDNA3.1+(Invitrogen Corp.). The resultant recombinant was sequence confirmed, with the start Met of the Spcon2 signal peptide proximal to the putative transcriptional start of pcDNA3.1+ vector. The remaining viable NheI site was now just upstream of the PmeI site of pcDNA3.1+.

The C-terminal segment of the vector—from the NheI site through the stop codon and 3′ cloning sites—was generated by PCR, using Invitrogen Corp.'s AFP cDNA clone #5208299 (MGC:34639; IMAGE:5208299; GenBank BC027881) as template (FIG. 1), and Oligo #7 (5′) (TATGGAATAGCTAGCATATTGGATTCTTACCA) and Oligo #8 (3′) (GCTAGCAGATCTGGTACCGGCGCGCCTTAAACTCCTAAGGCAGCACGAGT) as primers. The 5′ primer (Oligo #7) introduced an NheI site into AFP and the 3′ primer (Oligo #8) introduced a silent Bsu36I site, followed by the remaining amino acids of AFP, the stop codon and AscI, Asp718, and BglII sites.

The PCR product was digested with NheI and BglII and ligated into the NheI and BamHI sites of the pcDNA3.1+ clone containing the N-terminal segment of the vector. (The BamHI and BglII site are compatible, but destroy the site once ligated together.)

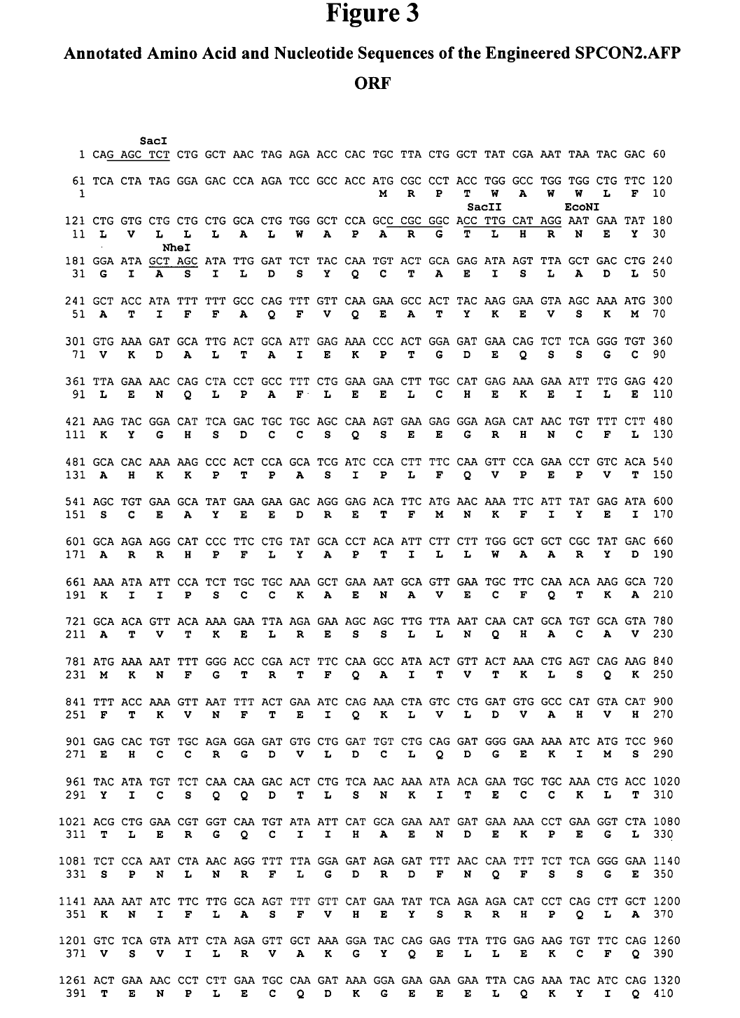

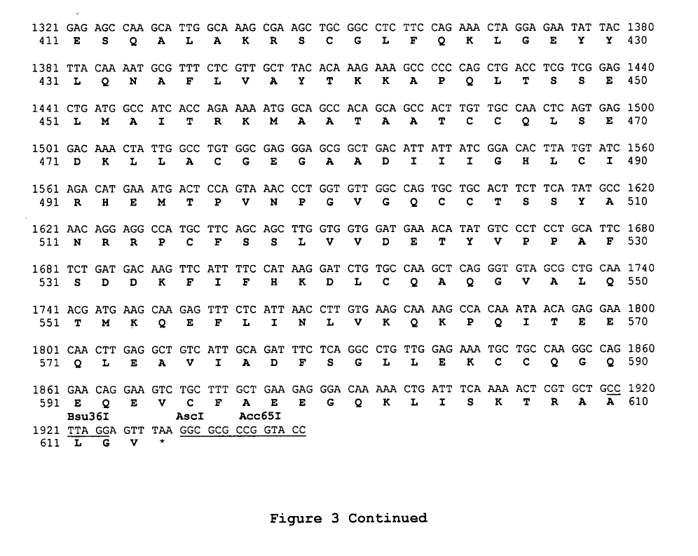

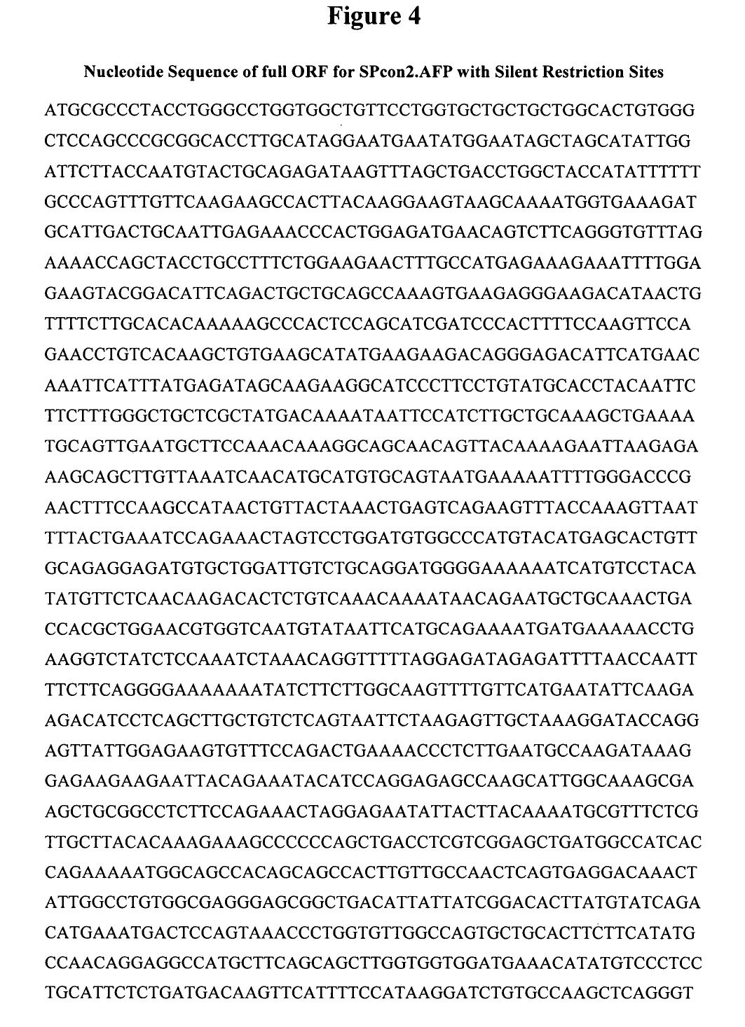

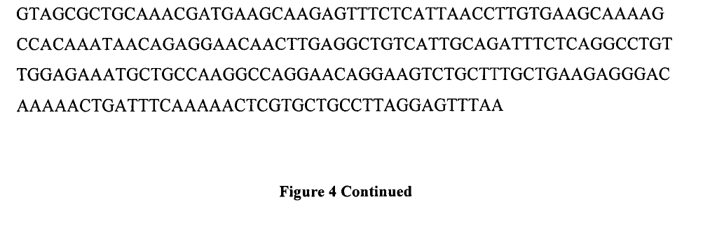

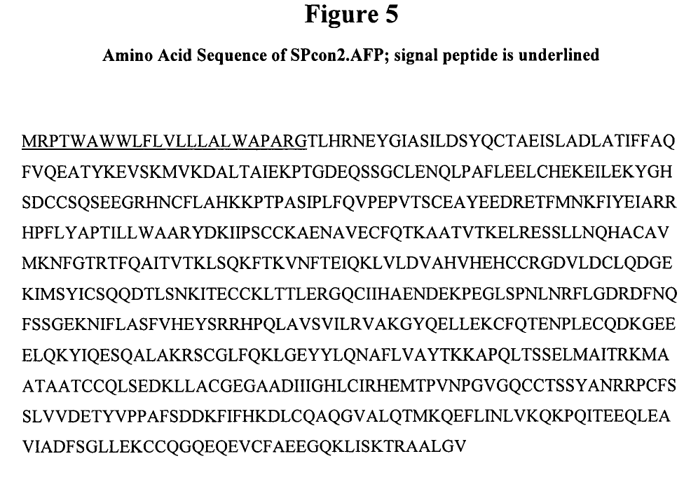

This vector, pcDNA3.1+:SPcon2.AFP, contained an SPCON2 signal peptide, followed by the mature AFP peptide (i.e. starting with the amino acid sequence TLHRN . . . ), the sequence of the final engineered expression ORF with the described restriction sites is shown in FIG. 3.

Subsequent fusions to the N-terminus of AFP can be cloned as Bam/EcoNI or Bam/NheI—if the endogenous signal peptide is to be used, or SacII/EcoNI or SacII/NheI if the SPCON2 signal peptide is to be used. C-terminal fusions can be cloned as Bsu36I alone, Bsu/AscI, Bsu/Asp718, or Bsu/XbaI. An example of a generic C-terminal fusion oligo tag Bsu36I useful as a forward primer for inserting a therapeutic protein is: CGTGCTGCCTTAGGAGTT. An example of a generic C-terminal fusion oligo tag AscI useful as a reverse primer is: TACCGGCGCGCCTTA. An example of a generic N-terminal fusion oligo tag SacII useful as a forward primer is: GGGCTCCAGCCCGCGGC. An example of a generic N-terminal fusion oligo tag NheI useful as a reverse primer is: CAATATGCTAGCTATTCCATATTCATTCCTATGCAAGGT.

To generate in-frame fusions without the addition of extraneous amino acids at the cloning junction, the ORF of the fusion partner (for example, G-CSF, IFN-alpha, butyrylcholinesterase, glucocerebrosidase (GBA), or IL-2) PCR primers are designed that recreate the restriction sites described above and, when digested and subcloned, create a clean fusion of the open reading frames.

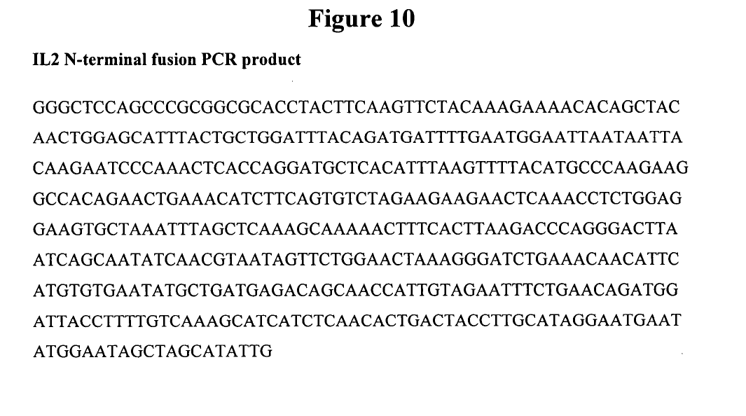

The oligos used to generate an IL2 N-terminal fusion PCR product are shown below, and the IL2 N-terminal fusion product is shown in FIG. 10.

IL2 Forward primer (the underlined portion is part of IL2)

GGGCTCCAGCCCGCGGCGCACCTACTTCAAGTTCTACAAAG

IL2 Reverse primer

CAATATGCTAGCTATTCCATATTCATTCCTATGCAAGGTAGTCAGTGTTG
AGATGATGCTTTG

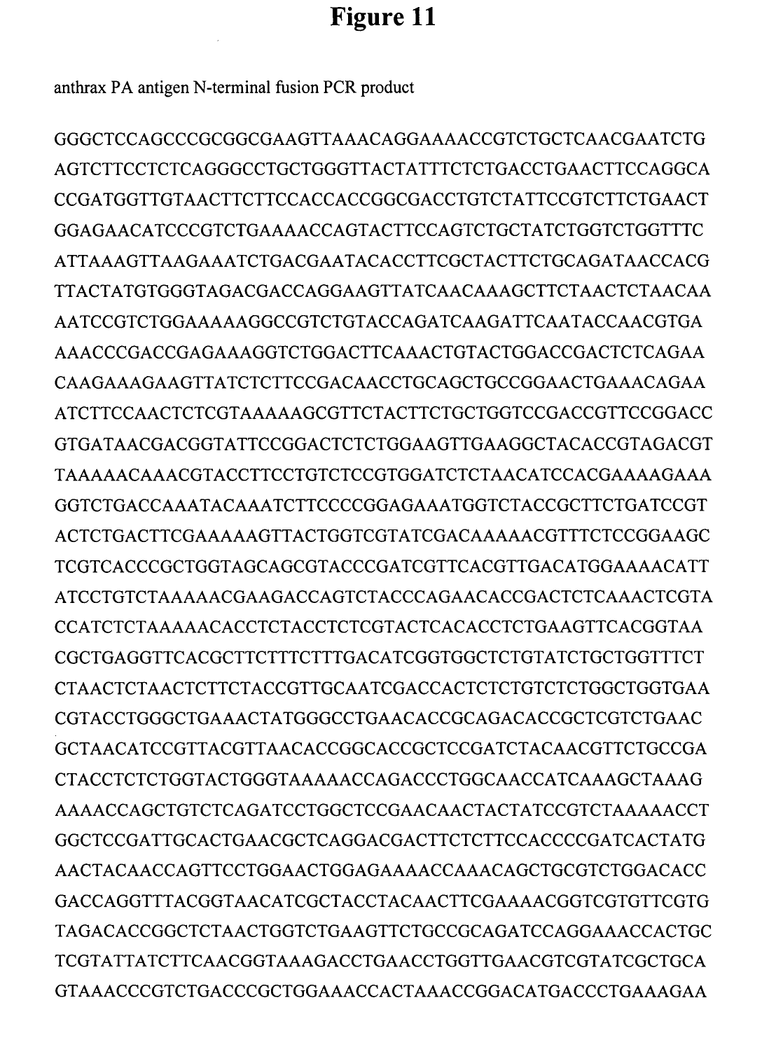

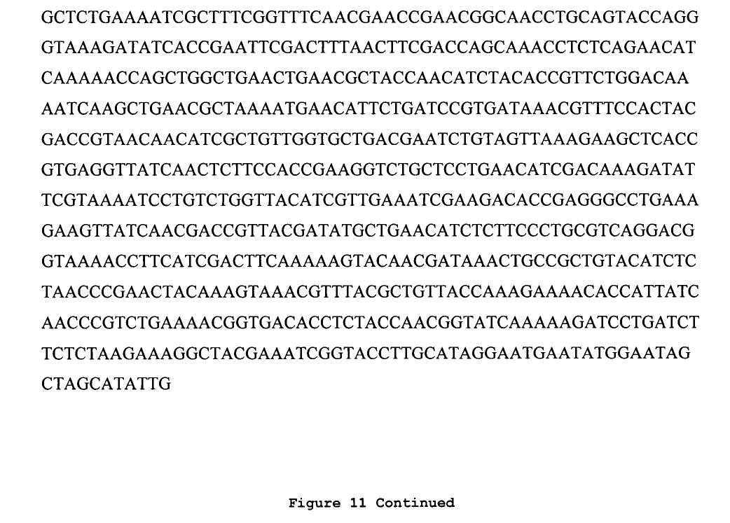

The oligos used to generate an anthrax PA antigen N-terminal fusion PCT product are shown below (two oligos were used to generate a PCR product for N-terminal fusion and two to generate the PCR product for the C-Terminal fusion) and the anthrax PA antigen N-terminal fusion product is shown in FIG. 11.

anthrax PA antigen N-terminal fusion Forward primer

GGGCTCCAGCCCGCGGC GAA GTT AAA CAG GAA AAC CGT CTG

anthrax PA antigen N-terminal fusion Reverse primer

CAATATGCTAGCTATTCCATATTCATTCCTATGCAAGGT
ACCGATTTCGTAGCCTTTCTTAG

anthrax PA antigen C-terminal fusion Forward primer

CGTGCTGCCTTAGGAGTT GAA GTT AAA CAG GAA AAC CGT CTG

anthrax PA antigen C-terminal fusion Reverse primer

TACCGGCGCGCCTTA ACCGATTTCGTAGCCTTTCTTAG

The oligos used to generate a glucocerebrosidase fusion PCR product are shown below.

Glucocerebrosidase Forward primer

GGGCTCCAGCCCGCGGCGCCCGCCCCTGCATCC

Glucocerebrosidase Reverse primer

CAATATGCTAGCTATTCCATATTCATTCCTATGCAAGGTCTGGCGACGCC
ACAGGTAG

### Example 9

### Protein Expression

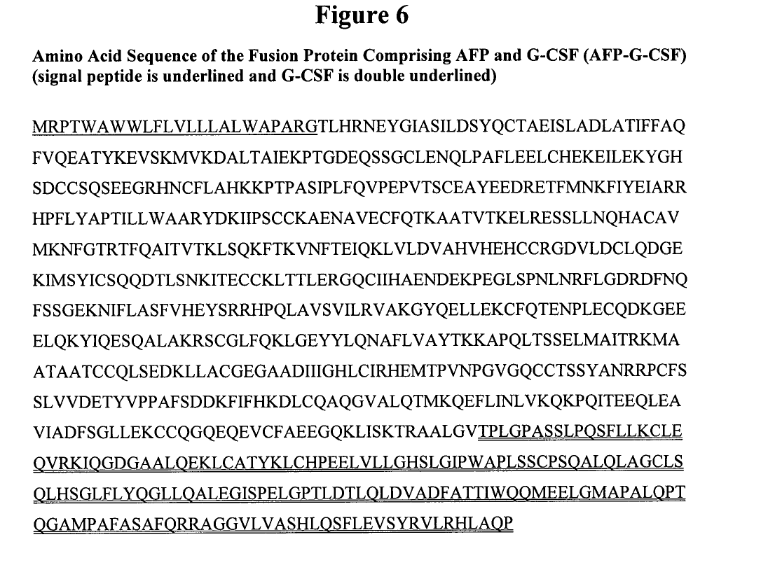

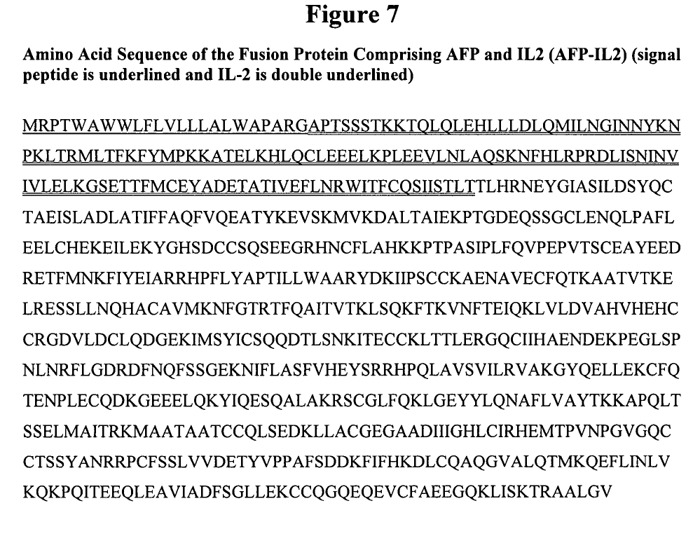

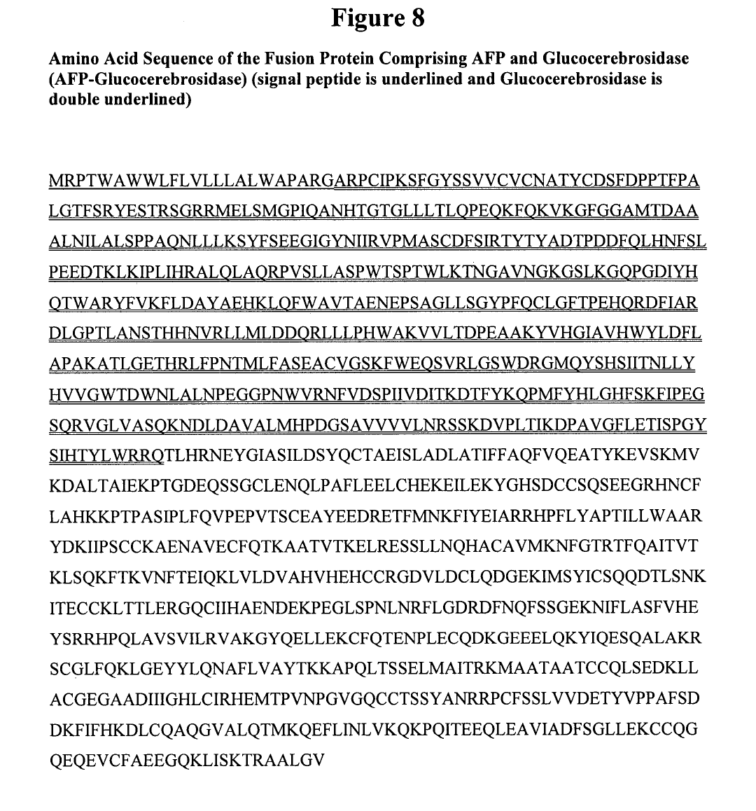

The purpose of this example was to demonstrate expression of the AFP fusion proteins AFP-G-CSF, IL2-AFP, and GBA-AFP. The amino acid sequences of the AFP-G-CSF, IL2-AFP, and GBA-AFP fusion proteins are shown in FIGS. 6, 7, and 8, respectively.

HEK293 cells (ATCC, CRL-1573) were transfected with pcDNA3.1 vectors encoding AFP as a reference and the AFP fusion proteins GBA-AFP, AFP-G-CSF, and IL2-AFP (using lipofectamine-2000 (Invitrogen Corp.)) according to the manufacturers instructions. “Charged” media was collected after 48 hrs of incubation and used directly in cell-based and enzymatic assays.

A Western blot was performed to identify expression of AFP and the fusion proteins GBA-AFP, AFP-G-CSF, IL2-AFP. Charged media was subjected to SDS PAGE and transferred to a PVDF membrane according to standard methods (Invitrogen Corp.). A mouse monoclonal antibody against AFP(R&D# MAB 1368) was used to probe the blot and detected with rabbit anti-mouse-HRP conjugate (Sigma) according to standard methods.

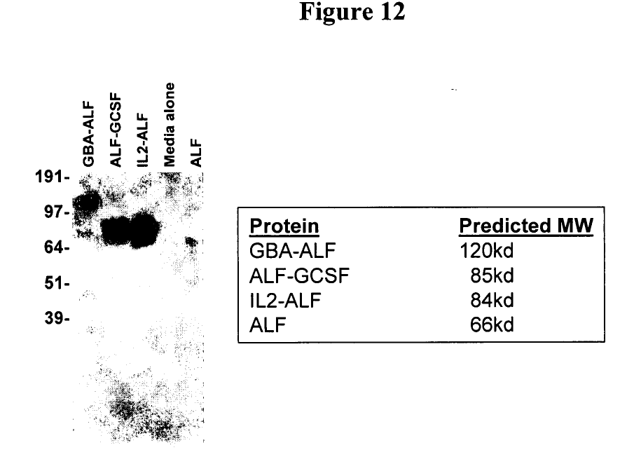

The results, shown in FIG. 12, demonstrate successful expression of the fusion proteins, as evidenced by the protein bands at the predicted molecular weights of 120 kD for GBA-AFP, 85 kD for AFP-G-CSF, 84 kD for IL2-AFP, and 66 kD for AFP.

### Example 10

### Demonstration of Activity

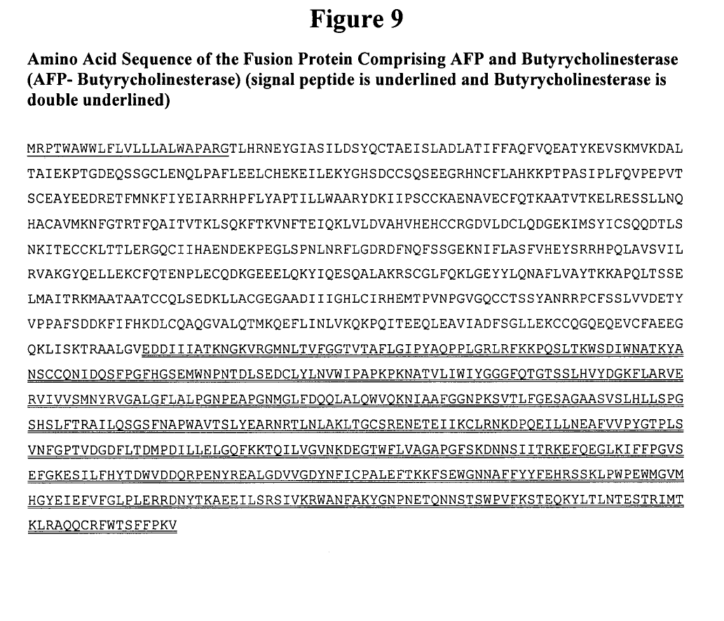

To demonstrate the biologic activity and versatility of AFP fusion proteins, fusions of butyrylcholinesterase, G-CSF, IL-2, Glucocerebrosidase, and IFN-alpha were made to either the N- or C-terminus of AFP utilizing the methodology of Example 8. The amino acid sequences of the AFP-G-CSF, IL2-AFP, GBA-AFP, and AFP-butyrylcholinesterase fusion proteins are shown in FIGS. 6, 7, 8, and 9, respectively.

Each of these proteins demonstrated robust activity in cell or enzyme bioassays demonstrating that AFP is a useful platform for fusion of therapeutic proteins and that fusions to both the N- and C-terminus of AFP are active.

A. AFP-GCSF Fusion Protein

A G-CSF.AFP fusion protein was generated by transient expression in HEK293 and assayed for effect on proliferation of AML-193 cells (ATCC, CRL-9589). AML-193 cells are a GM-CSF and G-CSF dependent cell line (J. Immunol., 139: 3348-3354 (1987)).

The GM-CSF-dependent cell line AML-193 (passage 4) was starved of GM-CSF for 24 hrs in 50 μL of “blank media” (20,000/well). To measure proliferation in response to G-CSF and AFP.G-CSF fusion protein, triplicate wells were treated with an equal volume of “charged” media, blank media, or serial 2-fold dilutions of charged media, and grown for 48 hrs. Charged media was collected from HEK293 cells transiently transfected with AFP-G-CSF or AFP expressing pcDNA3.1. Cell titers were measured by luminescence using the Cell-Titer-Glow Assay (Promega).

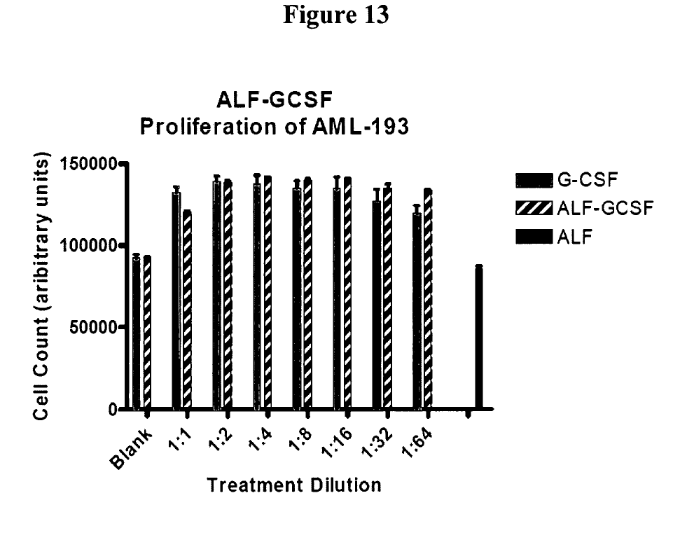

Media mixed 1:1 with media from cells expressing AFP had no effect on proliferation, demonstrating that the presence of AFP alone does not trigger proliferation of AML-193 cells. Media spiked with recombinant human G-CSF (R&D Systems, # 214-CS-005) at 5 ng/ml (1:1) caused a 1,5-fold increase in proliferation of AML-193 cells. Similarly, media from cells expressing AFP.G-CSF fusion protein caused an ˜1.5-fold increase in proliferation of these cells. These effects were not diminished by up to a 64-fold dilution of the charged media, showing that significant amounts of highly active fusion protein and G-CSF were present in the media. The results of the cell proliferation assays are shown in FIG. 13.

AFP.G-CSF is clearly active in a recognized bioassay for G-CSF activity and should be useful as a therapeutic protein.

B. IL2-AFP Fusion Protein

An IL2-AFP fusion protein was generated by transient expression in HEK293 and assayed for effect on proliferation of CTLL-2 cells (ATCC, TIB-214). CTLL-2 is an IL-2 dependent cell line (Immunology, 87: 271-274 (1996)).

The IL-2-dependent cell line CTLL-2 (passage 6) was starved of IL-2 for 24 hrs in 50 μL of the growth medium recommended by ATCC (20,000/well). To measure IL-2-dependent proliferation, cells were treated with an equal volume of “charged” media form a 293T culture containing cells transiently transfected with pcDNA vectors expressing AFP or IL2-AFP 48 hrs prior to media collection, or the same “blank” media spiked with 5 ng/mL of recombinant human IL2 (R&D Systems, # 202-IL-010). Charged media was serially diluted in 2-fold steps across a 96 well plate and then the cells were grown for 48 hrs. Treatments were made in triplicate. Cell titers were measured by luminescence using the Cell-Titer-Glow assay (Promega).

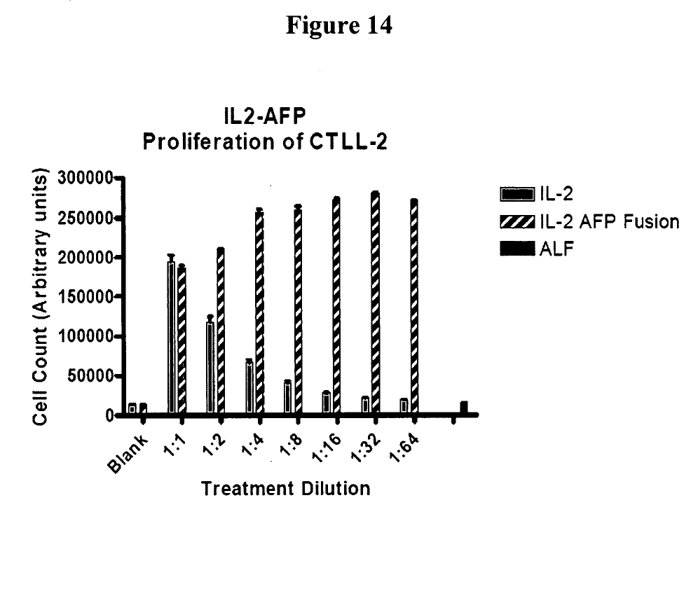

Media mixed 1:1 with media from cells expressing AFP was without effect on proliferation, demonstrating that the presence of AFP alone does not trigger proliferation of AML-193 cells. Media spiked with IL-2 at 5 ng/ml (1:1) caused a 20-fold increase in proliferation of CTLL-2 cells, an effect that could be diminished by dilution of the IL-2. Similarly, media from cells expressing IL2-AFP fusion protein caused an ˜20-fold increase in proliferation of these cells and, moreover, this effect was not diminished by up to a 64-fold dilution of the charged media, showing that significant amounts of highly active fusion protein was present in the media from 293 cells transfected with pcDNA3.1-IL2.AFP. The results of the cell proliferation assays are shown in FIG. 14.

IL2-AFP is clearly active in a recognized bioassay for IL2 activity and should be useful as a therapeutic protein.

C. Glucocerebrosidase-AFP Fusion Protein

To measure the activity of Glucocerebrosidase (GBA) and GBA-AFP fusion protein, media from HEK293 cells transiently transfected with plasmids encoding these proteins or irrelevant control proteins were assayed for their ability to hydrolyze 4-Methylumbelliferone (4-MU), 4-methylumbelliferyl-β-D-glucoside (4-MUG) obtained from Sigma Chemical Co. (St. Louis, Mo.). This assay is well known to demonstrate the activity of GBA (Proc. Natl. Acad. Sci., U.S.A., 73(12):4672-4 (December 1976)).

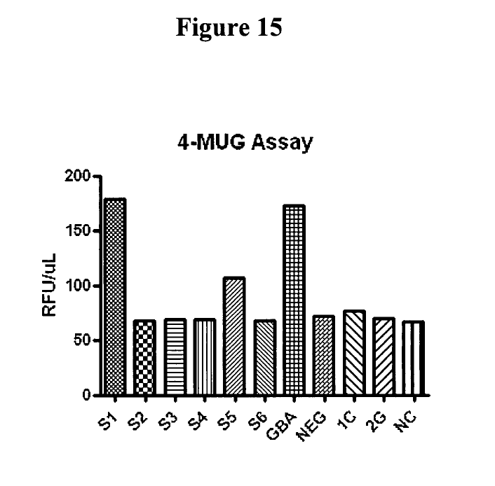

As shown in FIG. 15 (where S1=Glucocerbrosidase-AFP fusion protein, S2-4, 6=irrelevant controls, GBA=intact Glucocerebrosidase, and NEG=AFP control transfection), the activity of both intact Glucocerebrosidase (GBA) and the GBA-AFP fusion protein is clearly apparent in media collected from cells expressing the proteins and not from AFP alone.

Production of AFP-GBA fusion proteins in yeasts such as Saccharomyces cerevisiae and Pichia pastoris should have the advantage of producing a long acting form of the enzyme with enhanced uptake by the target cells, macrophages, via the mannose receptors on their surface. Yeasts naturally produce mannose-terminated sugars on glycoproteins such as AFP and this should enhance uptake in lysosomal storage diseases, including but not limited to, Gaucher's disease, Fabry's disease and other enzyme deficiencies like Krabbes disease.

D. AFP-IFN-α Fusion Protein

To measure the activity of AFP-IFN-α fusion protein, media from cells expressing AFP-IFNα or AFP alone was incubated for 48 hrs with an HEK293 cell line carrying a stable reporter gene (secreted alkaline phosphatase, SEAP) under the control of the “ISRE” element (as described in (J. Biol. Chem., 276(43):39765-39771 (2001)). Expression of SEAP and thus SEAP activity in this assay is indicative of IFN-α activity (J. Biol. Chem., 276(43):39765-39771 (2001)).

As shown in FIG. 14, dose-dependent activation of ISRE-SEAP is clearly apparent in response to AFP-IFNα but not AFP alone, demonstrating that the AFP-IFNα fusion protein is active.

E. AFP-Butyrylcholinesterase Fusion Protein

To measure the activity of AFP—Butyrylcholinesterase (BChE) fusion protein, the Ellman-based DTNB assay for esterase activity was performed on media collected from cells expressing these proteins or control vector. This assay is commonly used as a bioassay for BChE activity (Biochem. Pharmacol., 7:88-95 (1961)).

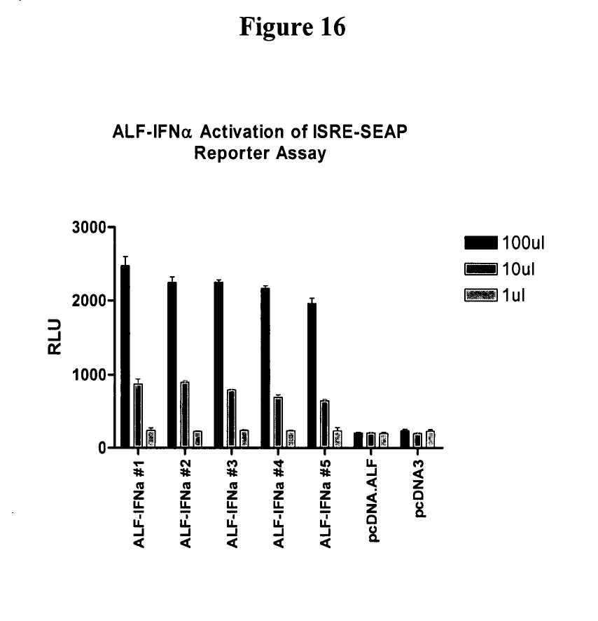

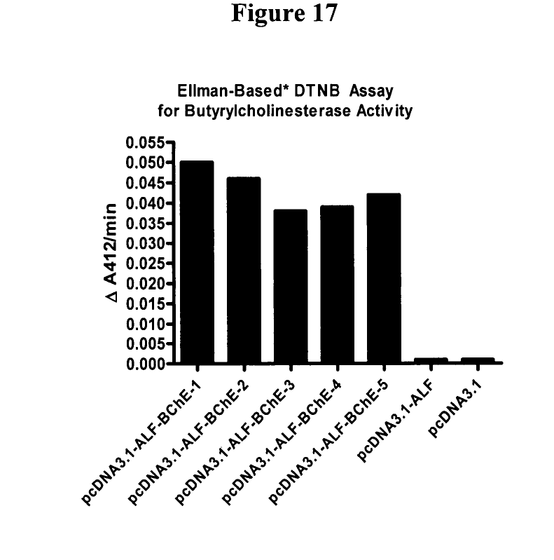

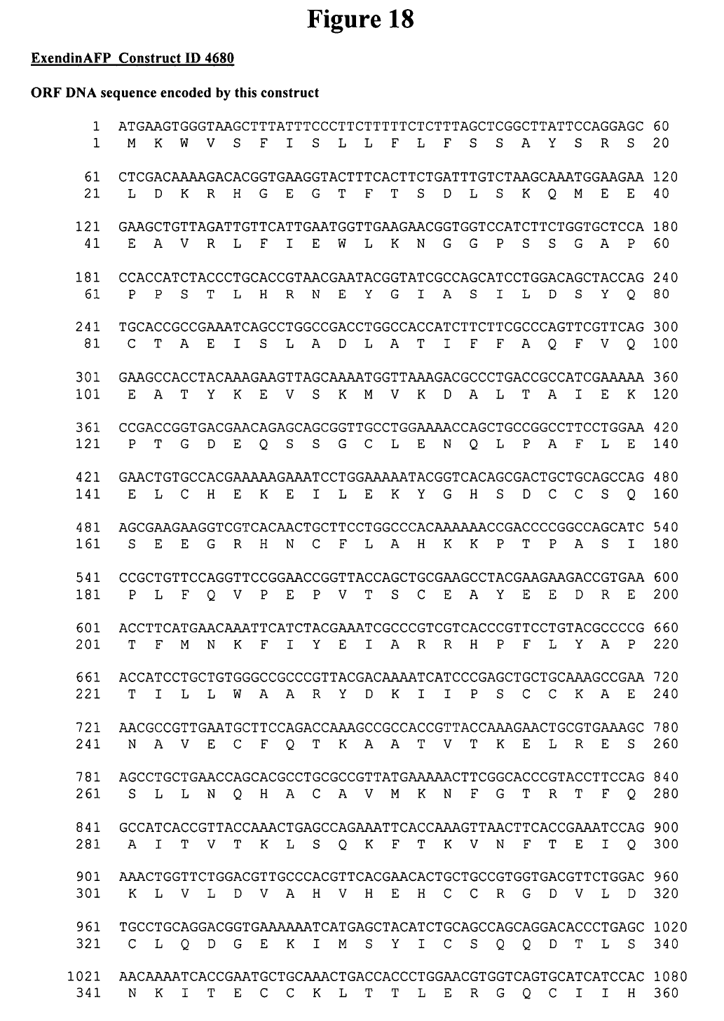

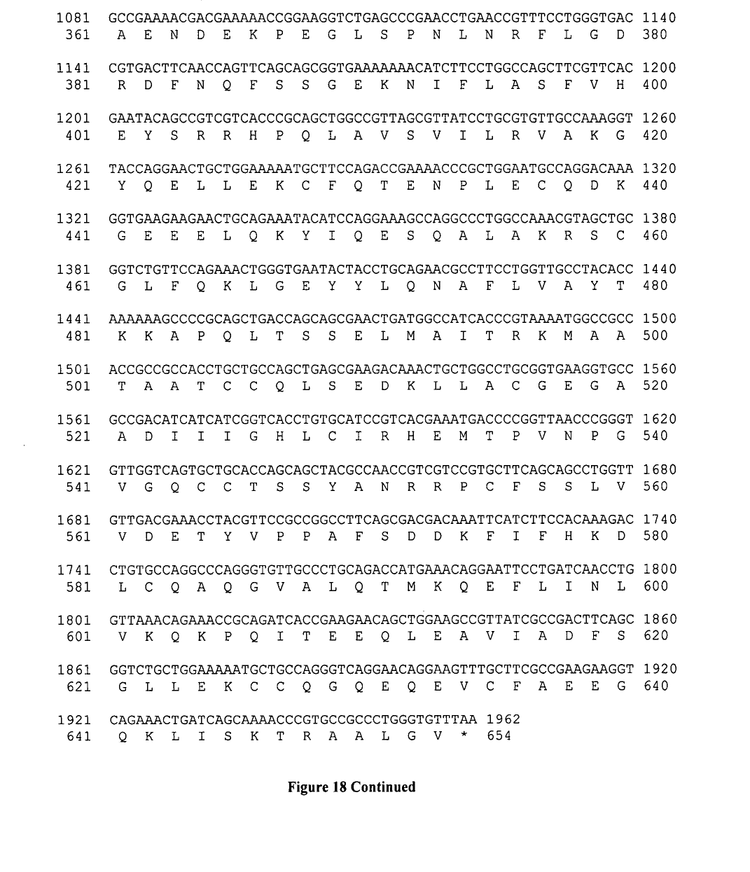

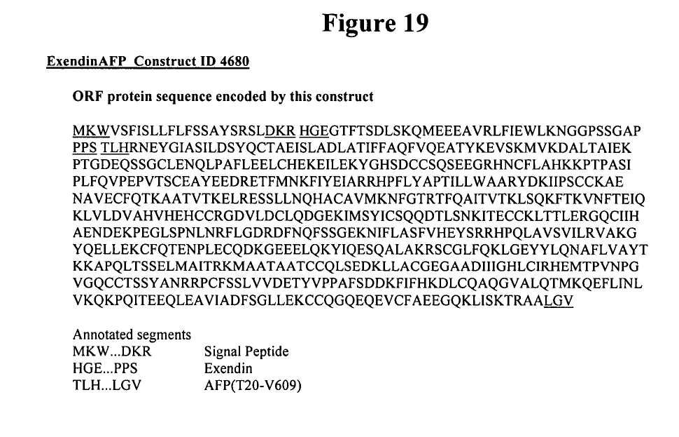

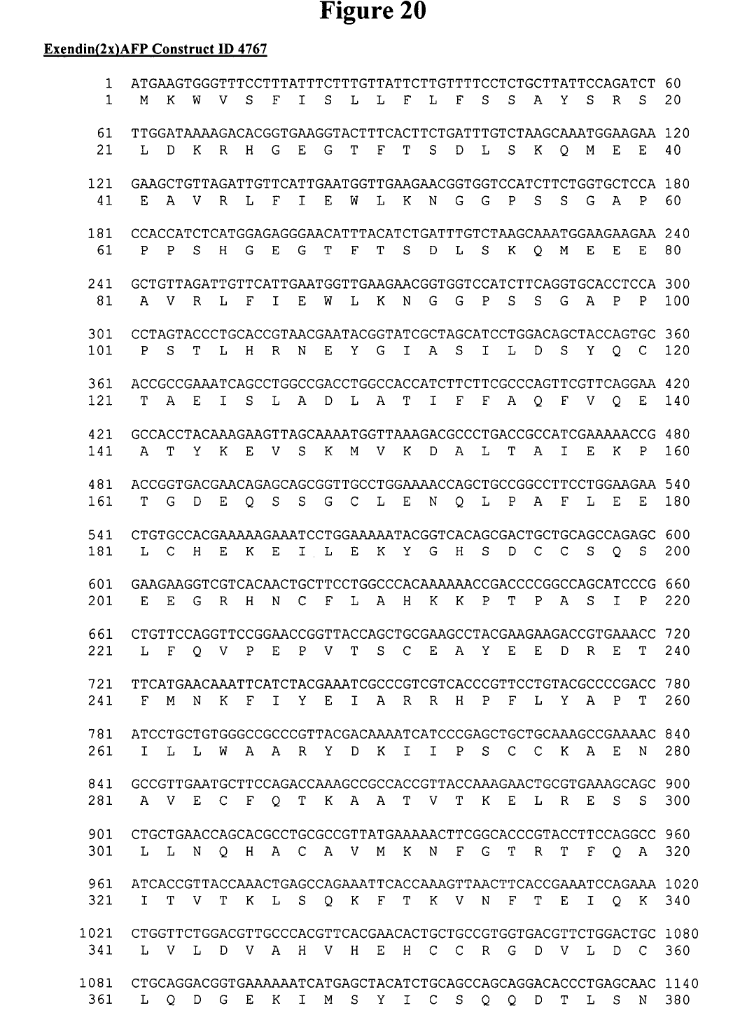

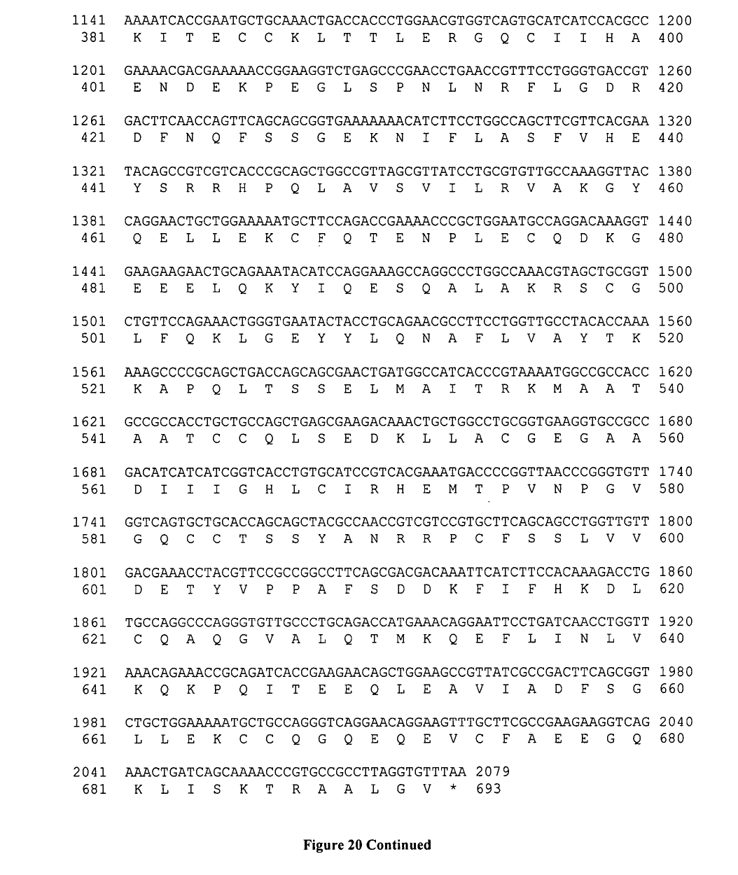

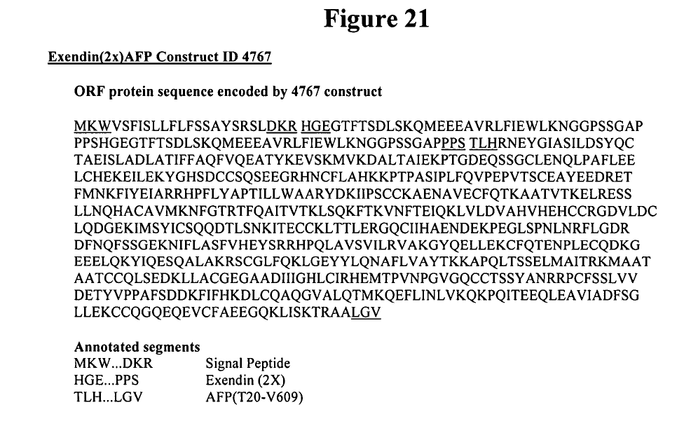

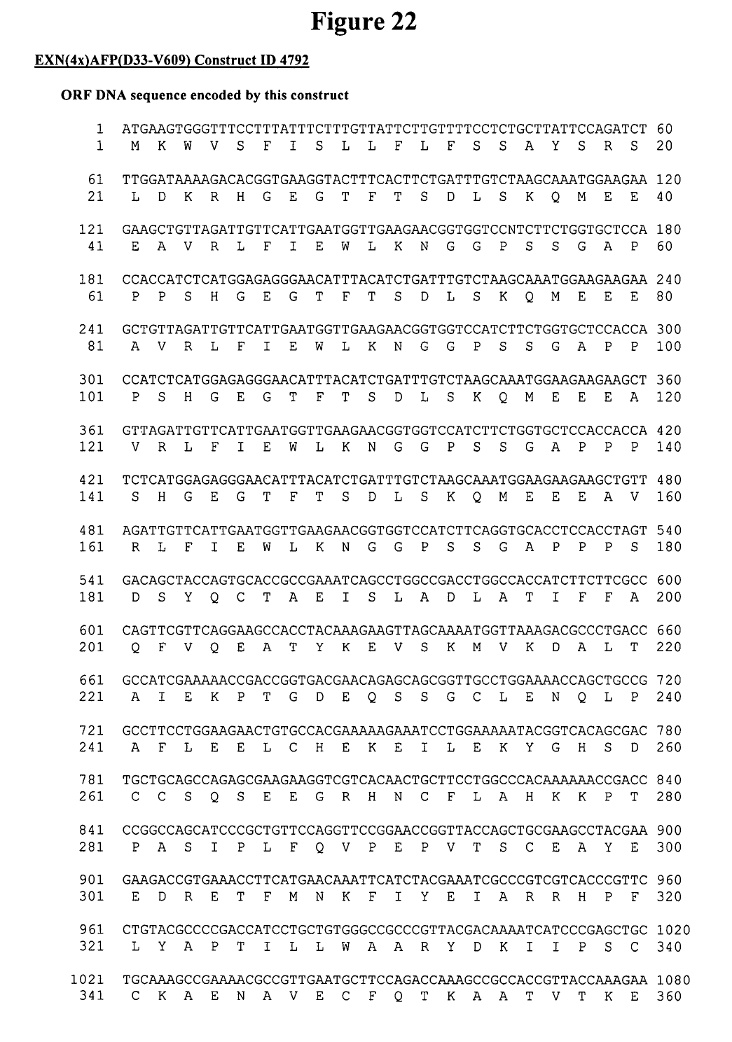

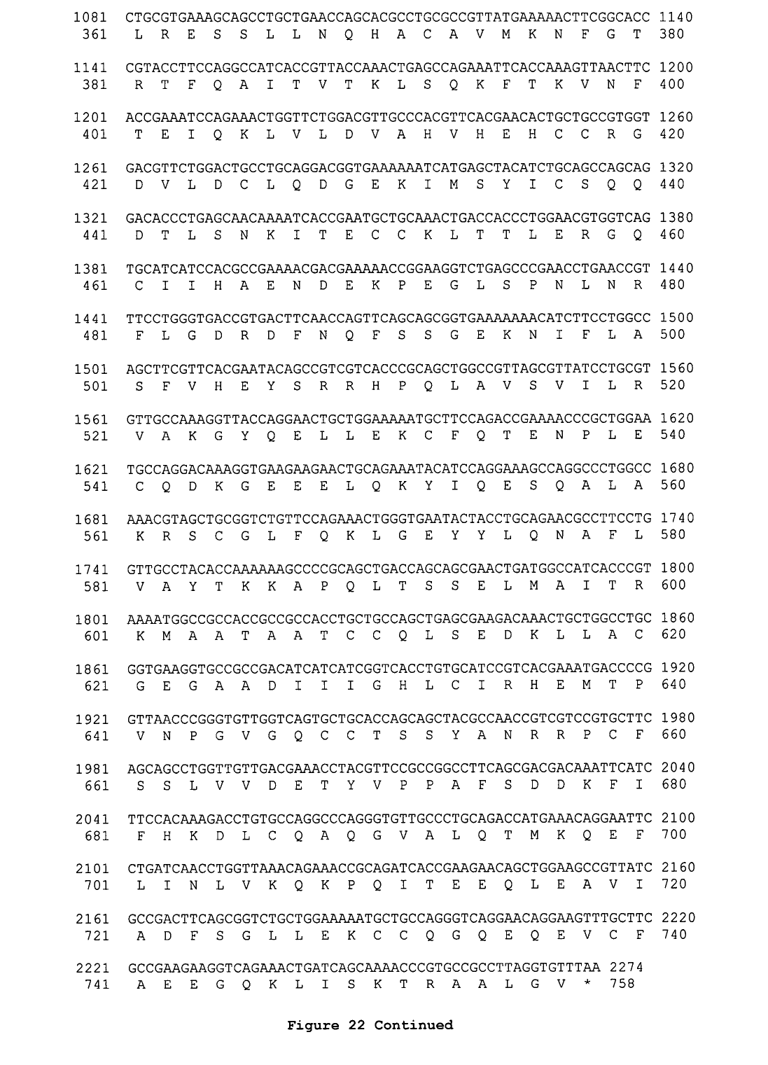

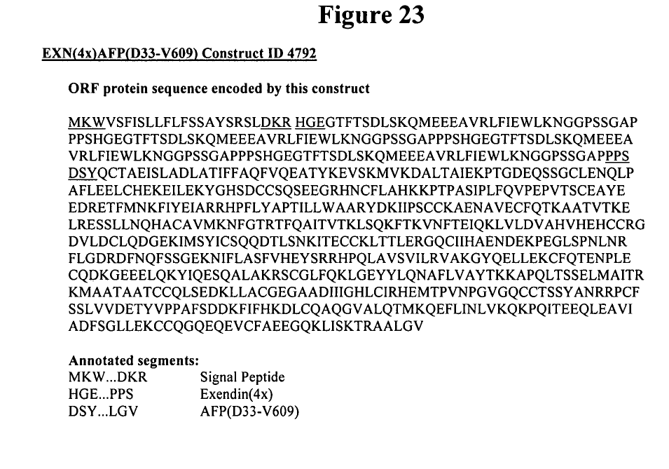

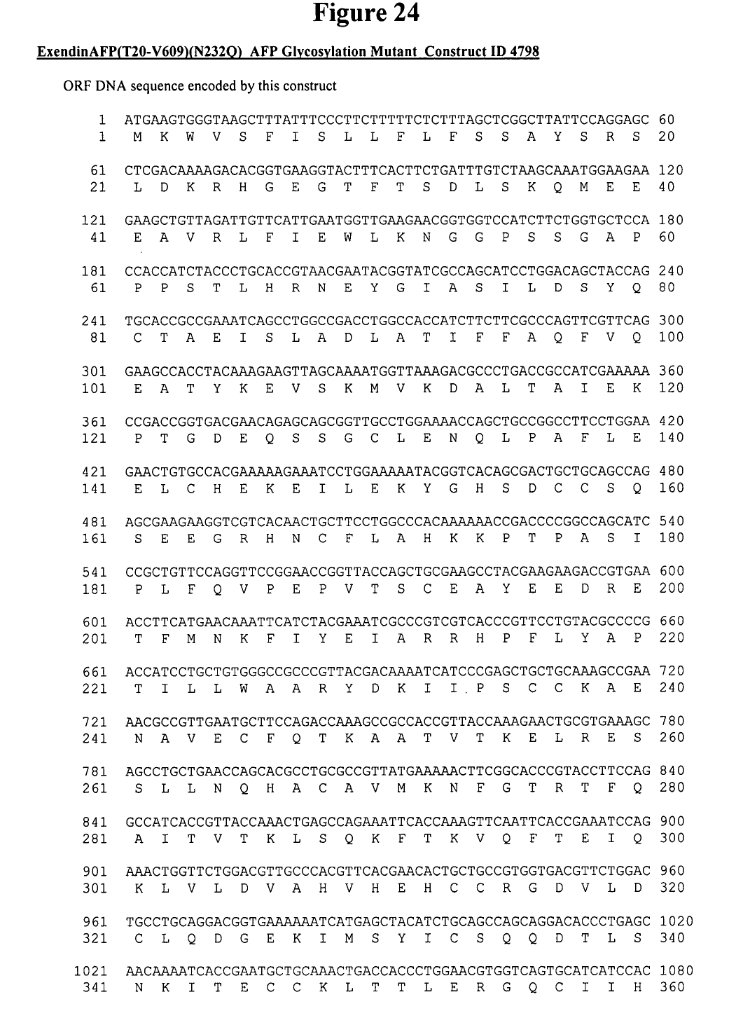

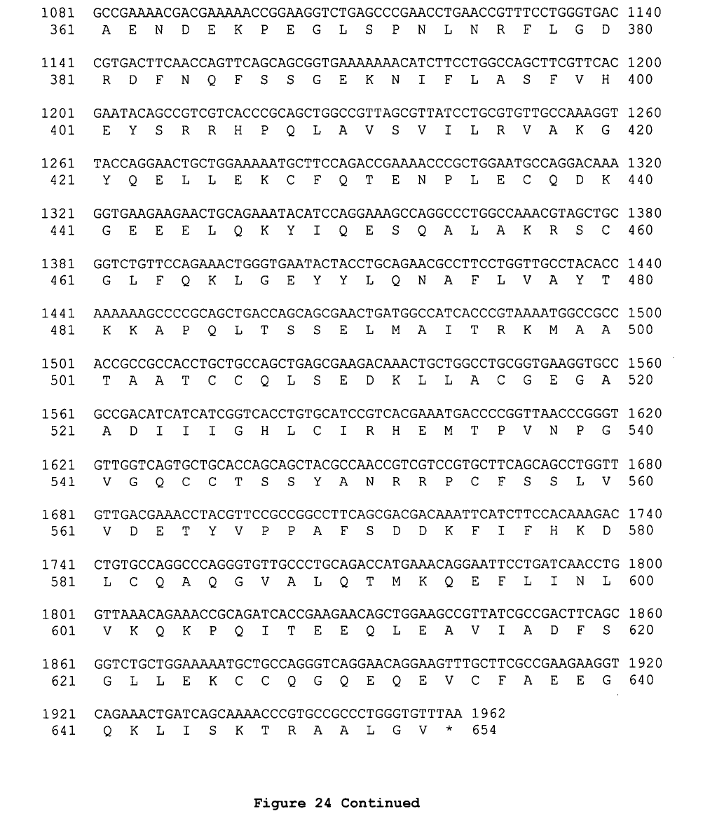

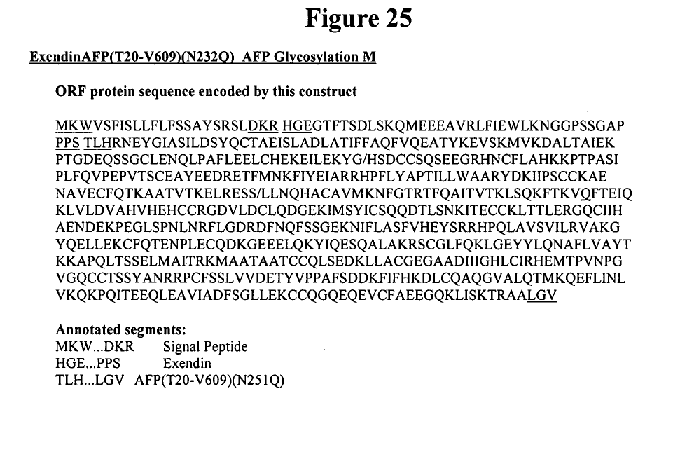

As shown in FIG. 16, the activity of AFP-BChE is clearly apparent and not attributable to AFP alone.

### Example 11

### Exendin-4/AFP Fusion Proteins (ExendinAFP)

Recombinant AFP was genetic fused to exendin-4 proteins to create exendin-4/AFP fusion proteins (i.e., “ExendinAFP”).

### A. PROTEIN EXPRESSION

ExendinAFP fusion constructs were stably transfected in a recombinant strain of the yeast Saccharomyces cerevisiae and expressed using 5-L scale fermentors. The protein was constitutively secreted into the media during fermentation. After fermentation, the supernatant was separated from the cells by centrifugation and prepared for purification by 0.2 m filtration. ExendinAFP was purified by a two-step process. The initial capture step utilized Blue Sepharose Fast Flow with sodium caprylate/sodium chloride for the elution. This provides selectivity for both binding and elution, which significantly reduces yeast host cell protein (yHCP) levels. A Hydroxyapatite column was subsequently used for removing residue DNA and further reducing yHCP level. The purified ExendinAFP proteins were characterized and shown to be endotoxin free and to have a purity >90% as determined by SDS-PAGE and N-terminal sequencing.

### B. IN VITRO STUDIES

ExendinAFP activity was characterized in vitro by assaying for activation of the GLP 1-receptor. Exendin-AFP was observed to effect an increase in intracellular cAMP levels in HEK293F cells expressing GLP1R.

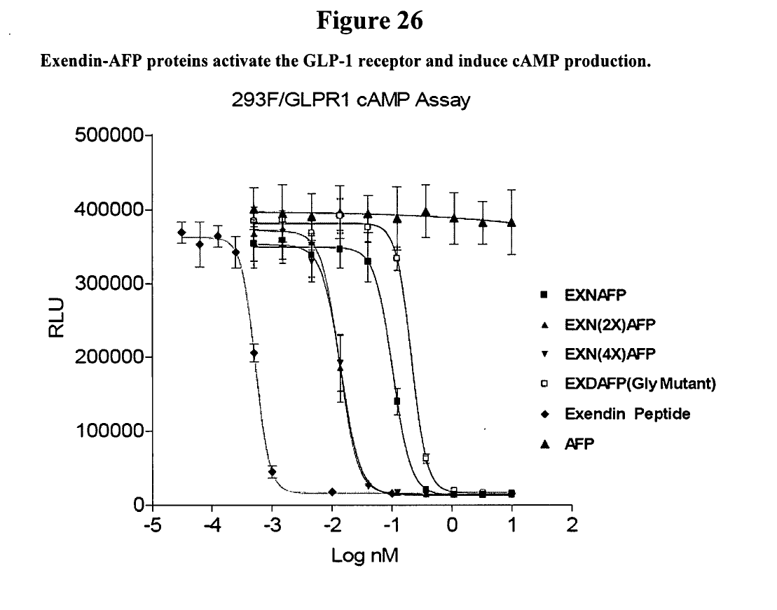

Methods. 293F cells stably transfected with human GLP-1R were generated and cultured in DMEM medium containing 10% FBS, L-glutamine (2 mM) Penicillin (100 U/mL), Streptomycin (100 μg/mL) Geneticin (500 μg/mL). For analysis, 293-GLP-1R cells were plated at 10,000 cells/well in poly D lysine coated 96 well plates and cultured overnight. Cells were washed once Krebs-Ringer Bicarbonate Buffer containing 0.5% BSA. Exendin-AFP proteins or controls were added in KRB/BSA buffer containing 100 μM 3-isobutyl-1-methylxanthine (IBMX). Proteins were added to triplicate wells and cells were incubated for 30 min at 37° C. Cellular levels of cAMP were determined using the homogeneous bioluminescent cAMP-Glo Assay (Promega, Cat. # V1502) using the manufacturers suggested protocol. In this assay the measured signal is inversely proportional to intracellular cAMP levels. For the statistical analysis, the measurement cAMP as a function of Exendin-AFP protein concentration is modeled using a four-parameter logistic model and IC50 values (nM) are determined. Results are shown in FIG. 26.

### C. IN VIVO STUDIES

ExendinAFP activity was characterized in vivo using mouse models.

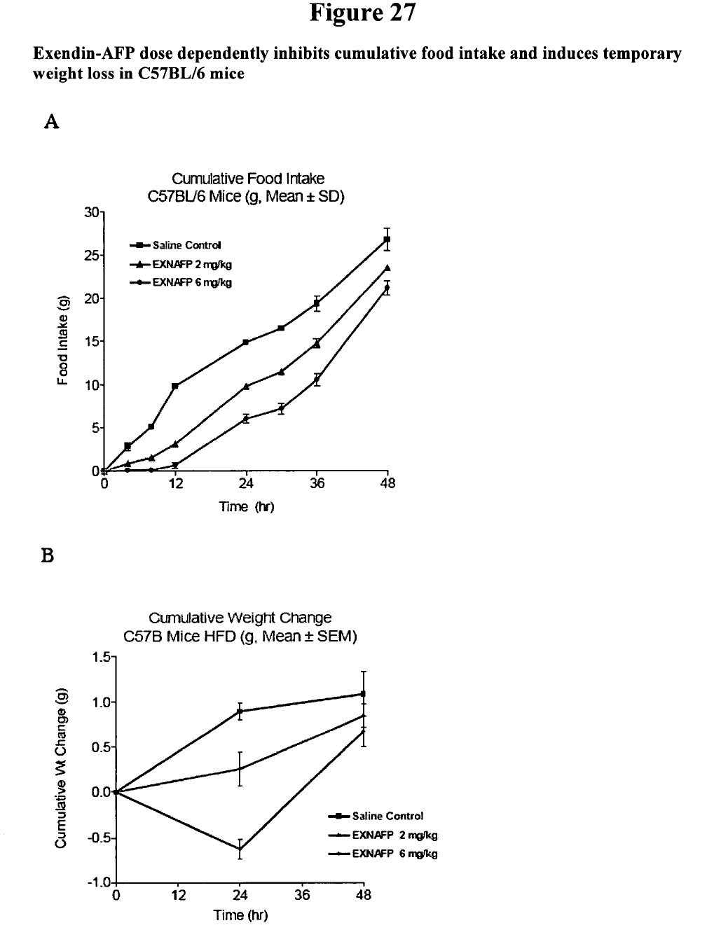

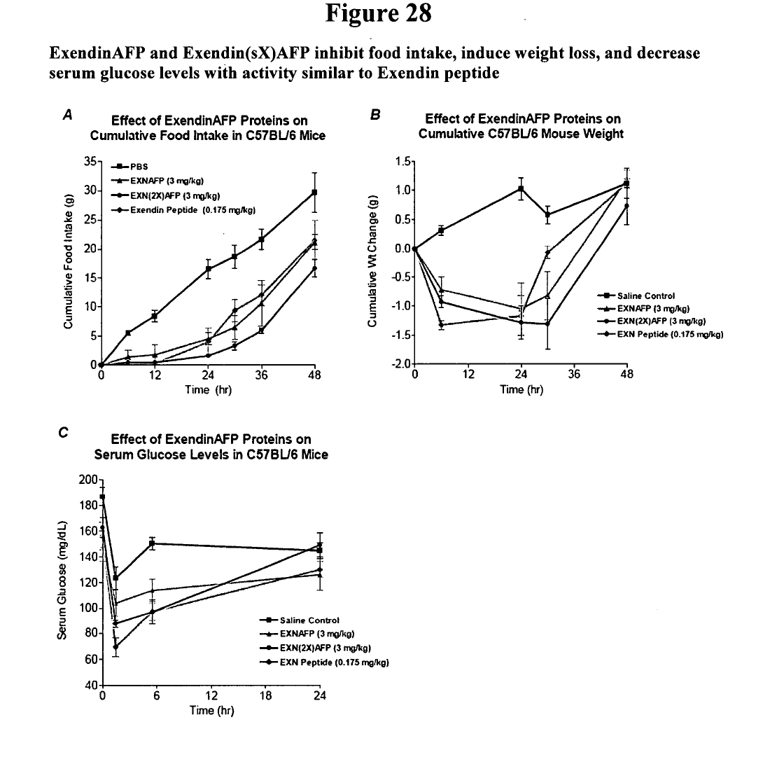

Methods. Adult female C57BL/6 mice (8-12 weeks of age) were maintained at 4 mice per cage under controlled conditions of temperature (21-23 C) and light with a consistent 12-h light: 12-h dark cycle and with ad libitum access to standard food and water. All procedures were done using IACUC approved animal protocols in accordance with established animal use guidelines (3). A single bolus injection of Exendin-AFP proteins at indicated doses or saline controlled was given subcutaneously (8 mice per group). Immediately after injection, mice were weighed and then placed into cages containing preweighed high-fat diet food pellets, with free access to water. Food intake was determined at indicated times by measuring the difference between the preweighed chow and the weight of the remaining chow at the end of each time interval. Mice were individually weighed at indicated times to determine changes in mouse weight. Cumulative food intake and mice weight was monitored for up to 48 hours. Serum glucose levels were tested by making a small nick in the tail of the treated or control animals and determining the serum glucose level in the resulting blood droplet using a Freestyle (Abbott) glucometer and test strips. To obtain background serum glucose levels all animals were sampled at time zero prior to administration of treatment. Results are shown in FIGS. 27 and 28.

### D. CONCLUSION

ExendinAFP proteins were observed to activate the GLP1 receptor and stimulate accumulation of intracellular cAMP. ExendinAFP proteins demonstrated activity in vivo that was similar to Exendin peptide. ExendinAFP inhibition of food intake and mouse weight was proportional to dosage at concentrations of 2 and 6 mg/kg in mice. A single bolus S.C. injection of 3 mg/kg ExendinAFP or Exendin(2×)AFP inhibited food intake, reduced mouse weight, and was observed to lower blood glucose levels in C57BL/6 mice at 2 and 6 hours after injection.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

1. An alpha-fetoprotein fusion protein comprising:
(a) a peptide comprising at least five contiguous amino acids and having therapeutic activity, immunological activity, or a combination thereof, and
(b) alpha-fetoprotein or a fragment or variant thereof, wherein the fragment consists of at least 305 contiguous amino acids, and wherein the variant consists of at least 305 contiguous amino acids and has at least 50% sequence identity to alpha-fetoprotein;
wherein: (i) the fusion protein has a higher plasma stability than unfused peptide, (ii) the fusion protein retains the therapeutic and/or immunologic activity of the unfused peptide, and/or (iii) the peptide is located either at the N-terminus or C-terminus of the alpha-fetoprotein or fragment thereof.

2. The fusion protein of claim 1, comprising the full length alpha-fetoprotein.

3. The fusion protein of claim 1, wherein the alpha-fetoprotein variant has a mutation, insertion, addition, and/or deletion of one or more residues.

4. The fusion protein of claim 1, wherein the fusion protein comprises a peptide linker.

5. The fusion protein of claim 1, wherein the fusion protein comprises a secretion signal sequence.

6. The fusion protein of claim 5, wherein the secretion signal sequence is the natural leader sequence of the peptide.

7. The fusion protein of claim 1, wherein the peptide is fused to the N-terminal end of the alpha-fetoprotein or fragment of variant thereof.

8. The fusion protein of claim 1, wherein the peptide is fused to the C-terminal end of the alpha-fetoprotein or fragment or variant thereof.

9. The fusion protein of claim 1, wherein the fusion protein is expressed by a prokaryotic cell.

10. The fusion protein of claim 1, wherein the fusion protein is expressed by a bacteria, yeast, or fungi.

11. The fusion protein of claim 1, wherein the fusion protein is expressed by a eukaryotic cell.

12. The fusion protein of claim 11, wherein the fusion protein is expressed by an animal cell.

13. The fusion protein of claim 12, wherein the animal cell is a CHO cell or a COS cell.

14. The fusion protein of claim 1, wherein the peptide encodes an antigen that generates an immune response and that is useful in a vaccine.

15. The fusion protein of claim 14, wherein the antigen is selected from the group consisting of PA-toxin, Human Immunodeficiency Virus, H5N1, cancer, Severe Acute Respiratory Syndrome (SARS), measles, mumps, rubella, polio, varicella, tetanus/diptheria, hepatitis A, hepatitis B, Haemophilus influenzae B, whooping cough, pneumonia, influenza, Lyme's Disease, rotavirus, cholera, yellow fever, Japanese encephalitis, poliomyelitis, rabies, typhoid fever, and pertussis.

16. The fusion protein of claim 14, wherein the antigen is selected from the group consisting of viral antigens, prions, bacterial antigens, parasitic antigens, and mycotic antigens.

17. The fusion protein of claim 1, wherein T or B cell stimulating epitopes are attached to the N or C terminus of the alpha-fetoprotein fusion protein.

18. A nucleic acid molecule comprising a polynucleotide encoding the fusion protein of claim 1.

19. A nucleic acid molecule of claim 18, which comprises a heterologous polynucleotide.

20. The nucleic acid molecule of claim 19, wherein said heterologous polynucleotide is a vector sequence, promoter sequence, a selectable marker, and/or a region for termination of transcription.

21. The nucleic acid molecule of claim 20, wherein the promoter sequence is any one selected from the group consisting of:
(a) a hybrid promoter;
(b) a constitutive promoter;
(c) a regulatable promoter;
(d) a yeast phosphoglycerate kinase (PGK) promoter;
(e) a yeast glyceraldehyde-3-phosphate dehydrogenase (GDP) promoter;
(f) a yeast lactase (LAC4) promoter;
(g) a yeast enolase (ENO) promoter;
(h) a yeast alcohol dehydrogenase (ADH) promoter;
(i) a yeast acid phosphatase (PHO5) promoter;
(j) a lambda bacteriophage PL promoter;
(k) a lambda bacteriophage PR promoter;
(l) a tryptophan Ptrp promoter; and
(m) a lactose Plac promoter.

22. The nucleic acid molecule of claim 20, wherein the selectable marker is any one selected from the group consisting of:
(a) the URA3 gene;
(b) geneticin resistance;
(c) metal ion resistance; and
(d) ampicillin resistance.

23. An isolated host cell comprising the nucleic acid molecule of claim 18.

24. A method for producing a fusion protein, comprising:
(a) culturing the host cell of claim 23 under conditions suitable to produce the fusion protein encoded by the polynucleotide; and
(b) recovering the fusion protein.

25. A vaccine comprising the fusion protein of claim 14.

26. A method of preventing, ameliorating, or treating a disease comprising administering an effective amount of the vaccine of claim 25 to a subject to prevent, ameliorate or treat a disease in the subject.

27. The method of claim 26, wherein the disease is selected from the group consisting of anthrax, Human Immunodeficiency Virus, Avian flu (H5N1), cancer, Severe Acute Respiratory Syndrome (SARS), measles, mumps, rubella, polio, varicella, tetanus/diptheria, hepatitis A, hepatitis B, Haemophilus influenzae B, whooping cough, pneumonia, influenza, Lyme's Disease, rotavirus, cholera, yellow fever, Japanese encephalitis, poliomyelitis, rabies, typhoid fever, and pertussis.

28. An alpha-fetoprotein fusion protein comprising:
(a) alpha-fetoprotein or a fragment or variant thereof;
(b) at least one therapeutic protein or a fragment or variant thereof, wherein the therapeutic protein is selected from the group consisting of human growth hormone, interleukin-2, granulocyte macrophage colony stimulating factor, calcitonin, interferon-beta, interferon-alpha, granulocyte colony stimulating factor, glucagon like peptide 1, glucagon like peptide 2, PA toxin, parathyroid hormone, butyrylcholinesterase, glucocerebrosidase, and exendin-4;
wherein: (i) the fusion protein has a higher plasma stability than unfused therapeutic protein; and (ii) the fusion protein retains the therapeutic and/or immunologic activity of the unfused therapeutic protein.

29. The fusion protein of claim 28, comprising alpha-fetoprotein contiguous amino acid sequence D33-V609.

30. The fusion protein of claim 28, comprising alpha-fetoprotein contiguous amino acid sequence T20-V609.

31. The fusion protein of claim 30, having amino acid substitution N251Q.

32. The fusion protein of claim 28, further comprising a leader peptide.

33. The fusion protein of claim 28, wherein the therapeutic protein is fused to the N-terminus, the C-terminus, or both termini of the alpha-fetoprotein or the fragment or variant thereof.
